(19) United States
(12) Patent Application Publication
(10) Pub. No.: US 2023/0416264 A1
(43) Pub. Date: Dec. 28, 2023

(54) HETEROCYCLIC COMPOUNDS FOR ORGANIC ELECTROLUMINESCENT DEVICES

(71) Applicant: Merck Patent GmbH, Darmstadt (DE)

(72) Inventors: Amir Hossain PARHAM, Darmstadt (DE); Christian EHRENREICH, Darmstadt (DE)

(21) Appl. No.: 18/039,303

(22) PCT Filed: Nov. 29, 2021
(86) PCT No.: PCT/EP2021/083269

§ 371 Date: May 30, 2023

(30) Foreign Application Priority Data
Dec. 2, 2020 (EP) 20211256.1

(51) Int. Cl. C07D 487/14; C07D 519/00; C07D 487/04; C07D 487/22; H10K 85/60; H10K 50/11; H10K 50/16; H10K 50/18; C09K 11/06

(52) U.S. Cl. CPC C07D 487/14; C07D 519/00; C07D 487/04; C07D 487/22; H10K 85/6572; H10K 50/11; H10K 50/16; H10K 50/181; C09K 11/06

(57) ABSTRACT

The present invention relates to heterocyclic compounds which are suitable for use in electronic devices, and to electronic devices, in particular organic electroluminescent devices, containing said compounds.

The present invention relates to heterocyclic compounds for use in electronic devices, especially in organic electroluminescent devices, and to electronic devices, especially organic electroluminescent devices comprising these materials.

Emitting materials used in organic electroluminescent devices are frequently phosphorescent organometallic complexes. For quantum-mechanical reasons, up to four times the energy efficiency and power efficiency is possible using organometallic compounds as phosphorescent emitters. In electroluminescent devices, especially also in electroluminescent devices that exhibit triplet emission (phosphorescence), there is generally still a need for improvement. The properties of phosphorescent electroluminescent devices are not just determined by the triplet emitters used. More particularly, the other materials used, such as matrix materials, are also of particular significance here. Improvements in these materials can thus also lead to distinct improvements in the properties of the electroluminescent devices.

WO 2010/062065 A2, KR 101869673 B1 WO 2016/140549 A2 describes imidazole derivatives that can be used as matrix materials for phosphorescent emitters.

In general terms, in the case of these materials, for example for use as matrix materials, there is still a need for improvement, particularly in relation to the lifetime, but also in relation to the efficiency and operating voltage of the device.

It is therefore an object of the present invention to provide compounds which are suitable for use in an organic electronic device, especially in an organic electroluminescent device, and which lead to good device properties when used in this device, and to provide the corresponding electronic device.

More particularly, the problem addressed by the present invention is that of providing compounds which lead to a high lifetime, good efficiency and low operating voltage. Particularly the properties of the matrix materials too have a major influence on the lifetime and efficiency of the organic electroluminescent device.

A further problem addressed by the present invention can be considered that of providing compounds suitable for use in phosphorescent or fluorescent electroluminescent devices, especially as a matrix material. A particular problem addressed by the present invention is that of providing matrix materials that are suitable for red- and yellow-phosphorescing electroluminescent devices, especially for red-phosphorescing electroluminescent devices, and if appropriate also for blue-phosphorescing electroluminescent devices.

In addition, the compounds, especially when they are used as matrix materials, as electron transport materials or as hole blocker materials in organic electroluminescent devices, should lead to devices having excellent color purity.

A further problem can be considered that of providing electronic devices having excellent performance very inexpensively and in constant quality.

Furthermore, it should be possible to use or adapt the electronic devices for many purposes. More particularly, the performance of the electronic devices should be maintained over a broad temperature range.

It has been found that, surprisingly, particular compounds described in detail below solve this problem and are of good suitability for use in electroluminescent devices and lead to improvements in the organic electroluminescent device, especially in relation to lifetime, color purity, efficiency and operating voltage. The present invention therefore provides these compounds and electronic devices, especially organic electroluminescent devices, comprising such compounds.

The present invention provides a compound comprising at least one structure of the formula (I), preferably a compound of the formula (I),

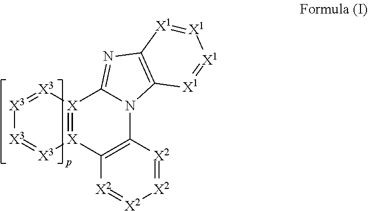

where the symbols and indices used are as follows:
X is N, CR or, if p=1, C; preferably, X is CR or C;
X¹ is the same or different at each instance and is N, CAr^{a} or CR¹, with the proviso that not more than two of the X¹ groups in one cycle are N;
X² is the same or different at each instance and is N, CR^{b} or CR², with the proviso that not more than two of the X² groups in one cycle are N;
X³ is the same or different at each instance and is N, CAr^{c} or CR³ with the proviso that not more than two of the X³ groups in one cycle are N;
p is 0 or 1, where the aromatic or heteroaromatic 6-membered ring with the X³ radicals is absent if p=0;
Ar^{a} is the same or different at each instance and is an aromatic or heteroaromatic ring system which has 5 to 40 aromatic ring atoms and may be substituted by one or more R¹ radicals;
Ar^{b} is the same or different at each instance and is an aromatic or heteroaromatic ring system which has 5 to 40 aromatic ring atoms and may be substituted by one or more R² radicals;
Ar^{c} is the same or different at each instance and is an aromatic or heteroaromatic ring system which has 5 to 40 aromatic ring atoms and may be substituted by one or more R³ radicals;
R is the same or different at each instance and is H, D, F, Cl, Br, I, N(R⁴)₂, N(Ar′)₂, CN, NO₂, OR⁴, OAr′, SR⁴, SAr′, COOR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R⁴ radicals, where one or more nonadjacent CH₂ groups may be replaced by Si(R⁴)₂, C═O, NR⁴, O, S or CONR⁴, or an aromatic or heteroaromatic ring system which has 5 to 60 aromatic ring atoms, preferably 5 to 40 aromatic ring atoms, and may be substituted in each case by one or more R⁴ radicals; at the same time, two R radicals together or one R radical together with one R² radical may also form an aliphatic, heteroaliphatic, aromatic or heteroaromatic ring system; preferably, the R radicals do not form any such ring system;
R¹ is the same or different at each instance and is H, D, F, Cl, Br, I, N(R⁴)₂, N(Ar′)₂, CN, NO₂, OR⁴, OAr′, SR⁴, SAr′, COOR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R⁴ radicals, where one or more nonadjacent CH₂ groups may be replaced by Si(R⁴)₂, C═O, NR⁴, O, S or CONR⁴, or an aromatic or heteroaromatic ring system which has 5 to 60 aromatic ring atoms, preferably 5 to 40 aromatic ring atoms, and may be substituted in each case by one or more R⁴ radicals; at the same time, two R¹ radicals together or one R¹ radical together with one R² radical may also form an aliphatic, heteroaliphatic, aromatic or heteroaromatic ring system, preferably an aliphatic, heteroaliphatic or heteroaromatic ring system; more preferably, the R¹ radicals do not form any such ring system;
R² is the same or different at each instance and is H, D, F, Cl, Br, I, N(R⁴)₂, N(Ar′)₂, CN, NO₂, OR⁴, OAr′, SR⁴, SAr′, COOR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R⁴ radicals, where one or more nonadjacent CH₂ groups may be replaced by Si(R⁴)₂, C═O, NR⁴, O, S or CONR⁴, or an aromatic or heteroaromatic ring system which has 5 to 60 aromatic ring atoms, preferably 5 to 40 aromatic ring atoms, and may be substituted in each case by one or more R⁴ radicals; at the same time, two R² radicals together or one R² radical together with one R, R¹, R³ radical may also form an aromatic, heteroaromatic, aliphatic or heteroaliphatic ring system; preferably, the R² radicals do not form any such ring system;
R³ is the same or different at each instance and is H, D, F, Cl, Br, I, N(R⁴)₂, N(Ar′)₂, CN, NO₂, OR⁴, OAr′, SR⁴, SAr′, COOR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R⁴ radicals, where one or more nonadjacent CH₂ groups may be replaced by Si(R⁴)₂, C═O, NR⁴, O, S or CONR⁴, or an aromatic or heteroaromatic ring system which has 5 to 60 aromatic ring atoms, preferably 5 to 40 aromatic ring atoms, and may be substituted in each case by one or more R⁴ radicals; at the same time, two R³ radicals together or one R³ radical together with one R² radical may also form an aromatic, heteroaromatic, aliphatic or heteroaliphatic ring system; preferably, the R³ radicals do not form any such ring system;
Ar′ is the same or different at each instance and is an aromatic or heteroaromatic ring system which has 5 to 40 aromatic ring atoms and may be substituted by one or more R⁴ radicals;
R⁴ is the same or different at each instance and is H, D, F, Cl, Br, I, N(R⁵)₂, CN, NO₂, OR⁵, SR⁵, Si(R⁵)₃, B(OR⁵)₂, C(═O)R⁵, P(═O)(R⁵)₂, S(═O)R⁵, S(═O)₂R⁵, OSO₂R⁵, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R⁵ radicals and where one or more nonadjacent CH₂ groups may be replaced by Si(R⁵)₂, C═O, NR⁵, O, S or CONR⁵, or an aromatic or heteroaromatic ring system which has 5 to 40 aromatic ring atoms and may be substituted in each case by one or more R⁵ radicals; at the same time, two or more R⁴ radicals together may form an aromatic, heteroaromatic, aliphatic or heteroaliphatic ring system, preferably an aliphatic ring system; more preferably, the R⁴ radicals do not form any such ring system;
R⁵ is the same or different at each instance and is H, D, F or an aliphatic, aromatic or heteroaromatic organic radical, especially a hydrocarbyl radical, having 1 to 20 carbon atoms, in which one or more hydrogen atoms may also be replaced by F;
wherein, in at least one of the rings having the X¹, X² or X³ groups, two nonadjacent X¹, X² or X³ groups in one ring are N.

It may preferably be the case that, in at least one of the rings having the X¹, X² or X³ groups, two nonadjacent X¹, X² or X³ groups in one ring are N, and the X¹, X² or X³ groups adjacent to the respective N in a ring having at least two nonadjacent nitrogen atoms are CAr^{a}, CAr^{b}, CAr^{c} or CR¹, CR², CR³, where R¹, R², R³ is an aromatic or heteroaromatic ring system which has 5 to 60 aromatic ring atoms, preferably 5 to 40 aromatic ring atoms, and may be substituted in each case by one or more R⁴ radicals.

It may further be the case that, in at least one of the rings having the X¹, X² or X³ groups, two nonadjacent X¹, X² or X³ groups in one ring are N, where the two nonadjacent X¹, X² or X³ groups in one ring that are N are in meta positions to one another.

In one embodiment of the present invention, it may be the case that two nonadjacent X¹ groups are N, two X¹ groups are Ar^{a}, and at least two, preferably at least 4 and more preferably all X, X² or X³ groups are CR, CR² or CR³.

In a further configuration of the present invention, it may be the case that two nonadjacent X² groups are N, two X² groups are Ar^{b}, and at least two, preferably at least 4 and more preferably all X, X¹ or X³ groups are CR, CR¹ or CR³.

In a further configuration of the present invention, it may be the case that two nonadjacent X³ groups are N, two X³ groups are Ar^{c}, and at least two, preferably at least 4 and more preferably all X, X¹ or X² groups are CR, CR¹ or CR².

An aryl group in the context of this invention contains 6 to 40 carbon atoms, a heteroaryl group in the context of this invention contains 2 to 40 carbon atoms and at least one heteroatom, with the proviso that the sum total of carbon atoms and heteroatoms is at least 5. The heteroatoms are preferably selected from N, O and/or S. An aryl group or heteroaryl group is understood here to mean either a simple aromatic cycle, i.e. benzene, or a simple heteroaromatic cycle, for example pyridine, pyrimidine, thiophene, etc., or a fused (annelated) aryl or heteroaryl group, for example naphthalene, anthracene, phenanthrene, quinoline, isoquinoline, etc. Aromatics joined to one another by a single bond, for example biphenyl, by contrast, are not referred to as an aryl or heteroaryl group but as an aromatic ring system.

An electron-deficient heteroaryl group in the context of the present invention is a heteroaryl group having at least one heteroaromatic six-membered ring having at least one nitrogen atom. Further aromatic or heteroaromatic five-membered or six-membered rings may be fused onto this six-membered ring. Examples of electron-deficient heteroaryl groups are pyridine, pyrimidine, pyrazine, pyridazine, triazine, quinoline, quinazoline or quinoxaline.

An aromatic ring system in the context of this invention contains 6 to 60 carbon atoms in the ring system. A heteroaromatic ring system in the context of this invention contains 2 to 60 carbon atoms and at least one heteroatom in the ring system, with the proviso that the sum total of carbon atoms and heteroatoms is at least 5. The heteroatoms are preferably selected from N, O and/or S. An aromatic or heteroaromatic ring system in the context of this invention shall be understood to mean a system which does not necessarily contain only aryl or heteroaryl groups, but in which it is also possible for two or more aryl or heteroaryl groups to be joined by a non-aromatic unit, for example a carbon, nitrogen or oxygen atom. For example, systems such as fluorene, 9,9′-spirobifluorene, 9,9-diarylfluorene, triarylamine, diaryl ethers, stilbene, etc. shall also be regarded as aromatic ring systems in the context of this invention, and likewise systems in which two or more aryl groups are joined, for example, by a short alkyl group. Preferably, the aromatic ring system is selected from fluorene, 9,9′-spirobifluorene, 9,9-diarylamine or groups in which two or more aryl and/or heteroaryl groups are joined to one another by single bonds.

In the context of the present invention, an aliphatic hydrocarbyl radical or an alkyl group or an alkenyl or alkynyl group which may contain 1 to 20 carbon atoms and in which individual hydrogen atoms or CH₂ groups may also be substituted by the abovementioned groups is preferably understood to mean the methyl, ethyl, n-propyl, i-propyl, n-butyl, i-butyl, s-butyl, t-butyl, 2-methylbutyl, n-pentyl, s-pentyl, neopentyl, cyclopentyl, n-hexyl, neohexyl, cyclohexyl, n-heptyl, cycloheptyl, n-octyl, cyclooctyl, 2-ethylhexyl, trifluoromethyl, pentafluoroethyl, 2,2,2-trifluoroethyl, ethenyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl, heptynyl or octynyl radicals. An alkoxy group having 1 to 40 carbon atoms is preferably understood to mean methoxy, trifluoromethoxy, ethoxy, n-propoxy, i-propoxy, n-butoxy, i-butoxy, s-butoxy, t-butoxy, n-pentoxy, s-pentoxy, 2-methylbutoxy, n-hexoxy, cyclohexyloxy, n-heptoxy, cycloheptyloxy, n-octyloxy, cyclooctyloxy, 2-ethylhexyloxy, pentafluoroethoxy and 2,2,2-trifluoroethoxy. A thioalkyl group having 1 to 40 carbon atoms is understood to mean especially methylthio, ethylthio, n-propylthio, i-propylthio, n-butylthio, i-butylthio, s-butylthio, t-butylthio, n-pentylthio, s-pentylthio, n-hexylthio, cyclohexylthio, n-heptylthio, cycloheptylthio, n-octylthio, cyclooctylthio, 2-ethylhexylthio, trifluoromethylthio, pentafluoroethylthio, 2,2,2-trifluoroethylthio, ethenylthio, propenylthio, butenylthio, pentenylthio, cyclopentenylthio, hexenylthio, cyclohexenylthio, heptenylthio, cycloheptenylthio, octenylthio, cyclooctenylthio, ethynylthio, propynylthio, butynylthio, pentynylthio, hexynylthio, heptynylthio or octynylthio. In general, alkyl, alkoxy or thioalkyl groups according to the present invention may be straight-chain, branched or cyclic, where one or more nonadjacent CH₂ groups may be replaced by the abovementioned groups; in addition, it is also possible for one or more hydrogen atoms to be replaced by D, F, Cl, Br, I, CN or NO₂, preferably F, Cl or CN, further preferably F or CN, especially preferably CN.

An aromatic or heteroaromatic ring system which has 5-60 or 5-40 aromatic ring atoms and may also be substituted in each case by the abovementioned radicals and which may be joined to the aromatic or heteroaromatic system via any desired positions is understood to mean especially groups derived from benzene, naphthalene, anthracene, benzanthracene, phenanthrene, pyrene, chrysene, perylene, fluoranthene, naphthacene, pentacene, benzopyrene, biphenyl, biphenylene, terphenyl, triphenylene, fluorene, spirobifluorene, dihydrophenanthrene, dihydropyrene, tetrahydropyrene, cis- or trans-indenofluorene, cis- or trans-indenocarbazole, cis- or trans-indolocarbazole, truxene, isotruxene, spirotruxene, spiroisotruxene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, hexaazatriphenylene, benzopyridazine, pyrimidine, benzopyrimidine, quinoxaline, 1,5-diazaanthracene, 2,7-diazapyrene, 2,3-diazapyrene, 1,6-diazapyrene, 1,8-diazapyrene, 4,5-diazapyrene, 4,5,9,10-tetraazaperylene, pyrazine, phenazine, phenoxazine, phenothiazine, fluorubine, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole, or groups derived from combinations of these systems.

The wording that two or more radicals together may form a ring, in the context of the present description, should be understood to mean, inter alia, that the two radicals are joined to one another by a chemical bond with formal elimination of two hydrogen atoms. This is illustrated by the following scheme:

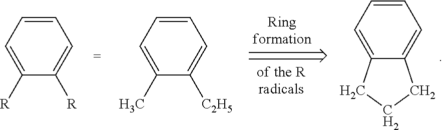

In addition, however, the abovementioned wording shall also be understood to mean that, if one of the two radicals is hydrogen, the second radical binds to the position to which the hydrogen atom was bonded, forming a ring. This will be illustrated by the following scheme:

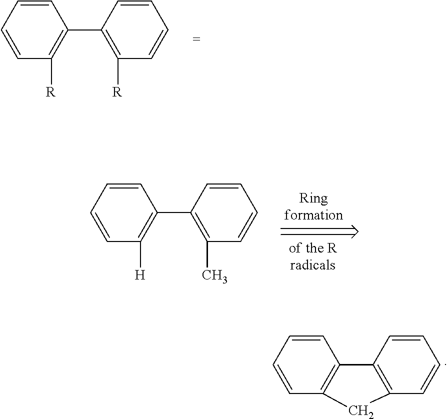

In a preferred configuration, the compounds of the invention may preferably comprise at least one structure of the formulae (II-1) to (II-25) and are more preferably selected from the compounds of the formulae (II-1) to (II-25):

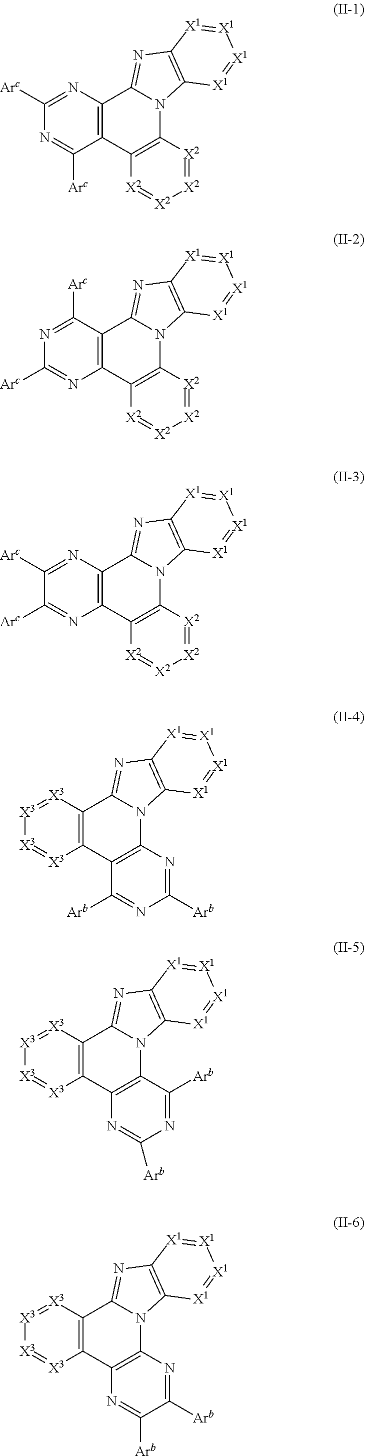

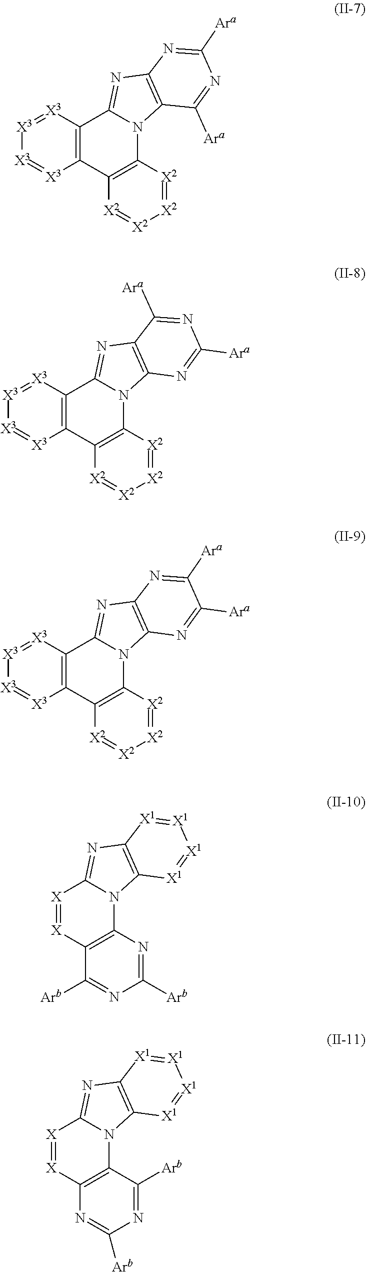

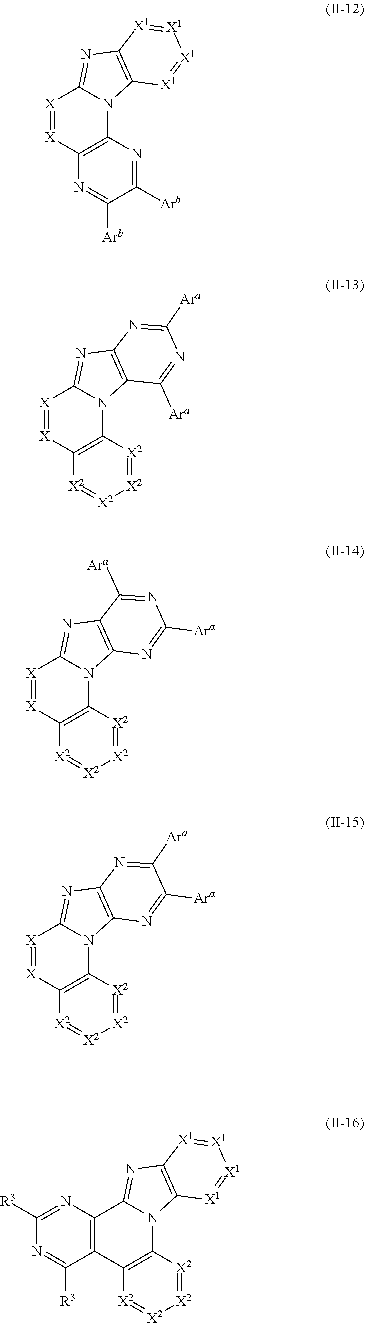

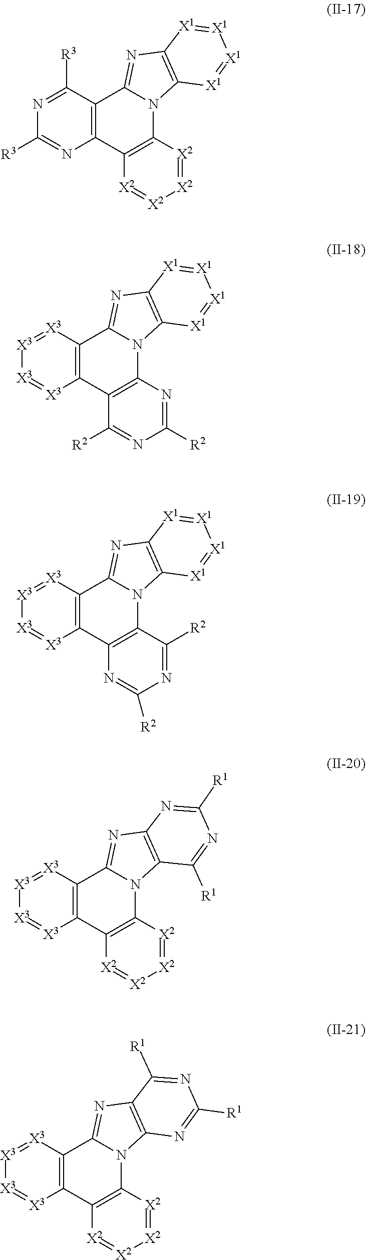

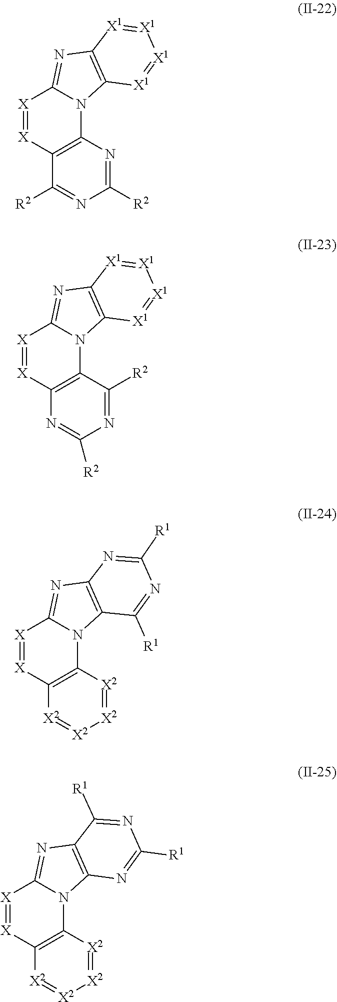

where the symbols X, X¹, X², X³, R¹, R², R³, Ar^{a}, Ar^{b} and Ar^{c} have the definitions given above, especially for formula (I). Preference is given here to structures/compounds of the formulae (II-1) to (II-9), particular preference to structures/compounds of the formulae (II-1), (II-2), (II-4), (II-5), (II-7) and (II-8).

It may preferably be the case that, in structures/compounds of the formulae (II-1) to (II-25), not more than four, preferably not more than two, X, X¹, X², X³ groups are N, preferably all X, X¹, X², X³ groups are CR, CR¹, CR² or CR³, where preferably not more than 6, more preferably not more than 4 and especially preferably not more than 2 of the CR, CR¹, CR² or CR³ groups represented by X, X¹, X², X³ are not the CH group.

In a further preferred embodiment, it may be the case that the compounds of the invention include a structure of the formulae (III-1) to (III-50), where the compounds of the invention may more preferably be selected from the compounds of the formulae (III-1) to (III-50)

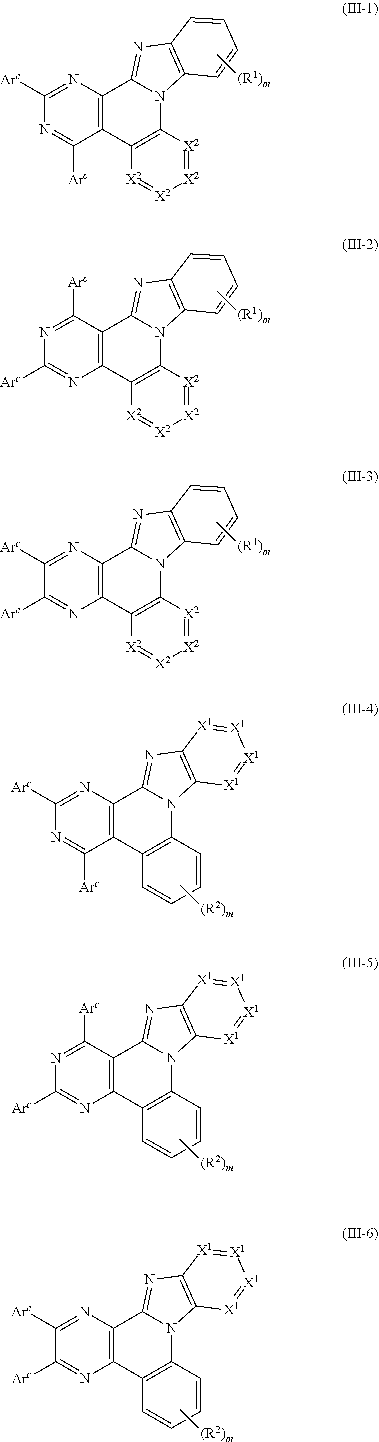

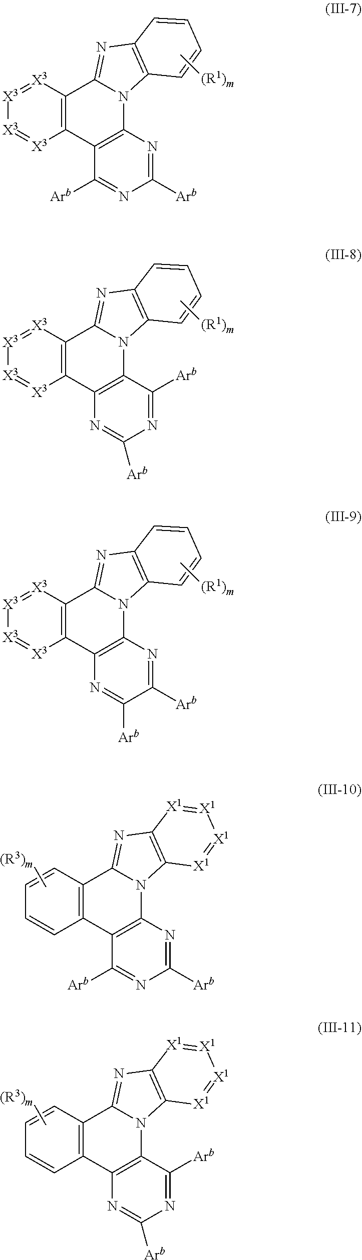

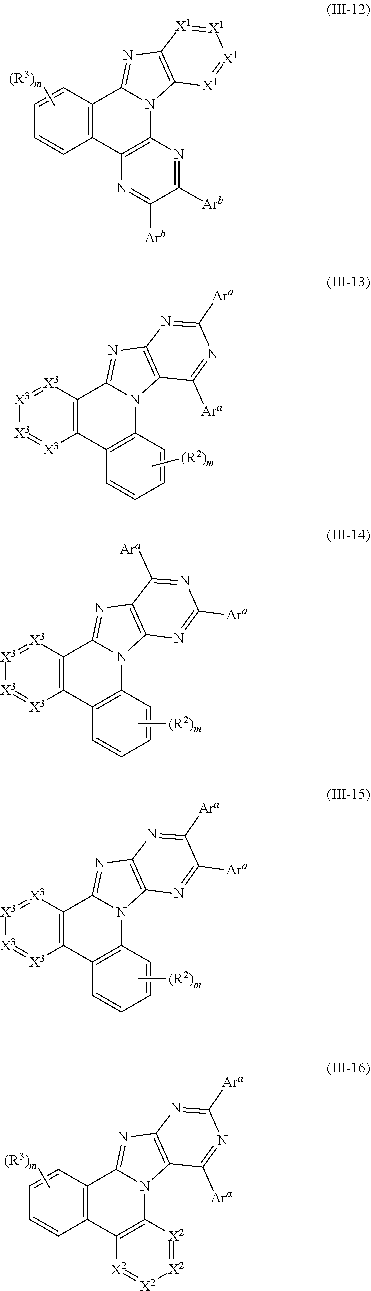

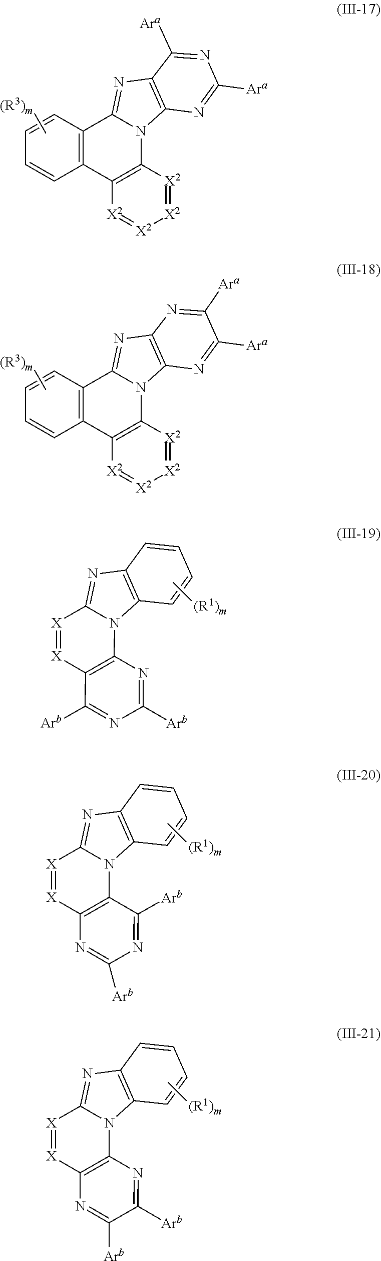

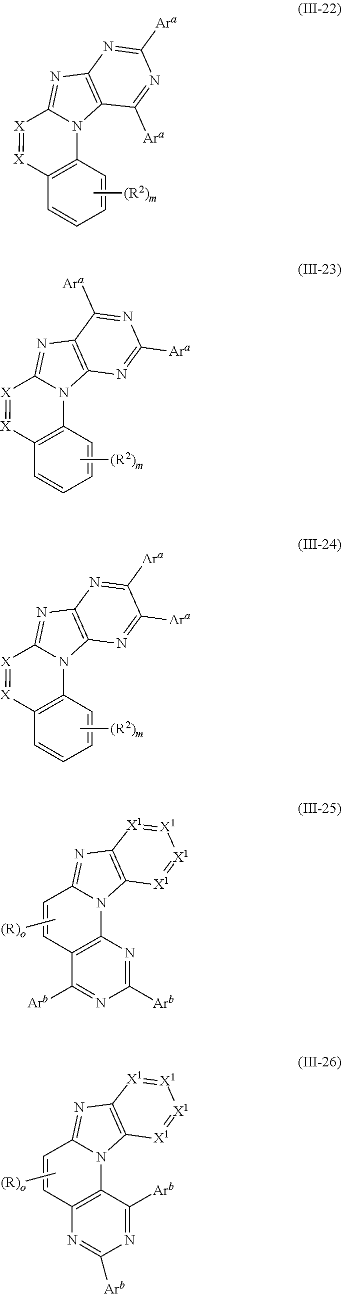

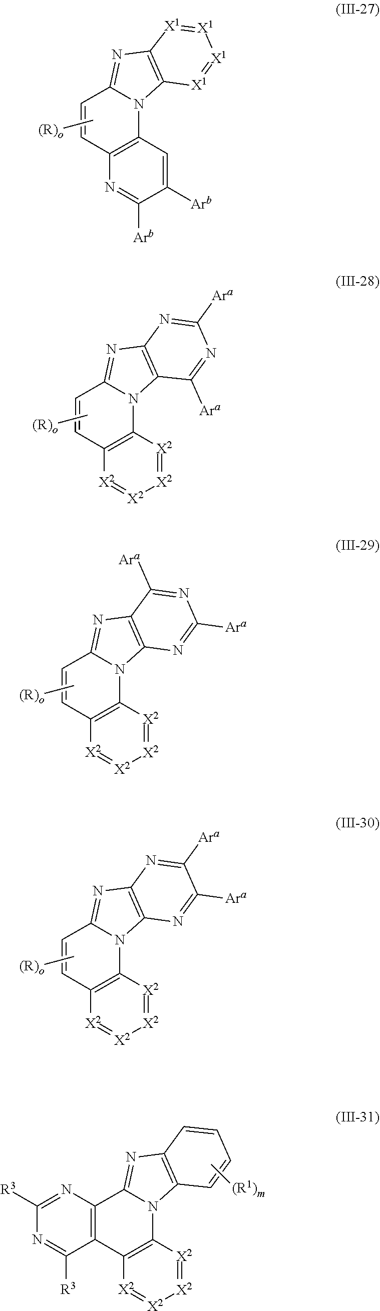

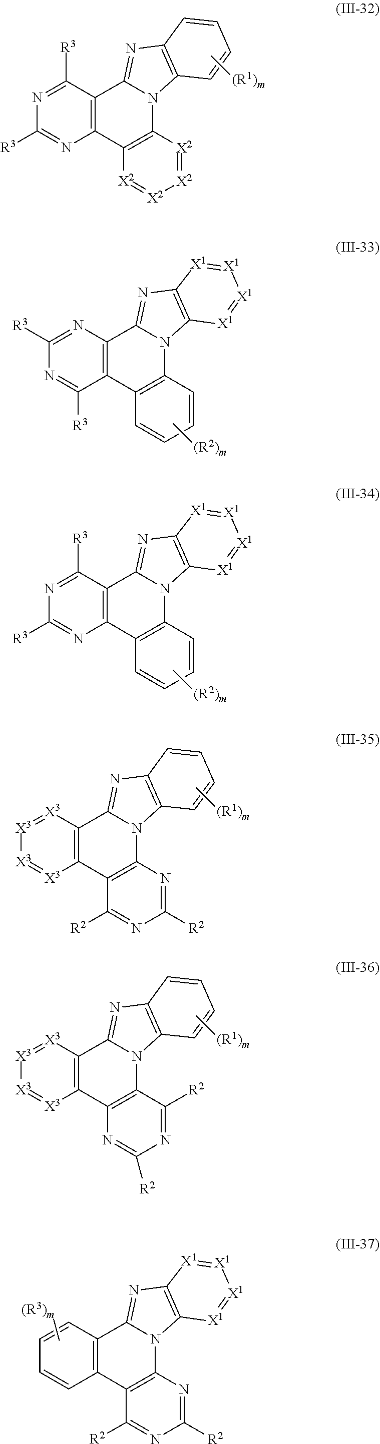

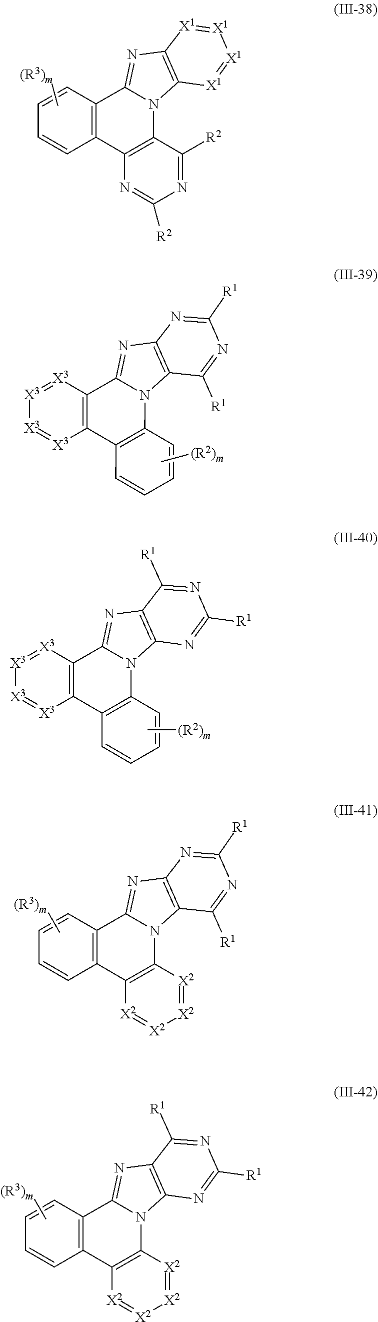

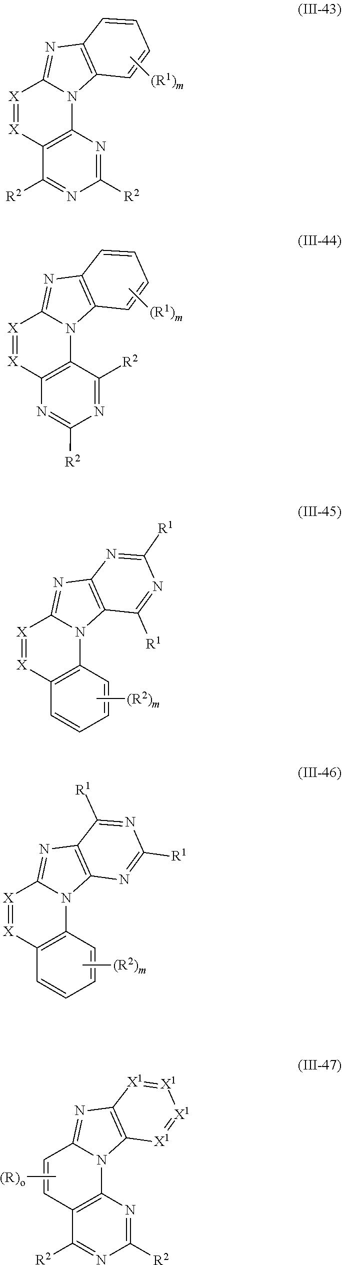

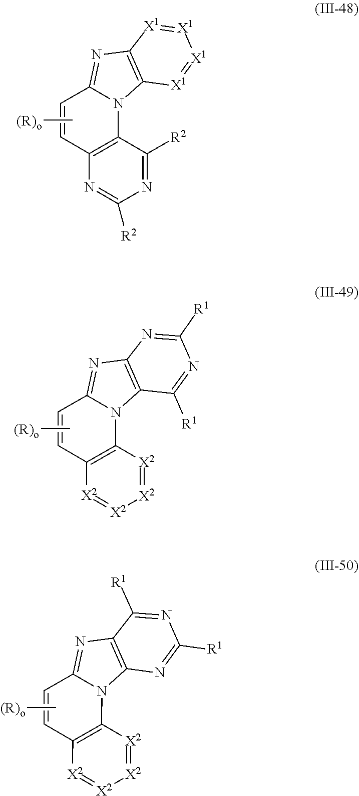

where the symbols R, R¹, R², R³, X, X¹, X², X³, Ar^{a}, Ar^{b} and Ar^{c} have the definitions given above, especially for formula (I), the index o is 0, 1 or 2, preferably 0 or 1, the index m is 0, 1, 2, 3 or 4, preferably 0, 1 or 2. Preference is given here to structures/compounds of the formulae (III-1) to (III-18), particular preference to structures/compounds of the formulae (III-1), (III-2), (III-4), (III-5), (III-7), (III-8), (III-10), (III-11), (III-13), (III-14), (III-16) and (III-17).

It may further be the case that, in structures/compounds of the formulae (I), (II-1) to (II-25) and/or (III-1) to (III-25), there are no two adjacent X, X¹, X², X³ that are N.

In a further preferred embodiment, it may be the case that the compounds of the invention include a structure of the formulae (IV-1) to (IV-25), where the compounds of the invention may more preferably be selected from the compounds of the formulae (IV-1) to (IV-25)

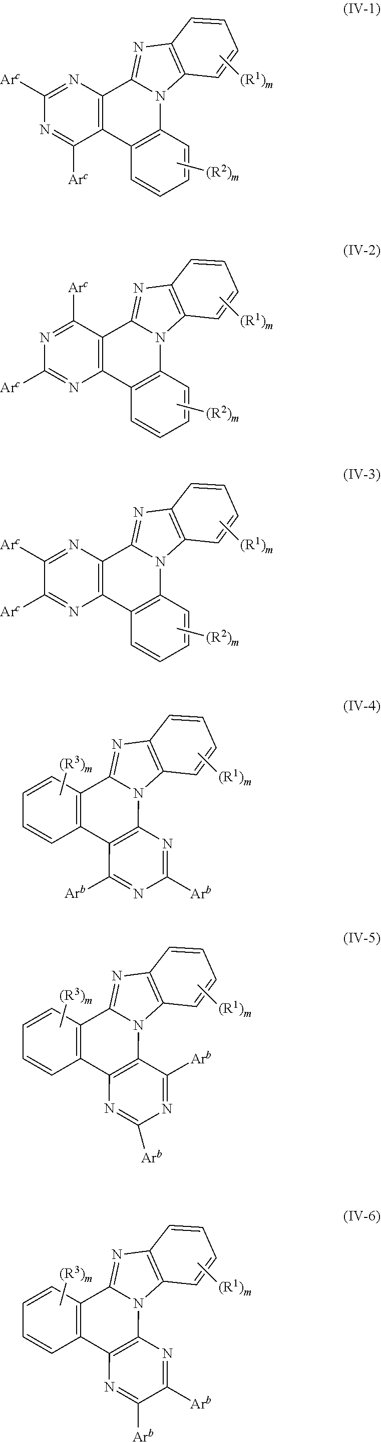

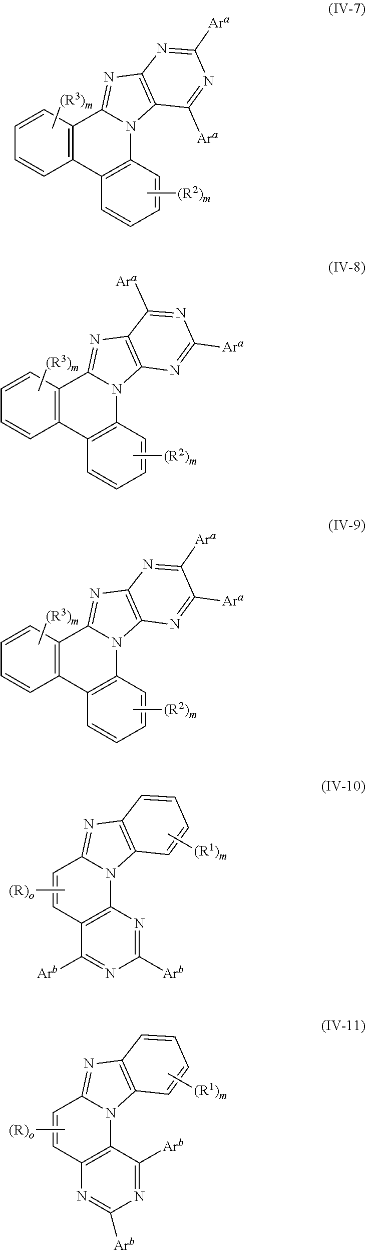

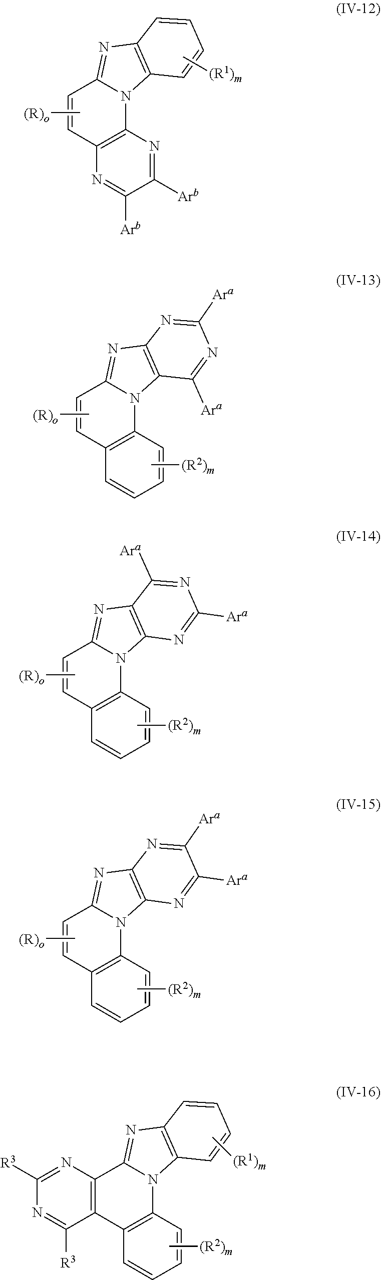

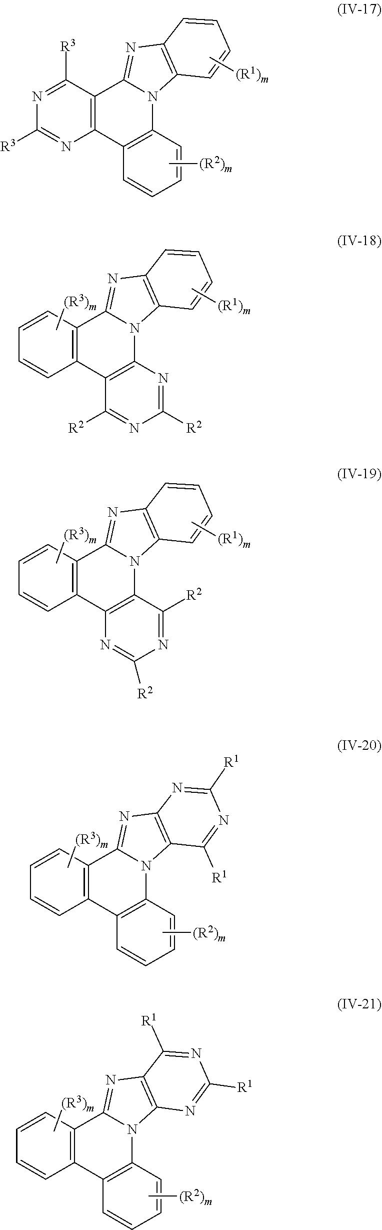

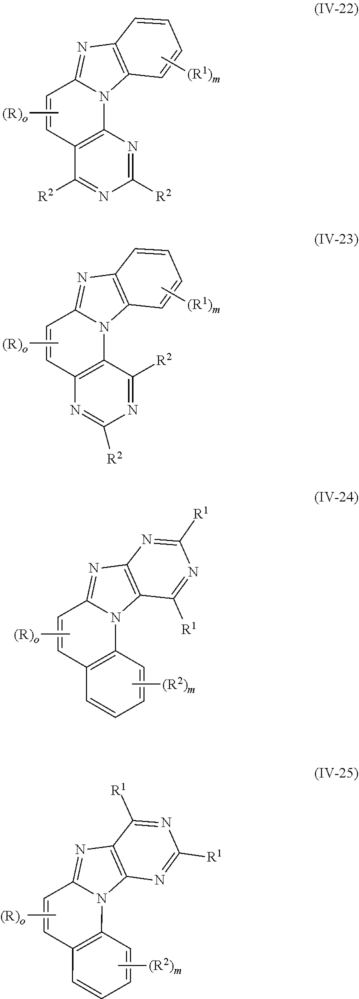

where the symbols R, R¹, R², R³, Ar^{a}, Ar^{b} and Ar^{c} have the definitions given above, especially for formula (I), the index o is 0, 1 or 2, preferably 0 or 1, the index m is 0, 1, 2, 3 or 4, preferably 0, 1 or 2. Preference is given here to structures/compounds of the formulae (IV-1) to (IV-9), particular preference to structures/compounds of the formulae (IV-1), (IV-2), (IV-4), (IV-5), (IV-7) and (IV-8).

In a further preferred embodiment, it may be the case that the compounds of the invention include a structure of the formulae (V-1) to (V-15), where the compounds of the invention may more preferably be selected from the compounds of the formulae (V-1) to (V-15)

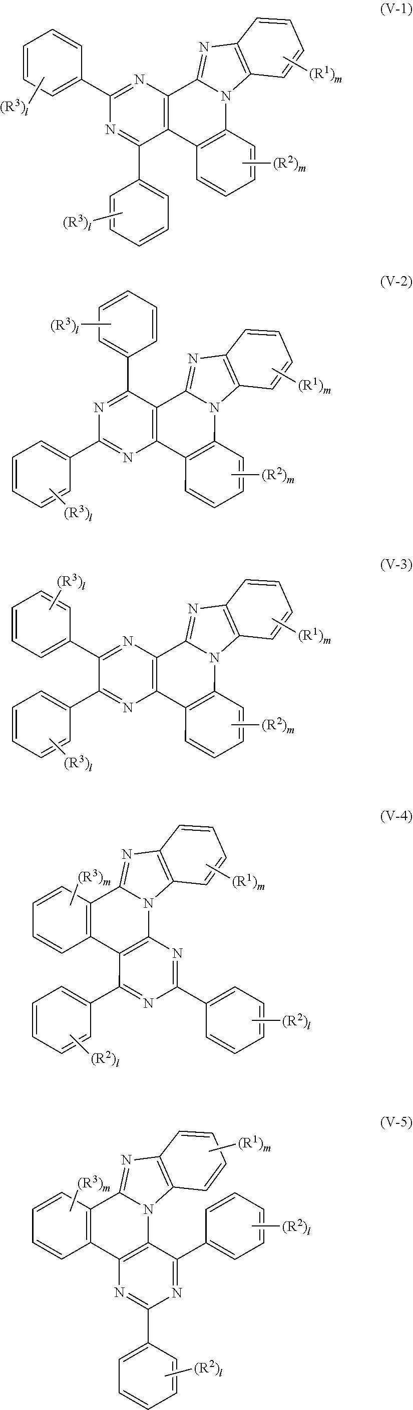

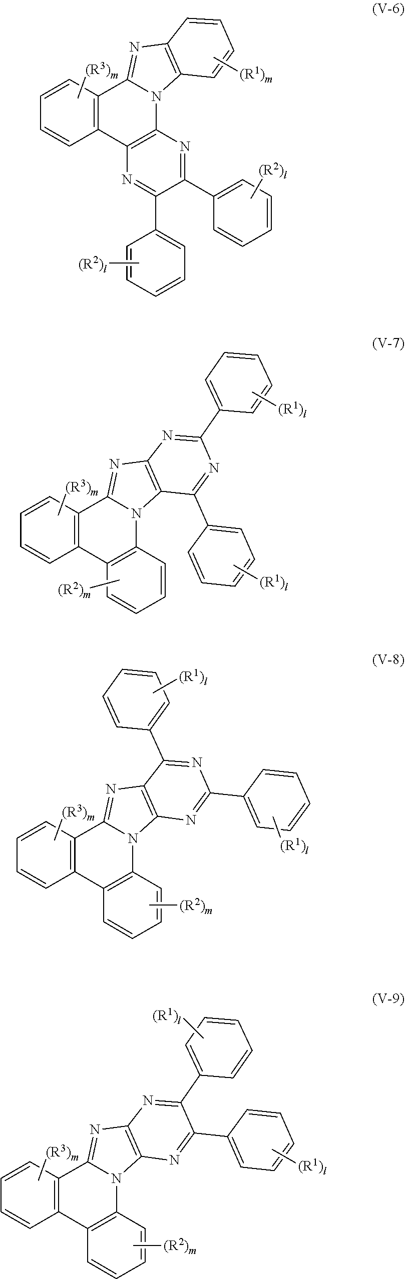

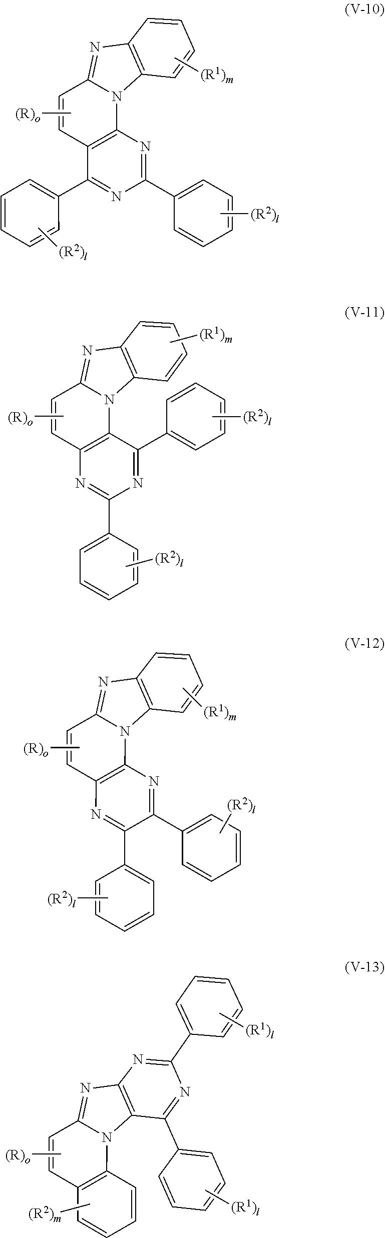

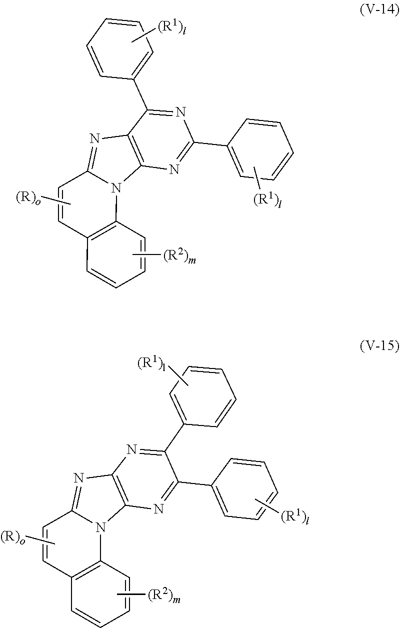

where the symbols R, R¹, R² and R³ have the definitions given above, especially for formula (I), the index o is 0, 1 or 2, preferably 0 or 1, the index m is 0, 1, 2, 3 or 4, preferably 0, 1 or 2, and the index I is 0, 1, 2, 3, 4 or 5, preferably 0, 1 or 2. Preference is given here to structures/compounds of the formulae (V-1) to (V-9), particular preference to structures/compounds of the formulae (V-1), (V-2), (V-4), (V-5), (V-7) and (V-8).

The sum total of the indices I, m and o in structures/compounds of the formulae (IV-1) to (IV-25) and (V-1) to (V-15) is preferably at most 6, especially preferably at most 4 and more preferably at most 2.

Preferred aromatic or heteroaromatic ring systems Ar^{a}, Ar^{b}, Ar^{c} are selected from phenyl, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorene which may be joined via the 1, 2, 3 or 4 position, spirobifluorene which may be joined via the 1, 2, 3 or 4 position, naphthalene, especially 1- or 2-bonded naphthalene, indole, benzofuran, benzothiophene, carbazole which may be joined via the 1, 2, 3, 4 or 9 position, dibenzofuran which may be joined via the 1, 2, 3 or 4 position, dibenzothiophene which may be joined via the 1, 2, 3 or 4 position, indenocarbazole, indolocarbazole, pyridine, pyrimidine, pyrazine, pyridazine, triazine, quinoline, isoquinoline, quinazoline, quinoxaline, phenanthrene or triphenylene, each of which may be substituted by one or more R¹, R², R³ radicals.

It may further be the case that the substituents R, R¹, R² and R³ according to the above formulae do not form a fused aromatic or heteroaromatic ring system, preferably any fused ring system, with the ring atoms of the ring system. This includes the formation of a fused ring system with possible substituents R⁴, R⁵ which may be bonded to the R, R¹, R², R³ radicals.

When two radicals that may especially be selected from R, R¹, R², R³, R⁴, R⁵, R⁶, R⁷, R⁸ and/or R⁹ form a ring system with one another, this ring system may be mono- or polycyclic, aliphatic, heteroaliphatic, aromatic or heteroaromatic. In this case, the radicals which together form a ring system may be adjacent, meaning that these radicals are bonded to the same carbon atom or to carbon atoms directly bonded to one another, or they may be further removed from one another. In addition, the ring systems provided with the substituents R, R¹, R², R³, R⁴, R⁵, R⁶, R⁷, R⁸ and/or R⁹ may also be joined to one another via a bond, such that this can bring about a ring closure. In this case, each of the corresponding bonding sites has preferably been provided with a substituent R, R¹, R², R³, R⁴, R⁵, R⁶, R⁷, R⁸ and/or R⁹.

It may further be the case that R, R¹, R² and/or R³ are the same or different at each instance and are selected from the group consisting of H, D or an aromatic or heteroaromatic ring system selected from the groups of the following formulae Ar-1 to Ar-75, and/or the Ar^{a}, Ar^{b}, Ar^{c} and/or Ar′ group is the same or different at each instance and is selected from the groups of the following formulae Ar-1 to Ar-75:

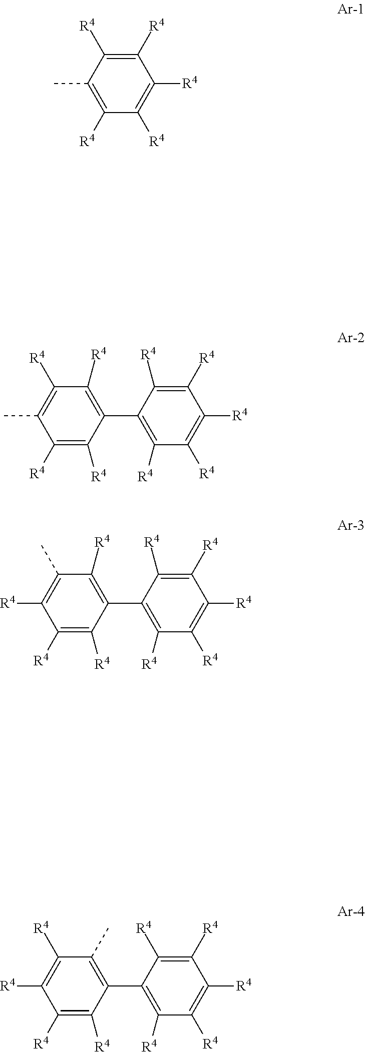

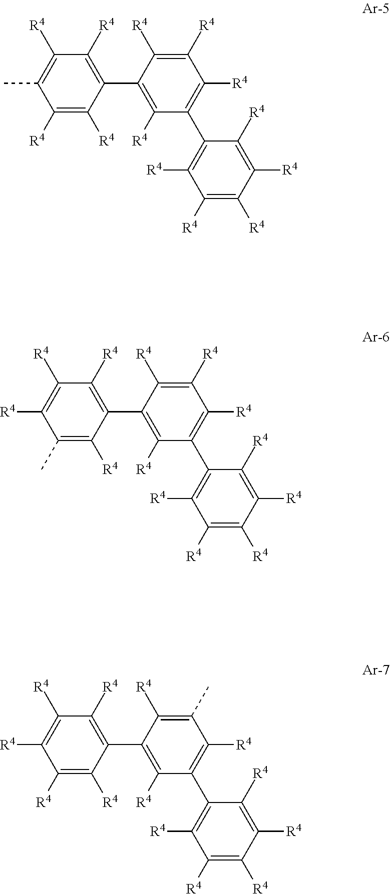

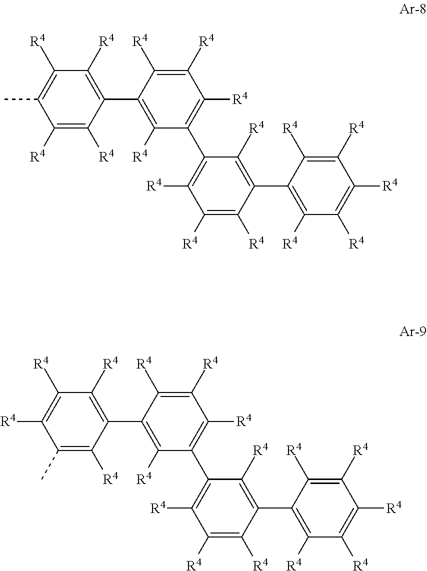

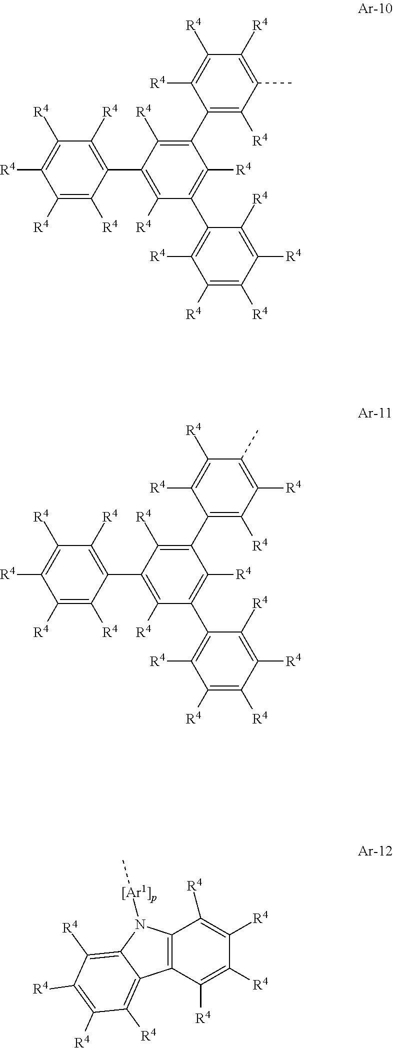

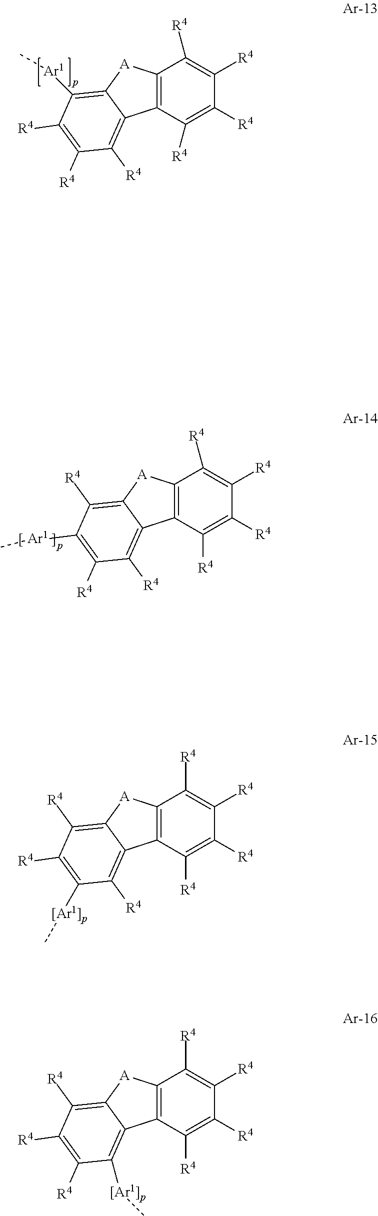

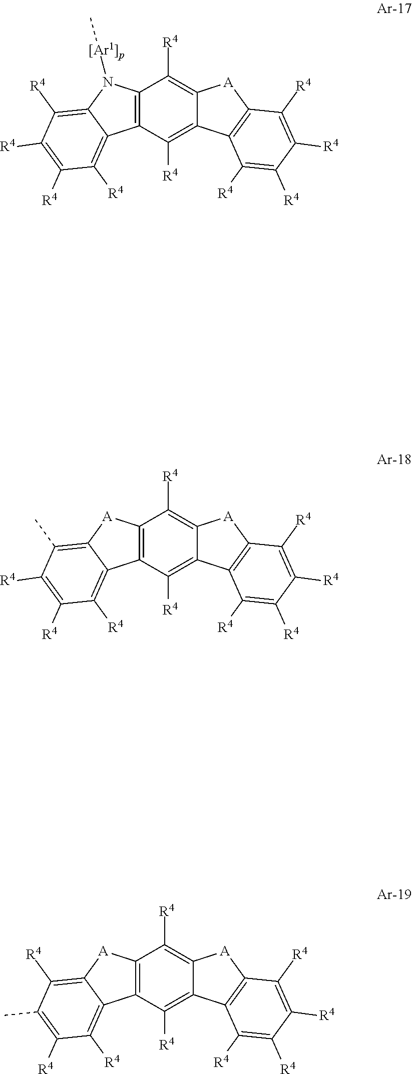

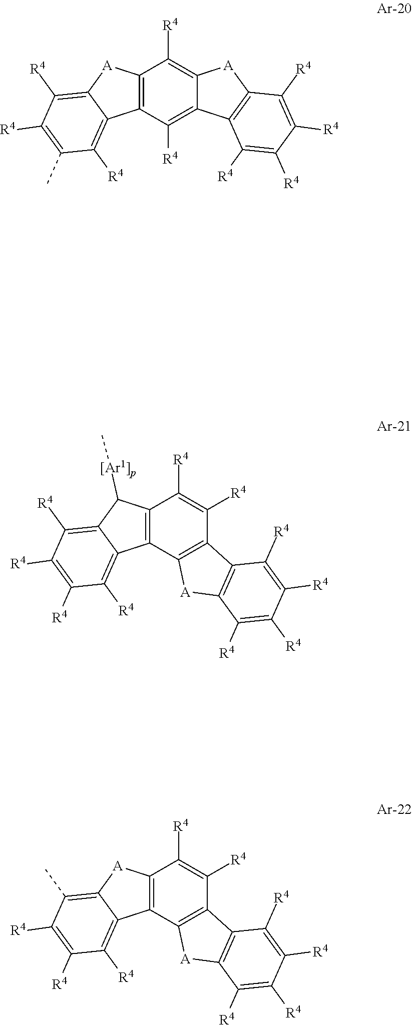

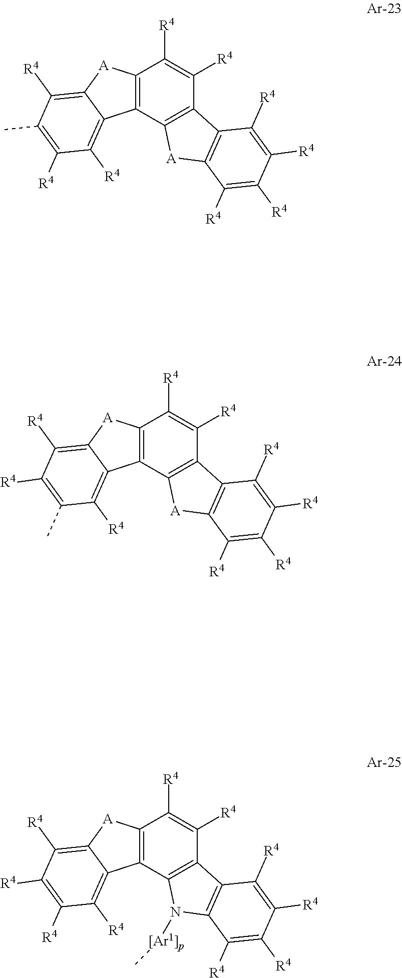

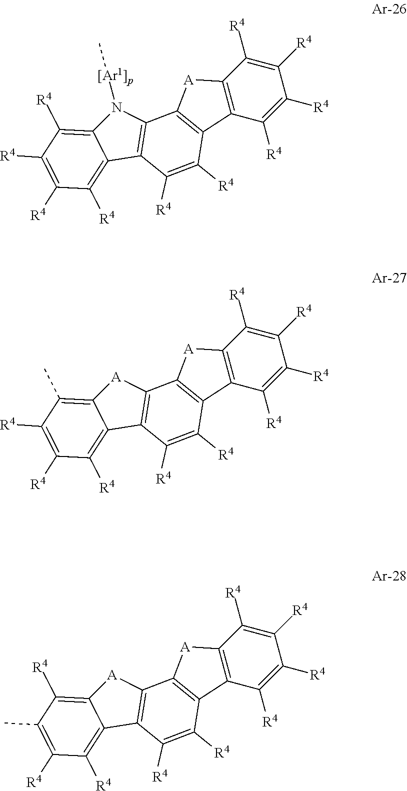

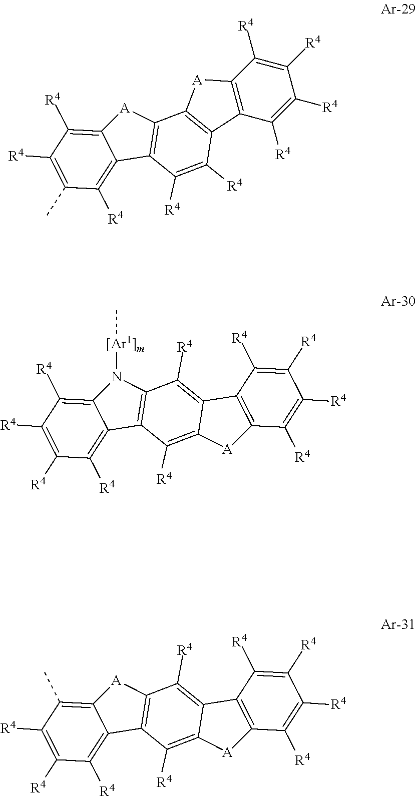

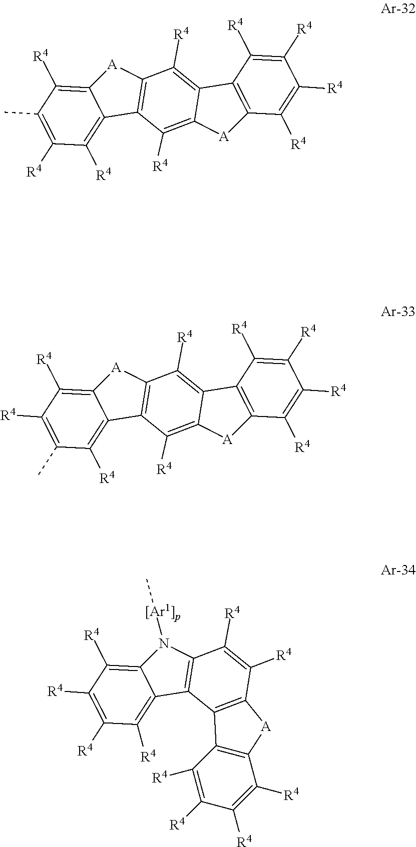

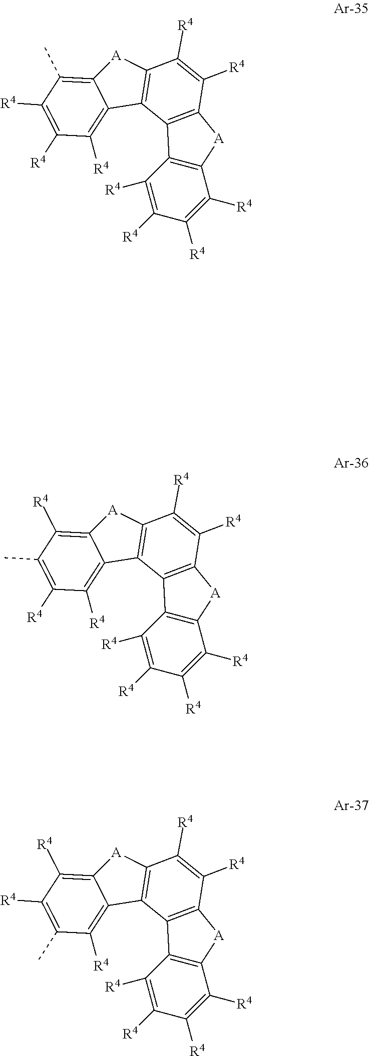

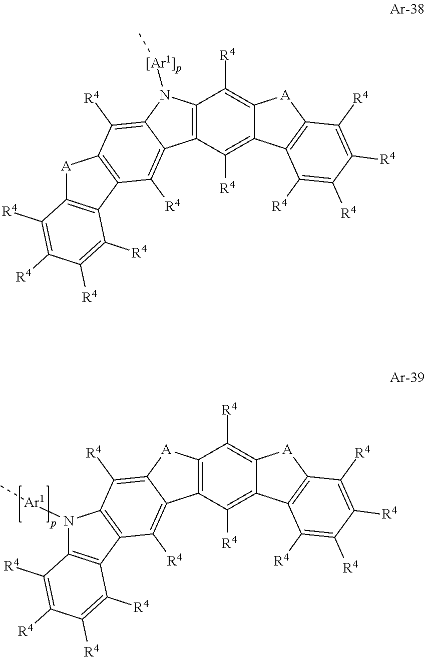

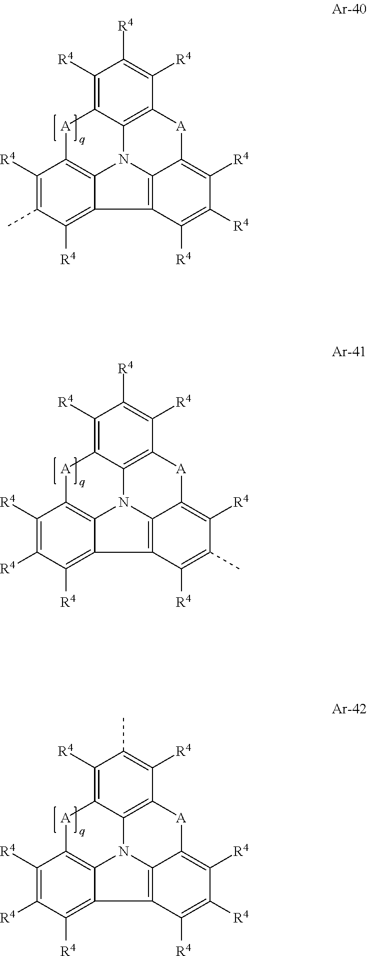

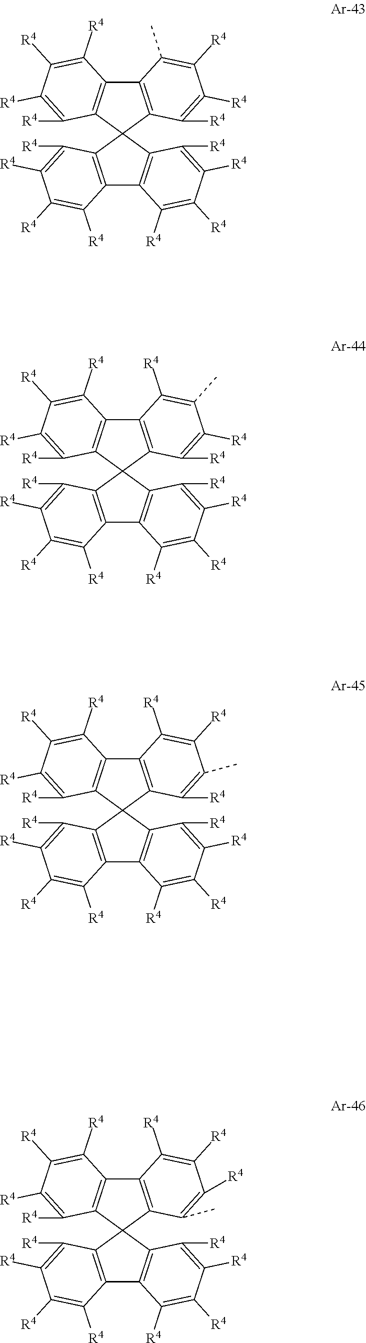

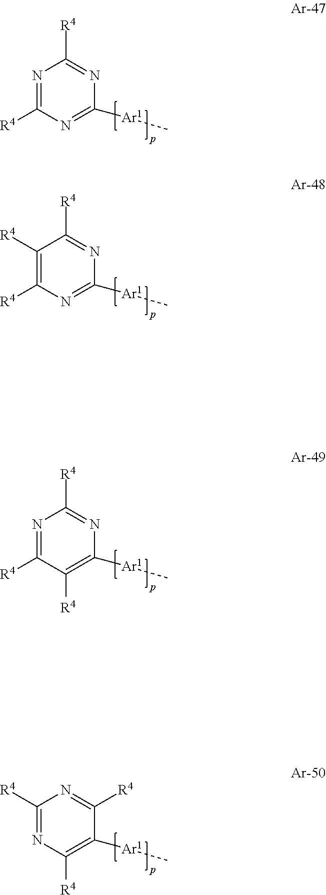

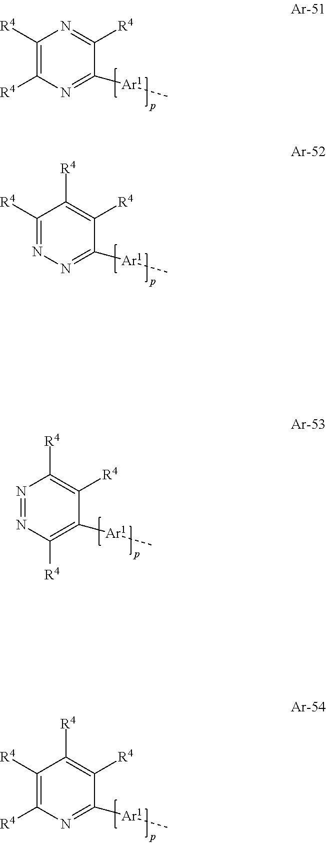

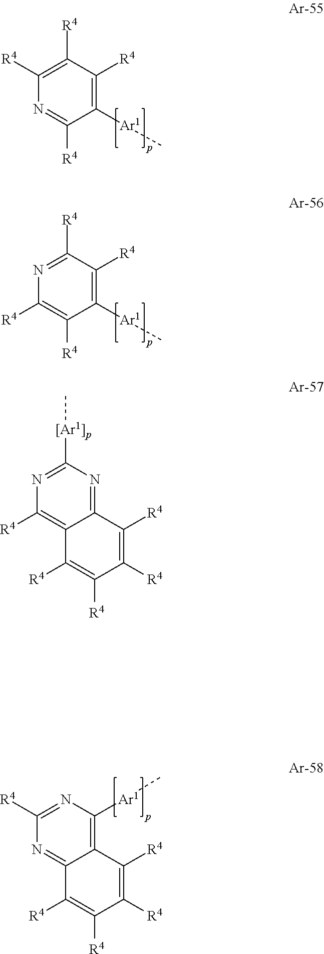

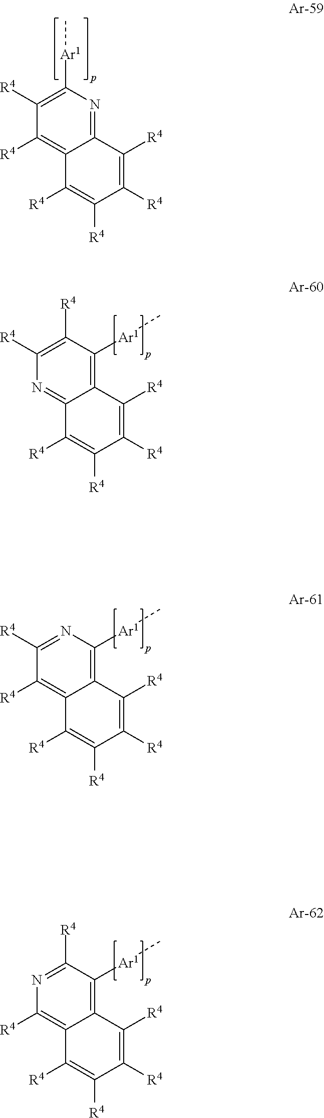

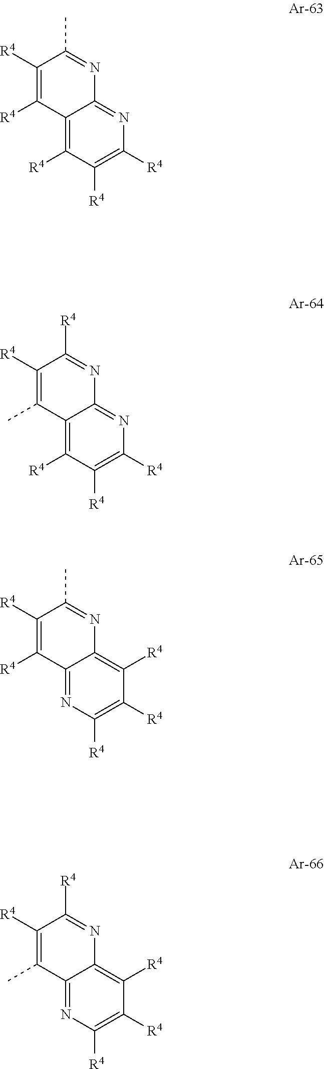

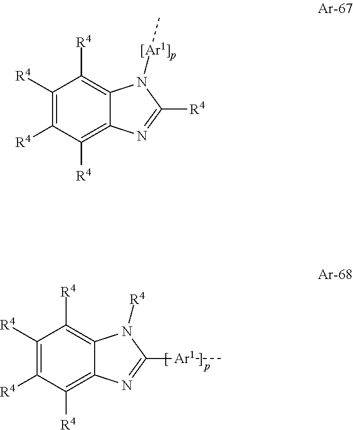

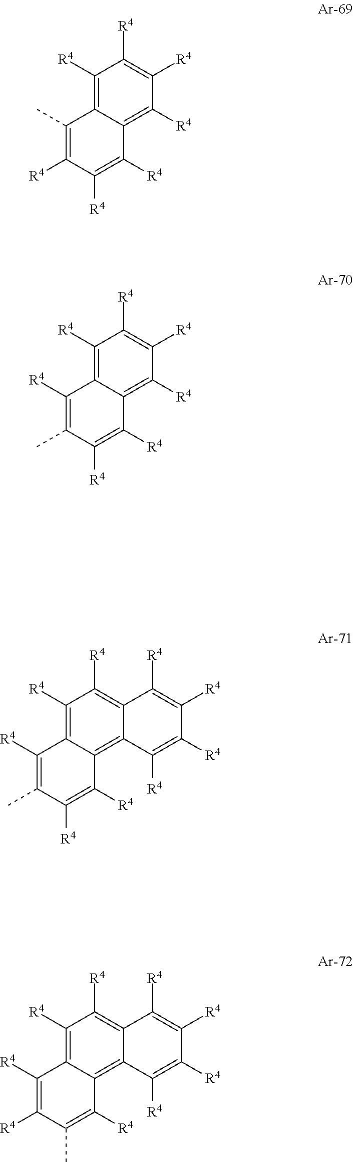

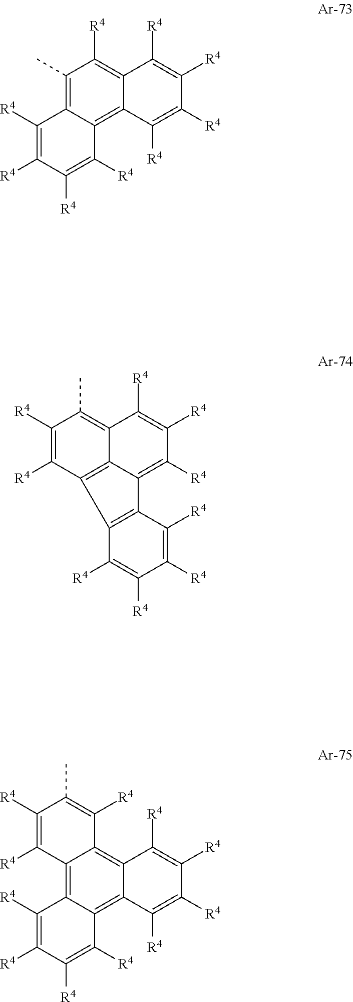

where R⁴ has the definitions given above, the dotted bond represents the bond of the corresponding group and in addition:
Ar¹ is the same or different at each instance and is a bivalent aromatic or heteroaromatic ring system which has 6 to 18 aromatic ring atoms and may be substituted in each case by one or more R⁴ radicals;
A is the same or different at each instance and is C(R⁴)₂, NR⁴, O or S;
p is 0 or 1, where p=0 means that the Ar¹ group is absent and that the corresponding aromatic or heteroaromatic group is bonded directly to the corresponding radical;
q is 0 or 1, where q=0 means that no A group is bonded at this position and R⁴ radicals are bonded to the corresponding carbon atoms instead.

The above-detailed structures of the formulae (Ar-1) to (Ar-75) are preferred configurations of the Ar^{a}, Ar^{b} and Ar^{c} radicals, in the case of which the substituents R⁴ in formulae (Ar-1) to (Ar-75) should be replaced by R¹, R² or R³, where R¹, R², R³ has the definition set out above, especially for formula (I).

Especially in relation to preferred configurations of the Ar^{a}, Ar^{b} and Ar^{c} radicals and of the R, R¹, R² and/or R³ radicals, preference is given to structures of the formulae (Ar-1), (Ar-2), (Ar-3), (Ar-12), (Ar-13), (Ar-14), (Ar-15), (Ar-16), (Ar-69), (Ar-70), (Ar-75), and particular preference to structures of the formulae (Ar-1), (Ar-2), (Ar-3), (Ar-12), (Ar-13), (Ar-14), (Ar-15), (Ar-16).

When the abovementioned groups for Ar have two or more A groups, possible options for these include all combinations from the definition of A. Preferred embodiments in that case are those in which one A group is NR⁴ and the other A group is C(R⁴)₂ or in which both A groups are NR⁴ or in which both A groups are O.

When A is NR⁴, the substituent R⁴ bonded to the nitrogen atom is preferably an aromatic or heteroaromatic ring system which has 5 to 24 aromatic ring atoms and may also be substituted by one or more R⁵ radicals. In a particularly preferred embodiment, this R⁴ substituent is the same or different at each instance and is an aromatic or heteroaromatic ring system which has 6 to 24 aromatic ring atoms, especially 6 to 18 aromatic ring atoms, which does not have any fused aryl groups and which does not have any fused heteroaryl groups in which two or more aromatic or heteroaromatic 6-membered ring groups are fused directly to one another, and which may also be substituted in each case by one or more R⁵ radicals. Preference is given to phenyl, biphenyl, terphenyl and quaterphenyl having bonding patterns as listed above for Ar-1 to Ar-11, where these structures, rather than by R⁴, may be substituted by one or more R⁵ radicals, but are preferably unsubstituted. Preference is further given to triazine, pyrimidine and quinazoline as listed above for Ar-47 to Ar-50, Ar-57 and Ar-58, where these structures, rather than by R⁴, may be substituted by one or more R⁵ radicals.

When A is C(R⁴)₂, the substituents R⁴ bonded to this carbon atom are preferably the same or different at each instance and are a linear alkyl group having 1 to 10 carbon atoms or a branched or cyclic alkyl group having 3 to 10 carbon atoms or an aromatic or heteroaromatic ring system having 5 to 24 aromatic ring atoms, which may also be substituted by one or more R⁵ radicals. Most preferably, R⁴ is a methyl group or a phenyl group. In this case, the R⁴ radicals together may also form a ring system, which leads to a spiro system.

There follows a description of preferred substituents R, R¹, R² and R³.

In a preferred embodiment of the invention, R, R¹, R² and R³ are the same or different at each instance and are selected from the group consisting of H, D, F, CN, NO₂, Si(R⁴)₃, B(OR⁴)₂, a straight-chain alkyl group having 1 to 20 carbon atoms or a branched or cyclic alkyl group having 3 to 20 carbon atoms, where the alkyl group may be substituted in each case by one or more R⁴ radicals, or an aromatic or heteroaromatic ring system which has 5 to 60 aromatic ring atoms, preferably 5 to 40 aromatic ring atoms, and may be substituted in each case by one or more R⁴ radicals.

In a further-preferred embodiment of the invention, R, R¹, R² and R³ are the same or different at each instance and are selected from the group consisting of H, D, F, a straight-chain alkyl group having 1 to 20 carbon atoms or a branched or cyclic alkyl group having 3 to 20 carbon atoms, where the alkyl group may be substituted in each case by one or more R⁴ radicals, or an aromatic or heteroaromatic ring system which has 5 to 60 aromatic ring atoms, preferably 5 to 40 aromatic ring atoms, and may be substituted in each case by one or more R⁴ radicals.

In a further-preferred embodiment of the invention, R, R¹, R² and R³ are the same or different at each instance and is selected from the group consisting of H, D, an aromatic or heteroaromatic ring system which has 6 to 30 aromatic ring atoms and may be substituted by one or more R⁴ radicals, and an N(Ar′)₂ group. More preferably, R, R¹, R² are the same or different at each instance and is selected from the group consisting of H or an aromatic or heteroaromatic ring system which has 6 to 24 aromatic ring atoms, preferably 6 to 18 aromatic ring atoms, more preferably 6 to 13 aromatic ring atoms, and may be substituted in each case by one or more R⁴ radicals.

Preferred aromatic or heteroaromatic ring systems R, R¹, R², R³ and Ar′ are selected from phenyl, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorene which may be joined via the 1, 2, 3 or 4 position, spirobifluorene which may be joined via the 1, 2, 3 or 4 position, naphthalene, especially 1- or 2-bonded naphthalene, indole, benzofuran, benzothiophene, carbazole which may be joined via the 1, 2, 3 or 4 position, dibenzofuran which may be joined via the 1, 2, 3 or 4 position, dibenzothiophene which may be joined via the 1, 2, 3 or 4 position, indenocarbazole, indolocarbazole, pyridine, pyrimidine, pyrazine, pyridazine, triazine, quinoline, isoquinoline, quinazoline, quinoxaline, phenanthrene or triphenylene, each of which may be substituted by one or more R⁴ radicals. The structures Ar-1 to Ar-75 listed above are particularly preferred, preference being given to structures of the formulae (Ar-1), (Ar-2), (Ar-3), (Ar-12), (Ar-13), (Ar-14), (Ar-15), (Ar-16), (Ar-69), (Ar-70), (Ar-75), and particular preference to structures of the formulae (Ar-1), (Ar-2), (Ar-3), (Ar-12), (Ar-13), (Ar-14), (Ar-15), (Ar-16).

Further suitable R, R¹, R² and R³ groups are groups of the formula —Ar⁴—N(Ar²)(Ar³) where Ar², Ar³ and Ar⁴ are the same or different at each instance and are an aromatic or heteroaromatic ring system which has 5 to 24 aromatic ring atoms and may be substituted in each case by one or more R⁴ radicals. The total number of aromatic ring atoms in Ar², Ar³ and Ar⁴ here is not more than 60 and preferably not more than 40.

Ar⁴ and Ar² here may also be bonded to one another and/or Ar² and Ar³ to one another by a group selected from C(R⁴)₂, NR⁴, O and S. Preferably, Ar⁴ and Ar² are joined to one another and Ar² and Ar³ to one another in the respective ortho position to the bond to the nitrogen atom. In a further embodiment of the invention, none of the Ar², Ar³ and Ar⁴ groups are bonded to one another.

Preferably, Ar⁴ is an aromatic or heteroaromatic ring system which has 6 to 24 aromatic ring atoms, preferably 6 to 12 aromatic ring atoms, and may be substituted in each case by one or more R⁴ radicals. More preferably, Ar⁴ is selected from the group consisting of ortho-, meta- or para-phenylene or ortho-, meta- or para-biphenyl, each of which may be substituted by one or more R⁴ radicals, but are preferably unsubstituted. Most preferably, Ar⁴ is an unsubstituted phenylene group.

Preferably, Ar² and Ar³ are the same or different at each instance and are an aromatic or heteroaromatic ring system which has 6 to 24 aromatic ring atoms and may be substituted in each case by one or more R⁴ radicals. Particularly preferred Ar² and Ar³ groups are the same or different at each instance and are selected from the group consisting of benzene, ortho-, meta- or para-biphenyl, ortho-, meta- or para-terphenyl or branched terphenyl, ortho-, meta- or para-quaterphenyl or branched quaterphenyl, 1-, 2-, 3- or 4-fluorenyl, 1-, 2-, 3- or 4-spirobifluorenyl, 1- or 2-naphthyl, indole, benzofuran, benzothiophene, 1-, 2-, 3- or 4-carbazole, 1-, 2-, 3- or 4-dibenzofuran, 1-, 2-, 3- or 4-dibenzothiophene, indenocarbazole, indolocarbazole, 2-, 3- or 4-pyridine, 2-, 4- or 5-pyrimidine, pyrazine, pyridazine, triazine, phenanthrene or triphenylene, each of which may be substituted by one or more R¹ radicals. Most preferably, Ar² and Ar³ are the same or different at each instance and are selected from the group consisting of benzene, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorene, especially 1-, 2-, 3- or 4-fluorene, or spirobifluorene, especially 1-, 2-, 3- or 4-spirobifluorene.

In a further preferred embodiment of the invention, R⁴ is the same or different at each instance and is selected from the group consisting of H, D, F, CN, a straight-chain alkyl group having 1 to 10 carbon atoms or a branched or cyclic alkyl group having 3 to 10 carbon atoms, where the alkyl group may be substituted in each case by one or more R² radicals, or an aromatic or heteroaromatic ring system which has 6 to 24 aromatic ring atoms and may be substituted in each case by one or more R⁵ radicals. In a particularly preferred embodiment of the invention, R⁴ is the same or different at each instance and is selected from the group consisting of H, a straight-chain alkyl group having 1 to 6 carbon atoms, especially having 1, 2, 3 or 4 carbon atoms, or a branched or cyclic alkyl group having 3 to 6 carbon atoms, where the alkyl group may be substituted by one or more R⁵ radicals, but is preferably unsubstituted, or an aromatic or heteroaromatic ring system which has 6 to 13 aromatic ring atoms and may be substituted in each case by one or more R⁵ radicals, but is preferably unsubstituted.

In a further preferred embodiment of the invention, R⁵ is the same or different at each instance and is H, an alkyl group having 1 to 4 carbon atoms or an aryl group having 6 to 10 carbon atoms, which may be substituted by an alkyl group having 1 to 4 carbon atoms, but is preferably unsubstituted.

At the same time, in compounds of the invention that are processed by vacuum evaporation, the alkyl groups preferably have not more than five carbon atoms, more preferably not more than 4 carbon atoms, most preferably not more than 1 carbon atom. For compounds that are processed from solution, suitable compounds are also those substituted by alkyl groups, especially branched alkyl groups, having up to 10 carbon atoms or those substituted by oligoarylene groups, for example ortho-, meta- or para-terphenyl or branched terphenyl or quaterphenyl groups.

It may further be the case that the compound comprises exactly two or exactly three structures of formula (I), (II-1) to (II-25), (III-1) to (III-50), (IV-1) to (IV-25) and/or (V-1) to (V-15), where preferably one of the aromatic or heteroaromatic ring systems that can be represented by at least one of the R¹, R², R³ groups or to which the R¹, R², R³ groups bind is shared by the two structures. It may additionally be the case that the compound comprises a connecting group via which the exactly two or three structures of formula (I), (II-1) to (II-25), (III-1) to (III-50), (IV-1) to (IV-25), and/or (V-1) to (V-15) are bonded to one another. These connecting groups are preferably derived from groups that are defined for the R¹, R², R³ groups, but where one or two hydrogen atoms should be replaced by bonding sites. In a further configuration, an inventive compound comprising structures of formula (I), (II-1) to (II-25), (III-1) to (III-50), (IV-1) to (IV-25), and/or (V-1) to (V-15) may be configured as an oligomer, polymer or dendrimer, where, in place of one hydrogen atom or one substituent, there are one or more bonds of the compounds to the polymer, oligomer or dendrimer.

When the compounds of the formula (I) or the preferred embodiments are used as matrix material for a phosphorescent emitter or in a layer directly adjoining a phosphorescent layer, it is further preferable when the compound does not contain any fused aryl or heteroaryl groups in which more than two six-membered rings are fused directly to one another. An exception to this is formed by phenanthrene and triphenylene, which, because of their high triplet energy, may be preferable in spite of the presence of fused aromatic six-membered rings.

In addition, it is a feature of preferred compounds of the invention that they are sublimable. These compounds generally have a molar mass of less than about 1200 g/mol.

The abovementioned preferred embodiments may be combined with one another as desired within the restrictions defined in claim 1. In a particularly preferred embodiment of the invention, the abovementioned preferences occur simultaneously.

Examples of preferred compounds according to the embodiments detailed above are the compounds detailed in the following table:

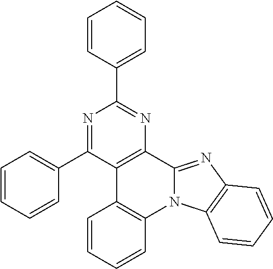 [SMILES: c1ccc(-c2nc(-c3ccccc3)c3c4ccccc4n4c5ccccc5nc4c3n2)cc1]

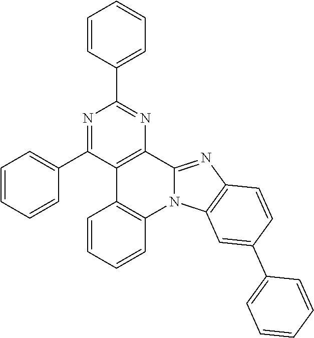 [SMILES: c1ccc(-c2ccc3nc4c5nc(-c6ccccc6)nc(-c6ccccc6)c5c5ccccc5n4c3c2)cc1]

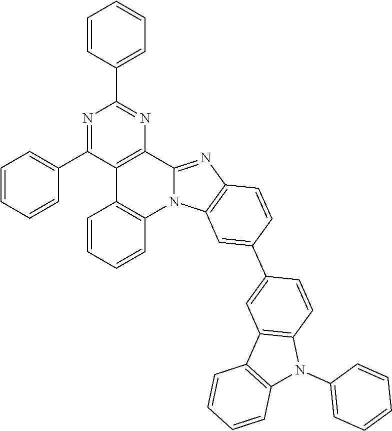 [SMILES: c1ccc(-c2nc(-c3ccccc3)c3c4ccccc4n4c5cc(-c6ccc7c(c6)c6ccccc6n7-c6ccccc6)ccc5nc4c3n2)cc1]

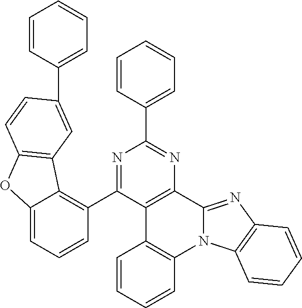 [SMILES: c1ccc(-c2ccc3oc4cccc(-c5nc(-c6ccccc6)nc6c5c5ccccc5n5c7ccccc7nc65)c4c3c2)cc1]

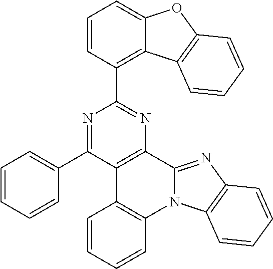 [SMILES: c1ccc(-c2nc(-c3cccc4oc5ccccc5c34)nc3c2c2ccccc2n2c4ccccc4nc32)cc1]

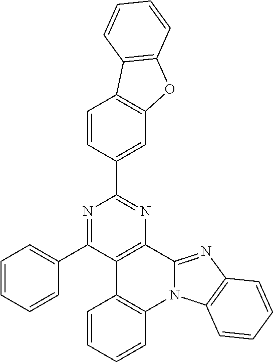 [SMILES: c1ccc(-c2nc(-c3ccc4c(c3)oc3ccccc34)nc3c2c2ccccc2n2c4ccccc4nc32)cc1]

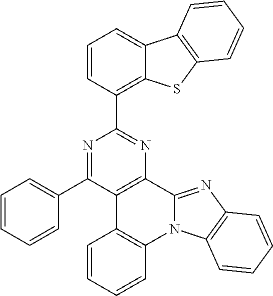 [SMILES: c1ccc(-c2nc(-c3cccc4c3sc3ccccc34)nc3c2c2ccccc2n2c4ccccc4nc32)cc1]

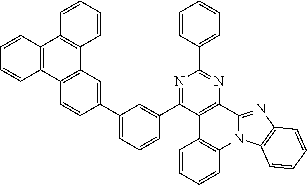 [SMILES: c1ccc(-c2nc(-c3cccc(-c4ccc5c6ccccc6c6ccccc6c5c4)c3)c3c4ccccc4n4c5ccccc5nc4c3n2)cc1]

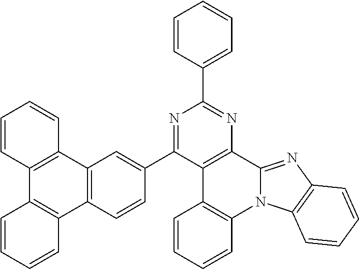 [SMILES: c1ccc(-c2nc(-c3ccc4c5ccccc5c5ccccc5c4c3)c3c4ccccc4n4c5ccccc5nc4c3n2)cc1]

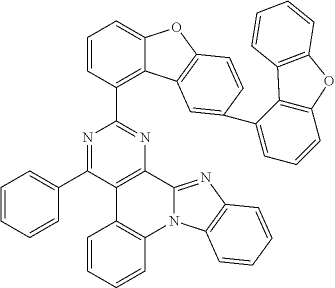 [SMILES: c1ccc(-c2nc(-c3cccc4oc5ccc(-c6cccc7oc8ccccc8c67)cc5c34)nc3c2c2ccccc2n2c4ccccc4nc32)cc1]

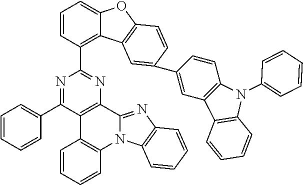 [SMILES: c1ccc(-c2nc(-c3cccc4oc5ccc(-c6ccc7c(c6)c6ccccc6n7-c6ccccc6)cc5c34)nc3c2c2ccccc2n2c4ccccc4nc32)cc1]

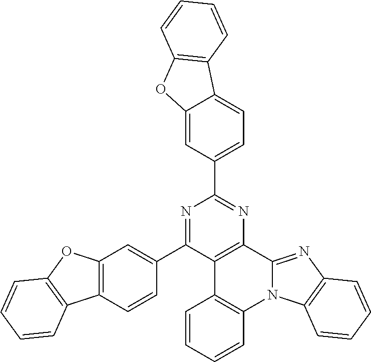 [SMILES: c1ccc2c(c1)nc1c3nc(-c4ccc5c(c4)oc4ccccc45)nc(-c4ccc5c(c4)oc4ccccc45)c3c3ccccc3n21]

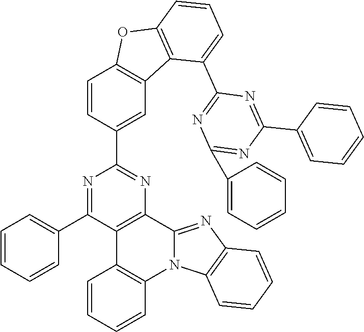 [SMILES: c1ccc(-c2nc(-c3ccccc3)nc(-c3cccc4oc5ccc(-c6nc(-c7ccccc7)c7c8ccccc8n8c9ccccc9nc8c7n6)cc5c34)n2)cc1]

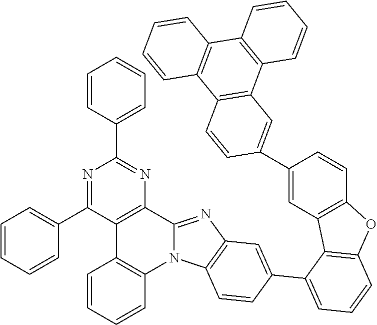 [SMILES: c1ccc(-c2nc(-c3ccccc3)c3c4ccccc4n4c5ccc(-c6cccc7oc8ccc(-c9ccc%10c%11ccccc%11c%11ccccc%11c%10c9)cc8c67)cc5nc4c3n2)cc1]

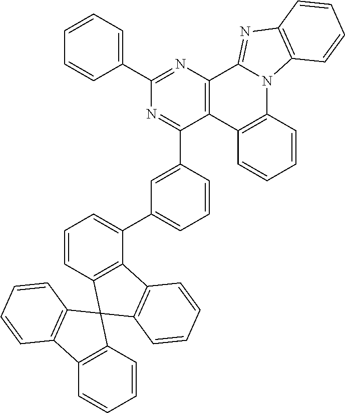 [SMILES: c1ccc(-c2nc(-c3cccc(-c4cccc5c4-c4ccccc4C54c5ccccc5-c5ccccc54)c3)c3c4ccccc4n4c5ccccc5nc4c3n2)cc1]

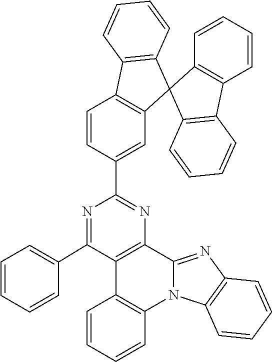 [SMILES: c1ccc(-c2nc(-c3ccc4c(c3)C3(c5ccccc5-c5ccccc53)c3ccccc3-4)nc3c2c2ccccc2n2c4ccccc4nc32)cc1]

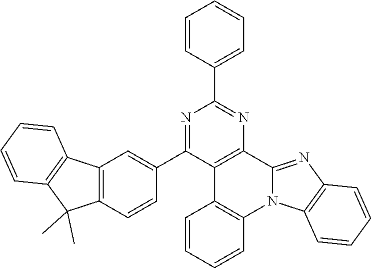 [SMILES: CC1(C)c2ccccc2-c2cc(-c3nc(-c4ccccc4)nc4c3c3ccccc3n3c5ccccc5nc43)ccc21]

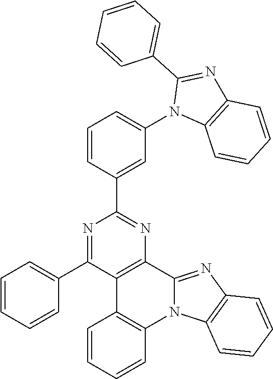 [SMILES: c1ccc(-c2nc(-c3cccc(-n4c(-c5ccccc5)nc5ccccc54)c3)nc3c2c2ccccc2n2c4ccccc4nc32)cc1]

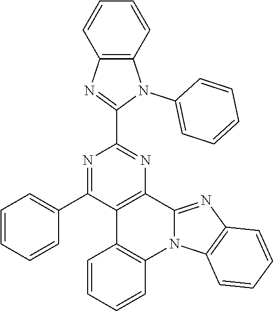 [SMILES: c1ccc(-c2nc(-c3nc4ccccc4n3-c3ccccc3)nc3c2c2ccccc2n2c4ccccc4nc32)cc1]

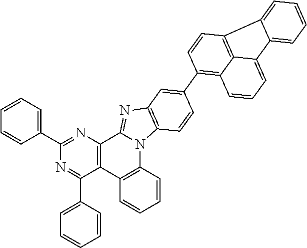 [SMILES: c1ccc(-c2nc(-c3ccccc3)c3c4ccccc4n4c5ccc(-c6ccc7c8c(cccc68)-c6ccccc6-7)cc5nc4c3n2)cc1]

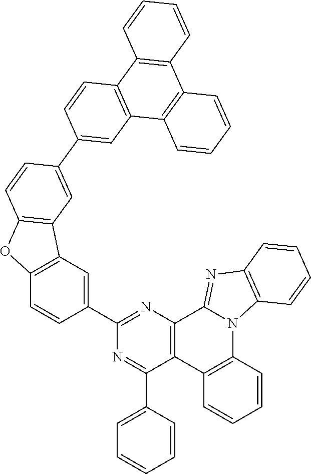 [SMILES: c1ccc(-c2nc(-c3ccc4oc5ccc(-c6ccc7c8ccccc8c8ccccc8c7c6)cc5c4c3)nc3c2c2ccccc2n2c4ccccc4nc32)cc1]

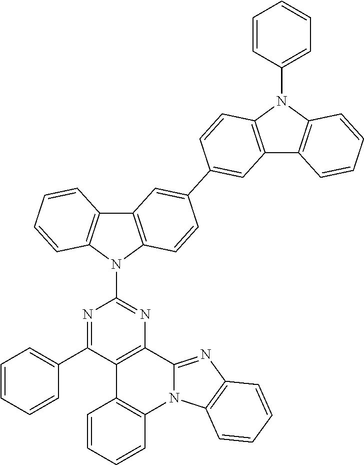 [SMILES: c1ccc(-c2nc(-n3c4ccccc4c4cc(-c5ccc6c(c5)c5ccccc5n6-c5ccccc5)ccc43)nc3c2c2ccccc2n2c4ccccc4nc32)cc1]

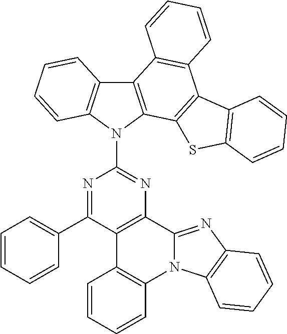 [SMILES: c1ccc(-c2nc(-n3c4ccccc4c4c5ccccc5c5c6ccccc6sc5c43)nc3c2c2ccccc2n2c4ccccc4nc32)cc1]

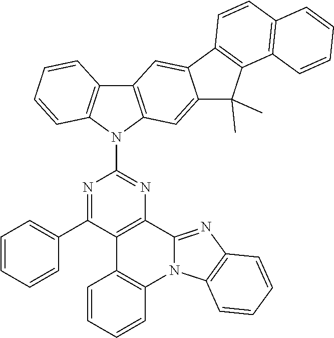 [SMILES: CC1(C)c2cc3c(cc2-c2ccc4ccccc4c21)c1ccccc1n3-c1nc(-c2ccccc2)c2c3ccccc3n3c4ccccc4nc3c2n1]

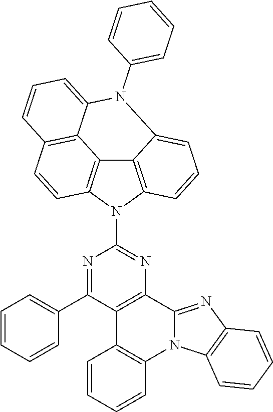 [SMILES: c1ccc(-c2nc(-n3c4cccc5c4c4c6c(cccc6ccc43)N5c3ccccc3)nc3c2c2ccccc2n2c4ccccc4nc32)cc1]

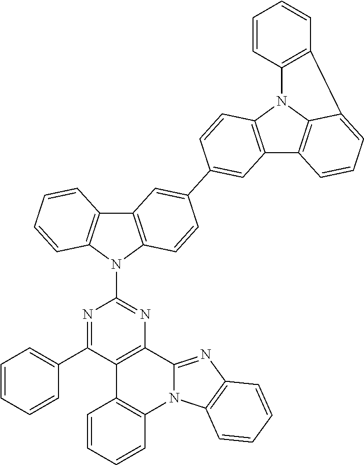 [SMILES: c1ccc(-c2nc(-n3c4ccccc4c4cc(-c5ccc6c(c5)c5cccc7c8ccccc8n6c75)ccc43)nc3c2c2ccccc2n2c4ccccc4nc32)cc1]

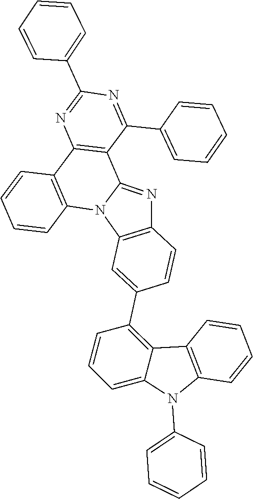 [SMILES: c1ccc(-c2nc(-c3ccccc3)c3c(n2)c2ccccc2n2c4cc(-c5cccc6c5c5ccccc5n6-c5ccccc5)ccc4nc32)cc1]

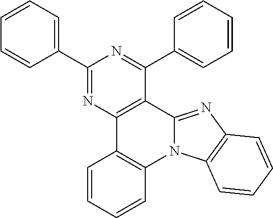 [SMILES: c1ccc(-c2nc(-c3ccccc3)c3c(n2)c2ccccc2n2c4ccccc4nc32)cc1]

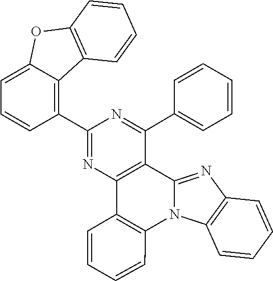 [SMILES: c1ccc(-c2nc(-c3cccc4oc5ccccc5c34)nc3c4ccccc4n4c5ccccc5nc4c23)cc1]

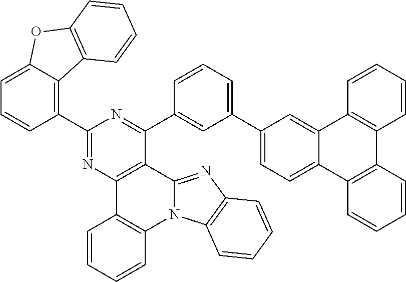 [SMILES: c1cc(-c2ccc3c4ccccc4c4ccccc4c3c2)cc(-c2nc(-c3cccc4oc5ccccc5c34)nc3c4ccccc4n4c5ccccc5nc4c23)c1]

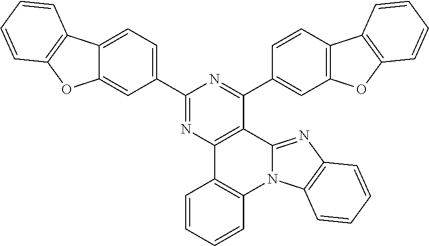 [SMILES: c1ccc2c(c1)nc1c3c(-c4ccc5c(c4)oc4ccccc45)nc(-c4ccc5c(c4)oc4ccccc45)nc3c3ccccc3n21]

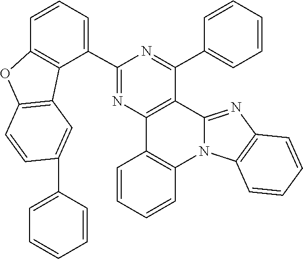 [SMILES: c1ccc(-c2ccc3oc4cccc(-c5nc(-c6ccccc6)c6c(n5)c5ccccc5n5c7ccccc7nc65)c4c3c2)cc1]

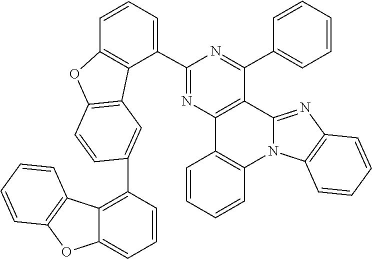 [SMILES: c1ccc(-c2nc(-c3cccc4oc5ccc(-c6cccc7oc8ccccc8c67)cc5c34)nc3c4ccccc4n4c5ccccc5nc4c23)cc1]

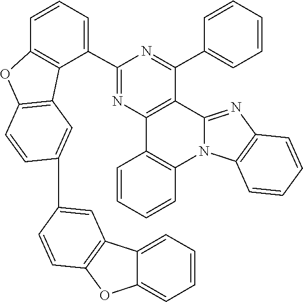 [SMILES: c1ccc(-c2nc(-c3cccc4oc5ccc(-c6ccc7oc8ccccc8c7c6)cc5c34)nc3c4ccccc4n4c5ccccc5nc4c23)cc1]

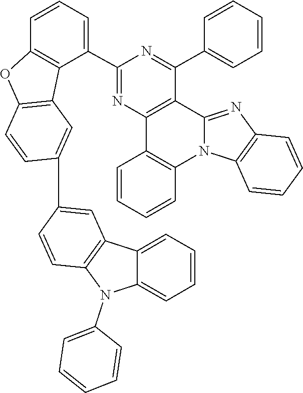 [SMILES: c1ccc(-c2nc(-c3cccc4oc5ccc(-c6ccc7c(c6)c6ccccc6n7-c6ccccc6)cc5c34)nc3c4ccccc4n4c5ccccc5nc4c23)cc1]

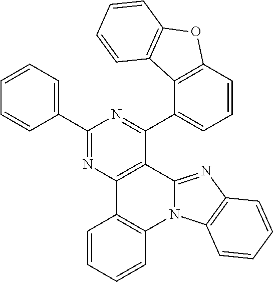 [SMILES: c1ccc(-c2nc(-c3cccc4oc5ccccc5c34)c3c(n2)c2ccccc2n2c4ccccc4nc32)cc1]

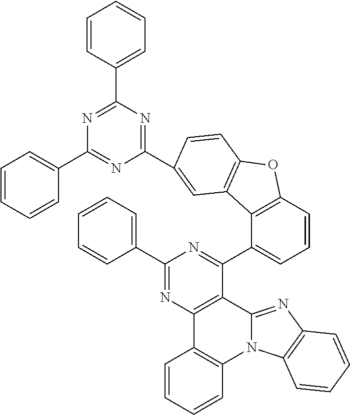 [SMILES: c1ccc(-c2nc(-c3ccccc3)nc(-c3ccc4oc5cccc(-c6nc(-c7ccccc7)nc7c8ccccc8n8c9ccccc9nc8c67)c5c4c3)n2)cc1]

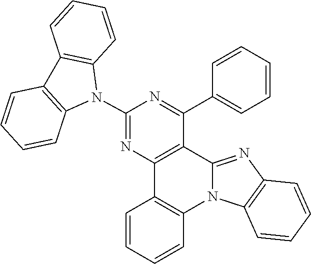 [SMILES: c1ccc(-c2nc(-n3c4ccccc4c4ccccc43)nc3c4ccccc4n4c5ccccc5nc4c23)cc1]

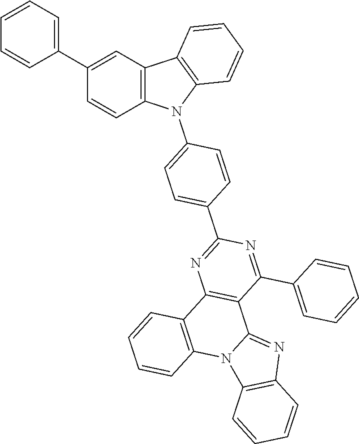 [SMILES: c1ccc(-c2ccc3c(c2)c2ccccc2n3-c2ccc(-c3nc(-c4ccccc4)c4c(n3)c3ccccc3n3c5ccccc5nc43)cc2)cc1]

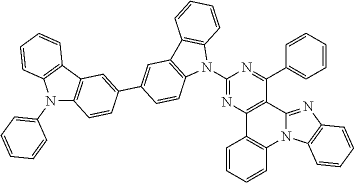 [SMILES: c1ccc(-c2nc(-n3c4ccccc4c4cc(-c5ccc6c(c5)c5ccccc5n6-c5ccccc5)ccc43)nc3c4ccccc4n4c5ccccc5nc4c23)cc1]

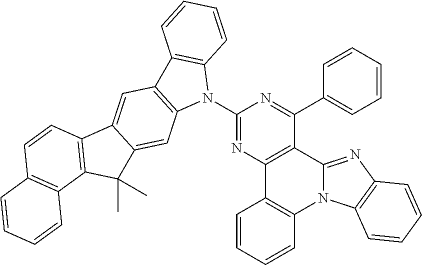 [SMILES: CC1(C)c2cc3c(cc2-c2ccc4ccccc4c21)c1ccccc1n3-c1nc(-c2ccccc2)c2c(n1)c1ccccc1n1c3ccccc3nc21]

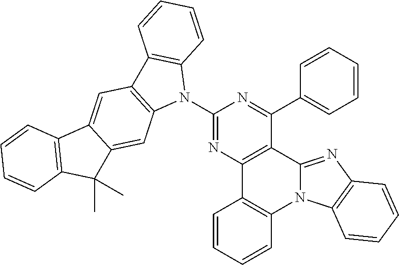 [SMILES: CC1(C)c2ccccc2-c2cc3c4ccccc4n(-c4nc(-c5ccccc5)c5c(n4)c4ccccc4n4c6ccccc6nc54)c3cc21]

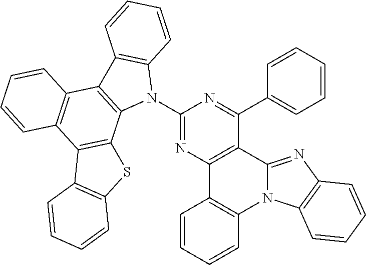 [SMILES: c1ccc(-c2nc(-n3c4ccccc4c4c5ccccc5c5c6ccccc6sc5c43)nc3c4ccccc4n4c5ccccc5nc4c23)cc1]

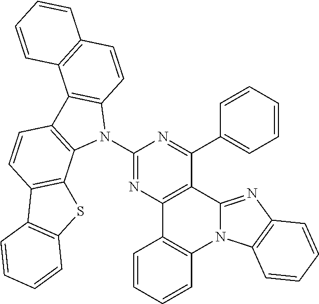 [SMILES: c1ccc(-c2nc(-n3c4ccc5ccccc5c4c4ccc5c6ccccc6sc5c43)nc3c4ccccc4n4c5ccccc5nc4c23)cc1]

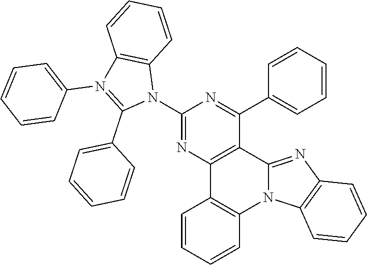

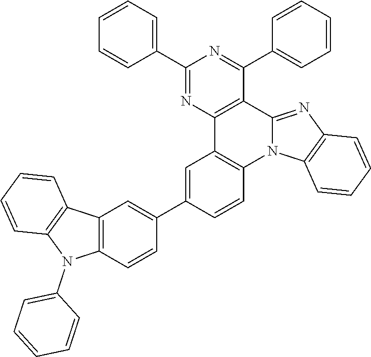 [SMILES: c1ccc(-c2nc(-c3ccccc3)c3c(n2)c2cc(-c4ccc5c(c4)c4ccccc4n5-c4ccccc4)ccc2n2c4ccccc4nc32)cc1]

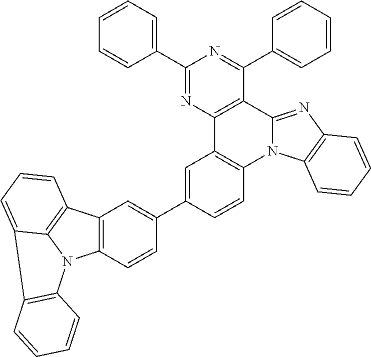 [SMILES: c1ccc(-c2nc(-c3ccccc3)c3c(n2)c2cc(-c4ccc5c(c4)c4cccc6c7ccccc7n5c64)ccc2n2c4ccccc4nc32)cc1]

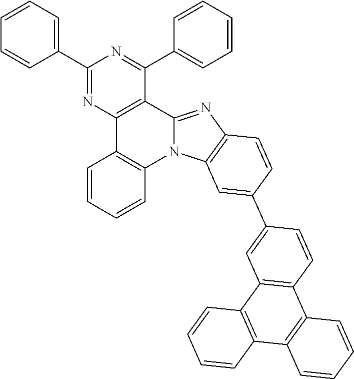 [SMILES: c1ccc(-c2nc(-c3ccccc3)c3c(n2)c2ccccc2n2c4cc(-c5ccc6c7ccccc7c7ccccc7c6c5)ccc4nc32)cc1]

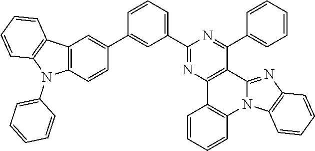 [SMILES: c1ccc(-c2nc(-c3cccc(-c4ccc5c(c4)c4ccccc4n5-c4ccccc4)c3)nc3c4ccccc4n4c5ccccc5nc4c23)cc1]

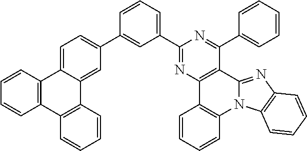 [SMILES: c1ccc(-c2nc(-c3cccc(-c4ccc5c6ccccc6c6ccccc6c5c4)c3)nc3c4ccccc4n4c5ccccc5nc4c23)cc1]

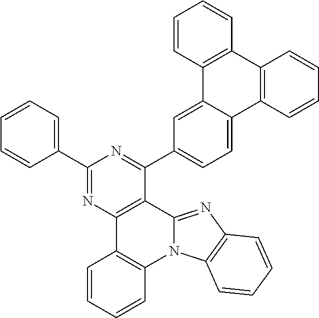 [SMILES: c1ccc(-c2nc(-c3ccc4c5ccccc5c5ccccc5c4c3)c3c(n2)c2ccccc2n2c4ccccc4nc32)cc1]

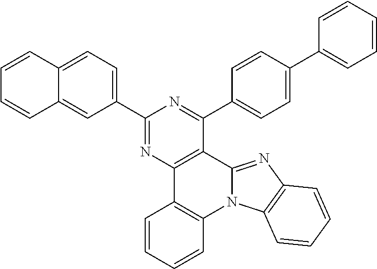 [SMILES: c1ccc(-c2ccc(-c3nc(-c4ccc5ccccc5c4)nc4c5ccccc5n5c6ccccc6nc5c34)cc2)cc1]

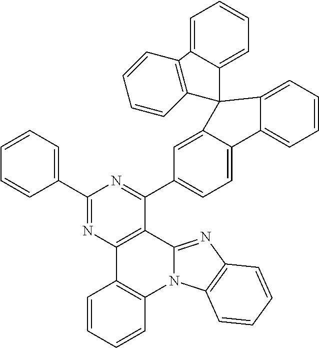 [SMILES: c1ccc(-c2nc(-c3ccc4c(c3)C3(c5ccccc5-c5ccccc53)c3ccccc3-4)c3c(n2)c2ccccc2n2c4ccccc4nc32)cc1]

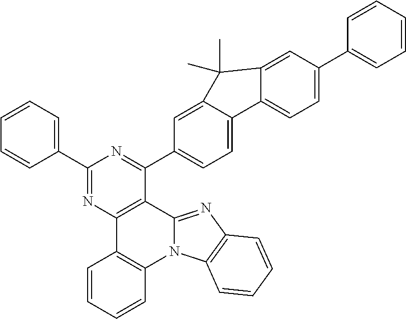 [SMILES: CC1(C)c2cc(-c3ccccc3)ccc2-c2ccc(-c3nc(-c4ccccc4)nc4c5ccccc5n5c6ccccc6nc5c34)cc21]

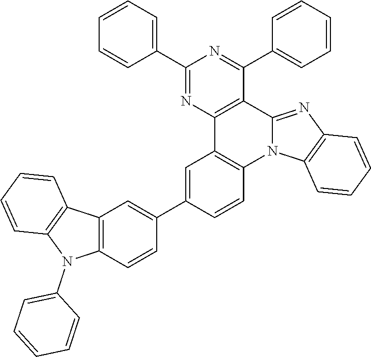 [SMILES: c1ccc(-c2nc(-c3ccccc3)c3c(n2)c2cc(-c4ccc5c(c4)c4ccccc4n5-c4ccccc4)ccc2n2c4ccccc4nc32)cc1]

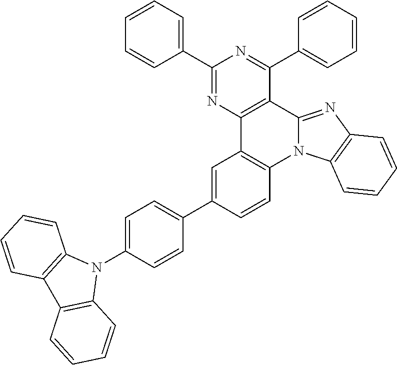 [SMILES: c1ccc(-c2nc(-c3ccccc3)c3c(n2)c2cc(-c4ccc(-n5c6ccccc6c6ccccc65)cc4)ccc2n2c4ccccc4nc32)cc1]

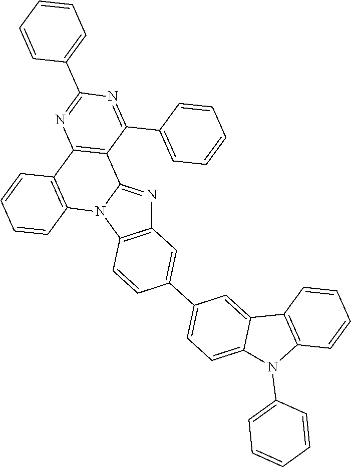 [SMILES: c1ccc(-c2nc(-c3ccccc3)c3c(n2)c2ccccc2n2c4ccc(-c5ccc6c(c5)c5ccccc5n6-c5ccccc5)cc4nc32)cc1]

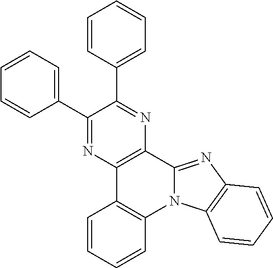 [SMILES: c1ccc(-c2nc3c4ccccc4n4c5ccccc5nc4c3nc2-c2ccccc2)cc1]

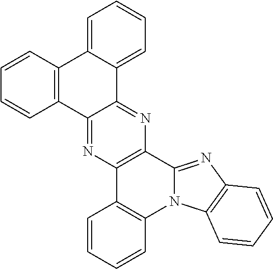 [SMILES: c1ccc2c(c1)nc1c3nc4c5ccccc5c5ccccc5c4nc3c3ccccc3n21]

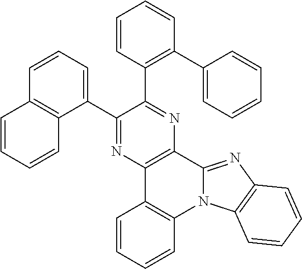 [SMILES: c1ccc(-c2ccccc2-c2nc3c(nc2-c2cccc4ccccc24)c2ccccc2n2c4ccccc4nc32)cc1]

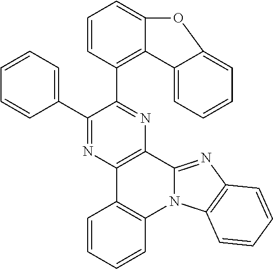 [SMILES: c1ccc(-c2nc3c4ccccc4n4c5ccccc5nc4c3nc2-c2cccc3oc4ccccc4c23)cc1]

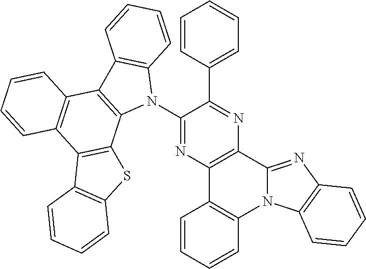 [SMILES: c1ccc(-c2nc3c(nc2-n2c4ccccc4c4c5ccccc5c5c6ccccc6sc5c42)c2ccccc2n2c4ccccc4nc32)cc1]

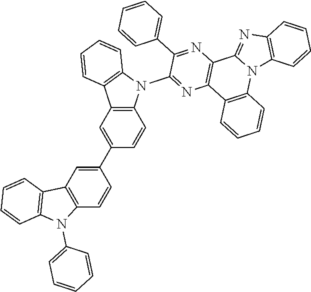 [SMILES: c1ccc(-c2nc3c(nc2-n2c4ccccc4c4cc(-c5ccc6c(c5)c5ccccc5n6-c5ccccc5)ccc42)c2ccccc2n2c4ccccc4nc32)cc1]

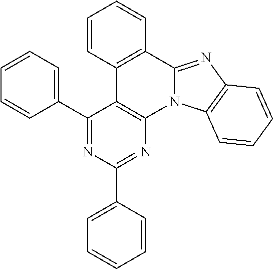 [SMILES: c1ccc(-c2nc(-c3ccccc3)c3c4ccccc4c4nc5ccccc5n4c3n2)cc1]

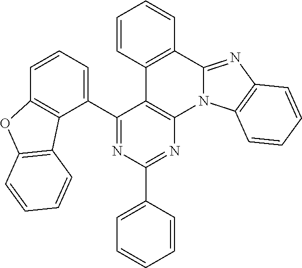 [SMILES: c1ccc(-c2nc(-c3cccc4oc5ccccc5c34)c3c4ccccc4c4nc5ccccc5n4c3n2)cc1]

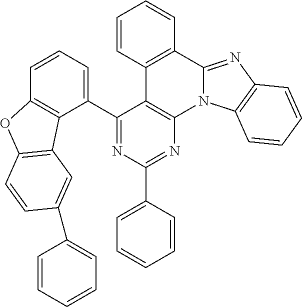 [SMILES: c1ccc(-c2ccc3oc4cccc(-c5nc(-c6ccccc6)nc6c5c5ccccc5c5nc7ccccc7n56)c4c3c2)cc1]

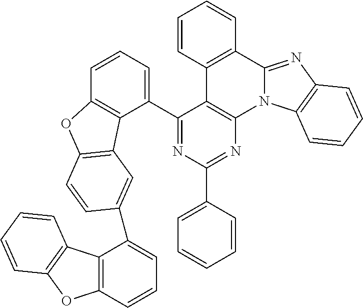 [SMILES: c1ccc(-c2nc(-c3cccc4oc5ccc(-c6cccc7oc8ccccc8c67)cc5c34)c3c4ccccc4c4nc5ccccc5n4c3n2)cc1]

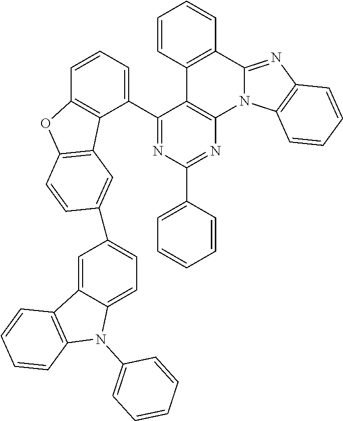 [SMILES: c1ccc(-c2nc(-c3cccc4oc5ccc(-c6ccc7c(c6)c6ccccc6n7-c6ccccc6)cc5c34)c3c4ccccc4c4nc5ccccc5n4c3n2)cc1]

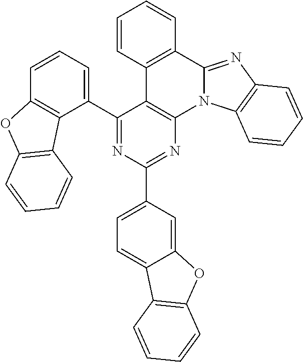 [SMILES: c1ccc2c(c1)nc1c3ccccc3c3c(-c4cccc5oc6ccccc6c45)nc(-c4ccc5c(c4)oc4ccccc45)nc3n21]

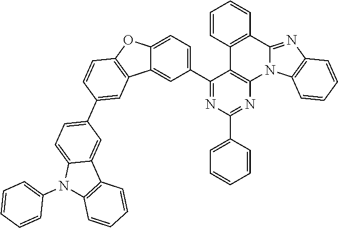 [SMILES: c1ccc(-c2nc(-c3ccc4oc5ccc(-c6ccc7c(c6)c6ccccc6n7-c6ccccc6)cc5c4c3)c3c4ccccc4c4nc5ccccc5n4c3n2)cc1]

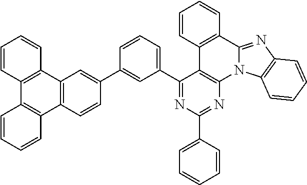 [SMILES: c1ccc(-c2nc(-c3cccc(-c4ccc5c6ccccc6c6ccccc6c5c4)c3)c3c4ccccc4c4nc5ccccc5n4c3n2)cc1]

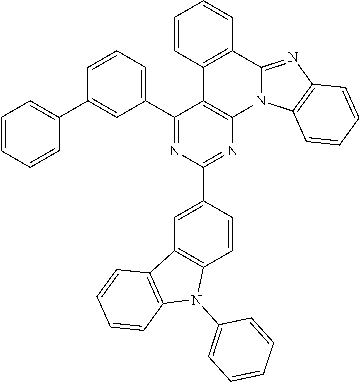 [SMILES: c1ccc(-c2cccc(-c3nc(-c4ccc5c(c4)c4ccccc4n5-c4ccccc4)nc4c3c3ccccc3c3nc5ccccc5n34)c2)cc1]

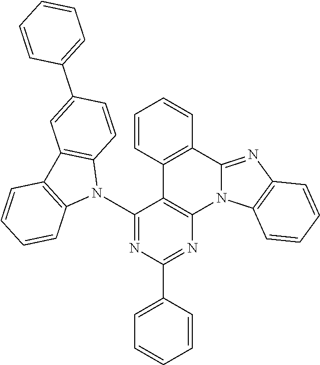 [SMILES: c1ccc(-c2ccc3c(c2)c2ccccc2n3-c2nc(-c3ccccc3)nc3c2c2ccccc2c2nc4ccccc4n23)cc1]

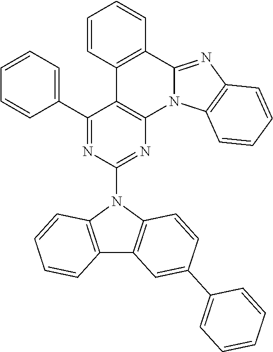 [SMILES: c1ccc(-c2ccc3c(c2)c2ccccc2n3-c2nc(-c3ccccc3)c3c4ccccc4c4nc5ccccc5n4c3n2)cc1]

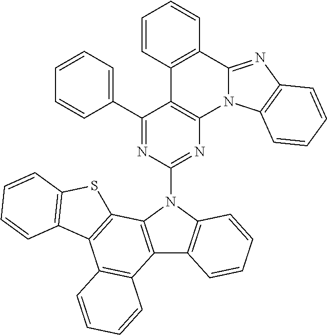 [SMILES: c1ccc(-c2nc(-n3c4ccccc4c4c5ccccc5c5c6ccccc6sc5c43)nc3c2c2ccccc2c2nc4ccccc4n23)cc1]

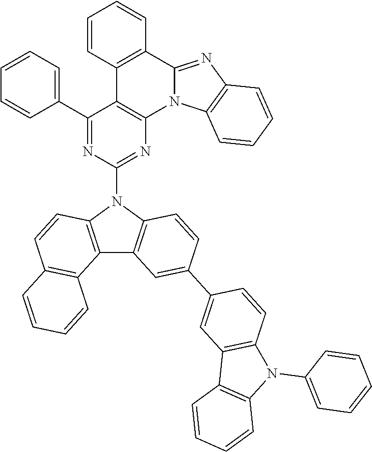 [SMILES: c1ccc(-c2nc(-n3c4ccc(-c5ccc6c(c5)c5ccccc5n6-c5ccccc5)cc4c4c5ccccc5ccc43)nc3c2c2ccccc2c2nc4ccccc4n23)cc1]

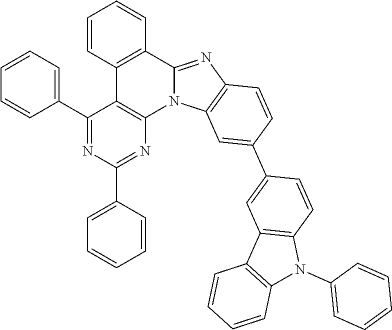 [SMILES: c1ccc(-c2nc(-c3ccccc3)c3c4ccccc4c4nc5ccc(-c6ccc7c(c6)c6ccccc6n7-c6ccccc6)cc5n4c3n2)cc1]

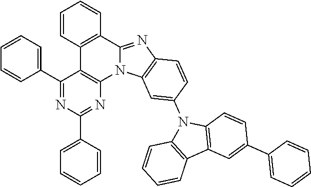 [SMILES: c1ccc(-c2ccc3c(c2)c2ccccc2n3-c2ccc3nc4c5ccccc5c5c(-c6ccccc6)nc(-c6ccccc6)nc5n4c3c2)cc1]

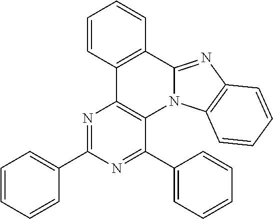 [SMILES: c1ccc(-c2nc(-c3ccccc3)c3c(n2)c2ccccc2c2nc4ccccc4n23)cc1]

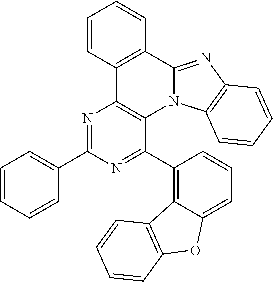 [SMILES: c1ccc(-c2nc(-c3cccc4oc5ccccc5c34)c3c(n2)c2ccccc2c2nc4ccccc4n23)cc1]

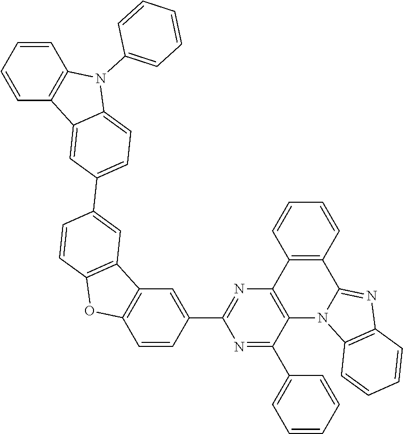 [SMILES: c1ccc(-c2nc(-c3ccc4oc5ccc(-c6ccc7c(c6)c6ccccc6n7-c6ccccc6)cc5c4c3)nc3c4ccccc4c4nc5ccccc5n4c23)cc1]

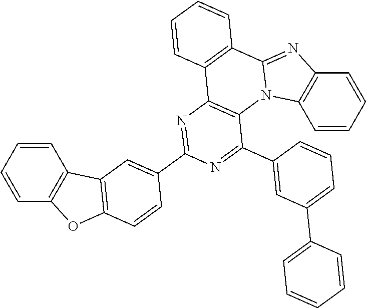 [SMILES: c1ccc(-c2cccc(-c3nc(-c4ccc5oc6ccccc6c5c4)nc4c5ccccc5c5nc6ccccc6n5c34)c2)cc1]

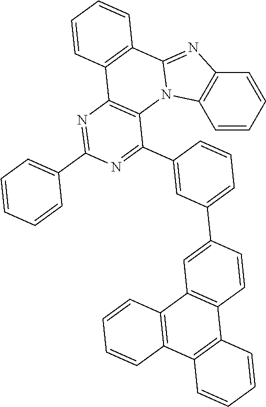 [SMILES: c1ccc(-c2nc(-c3cccc(-c4ccc5c6ccccc6c6ccccc6c5c4)c3)c3c(n2)c2ccccc2c2nc4ccccc4n23)cc1]

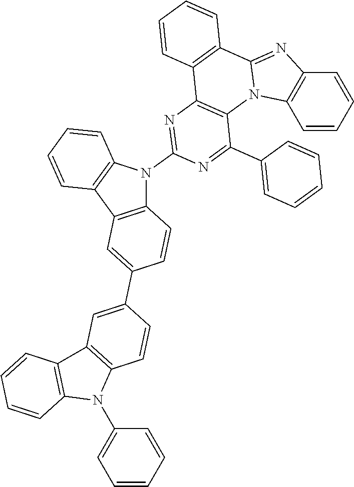 [SMILES: c1ccc(-c2nc(-n3c4ccccc4c4cc(-c5ccc6c(c5)c5ccccc5n6-c5ccccc5)ccc43)nc3c4ccccc4c4nc5ccccc5n4c23)cc1]

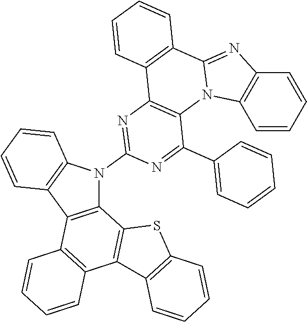 [SMILES: c1ccc(-c2nc(-n3c4ccccc4c4c5ccccc5c5c6ccccc6sc5c43)nc3c4ccccc4c4nc5ccccc5n4c23)cc1]

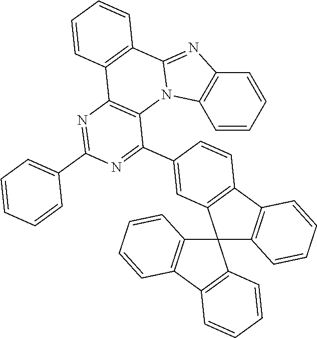 [SMILES: c1ccc(-c2nc(-c3ccc4c(c3)C3(c5ccccc5-c5ccccc53)c3ccccc3-4)c3c(n2)c2ccccc2c2nc4ccccc4n23)cc1]

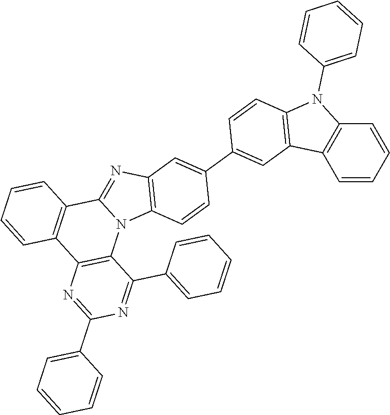 [SMILES: c1ccc(-c2nc(-c3ccccc3)c3c(n2)c2ccccc2c2nc4cc(-c5ccc6c(c5)c5ccccc5n6-c5ccccc5)ccc4n23)cc1]

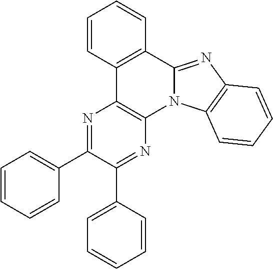 [SMILES: c1ccc(-c2nc3c4ccccc4c4nc5ccccc5n4c3nc2-c2ccccc2)cc1]

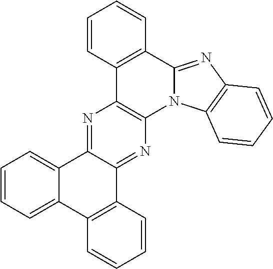 [SMILES: c1ccc2c(c1)nc1c3ccccc3c3nc4c5ccccc5c5ccccc5c4nc3n21]

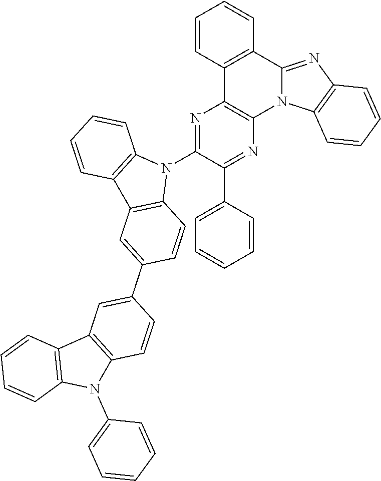 [SMILES: c1ccc(-c2nc3c(nc2-n2c4ccccc4c4cc(-c5ccc6c(c5)c5ccccc5n6-c5ccccc5)ccc42)c2ccccc2c2nc4ccccc4n23)cc1]

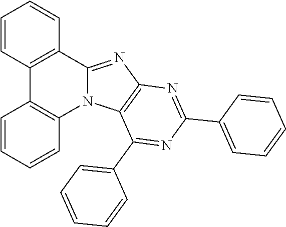 [SMILES: c1ccc(-c2nc(-c3ccccc3)c3c(n2)nc2c4ccccc4c4ccccc4n23)cc1]

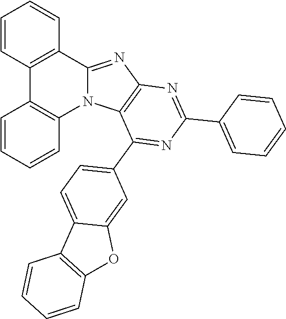 [SMILES: c1ccc(-c2nc(-c3ccc4c(c3)oc3ccccc34)c3c(n2)nc2c4ccccc4c4ccccc4n23)cc1]

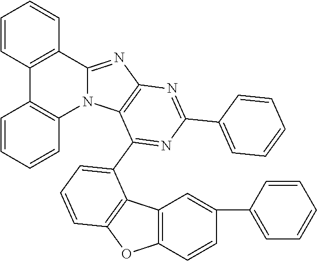 [SMILES: c1ccc(-c2ccc3oc4cccc(-c5nc(-c6ccccc6)nc6nc7c8ccccc8c8ccccc8n7c56)c4c3c2)cc1]

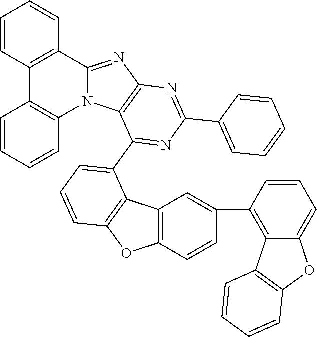 [SMILES: c1ccc(-c2nc(-c3cccc4oc5ccc(-c6cccc7oc8ccccc8c67)cc5c34)c3c(n2)nc2c4ccccc4c4ccccc4n23)cc1]

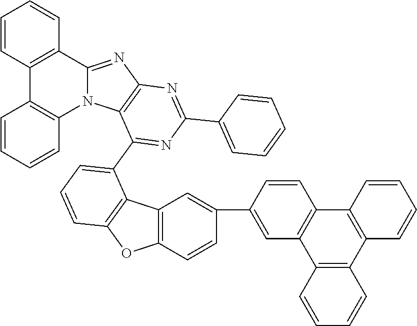 [SMILES: c1ccc(-c2nc(-c3cccc4oc5ccc(-c6ccc7c8ccccc8c8ccccc8c7c6)cc5c34)c3c(n2)nc2c4ccccc4c4ccccc4n23)cc1]

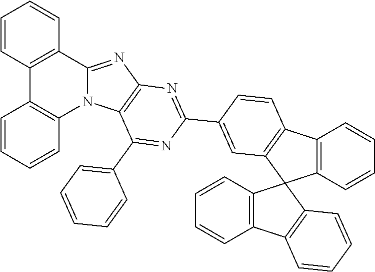 [SMILES: c1ccc(-c2nc(-c3ccc4c(c3)C3(c5ccccc5-c5ccccc53)c3ccccc3-4)nc3nc4c5ccccc5c5ccccc5n4c23)cc1]

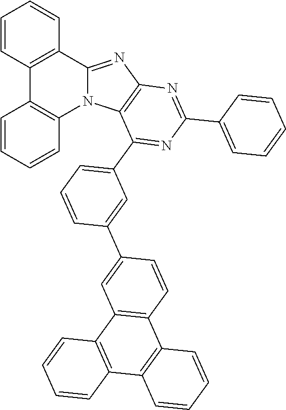 [SMILES: c1ccc(-c2nc(-c3cccc(-c4ccc5c6ccccc6c6ccccc6c5c4)c3)c3c(n2)nc2c4ccccc4c4ccccc4n23)cc1]

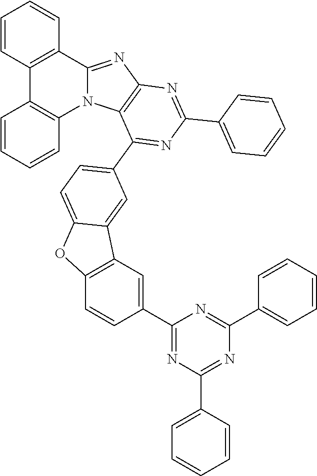 [SMILES: c1ccc(-c2nc(-c3ccccc3)nc(-c3ccc4oc5ccc(-c6nc(-c7ccccc7)nc7nc8c9ccccc9c9ccccc9n8c67)cc5c4c3)n2)cc1]

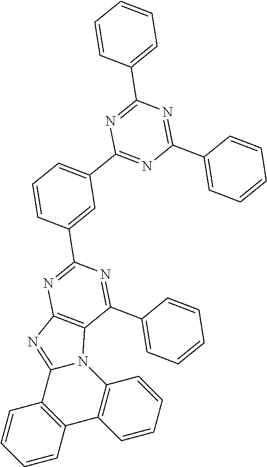 [SMILES: c1ccc(-c2nc(-c3ccccc3)nc(-c3cccc(-c4nc(-c5ccccc5)c5c(n4)nc4c6ccccc6c6ccccc6n45)c3)n2)cc1]

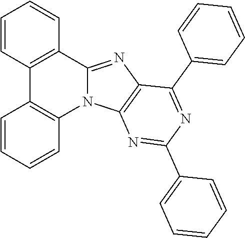 [SMILES: c1ccc(-c2nc(-c3ccccc3)c3nc4c5ccccc5c5ccccc5n4c3n2)cc1]

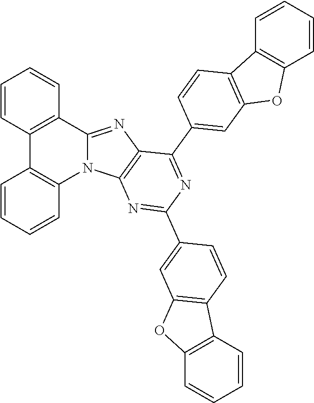 [SMILES: c1ccc2c(c1)oc1cc(-c3nc(-c4ccc5c(c4)oc4ccccc45)c4nc5c6ccccc6c6ccccc6n5c4n3)ccc12]

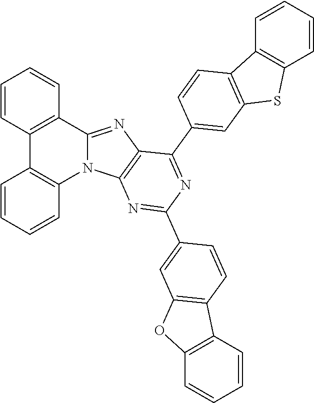 [SMILES: c1ccc2c(c1)oc1cc(-c3nc(-c4ccc5c(c4)sc4ccccc45)c4nc5c6ccccc6c6ccccc6n5c4n3)ccc12]

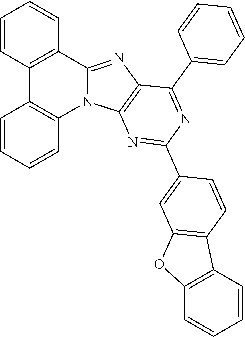 [SMILES: c1ccc(-c2nc(-c3ccc4c(c3)oc3ccccc34)nc3c2nc2c4ccccc4c4ccccc4n32)cc1]

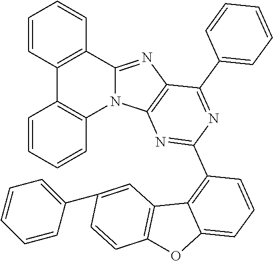 [SMILES: c1ccc(-c2ccc3oc4cccc(-c5nc(-c6ccccc6)c6nc7c8ccccc8c8ccccc8n7c6n5)c4c3c2)cc1]

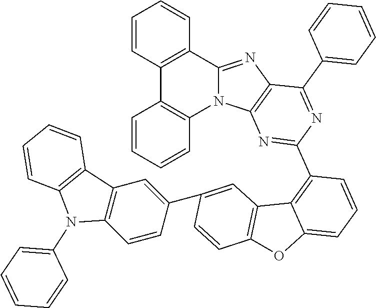 [SMILES: c1ccc(-c2nc(-c3cccc4oc5ccc(-c6ccc7c(c6)c6ccccc6n7-c6ccccc6)cc5c34)nc3c2nc2c4ccccc4c4ccccc4n32)cc1]

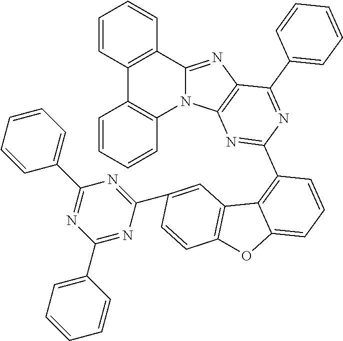 [SMILES: c1ccc(-c2nc(-c3ccccc3)nc(-c3ccc4oc5cccc(-c6nc(-c7ccccc7)c7nc8c9ccccc9c9ccccc9n8c7n6)c5c4c3)n2)cc1]

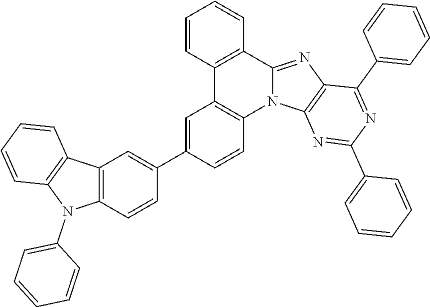 [SMILES: c1ccc(-c2nc(-c3ccccc3)c3nc4c5ccccc5c5cc(-c6ccc7c(c6)c6ccccc6n7-c6ccccc6)ccc5n4c3n2)cc1]

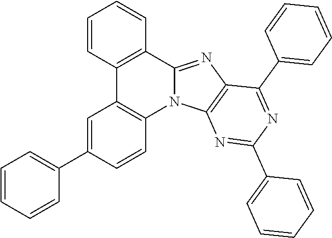 [SMILES: c1ccc(-c2ccc3c(c2)c2ccccc2c2nc4c(-c5ccccc5)nc(-c5ccccc5)nc4n32)cc1]

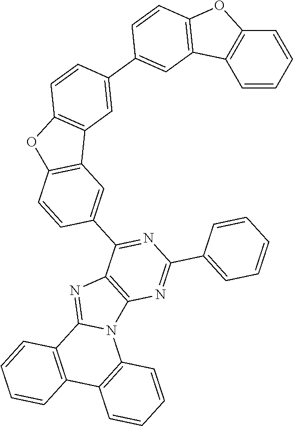 [SMILES: c1ccc(-c2nc(-c3ccc4oc5ccc(-c6ccc7oc8ccccc8c7c6)cc5c4c3)c3nc4c5ccccc5c5ccccc5n4c3n2)cc1]

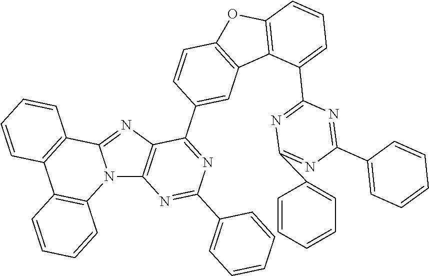 [SMILES: c1ccc(-c2nc(-c3ccccc3)nc(-c3cccc4oc5ccc(-c6nc(-c7ccccc7)nc7c6nc6c8ccccc8c8ccccc8n76)cc5c34)n2)cc1]

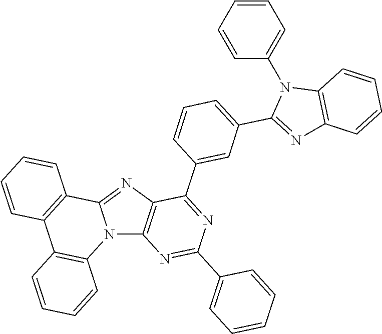 [SMILES: c1ccc(-c2nc(-c3cccc(-c4nc5ccccc5n4-c4ccccc4)c3)c3nc4c5ccccc5c5ccccc5n4c3n2)cc1]

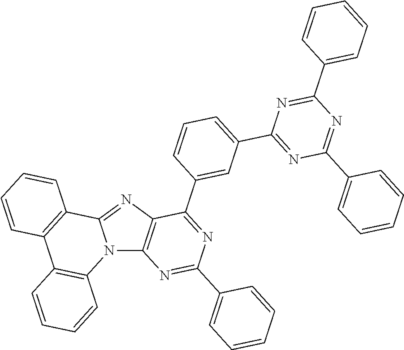 [SMILES: c1ccc(-c2nc(-c3ccccc3)nc(-c3cccc(-c4nc(-c5ccccc5)nc5c4nc4c6ccccc6c6ccccc6n54)c3)n2)cc1]

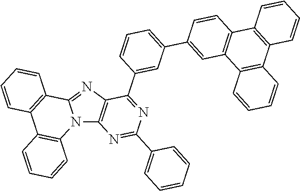 [SMILES: c1ccc(-c2nc(-c3cccc(-c4ccc5c6ccccc6c6ccccc6c5c4)c3)c3nc4c5ccccc5c5ccccc5n4c3n2)cc1]

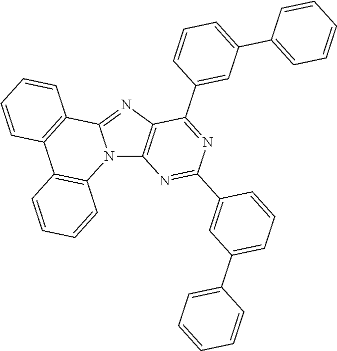 [SMILES: c1ccc(-c2cccc(-c3nc(-c4cccc(-c5ccccc5)c4)c4nc5c6ccccc6c6ccccc6n5c4n3)c2)cc1]

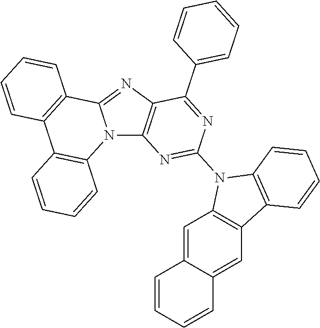 [SMILES: c1ccc(-c2nc(-n3c4ccccc4c4cc5ccccc5cc43)nc3c2nc2c4ccccc4c4ccccc4n32)cc1]

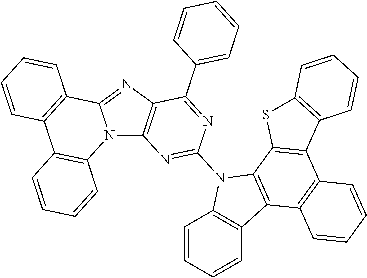 [SMILES: c1ccc(-c2nc(-n3c4ccccc4c4c5ccccc5c5c6ccccc6sc5c43)nc3c2nc2c4ccccc4c4ccccc4n32)cc1]

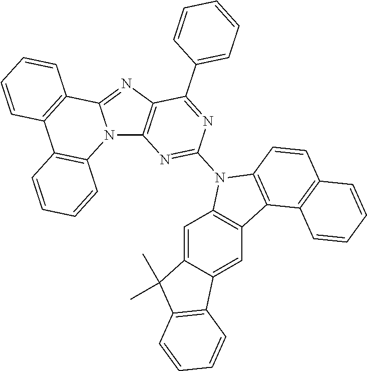 [SMILES: CC1(C)c2ccccc2-c2cc3c4c5ccccc5ccc4n(-c4nc(-c5ccccc5)c5nc6c7ccccc7c7ccccc7n6c5n4)c3cc21]

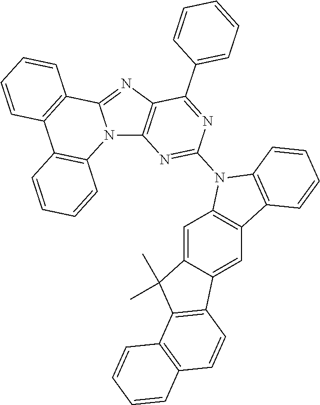 [SMILES: CC1(C)c2cc3c(cc2-c2ccc4ccccc4c21)c1ccccc1n3-c1nc(-c2ccccc2)c2nc3c4ccccc4c4ccccc4n3c2n1]

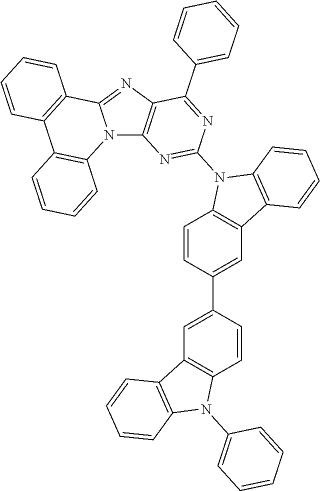 [SMILES: c1ccc(-c2nc(-n3c4ccccc4c4cc(-c5ccc6c(c5)c5ccccc5n6-c5ccccc5)ccc43)nc3c2nc2c4ccccc4c4ccccc4n32)cc1]

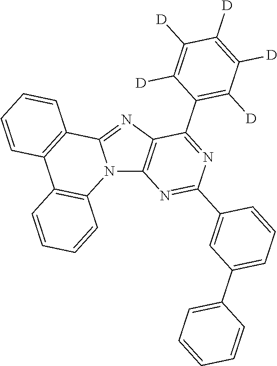 [SMILES: [2H]c1c([2H])c([2H])c(-c2nc(-c3cccc(-c4ccccc4)c3)nc3c2nc2c4ccccc4c4ccccc4n32)c([2H])c1[2H]]

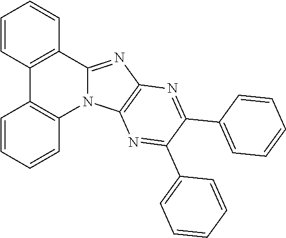 [SMILES: c1ccc(-c2nc3nc4c5ccccc5c5ccccc5n4c3nc2-c2ccccc2)cc1]

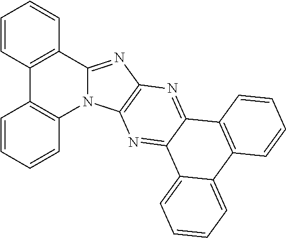 [SMILES: c1ccc2c(c1)c1ccccc1c1nc3c(nc21)nc1c2ccccc2c2ccccc2n31]

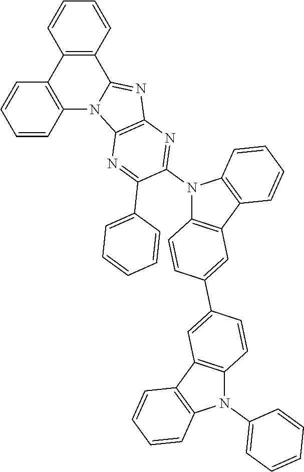 [SMILES: c1ccc(-c2nc3c(nc2-n2c4ccccc4c4cc(-c5ccc6c(c5)c5ccccc5n6-c5ccccc5)ccc42)nc2c4ccccc4c4ccccc4n32)cc1]

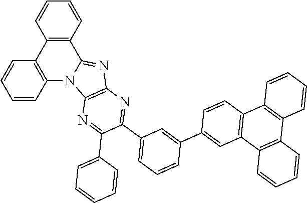 [SMILES: c1ccc(-c2nc3c(nc2-c2cccc(-c4ccc5c6ccccc6c6ccccc6c5c4)c2)nc2c4ccccc4c4ccccc4n32)cc1]

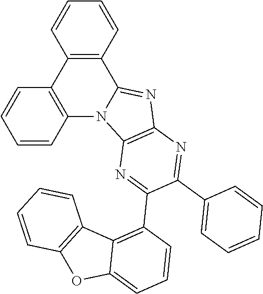 [SMILES: c1ccc(-c2nc3nc4c5ccccc5c5ccccc5n4c3nc2-c2cccc3oc4ccccc4c23)cc1]

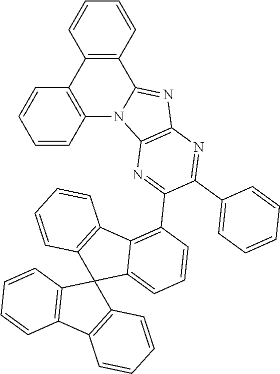 [SMILES: c1ccc(-c2nc3nc4c5ccccc5c5ccccc5n4c3nc2-c2cccc3c2-c2ccccc2C32c3ccccc3-c3ccccc32)cc1]

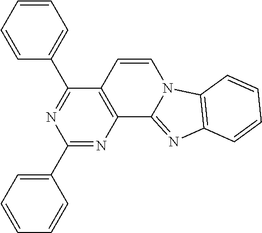 [SMILES: c1ccc(-c2nc(-c3ccccc3)c3ccn4c5ccccc5nc4c3n2)cc1]

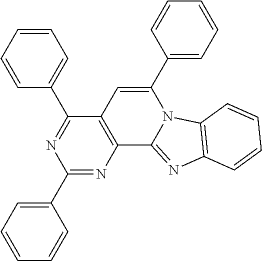 [SMILES: c1ccc(-c2nc(-c3ccccc3)c3cc(-c4ccccc4)n4c5ccccc5nc4c3n2)cc1]

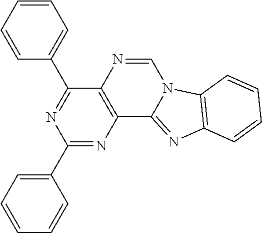 [SMILES: c1ccc(-c2nc(-c3ccccc3)c3ncn4c5ccccc5nc4c3n2)cc1]

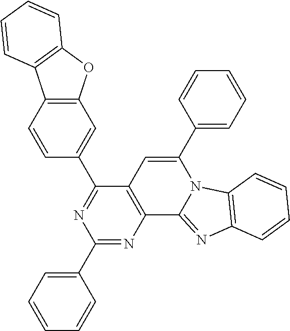 [SMILES: c1ccc(-c2nc(-c3ccc4c(c3)oc3ccccc34)c3cc(-c4ccccc4)n4c5ccccc5nc4c3n2)cc1]

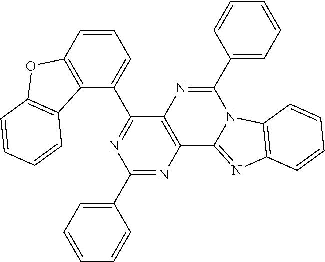 [SMILES: c1ccc(-c2nc(-c3cccc4oc5ccccc5c34)c3nc(-c4ccccc4)n4c5ccccc5nc4c3n2)cc1]

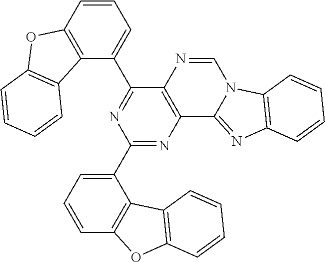 [SMILES: c1ccc2c(c1)nc1c3nc(-c4cccc5oc6ccccc6c45)nc(-c4cccc5oc6ccccc6c45)c3ncn21]

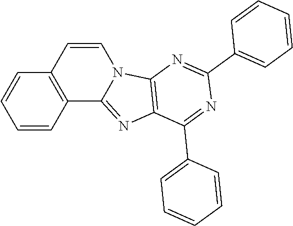 [SMILES: c1ccc(-c2nc(-c3ccccc3)c3nc4c5ccccc5ccn4c3n2)cc1]

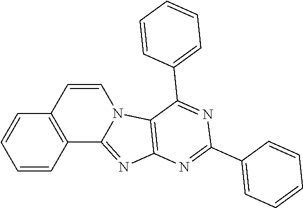 [SMILES: c1ccc(-c2nc(-c3ccccc3)c3c(n2)nc2c4ccccc4ccn23)cc1]

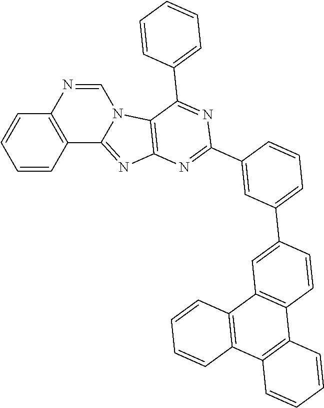 [SMILES: c1ccc(-c2nc(-c3cccc(-c4ccc5c6ccccc6c6ccccc6c5c4)c3)nc3nc4c5ccccc5ncn4c23)cc1]

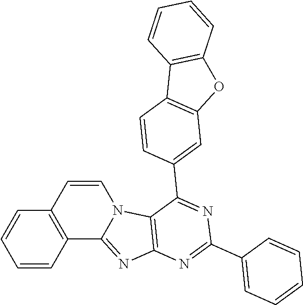 [SMILES: c1ccc(-c2nc(-c3ccc4c(c3)oc3ccccc34)c3c(n2)nc2c4ccccc4ccn23)cc1]

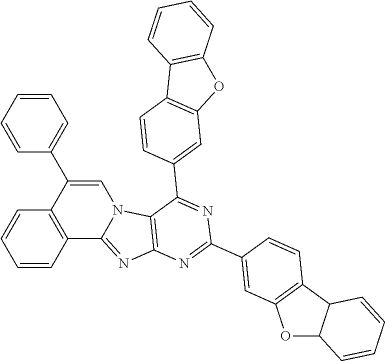 [SMILES: C1=CC2Oc3cc(-c4nc(-c5ccc6c(c5)oc5ccccc56)c5c(n4)nc4c6ccccc6c(-c6ccccc6)cn45)ccc3C2C=C1]

The base structure of the compounds of the invention can be prepared by the routes outlined in the schemes which follow. The individual synthesis steps, for example C—C coupling reactions according to Suzuki, C—N coupling reactions according to Hartwig-Buchwald or cyclization reactions, are known in principle to those skilled in the art. Further information relating to the synthesis of the compounds of the invention can be found in the synthesis examples. One possible synthesis of the base structure is shown in scheme 1. This can be effected by the reactions set out in Journal of Organic Chemistry 84(18), 12009-12020, 2019. Alternatively, a borane can be coupled to an amino group and a nitrile, followed by a ring closure reaction. Schemes 3 to 9 show various possible options for preparation of the base structure and for introduction of substituents.

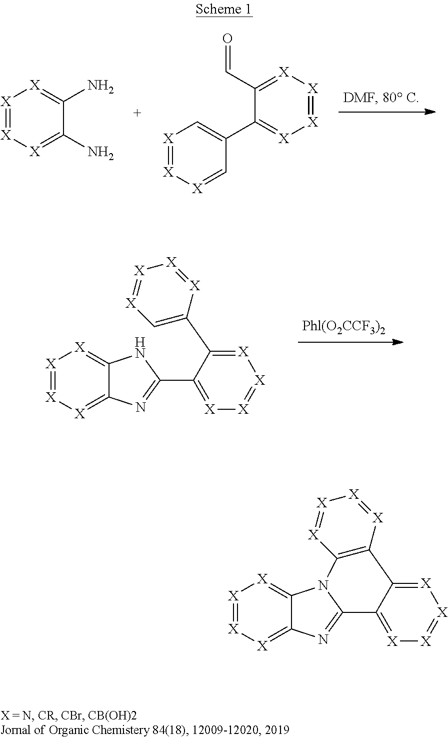

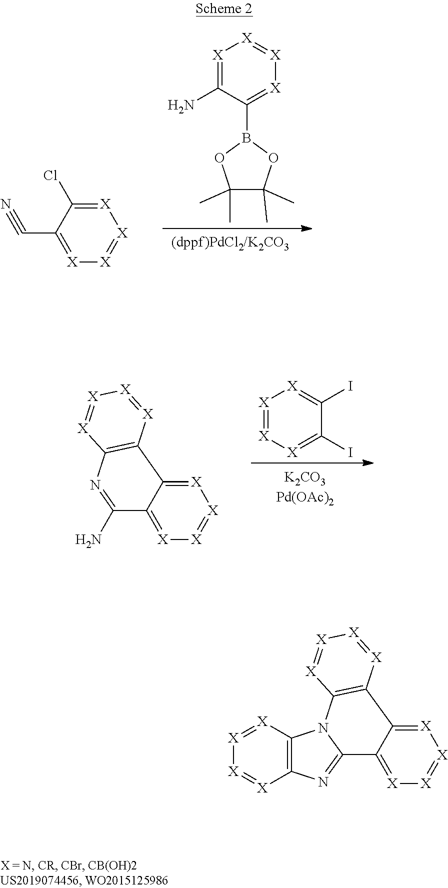

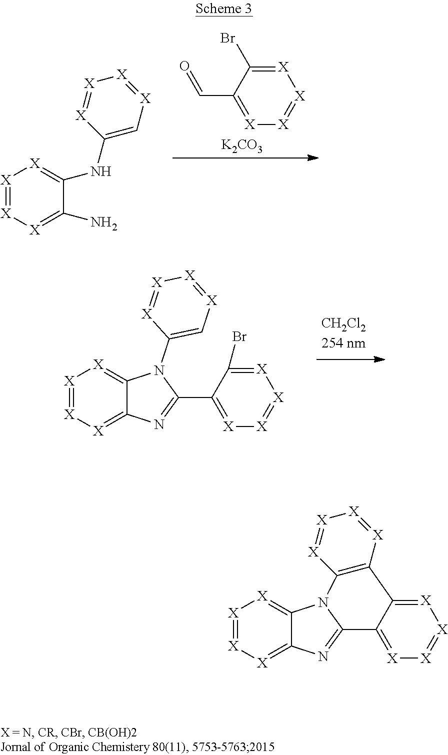

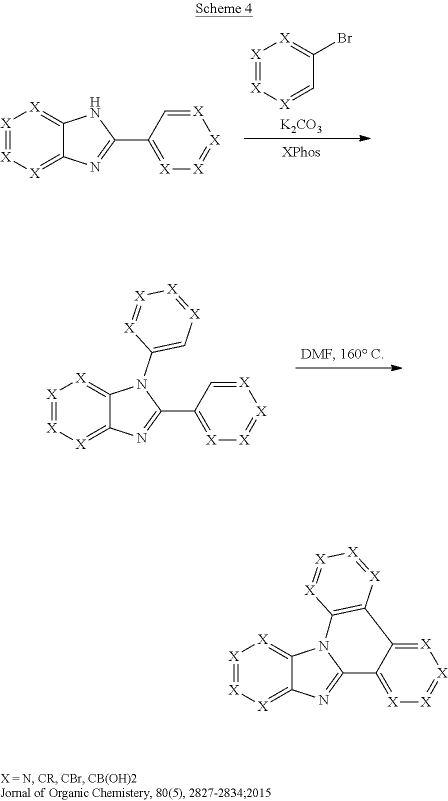

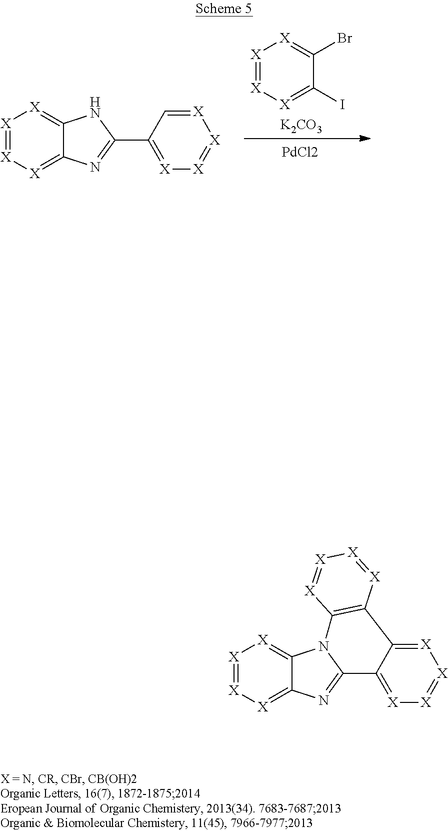

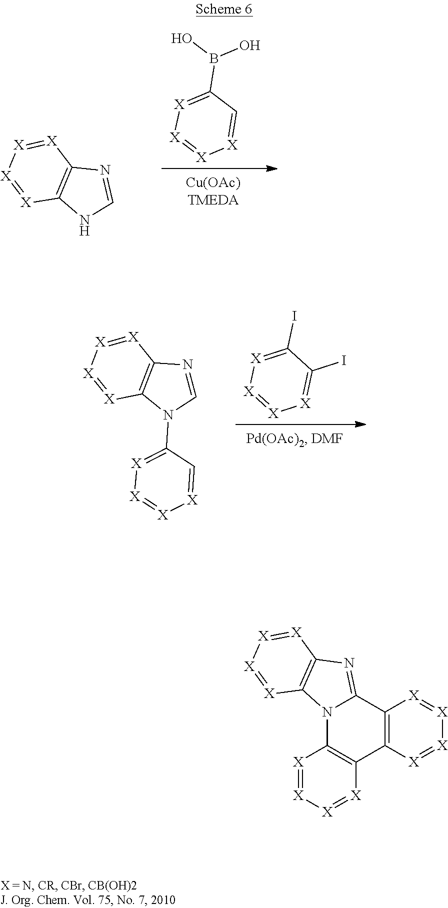

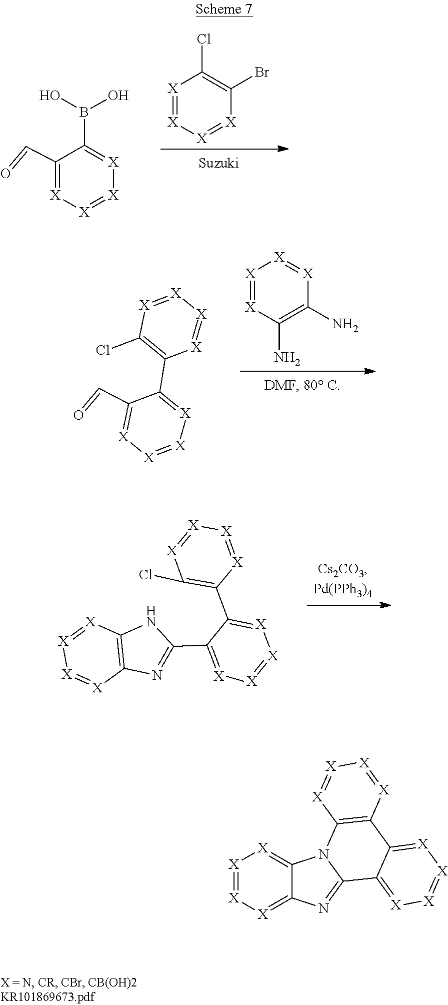

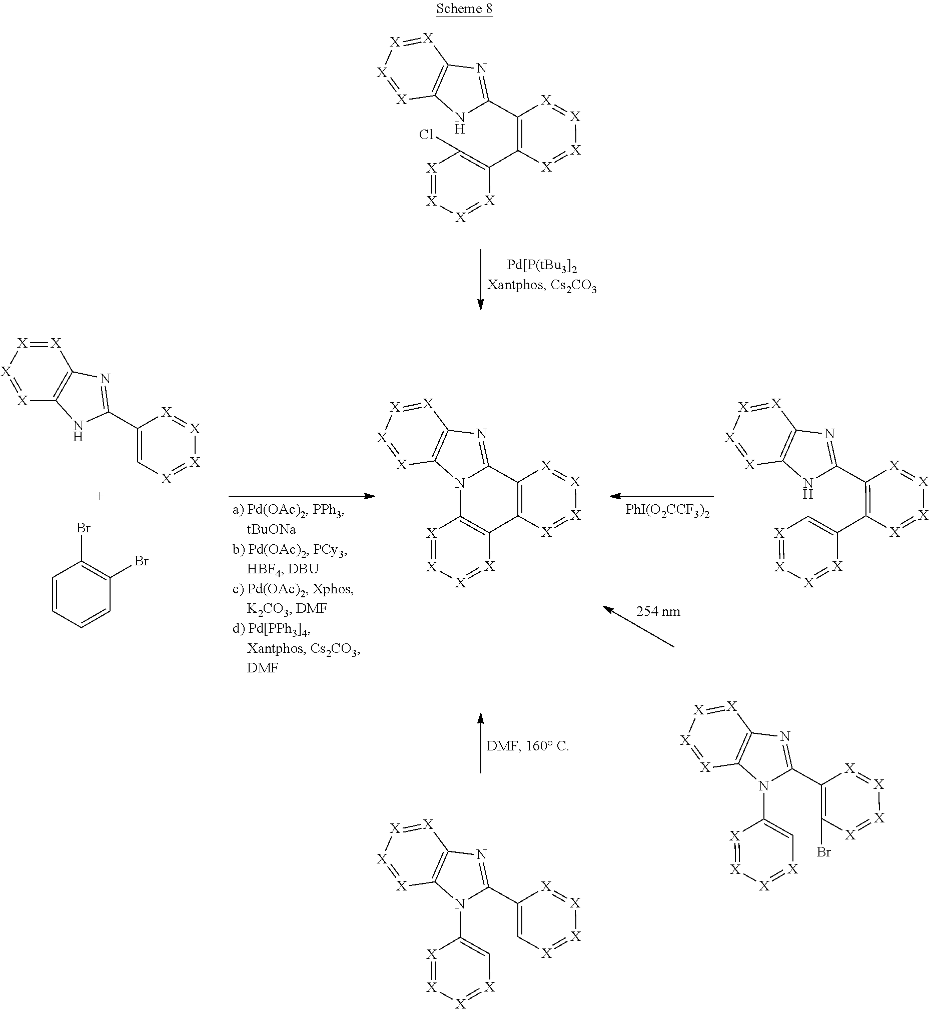

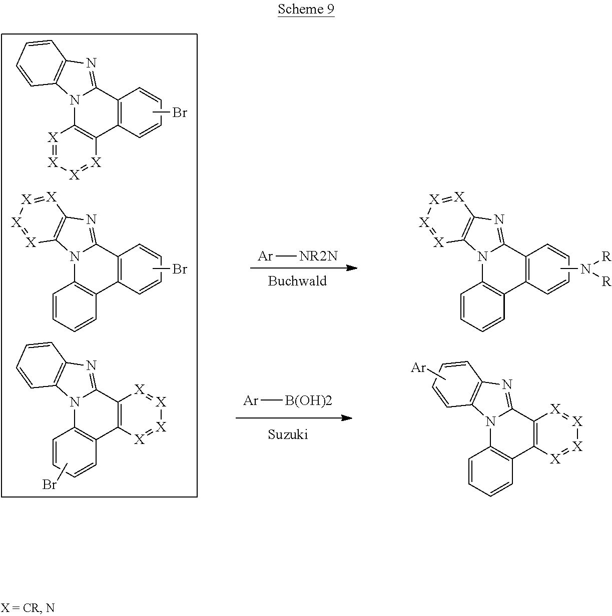

The definition of the symbols used in schemes 1 to 9 corresponds essentially to that which was defined for formula (I), dispensing with numbering and complete representation of all symbols for reasons of clarity. The symbol X has been used in each case as an abbreviation for the symbols X¹, X², X³ and X defined by formula (I), with specific enumeration of R, R¹, R² and/or R³ radicals used with preference for derivatization, for example CBr, CB(OH)₂. These details should therefore be considered to be illustrative.

The present invention therefore further provides a process for preparing a compound of the invention, wherein an aromatic or heteroaromatic compound is reacted with an aromatic or heteroaromatic diamino compound by a coupling reaction.

For the processing of the compounds of the invention from a liquid phase, for example by spin-coating or by printing methods, formulations of the compounds of the invention are required. These formulations may, for example, be solutions, dispersions or emulsions. For this purpose, it may be preferable to use mixtures of two or more solvents. Suitable and preferred solvents are, for example, toluene, anisole, o-, m- or p-xylene, methyl benzoate, mesitylene, tetralin, veratrole, THF, methyl-THF, THP, chlorobenzene, dioxane, phenoxytoluene, especially 3-phenoxytoluene, (−)-fenchone, 1,2,3,5-tetramethylbenzene, 1,2,4,5-tetramethylbenzene, 1-methylnaphthalene, 2-methylbenzothiazole, 2-phenoxyethanol, 2-pyrrolidinone, 3-methylanisole, 4-methylanisole, 3,4-dimethylanisole, 3,5-dimethylanisole, acetophenone, α-terpineol, benzothiazole, butyl benzoate, cumene, cyclohexanol, cyclohexanone, cyclohexylbenzene, decalin, dodecylbenzene, ethyl benzoate, indane, NMP, p-cymene, phenetole, 1,4-diisopropylbenzene, dibenzyl ether, diethylene glycol butyl methyl ether, triethylene glycol butyl methyl ether, diethylene glycol dibutyl ether, triethylene glycol dimethyl ether, diethylene glycol monobutyl ether, tripropylene glycol dimethyl ether, tetraethylene glycol dimethyl ether, 2-isopropylnaphthalene, pentylbenzene, hexylbenzene, heptylbenzene, octylbenzene, 1,1-bis(3,4-dimethylphenyl)ethane, 2-methylbiphenyl, 3-methylbiphenyl, 1-methylnaphthalene, 1-ethylnaphthalene, ethyl octanoate, diethyl sebacate, octyl octanoate, heptylbenzene, menthyl isovalerate, cyclohexyl hexanoate or mixtures of these solvents.

The present invention therefore further provides a formulation or a composition comprising at least one compound of the invention and at least one further compound. The further compound may, for example, be a solvent, especially one of the abovementioned solvents or a mixture of these solvents. If the further compound comprises a solvent, this mixture is referred to herein as formulation. The further compound may alternatively be at least one further organic or inorganic compound which is likewise used in the electronic device, for example an emitting compound and/or a further matrix material. Suitable emitting compounds and further matrix materials are listed at the back in connection with the organic electroluminescent device. The further compound may also be polymeric.

The present invention further provides for the use of a compound of the invention in an electronic device, especially in an organic electroluminescent device.

The present invention still further provides an electronic device comprising at least one compound of the invention. An electronic device in the context of the present invention is a device comprising at least one layer comprising at least one organic compound. This component may also comprise inorganic materials or else layers formed entirely from inorganic materials.

The electronic device is more preferably selected from the group consisting of organic electroluminescent devices (OLEDs, sOLED, PLEDs, LECs, etc.), preferably organic light-emitting diodes (OLEDs), organic light-emitting diodes based on small molecules (sOLEDs), organic light-emitting diodes based on polymers (PLEDs), light-emitting electrochemical cells (LECs), organic laser diodes (O-laser), organic plasmon-emitting devices (D. M. Koller et al., Nature Photonics 2008, 1-4), organic integrated circuits (O-ICs), organic field-effect transistors (O-FETs), organic thin-film transistors (O-TFTs), organic light-emitting transistors (O-LETs), organic solar cells (O-SCs), organic optical detectors, organic photoreceptors, organic field-quench devices (O-FQDs) and organic electrical sensors, preferably organic electroluminescent devices (OLEDs, sOLED, PLEDs, LECs, etc.), more preferably organic light-emitting diodes (OLEDs), organic light-emitting diodes based on small molecules (sOLEDs), organic light-emitting diodes based on polymers (PLEDs), especially phosphorescent OLEDs.

The organic electroluminescent device comprises cathode, anode and at least one emitting layer. Apart from these layers, it may also comprise further layers, for example in each case one or more hole injection layers, hole transport layers, hole blocker layers, electron transport layers, electron injection layers, exciton blocker layers, electron blocker layers and/or charge generation layers. It is likewise possible for interlayers having an exciton-blocking function, for example, to be introduced between two emitting layers. However, it should be pointed out that not necessarily every one of these layers need be present. In this case, it is possible for the organic electroluminescent device to contain an emitting layer, or for it to contain a plurality of emitting layers. If a plurality of emission layers are present, these preferably have several emission maxima between 380 nm and 750 nm overall, such that the overall result is white emission; in other words, various emitting compounds which may fluoresce or phosphoresce are used in the emitting layers. Especially preferred are systems having three emitting layers, where the three layers show blue, green and orange or red emission. The organic electroluminescent device of the invention may also be a tandem electroluminescent device, especially for white-emitting OLEDs.

The compound of the invention may be used in different layers, according to the exact structure. Preference is given to an organic electroluminescent device comprising a compound of formula (I) or the above-recited preferred embodiments in an emitting layer as matrix material for phosphorescent emitters or for emitters that exhibit TADF (thermally activated delayed fluorescence), especially for phosphorescent emitters. In addition, the compound of the invention can also be used in an electron transport layer or in a hole blocker layer and/or in an electron blocker layer, preferably in an electron transport layer and/or in a hole blocker layer. More preferably, the compound of the invention is used as matrix material for phosphorescent emitters, especially for red-, orange-, green- or yellow-phosphorescing, preferably green-phosphorescing, emitters in an emitting layer or as electron transport in an electron transport layer, more preferably as matrix material in an emitting layer.

When the compound of the invention is used as matrix material for a phosphorescent compound in an emitting layer, it is preferably used in combination with one or more phosphorescent materials (triplet emitters). Phosphorescence in the context of this invention is understood to mean luminescence from an excited state having higher spin multiplicity, i.e. a spin state >1, especially from an excited triplet state. In the context of this application, all luminescent complexes with transition metals or lanthanides, especially all iridium, platinum and copper complexes, shall be regarded as phosphorescent compounds.

The mixture of the compound of the invention and the emitting compound contains between 99% and 1% by volume, preferably between 98% and 10% by volume, more preferably between 97% and 60% by volume and especially between 95% and 80% by volume of the compound of the invention, based on the overall mixture of emitter and matrix material. Correspondingly, the mixture contains between 1% and 99% by volume, preferably between 2% and 90% by volume, more preferably between 3% and 40% by volume and especially between 5% and 20% by volume of the emitter, based on the overall mixture of emitter and matrix material.

In one embodiment of the invention, the compound of the invention is used here as the sole matrix material (“single host”) for the phosphorescent emitter.

A further embodiment of the present invention is the use of the compound of the invention as matrix material for a phosphorescent emitter in combination with a further matrix material. Suitable matrix materials which can be used in combination with the inventive compounds are aromatic ketones, aromatic phosphine oxides or aromatic sulfoxides or sulfones, for example according to WO 2004/013080, WO 2004/093207, WO 2006/005627 or WO 2010/006680, triarylamines, carbazole derivatives, e.g. CBP (N,N-biscarbazolylbiphenyl) or the carbazole derivatives disclosed in WO 2005/039246, US 2005/0069729, JP 2004/288381, EP 1205527, WO 2008/086851 or WO 2013/041176, indolocarbazole derivatives, for example according to WO 2007/063754 or WO 2008/056746, indenocarbazole derivatives, for example according to WO 2010/136109, WO 2011/000455, WO 2013/041176 or WO 2013/056776, azacarbazole derivatives, for example according to EP 1617710, EP 1617711, EP 1731584, JP 2005/347160, bipolar matrix materials, for example according to WO 2007/137725, silanes, for example according to WO 2005/111172, azaboroles or boronic esters, for example according to WO 2006/117052, triazine derivatives, for example according to WO 2007/063754, WO 2008/056746, WO 2010/015306, WO 2011/057706, WO 2011/060859 or WO 2011/060877, zinc complexes, for example according to EP 652273 or WO 2009/062578, diazasilole or tetraazasilole derivatives, for example according to WO 2010/054729, diazaphosphole derivatives, for example according to WO 2010/054730, bridged carbazole derivatives, for example according to WO 2011/042107, WO 2011/060867, WO 2011/088877 and WO 2012/143080, triphenylene derivatives, for example according to WO 2012/048781, dibenzofuran derivatives, for example according to WO 2015/169412, WO 2016/015810, WO 2016/023608, WO 2017/148564 or WO 2017/148565, or biscarbazoles, for example according to JP 3139321 B2.

It is likewise possible for a further phosphorescent emitter which emits at a shorter wavelength than the actual emitter to be present as co-host in the mixture. Particularly good results are achieved when the emitter used is a red-phosphorescing emitter and the co-host used in combination with the compound of the invention is a yellow-phosphorescing emitter.

In addition, the co-host used may be a compound that does not take part in charge transport to a significant degree, if at all, as described, for example, in WO 2010/108579. Especially suitable in combination with the compound of the invention as co-matrix material are compounds which have a large bandgap and themselves take part at least not to a significant degree, if any at all, in the charge transport of the emitting layer. Such materials are preferably pure hydrocarbons. Examples of such materials can be found, for example, in WO 2009/124627 or in WO 2010/006680.

Particularly preferred co-host materials which can be used in combination with the compounds of the invention are compounds of one of the formulae (H-1), (H-2), (H-3), (H-4) and (H-5):

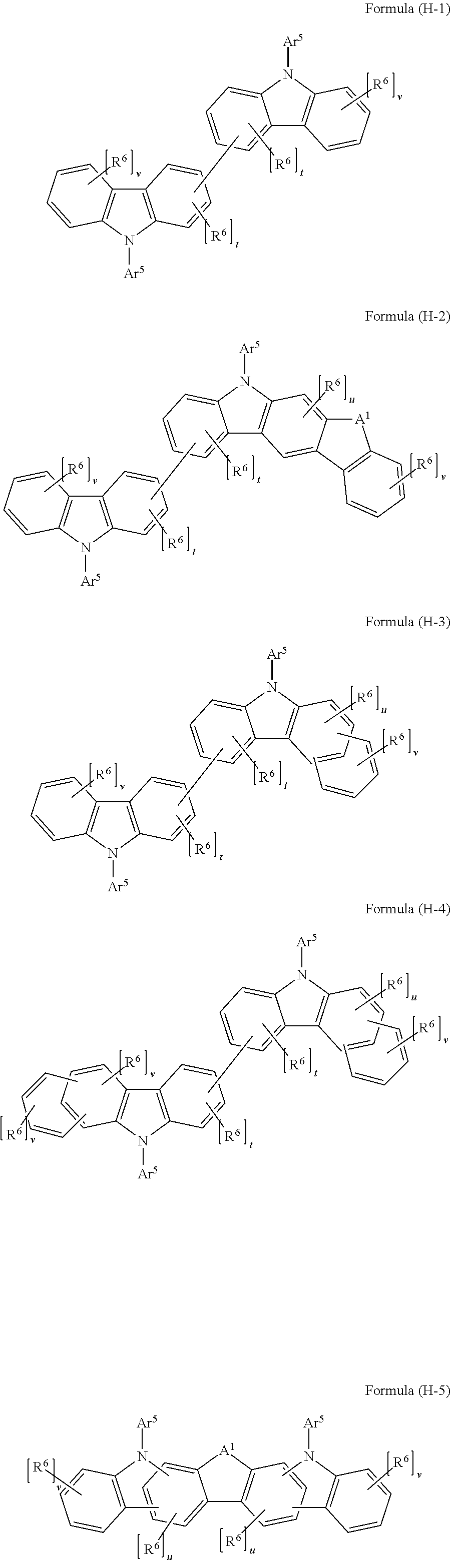

where the symbols and indices used are as follows:
R⁶ is the same or different at each instance and is H, D, F, Cl, Br, I, N(R⁷)₂, N(Ar″)₂, CN, NO₂, OR⁷, SR⁷, COOR⁷, C(═O)N(R⁷)₂, Si(R⁷)₃, B(OR⁷)₂, C(═O)R⁷, P(═O)(R⁷)₂, S(═O)R⁷, S(═O)₂R⁷, OSO₂R⁷, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R⁴ radicals, where one or more nonadjacent CH₂ groups may be replaced by Si(R⁷)₂, C═O, NR⁷, O, S or CONR⁷, or an aromatic or heteroaromatic ring system which has 5 to 60 aromatic ring atoms, preferably 5 to 40 aromatic ring atoms, and may be substituted in each case by one or more R⁷ radicals; at the same time, two R⁶ radicals together may also form an aromatic, heteroaromatic, aliphatic or heteroaliphatic ring system; preferably, the R⁶ radicals do not form any such ring system;
Ar″ is the same or different at each instance and is an aromatic or heteroaromatic ring system which has 5 to 40 aromatic ring atoms and may be substituted by one or more R⁷ radicals;
A¹ is C(R⁷)₂, NR⁷, O or S;
Ar⁵ is the same or different at each instance and is an aromatic or heteroaromatic ring system which has 5 to 40 aromatic ring atoms and may be substituted by one or more R⁷ radicals;
R⁷ is the same or different at each instance and is H, D, F, Cl, Br, I, N(R⁸)₂, CN, NO₂, OR⁸, SR⁸, Si(R⁸)₃, B(OR⁸)₂, C(═O)R⁸, P(═O)(R⁸)₂, S(═O)R⁸, S(═O)₂R⁸, OSO₂R⁸, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R⁸ radicals and where one or more nonadjacent CH₂ groups may be replaced by Si(R⁸)₂, C═O, NR^{B}, O, S or CONR⁸, or an aromatic or heteroaromatic ring system which has 5 to 40 aromatic ring atoms and may be substituted in each case by one or more R⁸ radicals; at the same time, two or more R⁷ radicals together may form an aromatic, heteroaromatic, aliphatic or heteroaliphatic ring system; preferably, the R⁷ radicals do not form any such ring system;
R⁸ is the same or different at each instance and is H, D, F or an aliphatic, aromatic or heteroaromatic organic radical, especially a hydrocarbyl radical, having 1 to 20 carbon atoms, in which one or more hydrogen atoms may also be replaced by F;
v is the same or different at each instance and is 0, 1, 2, 3 or 4, preferably 0 or 1 and very preferably 0;
t is the same or different at each instance and is 0, 1, 2 or 3, preferably 0 or 1 and very preferably 0;
u is the same or different at each instance and is 0, 1 or 2, preferably 0 or 1 and very preferably 0.

The sum total of the indices v, t and u in compounds of the formulae (H-1), (H-2), (H-3), (H-4) or (H-5) is preferably not more than 6, more preferably not more than 4 and especially preferably not more than 2.

In a preferred embodiment of the invention, R⁶ is the same or different at each instance and is selected from the group consisting of H, D, F, CN, NO₂, Si(R⁷)₃, B(OR⁷)₂, a straight-chain alkyl group having 1 to 20 carbon atoms or a branched or cyclic alkyl group having 3 to 20 carbon atoms, where the alkyl group may be substituted in each case by one or more R⁷ radicals, or an aromatic or heteroaromatic ring system which has 5 to 60 aromatic ring atoms, preferably 5 to 40 aromatic ring atoms, and may be substituted in each case by one or more R⁷ radicals.

In a further-preferred embodiment of the invention, R⁶ is the same or different at each instance and is selected from the group consisting of H, D, F, a straight-chain alkyl group having 1 to 20 carbon atoms or a branched or cyclic alkyl group having 3 to 20 carbon atoms, where the alkyl group may be substituted in each case by one or more R⁷ radicals, or an aromatic or heteroaromatic ring system which has 5 to 60 aromatic ring atoms, preferably 5 to 40 aromatic ring atoms, and may be substituted in each case by one or more R⁷ radicals.

In a further-preferred embodiment of the invention, R⁶ is the same or different at each instance and is selected from the group consisting of H, D, an aromatic or heteroaromatic ring system which has 6 to 30 aromatic ring atoms and may be substituted by one or more R⁷ radicals, and an N(Ar″)₂ group. More preferably, R⁶ is the same or different at each instance and is selected from the group consisting of H or an aromatic or heteroaromatic ring system which has 6 to 24 aromatic ring atoms, preferably 6 to 18 aromatic ring atoms, more preferably 6 to 13 aromatic ring atoms, and may be substituted in each case by one or more R⁷ radicals.

Preferred aromatic or heteroaromatic ring systems R⁶ or Ar″ are selected from phenyl, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorene which may be joined via the 1, 2, 3 or 4 position, spirobifluorene which may be joined via the 1, 2, 3 or 4 position, naphthalene, especially 1- or 2-bonded naphthalene, indole, benzofuran, benzothiophene, carbazole which may be joined via the 1, 2, 3 or 4 position, dibenzofuran which may be joined via the 1, 2, 3 or 4 position, dibenzothiophene which may be joined via the 1, 2, 3 or 4 position, indenocarbazole, indolocarbazole, pyridine, pyrimidine, pyrazine, pyridazine, triazine, quinoline, isoquinoline, quinazoline, quinoxaline, phenanthrene or triphenylene, each of which may be substituted by one or more R⁷ radicals. The structures Ar-1 to Ar-75 listed above are particularly preferred, preference being given to structures of the formulae (Ar-1), (Ar-2), (Ar-3), (Ar-12), (Ar-13), (Ar-14), (Ar-15), (Ar-16), (Ar-69), (Ar-70), (Ar-75), and particular preference to structures of the formulae (Ar-1), (Ar-2), (Ar-3), (Ar-12), (Ar-13), (Ar-14), (Ar-15), (Ar-16). In the structures Ar-1 to Ar-75 set out above, in relation to the R⁶ and Ar″ radicals, the substituents R⁴ should be replaced by the corresponding R⁷ radicals. The preferences set out above for the R¹, R² and R³ groups are correspondingly applicable to the R⁶ group.

Further suitable R⁶ groups are groups of the formula —Ar⁴—N(Ar²)(Ar³) where Ar², Ar³ and Ar⁴ are the same or different at each instance and are an aromatic or heteroaromatic ring system which has 5 to 24 aromatic ring atoms and may be substituted in each case by one or more R⁴ radicals. The total number of aromatic ring atoms in Ar², Ar³ and Ar⁴ here is not more than 60 and preferably not more than 40. Further preferences for the Ar², Ar³ and Ar⁴ groups have been set out above and are correspondingly applicable.

It may further be the case that the substituents R⁶ according to the above formulae do not form a fused aromatic or heteroaromatic ring system, preferably any fused ring system, with the ring atoms of the ring system. This includes the formation of a fused ring system with possible substituents R⁷, R⁸ which may be bonded to the R⁶ radicals.

When A¹ is NR⁷, the substituent R⁷ bonded to the nitrogen atom is preferably an aromatic or heteroaromatic ring system which has 5 to 24 aromatic ring atoms and may also be substituted by one or more R⁸ radicals. In a particularly preferred embodiment, this R⁷ substituent is the same or different at each instance and is an aromatic or heteroaromatic ring system which has 6 to 24 aromatic ring atoms, especially 6 to 18 aromatic ring atoms, which does not have any fused aryl groups and which does not have any fused heteroaryl groups in which two or more aromatic or heteroaromatic 6-membered ring groups are fused directly to one another, and which may also be substituted in each case by one or more R⁸ radicals. Preference is given to phenyl, biphenyl, terphenyl and quaterphenyl having bonding patterns as listed above for Ar-1 to Ar-11, where these structures, rather than by R⁴, may be substituted by one or more R⁸ radicals, but are preferably unsubstituted. Preference is further given to triazine, pyrimidine and quinazoline as listed above for Ar-47 to Ar-50, Ar-57 and Ar-58, where these structures, rather than by R⁴, may be substituted by one or more R⁸ radicals.

When A¹ is C(R⁷)₂, the substituents R⁷ bonded to this carbon atom are preferably the same or different at each instance and are a linear alkyl group having 1 to 10 carbon atoms or a branched or cyclic alkyl group having 3 to 10 carbon atoms or an aromatic or heteroaromatic ring system having 5 to 24 aromatic ring atoms, which may also be substituted by one or more R⁵ radicals. Most preferably, R⁷ is a methyl group or a phenyl group. In this case, the R⁷ radicals together may also form a ring system, which leads to a spiro system.

Preferred aromatic or heteroaromatic ring systems Ar⁵ are selected from phenyl, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorene which may be joined via the 1, 2, 3 or 4 position, spirobifluorene which may be joined via the 1, 2, 3 or 4 position, naphthalene, especially 1- or 2-bonded naphthalene, indole, benzofuran, benzothiophene, carbazole which may be joined via the 1, 2, 3 or 4 position, dibenzofuran which may be joined via the 1, 2, 3 or 4 position, dibenzothiophene which may be joined via the 1, 2, 3 or 4 position, indenocarbazole, indolocarbazole, pyridine, pyrimidine, pyrazine, pyridazine, triazine, quinoline, isoquinoline, quinazoline, quinoxaline, phenanthrene or triphenylene, each of which may be substituted by one or more R⁷ or R¹⁰ radicals.

The Ar⁵ groups here are more preferably independently selected from the groups of the formulae Ar-1 to Ar-75 set out above, preference being given to structures of the formulae (Ar-1), (Ar-2), (Ar-3), (Ar-12), (Ar-13), (Ar-14), (Ar-15), (Ar-16), (Ar-69), (Ar-70), (Ar-75), and particular preference to structures of the formulae (Ar-1), (Ar-2), (Ar-3), (Ar-12), (Ar-13), (Ar-14), (Ar-15), (Ar-16). In the structures Ar-1 to Ar-75 set out above, in relation to the Ar⁵ radicals, the substituents R⁴ by the corresponding R⁷ radicals.

In a further preferred embodiment of the invention, R⁷ is the same or different at each instance and is selected from the group consisting of H, D, F, CN, a straight-chain alkyl group having 1 to 10 carbon atoms or a branched or cyclic alkyl group having 3 to 10 carbon atoms, where the alkyl group may be substituted in each case by one or more R⁸ radicals, or an aromatic or heteroaromatic ring system which has 6 to 24 aromatic ring atoms and may be substituted in each case by one or more R⁸ radicals. In a particularly preferred embodiment of the invention, R⁷ is the same or different at each instance and is selected from the group consisting of H, a straight-chain alkyl group having 1 to 6 carbon atoms, especially having 1, 2, 3 or 4 carbon atoms, or a branched or cyclic alkyl group having 3 to 6 carbon atoms, where the alkyl group may be substituted by one or more R⁸ radicals, but is preferably unsubstituted, or an aromatic or heteroaromatic ring system which has 6 to 13 aromatic ring atoms and may be substituted in each case by one or more R⁸ radicals, but is preferably unsubstituted.

In a further preferred embodiment of the invention, R⁸ is the same or different at each instance and is H, an alkyl group having 1 to 4 carbon atoms or an aryl group having 6 to 10 carbon atoms, which may be substituted by an alkyl group having 1 to 4 carbon atoms, but is preferably unsubstituted.

Preferred embodiments of the compounds of the formulae (H-1) and (H-2) are the compounds of the following formulae (H-1a) and (H-2a):

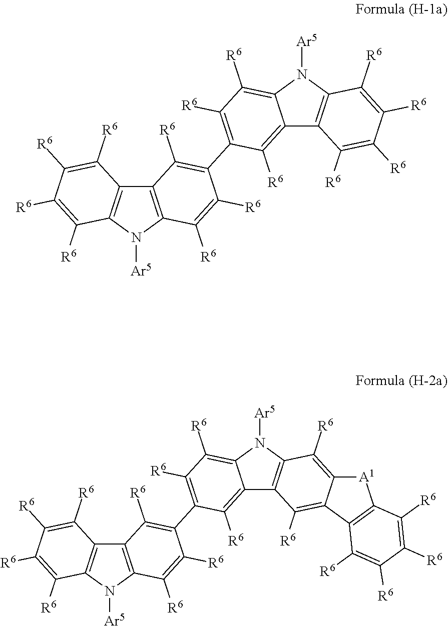

where R⁶, Ar⁵ and A¹ have the definitions given above, especially for formula (H-1) or (H-2). In a preferred embodiment of the invention, A¹ in formula (H-2a) is C(R⁷)₂.

Preferred embodiments of the compounds of the formulae (H-1a) and (H-2a) are the compounds of the following formulae (H-1 b) and (H-2b):

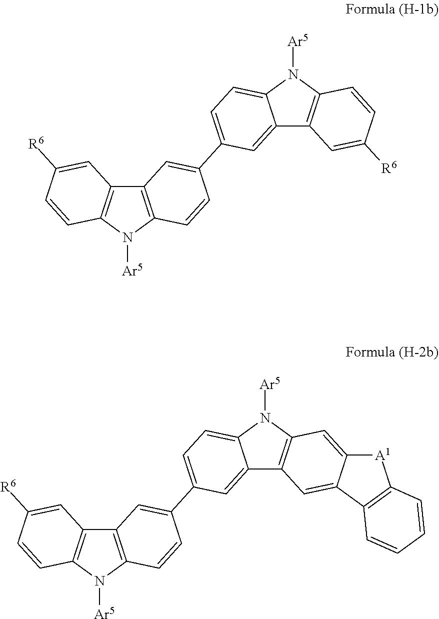

where R⁶, Ar⁵ and A¹ have the definitions given above, especially for formula (H-1) or (H-2). In a preferred embodiment of the invention, A¹ in formula (H-2b) is C(R⁷)₂.

Examples of suitable compounds of formulae (H-1), (H-2), (H-3), (H-4) and (H-5) are the compounds depicted below:

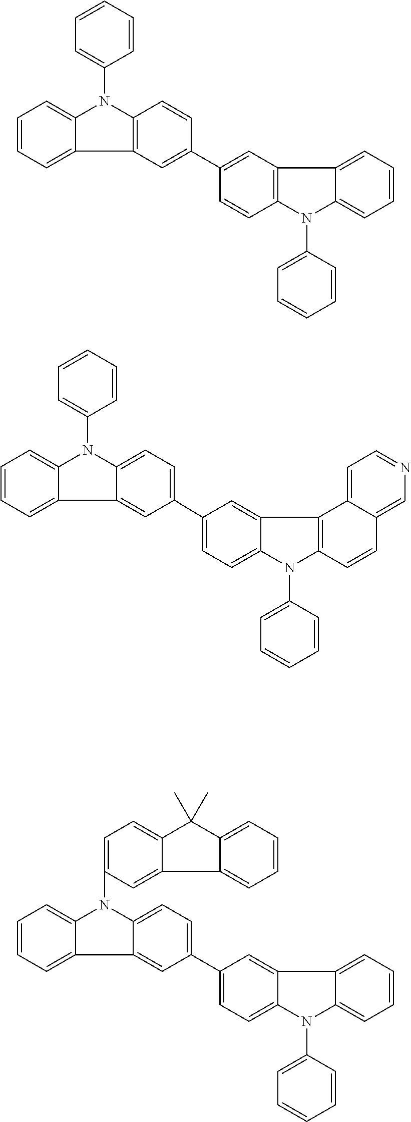 [SMILES: c1ccc(-n2c3ccccc3c3cc(-c4ccc5c(c4)c4ccccc4n5-c4ccccc4)ccc32)cc1 ; c1ccc(-n2c3ccccc3c3cc(-c4ccc5c(c4)c4c6ccncc6ccc4n5-c4ccccc4)ccc32)cc1 ; CC1(C)c2ccccc2-c2cc(-n3c4ccccc4c4cc(-c5ccc6c(c5)c5ccccc5n6-c5ccccc5)ccc43)ccc21]

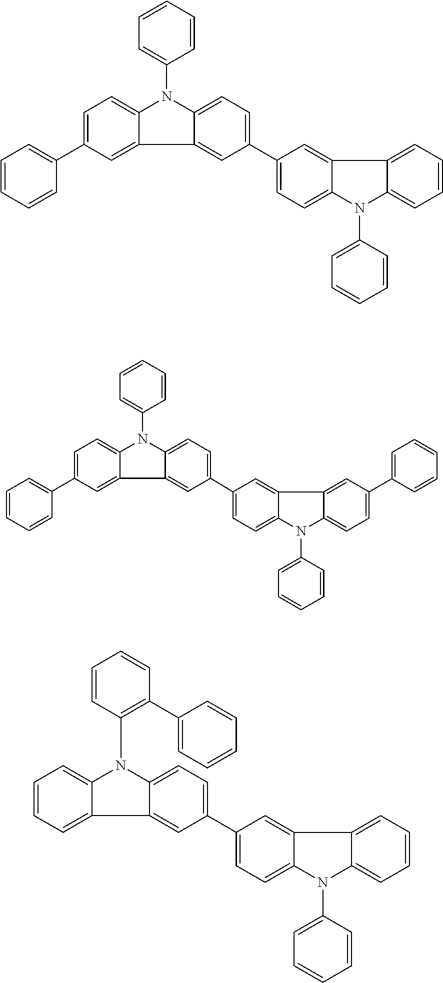 [SMILES: c1ccc(-c2ccc3c(c2)c2cc(-c4ccc5c(c4)c4ccccc4n5-c4ccccc4)ccc2n3-c2ccccc2)cc1 ; c1ccc(-c2ccc3c(c2)c2cc(-c4ccc5c(c4)c4cc(-c6ccccc6)ccc4n5-c4ccccc4)ccc2n3-c2ccccc2)cc1 ; c1ccc(-c2ccccc2-n2c3ccccc3c3cc(-c4ccc5c(c4)c4ccccc4n5-c4ccccc4)ccc32)cc1]

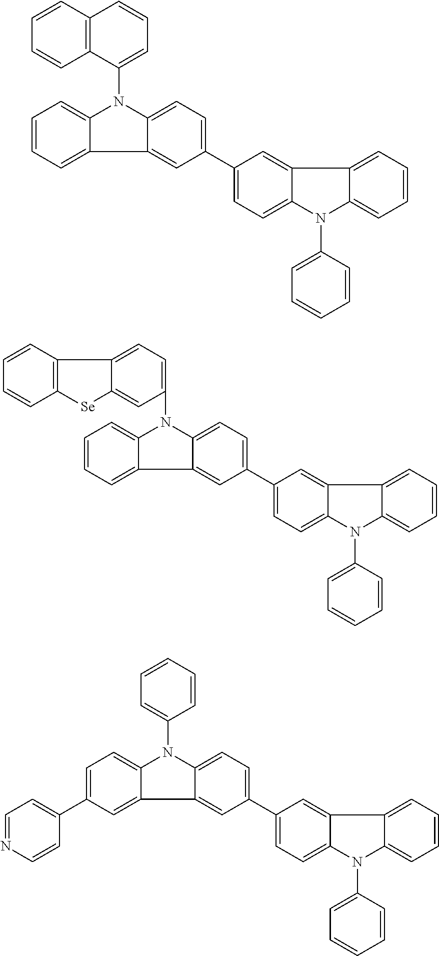 [SMILES: c1ccc(-n2c3ccccc3c3cc(-c4ccc5c(c4)c4ccccc4n5-c4cccc5ccccc45)ccc32)cc1 ; c1ccc(-n2c3ccccc3c3cc(-c4ccc5c(c4)c4ccccc4n5-c4ccc5c(c4)[se]c4ccccc45)ccc32)cc1 ; c1ccc(-n2c3ccccc3c3cc(-c4ccc5c(c4)c4cc(-c6ccncc6)ccc4n5-c4ccccc4)ccc32)cc1]

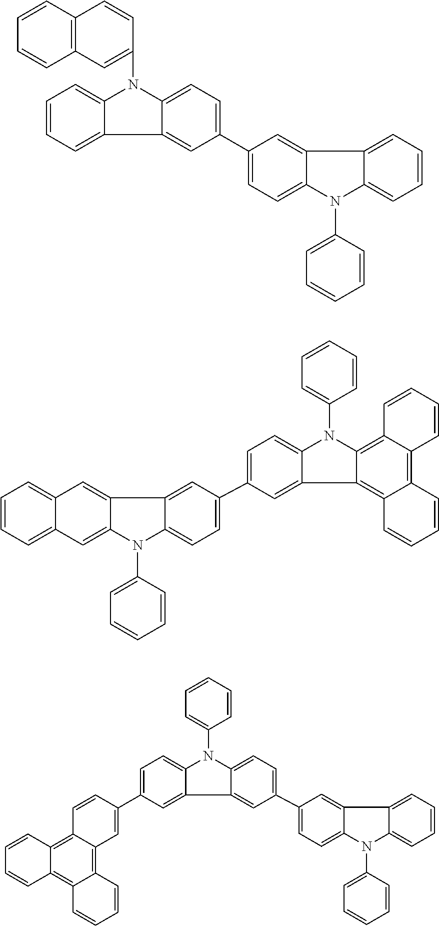

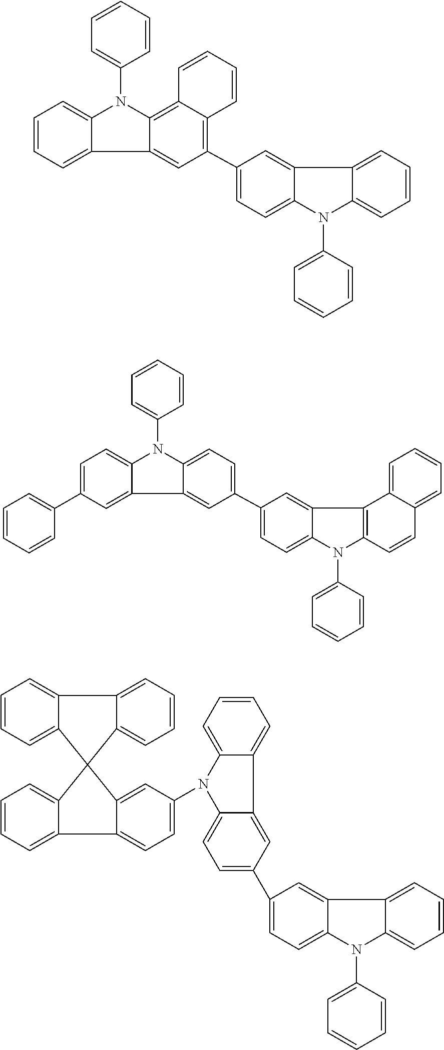 [SMILES: c1ccc(-n2c3ccccc3c3cc(-c4cc5c6ccccc6n(-c6ccccc6)c5c5ccccc45)ccc32)cc1 ; c1ccc(-c2ccc3c(c2)c2cc(-c4ccc5c(c4)c4c6ccccc6ccc4n5-c4ccccc4)ccc2n3-c2ccccc2)cc1 ; c1ccc(-n2c3ccccc3c3cc(-c4ccc5c(c4)c4ccccc4n5-c4ccc5c(c4)C4(c6ccccc6-c6ccccc64)c4ccccc4-5)ccc32)cc1]

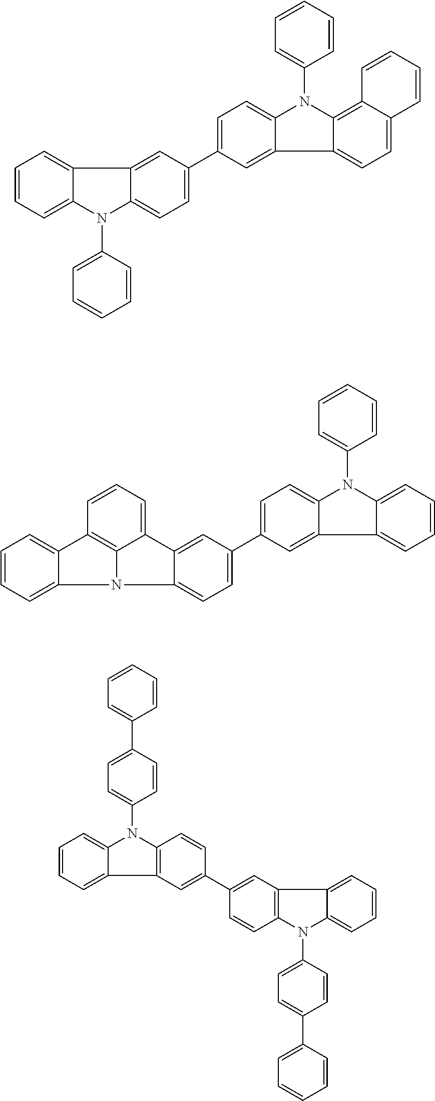 [SMILES: c1ccc(-n2c3ccccc3c3cc(-c4ccc5c(c4)c4ccc6ccccc6c4n5-c4ccccc4)ccc32)cc1 ; c1ccc(-n2c3ccccc3c3cc(-c4ccc5c(c4)c4cccc6c7ccccc7n5c64)ccc32)cc1 ; c1ccc(-c2ccc(-n3c4ccccc4c4cc(-c5ccc6c(c5)c5ccccc5n6-c5ccc(-c6ccccc6)cc5)ccc43)cc2)cc1]

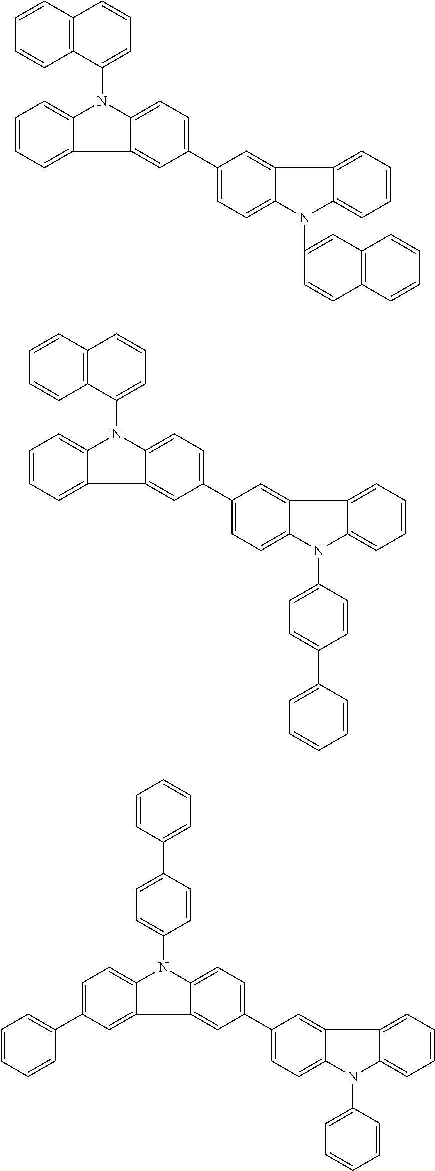 [SMILES: c1ccc2cc(-n3c4ccccc4c4cc(-c5ccc6c(c5)c5ccccc5n6-c5cccc6ccccc56)ccc43)ccc2c1 ; c1ccc(-c2ccc(-n3c4ccccc4c4cc(-c5ccc6c(c5)c5ccccc5n6-c5cccc6ccccc56)ccc43)cc2)cc1 ; c1ccc(-c2ccc(-n3c4ccc(-c5ccccc5)cc4c4cc(-c5ccc6c(c5)c5ccccc5n6-c5ccccc5)ccc43)cc2)cc1]

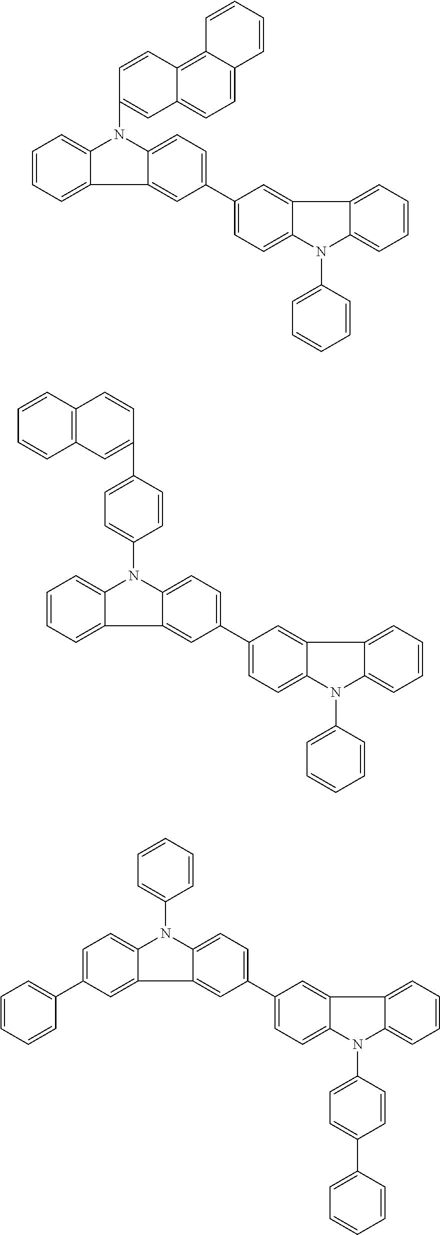 [SMILES: c1ccc(-n2c3ccccc3c3cc(-c4ccc5c(c4)c4ccccc4n5-c4ccc5c(ccc6ccccc65)c4)ccc32)cc1 ; c1ccc(-n2c3ccccc3c3cc(-c4ccc5c(c4)c4ccccc4n5-c4ccc(-c5ccc6ccccc6c5)cc4)ccc32)cc1 ; c1ccc(-c2ccc(-n3c4ccccc4c4cc(-c5ccc6c(c5)c5cc(-c7ccccc7)ccc5n6-c5ccccc5)ccc43)cc2)cc1]

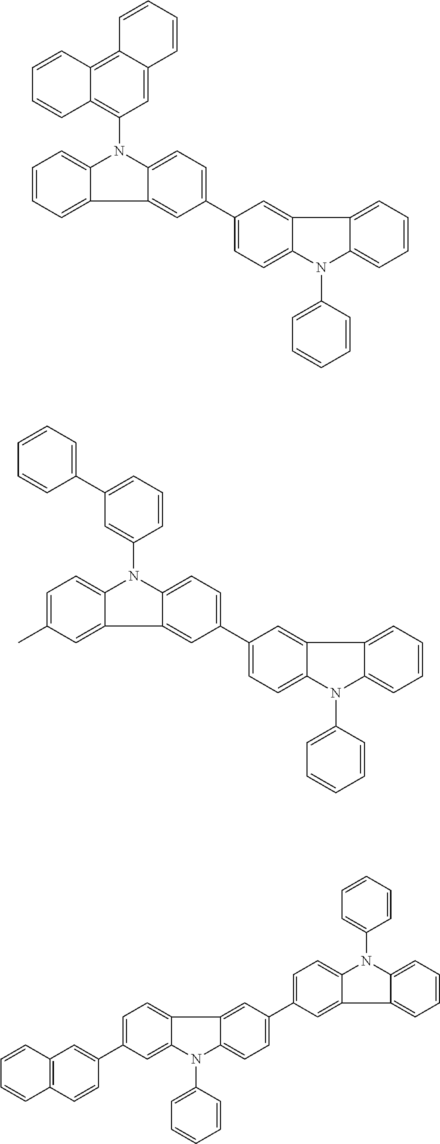 [SMILES: c1ccc(-n2c3ccccc3c3cc(-c4ccc5c(c4)c4ccccc4n5-c4cc5ccccc5c5ccccc45)ccc32)cc1 ; Cc1ccc2c(c1)c1cc(-c3ccc4c(c3)c3ccccc3n4-c3ccccc3)ccc1n2-c1cccc(-c2ccccc2)c1 ; c1ccc(-n2c3ccccc3c3cc(-c4ccc5c(c4)c4ccc(-c6ccc7ccccc7c6)cc4n5-c4ccccc4)ccc32)cc1]

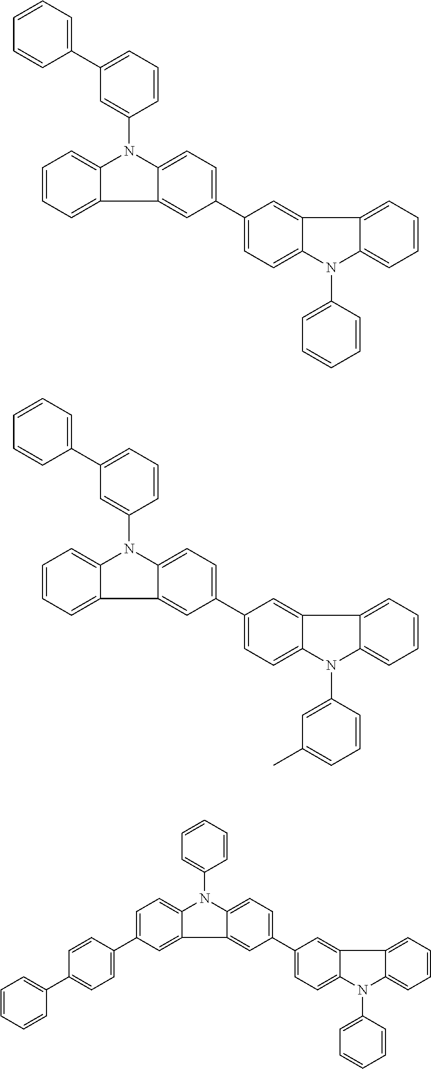 [SMILES: c1ccc(-c2cccc(-n3c4ccccc4c4cc(-c5ccc6c(c5)c5ccccc5n6-c5ccccc5)ccc43)c2)cc1 ; Cc1cccc(-n2c3ccccc3c3cc(-c4ccc5c(c4)c4ccccc4n5-c4cccc(-c5ccccc5)c4)ccc32)c1 ; c1ccc(-c2ccc(-c3ccc4c(c3)c3cc(-c5ccc6c(c5)c5ccccc5n6-c5ccccc5)ccc3n4-c3ccccc3)cc2)cc1]

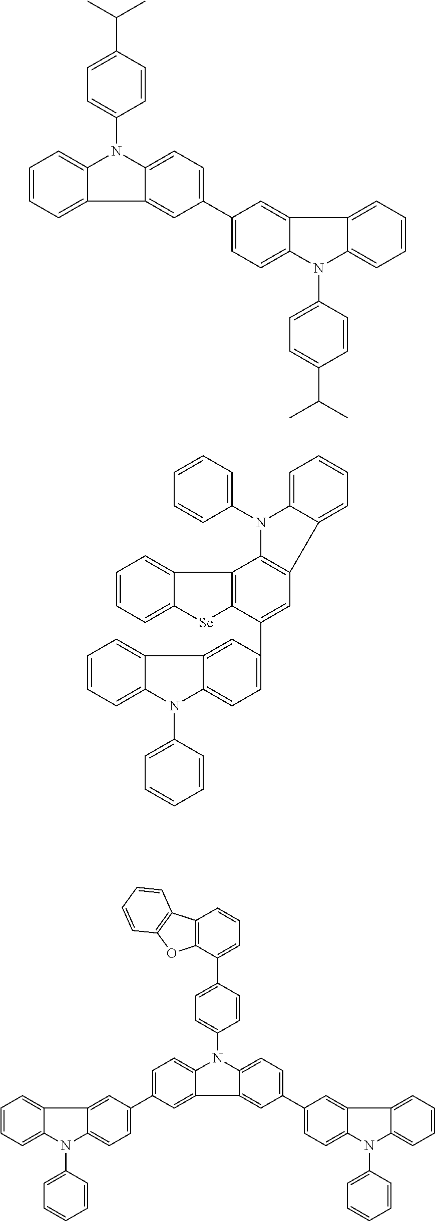 [SMILES: CC(C)c1ccc(-n2c3ccccc3c3cc(-c4ccc5c(c4)c4ccccc4n5-c4ccc(C(C)C)cc4)ccc32)cc1 ; c1ccc(-n2c3ccccc3c3cc(-c4cc5c6ccccc6n(-c6ccccc6)c5c5c4[se]c4ccccc45)ccc32)cc1 ; c1ccc(-n2c3ccccc3c3cc(-c4ccc5c(c4)c4cc(-c6ccc7c(c6)c6ccccc6n7-c6ccccc6)ccc4n5-c4ccc(-c5cccc6c5oc5ccccc56)cc4)ccc32)cc1]

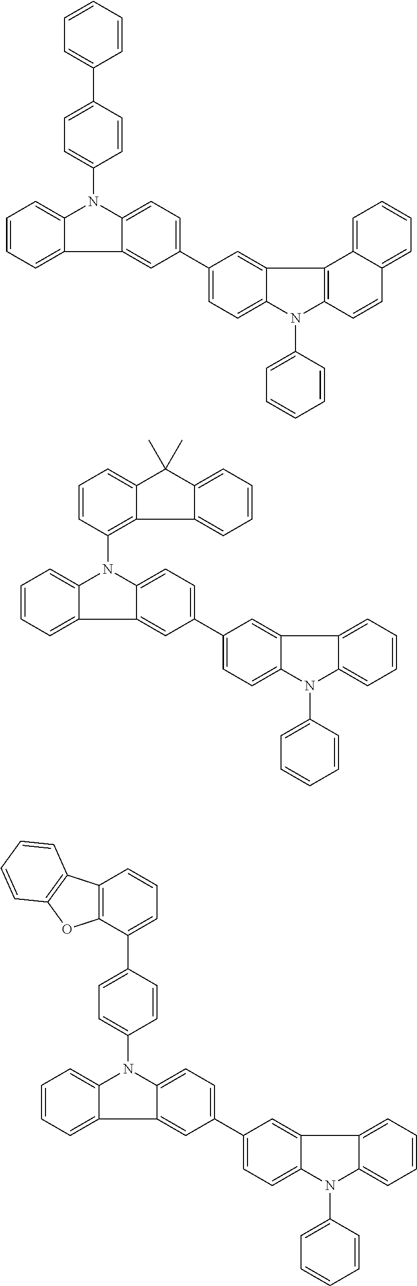 [SMILES: c1ccc(-c2ccc(-n3c4ccccc4c4cc(-c5ccc6c(c5)c5c7ccccc7ccc5n6-c5ccccc5)ccc43)cc2)cc1 ; CC1(C)c2ccccc2-c2c(-n3c4ccccc4c4cc(-c5ccc6c(c5)c5ccccc5n6-c5ccccc5)ccc43)cccc21 ; c1ccc(-n2c3ccccc3c3cc(-c4ccc5c(c4)c4ccccc4n5-c4ccc(-c5cccc6c5oc5ccccc56)cc4)ccc32)cc1]

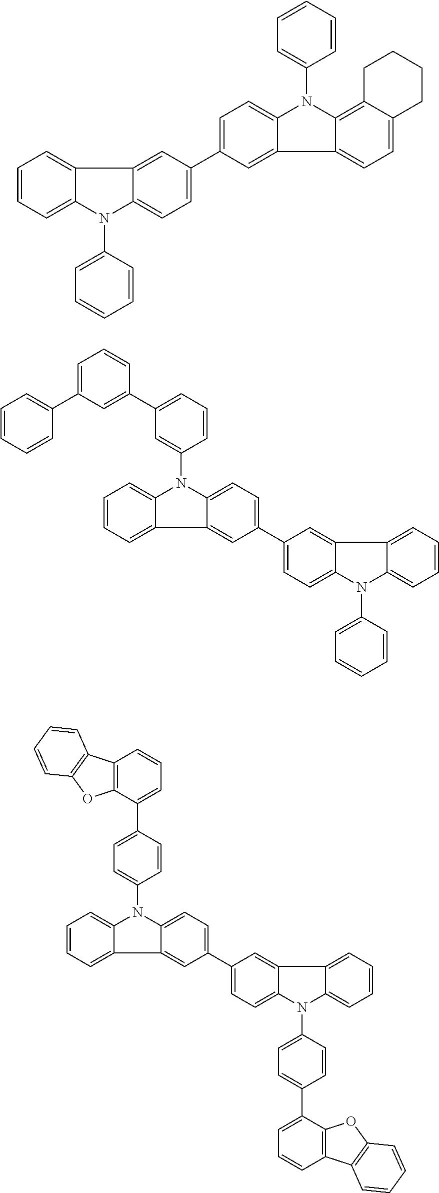

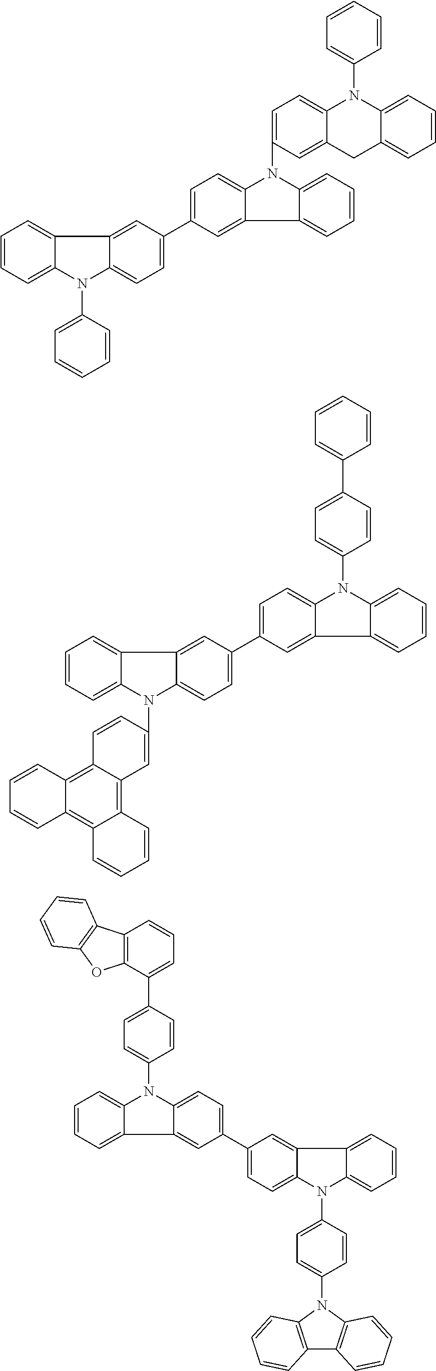

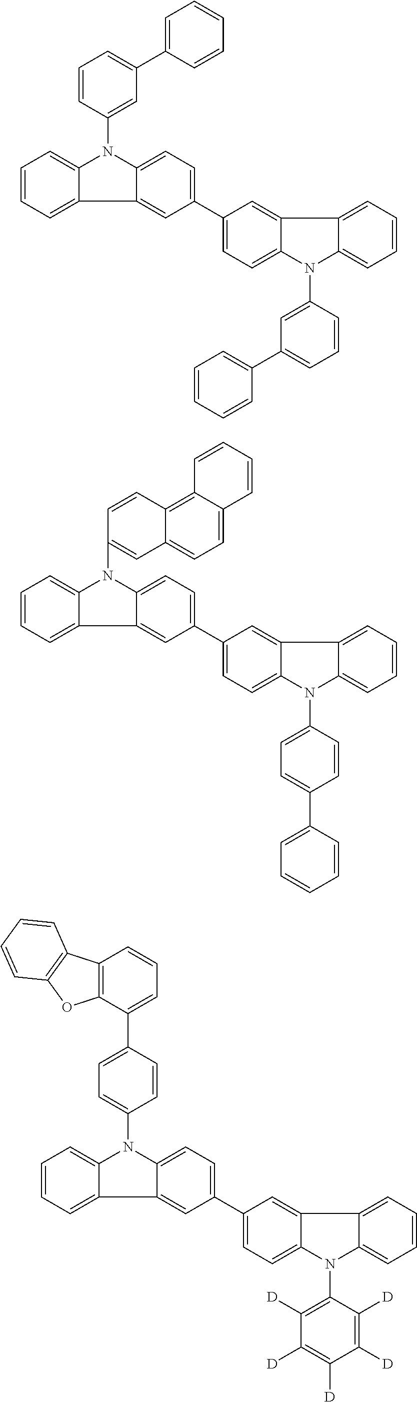

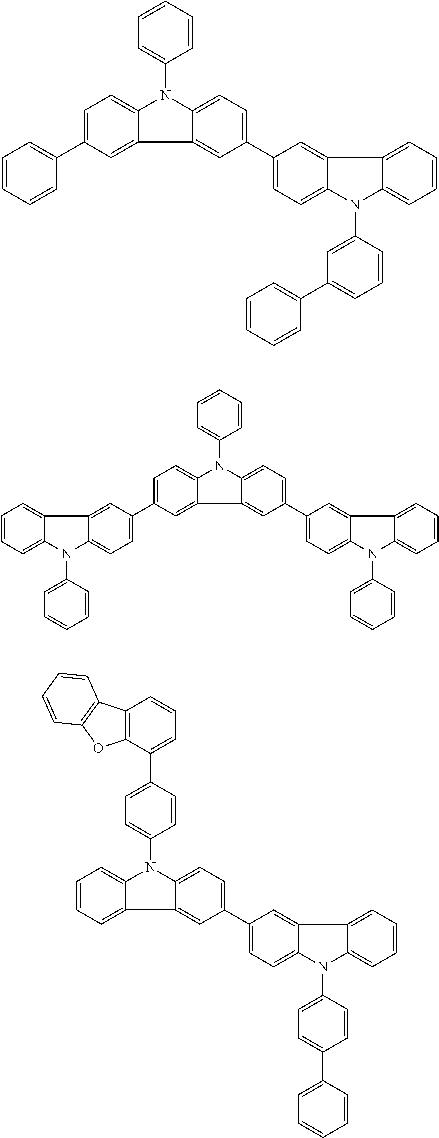 [SMILES: c1ccc(-c2cccc(-n3c4ccccc4c4cc(-c5ccc6c(c5)c5cc(-c7ccccc7)ccc5n6-c5ccccc5)ccc43)c2)cc1 ; c1ccc(-n2c3ccccc3c3cc(-c4ccc5c(c4)c4cc(-c6ccc7c(c6)c6ccccc6n7-c6ccccc6)ccc4n5-c4ccccc4)ccc32)cc1 ; c1ccc(-c2ccc(-n3c4ccccc4c4cc(-c5ccc6c(c5)c5ccccc5n6-c5ccc(-c6cccc7c6oc6ccccc67)cc5)ccc43)cc2)cc1]

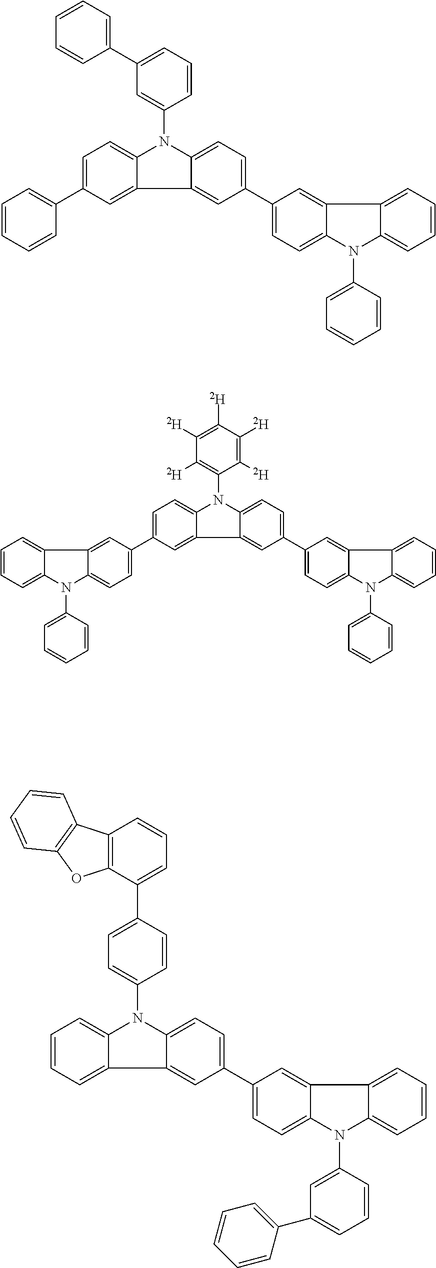 [SMILES: c1ccc(-c2cccc(-n3c4ccc(-c5ccccc5)cc4c4cc(-c5ccc6c(c5)c5ccccc5n6-c5ccccc5)ccc43)c2)cc1 ; [2H]c1c([2H])c([2H])c(-n2c3ccc(-c4ccc5c(c4)c4ccccc4n5-c4ccccc4)cc3c3cc(-c4ccc5c(c4)c4ccccc4n5-c4ccccc4)ccc32)c([2H])c1[2H] ; c1ccc(-c2cccc(-n3c4ccccc4c4cc(-c5ccc6c(c5)c5ccccc5n6-c5ccc(-c6cccc7c6oc6ccccc67)cc5)ccc43)c2)cc1]

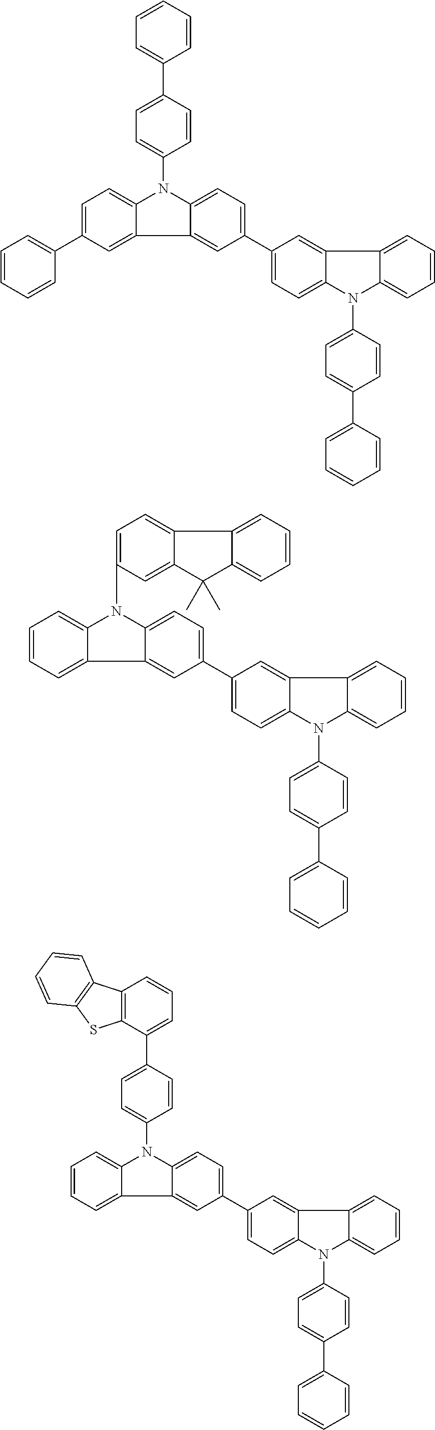 [SMILES: c1ccc(-c2ccc(-n3c4ccccc4c4cc(-c5ccc6c(c5)c5cc(-c7ccccc7)ccc5n6-c5ccc(-c6ccccc6)cc5)ccc43)cc2)cc1 ; CC1(C)c2ccccc2-c2ccc(-n3c4ccccc4c4cc(-c5ccc6c(c5)c5ccccc5n6-c5ccc(-c6ccccc6)cc5)ccc43)cc21 ; c1ccc(-c2ccc(-n3c4ccccc4c4cc(-c5ccc6c(c5)c5ccccc5n6-c5ccc(-c6cccc7c6sc6ccccc67)cc5)ccc43)cc2)cc1]

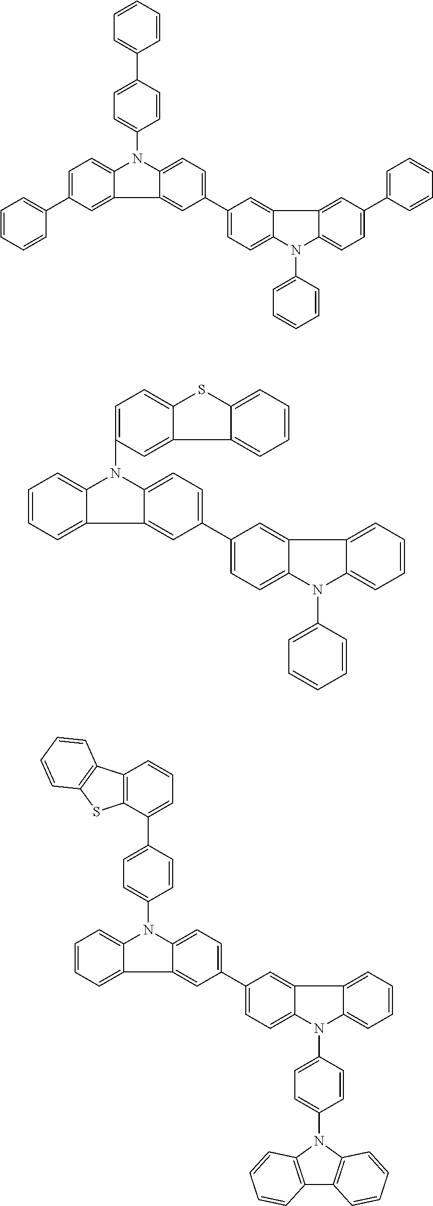 [SMILES: c1ccc(-c2ccc(-n3c4ccc(-c5ccccc5)cc4c4cc(-c5ccc6c(c5)c5cc(-c7ccccc7)ccc5n6-c5ccccc5)ccc43)cc2)cc1 ; c1ccc(-n2c3ccccc3c3cc(-c4ccc5c(c4)c4ccccc4n5-c4ccc5sc6ccccc6c5c4)ccc32)cc1 ; c1ccc2c(c1)sc1c(-c3ccc(-n4c5ccccc5c5cc(-c6ccc7c(c6)c6ccccc6n7-c6ccc(-n7c8ccccc8c8ccccc87)cc6)ccc54)cc3)cccc12]

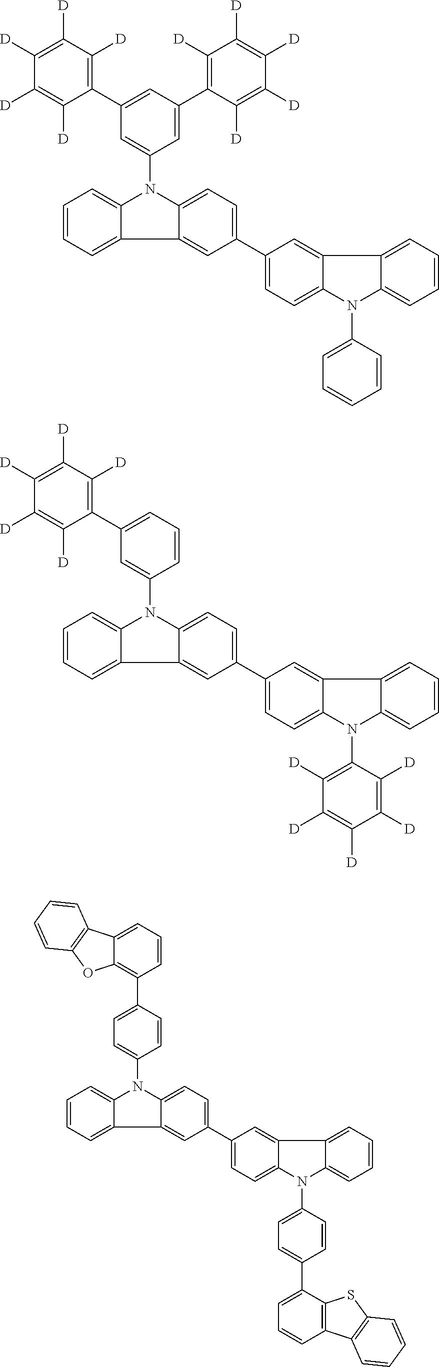 [SMILES: [2H]c1c([2H])c([2H])c(-c2cc(-c3c([2H])c([2H])c([2H])c([2H])c3[2H])cc(-n3c4ccccc4c4cc(-c5ccc6c(c5)c5ccccc5n6-c5ccccc5)ccc43)c2)c([2H])c1[2H] ; [2H]c1c([2H])c([2H])c(-c2cccc(-n3c4ccccc4c4cc(-c5ccc6c(c5)c5ccccc5n6-c5c([2H])c([2H])c([2H])c([2H])c5[2H])ccc43)c2)c([2H])c1[2H] ; c1ccc2c(c1)oc1c(-c3ccc(-n4c5ccccc5c5cc(-c6ccc7c(c6)c6ccccc6n7-c6ccc(-c7cccc8c7sc7ccccc78)cc6)ccc54)cc3)cccc12]

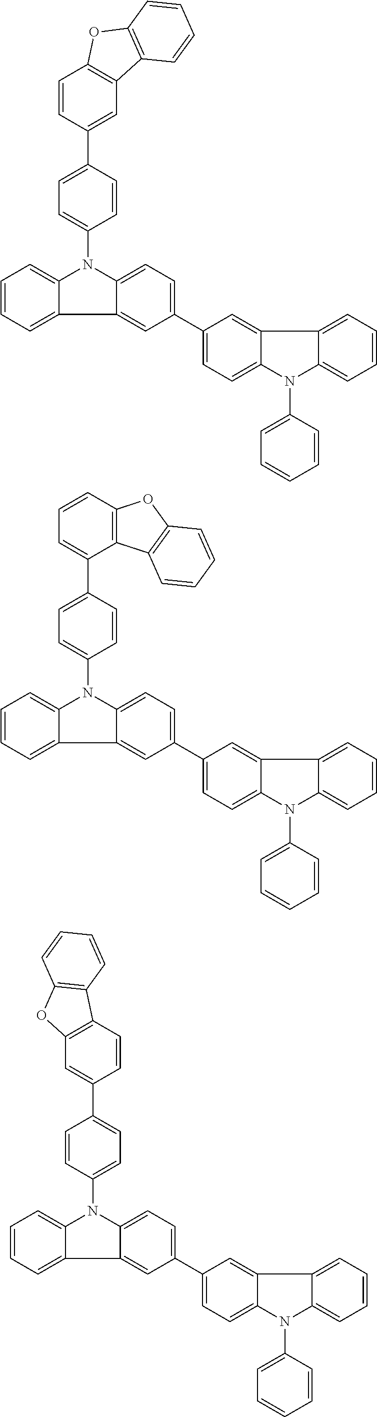 [SMILES: c1ccc(-n2c3ccccc3c3cc(-c4ccc5c(c4)c4ccccc4n5-c4ccc(-c5ccc6oc7ccccc7c6c5)cc4)ccc32)cc1 ; c1ccc(-n2c3ccccc3c3cc(-c4ccc5c(c4)c4ccccc4n5-c4ccc(-c5cccc6oc7ccccc7c56)cc4)ccc32)cc1 ; c1ccc(-n2c3ccccc3c3cc(-c4ccc5c(c4)c4ccccc4n5-c4ccc(-c5ccc6c(c5)oc5ccccc56)cc4)ccc32)cc1]

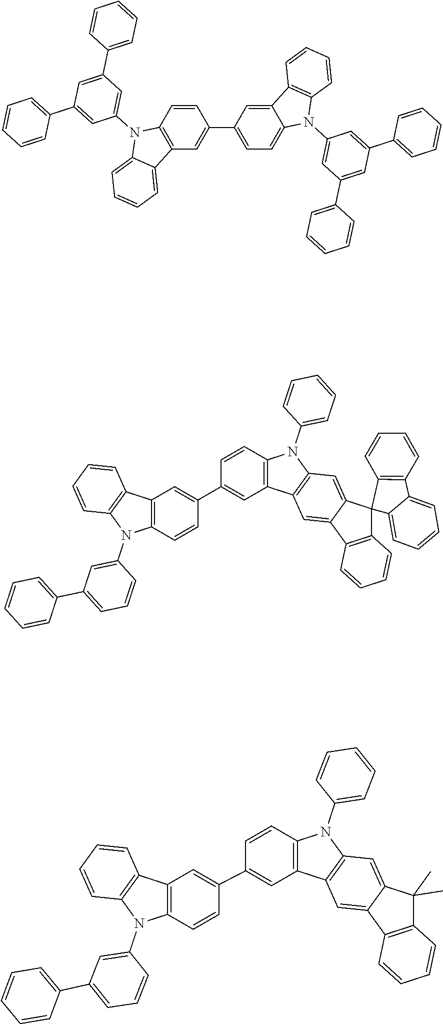 [SMILES: c1ccc(-c2cc(-c3ccccc3)cc(-n3c4ccccc4c4cc(-c5ccc6c(c5)c5ccccc5n6-c5cc(-c6ccccc6)cc(-c6ccccc6)c5)ccc43)c2)cc1 ; c1ccc(-c2cccc(-n3c4ccccc4c4cc(-c5ccc6c(c5)c5cc7c(cc5n6-c5ccccc5)C5(c6ccccc6-c6ccccc65)c5ccccc5-7)ccc43)c2)cc1 ; CC1(C)c2ccccc2-c2cc3c4cc(-c5ccc6c(c5)c5ccccc5n6-c5cccc(-c6ccccc6)c5)ccc4n(-c4ccccc4)c3cc21]

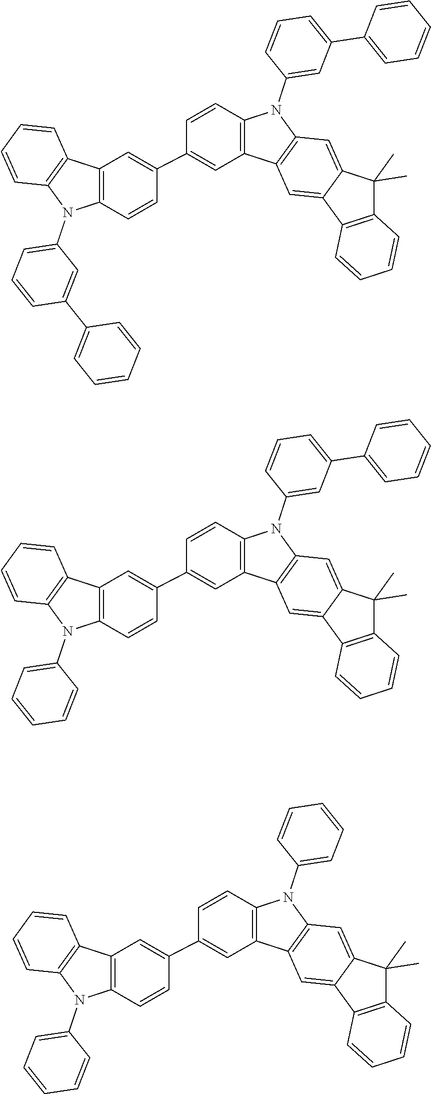 [SMILES: CC1(C)c2ccccc2-c2cc3c4cc(-c5ccc6c(c5)c5ccccc5n6-c5cccc(-c6ccccc6)c5)ccc4n(-c4cccc(-c5ccccc5)c4)c3cc21 ; CC1(C)c2ccccc2-c2cc3c4cc(-c5ccc6c(c5)c5ccccc5n6-c5ccccc5)ccc4n(-c4cccc(-c5ccccc5)c4)c3cc21 ; CC1(C)c2ccccc2-c2cc3c4cc(-c5ccc6c(c5)c5ccccc5n6-c5ccccc5)ccc4n(-c4ccccc4)c3cc21]

The combination of at least one compound of formula (I) or the preferred embodiments thereof that are set out above with a compound of one of the formulae (H-1), (H-2), (H-3), (H-4) and (H-5) can achieve surprising advantages. The present invention therefore provides a composition comprising at least one compound of formula (I) or the preferred embodiments thereof that are set out above and at least one further matrix material, wherein the further matrix material is selected from compounds of one of the formulae (H-1), (H-2), (H-3), (H-4) and (H-5).

In a preferred configuration, it may be the case that the inventive compound of formula (I) is used as matrix material for phosphorescent emitters in combination with a further matrix material selected from compounds of one of the formulae (H-1), (H-2), (H-3), (H-4) and (H-5).

It may preferably be the case that the composition consists of at least one compound of formula (I) or the preferred embodiments thereof that are set out above and at least one compound of one of the formulae (H-1), (H-2), (H-3), (H-4) and (H-5). These compositions are especially suitable as what are called pre-mixtures, which can be evaporated together.

In this context, the compounds of one of the formulae (H-1), (H-2), (H-3), (H-4) and (H-5) may each be used individually or as a mixture of two, three or more compounds of the respective structures.

In addition, the compounds of the formulae (H-1), (H-2), (H-3), (H-4) and (H-5) may be used individually or as a mixture of two, three or more compounds of different structures.

The compound of formula (I) or the preferred embodiments thereof that are set out above preferably has a proportion by mass in the composition in the range from 10% by weight to 95% by weight, more preferably in the range from 15% by weight to 90% by weight, and very preferably in the range from 40% by weight to 70% by weight, based on the total mass of the composition.

It may further be the case that the compounds of one of the formulae (H-1), (H-2), (H-3), (H-4) and (H-5) have a proportion by mass in the composition in the range from 5% by weight to 90% by weight, preferably in the range from 10% by weight to 85% by weight, more preferably in the range from 20% by weight to 85% by weight, even more preferably in the range from 30% by weight to 80% by weight, very particularly preferably in the range from 20% by weight to 60% by weight and most preferably in the range from 30% by weight to 50% by weight, based on the overall composition.

It may additionally be the case that the further matrix material is a hole-transporting matrix material of at least one of the formulae (H-1), (H-2), (H-3), (H-4) and (H-5), and the hole-transporting matrix material has a proportion by mass in the composition in the range from 10% by weight to 95% by weight, preferably in the range from 15% by weight to 90% by weight, more preferably in the range from 15% by weight to 80% by weight, even more preferably in the range from 20% by weight to 70% by weight, very particularly preferably in the range from 40% by weight to 80% by weight and most preferably in the range from 50% by weight to 70% by weight, based on the overall composition.

It may additionally be the case that the composition consists exclusively of the formula (I) or the preferred embodiments thereof that are set out above and one of the further matrix materials mentioned, preferably compounds of at least one of the formulae (H-1), (H-2), (H-3), (H-4) and (H-5).

Suitable phosphorescent compounds (=triplet emitters) are especially compounds which, when suitably excited, emit light, preferably in the visible region, and also contain at least one atom of atomic number greater than 20, preferably greater than 38 and less than 84, more preferably greater than 56 and less than 80, especially a metal having this atomic number. Preferred phosphorescence emitters used are compounds containing copper, molybdenum, tungsten, rhenium, ruthenium, osmium, rhodium, iridium, palladium, platinum, silver, gold or europium, especially compounds containing iridium or platinum.

Examples of the above-described emitters can be found in applications WO 00/70655, WO 2001/41512, WO 2002/02714, WO 2002/15645, EP 1191613, EP 1191612, EP 1191614, WO 05/033244, WO 05/019373, US 2005/0258742, WO 2009/146770, WO 2010/015307, WO 2010/031485, WO 2010/054731, WO 2010/054728, WO 2010/086089, WO 2010/099852, WO 2010/102709, WO 2011/032626, WO 2011/066898, WO 2011/157339, WO 2012/007086, WO 2014/008982, WO 2014/023377, WO 2014/094961, WO 2014/094960, WO 2015/036074, WO 2015/104045, WO 2015/117718, WO 2016/015815, WO 2016/124304, WO 2017/032439 and WO 2018/011186. In general, all phosphorescent complexes as used for phosphorescent electroluminescent devices according to the prior art and as known to those skilled in the art in the field of organic electroluminescence are suitable, and the person skilled in the art will be able to use further phosphorescent complexes without exercising inventive skill.

Examples of phosphorescent dopants are listed in the following table:

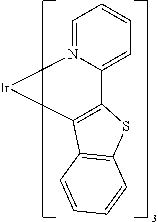

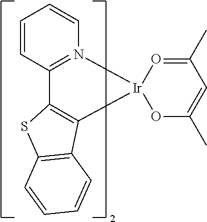

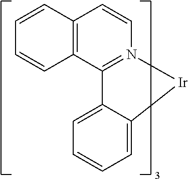

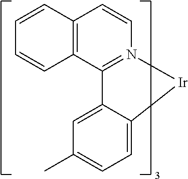

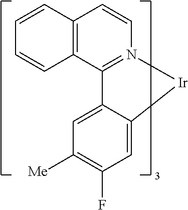

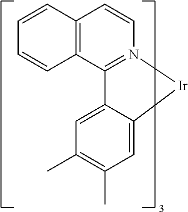

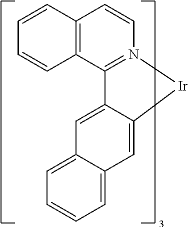

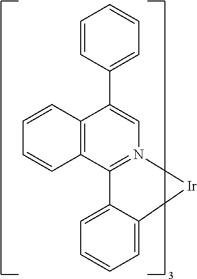

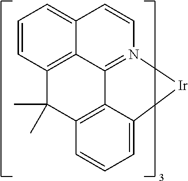

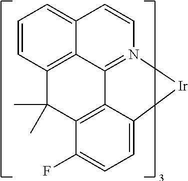

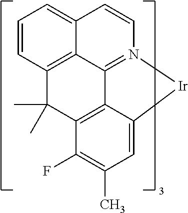

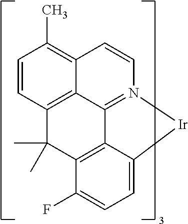

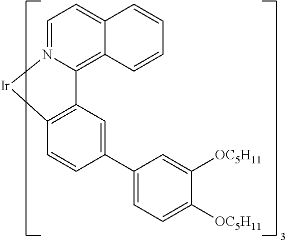

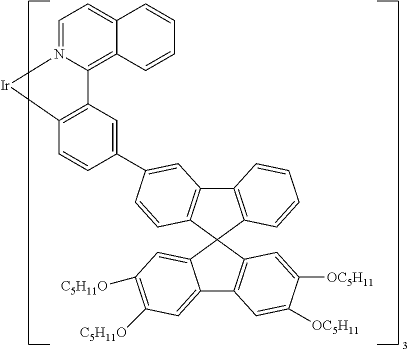

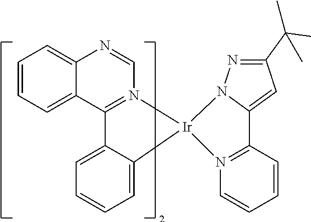

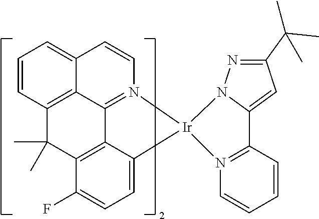

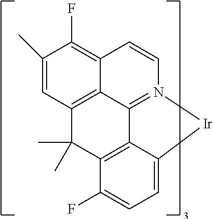

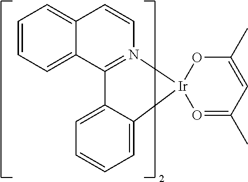

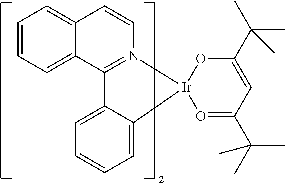

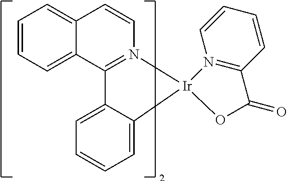

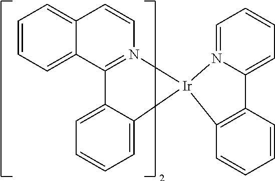

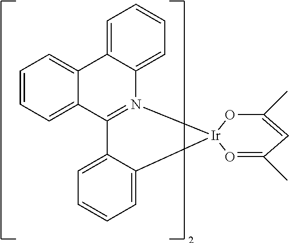

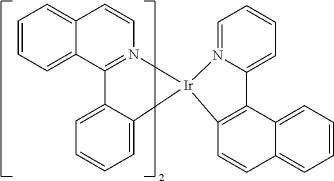

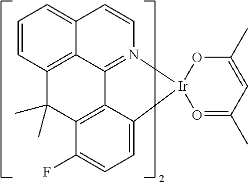

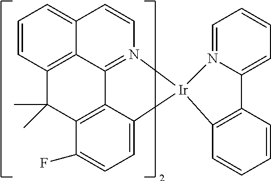

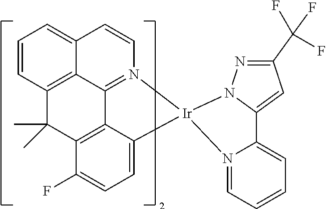

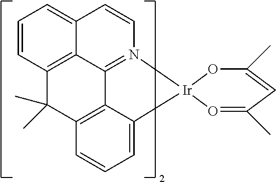

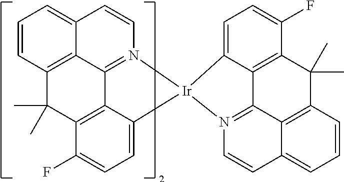

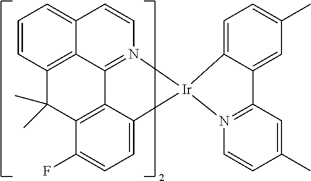

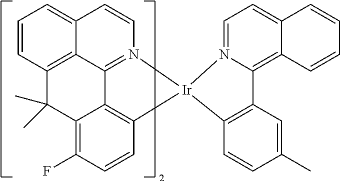

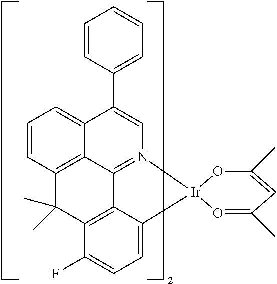

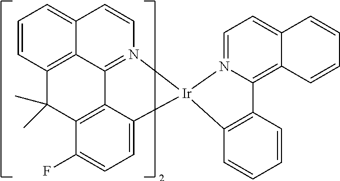

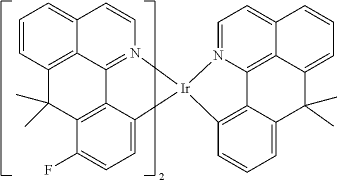

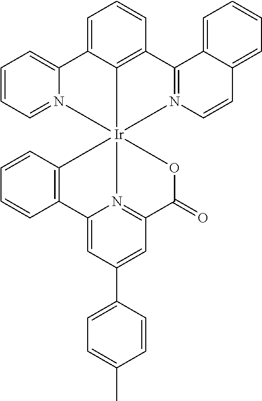

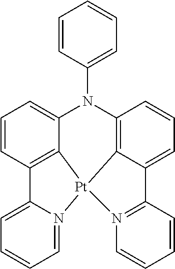

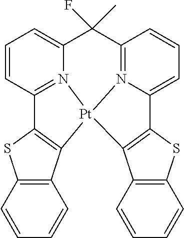

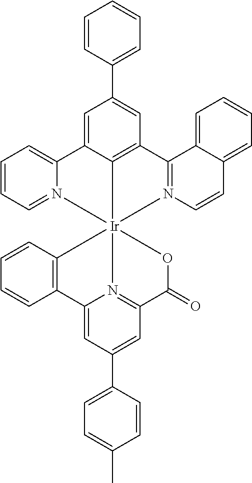

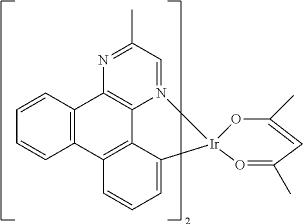

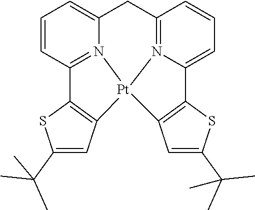

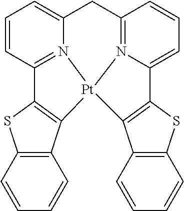

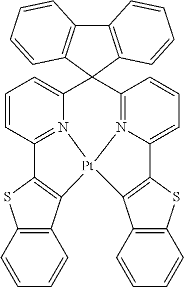

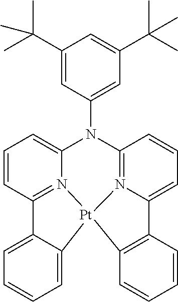

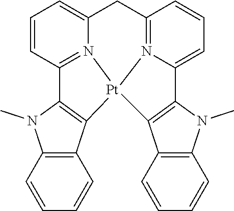

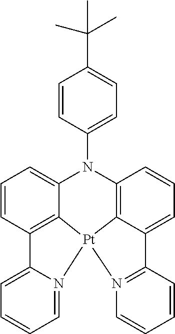

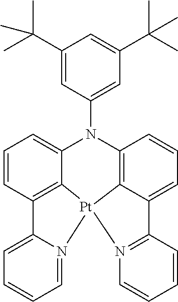

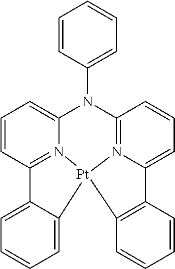

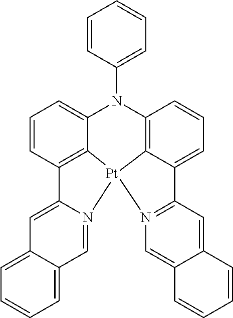

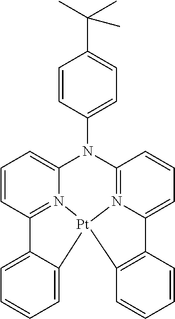

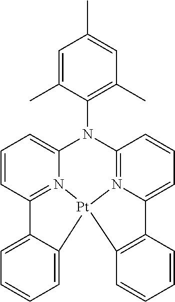

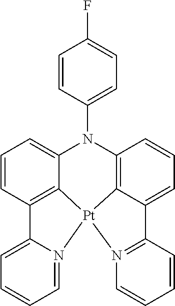

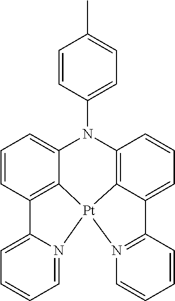

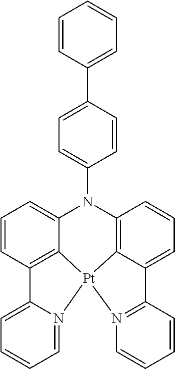

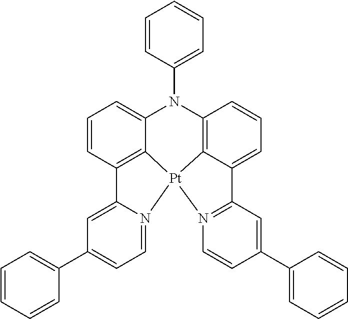

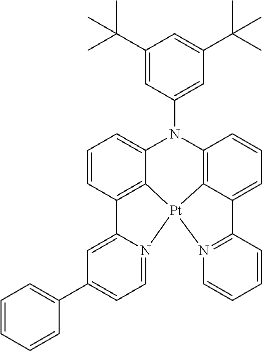

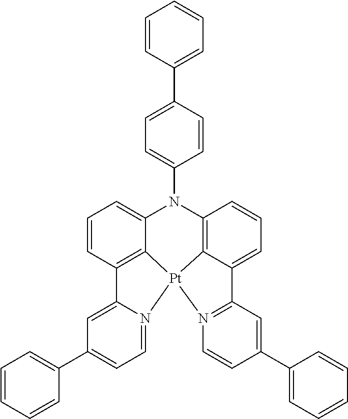

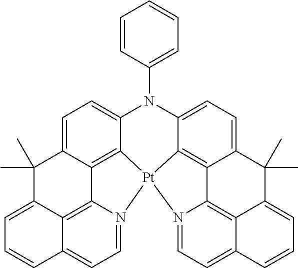

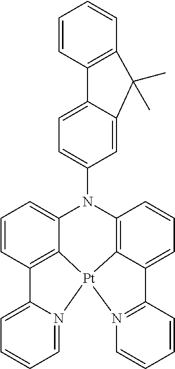

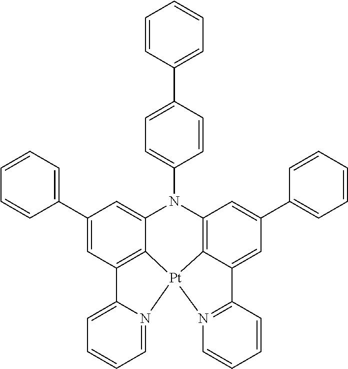

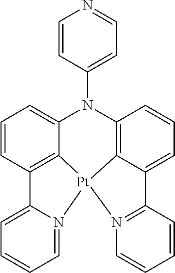

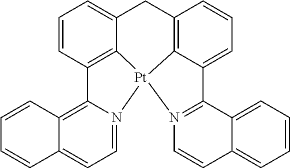

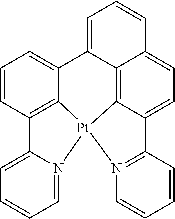

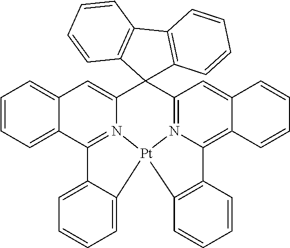

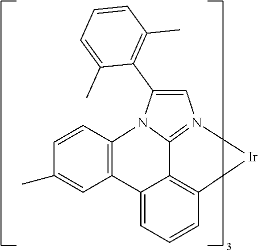

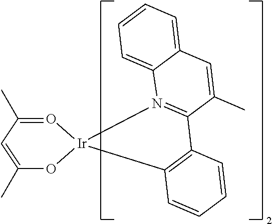

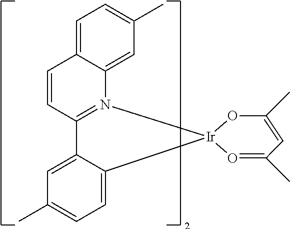

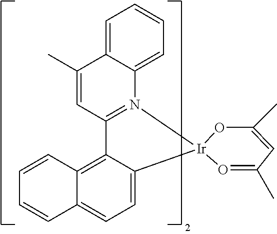

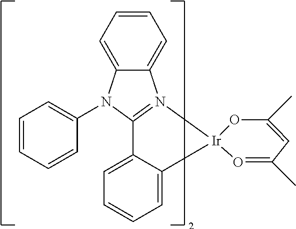

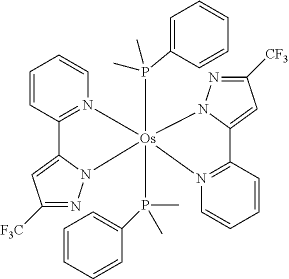

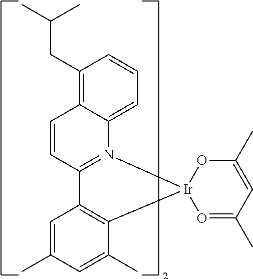

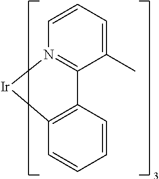

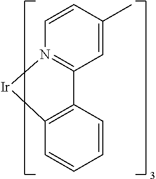

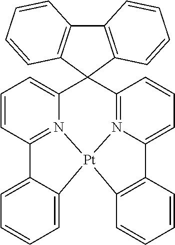

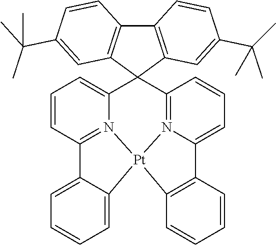

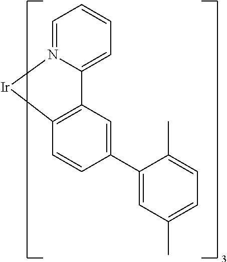

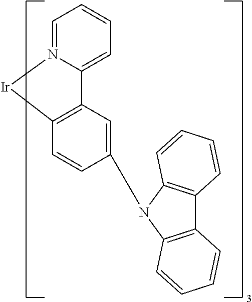

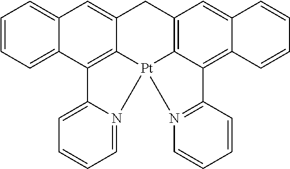

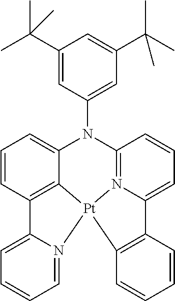

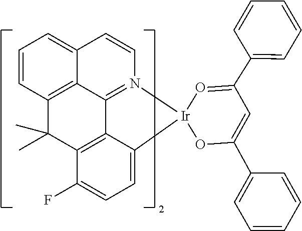

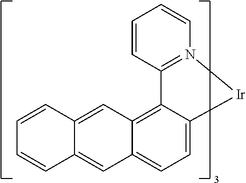

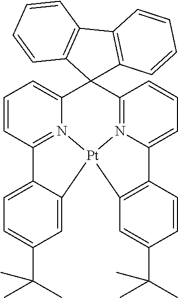

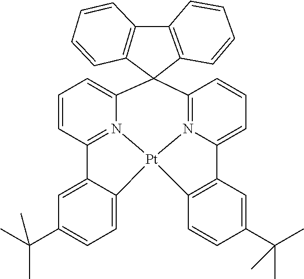

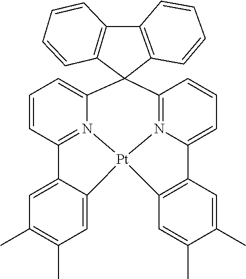

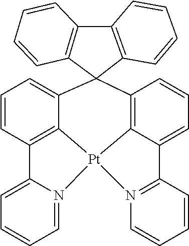

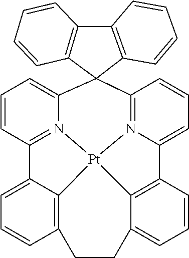

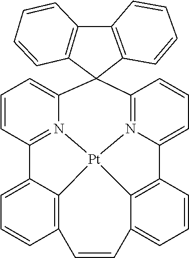

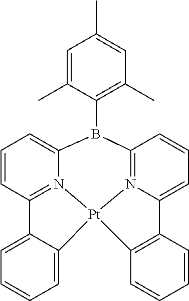

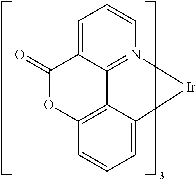

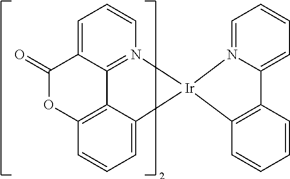

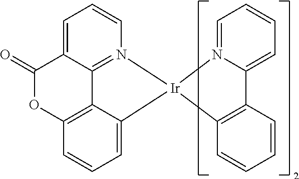

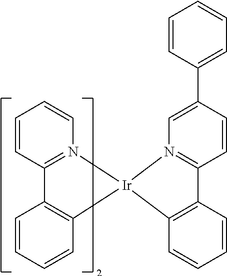

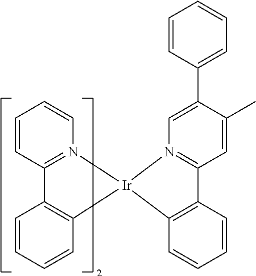

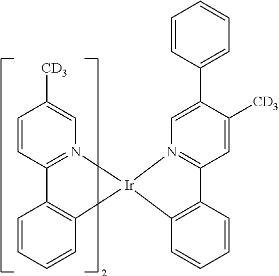

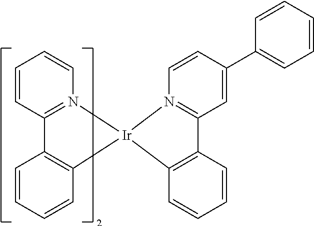

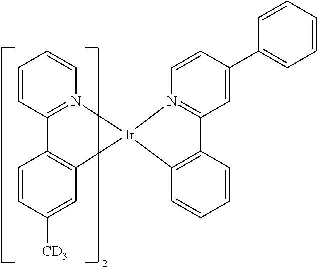

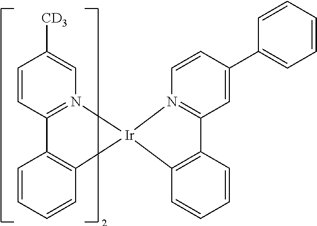

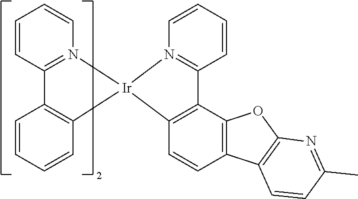

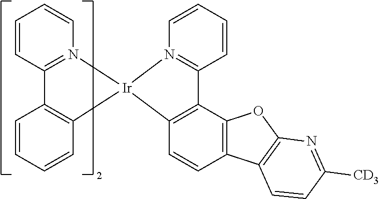

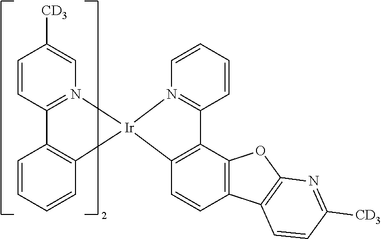

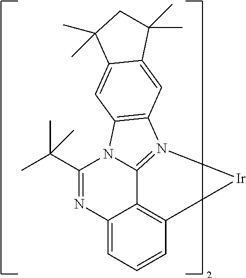

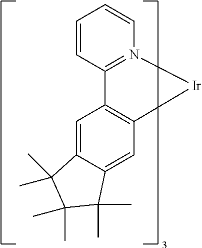

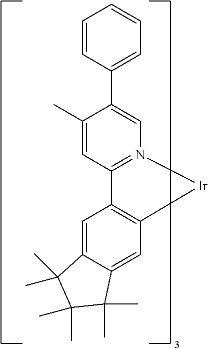

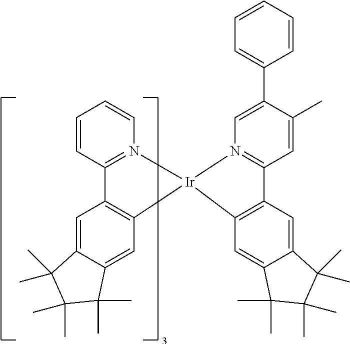

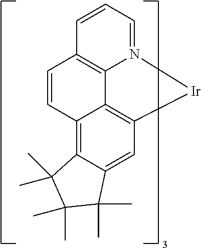

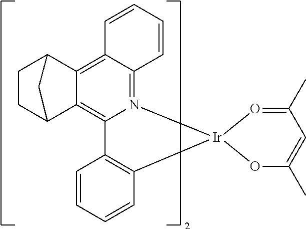

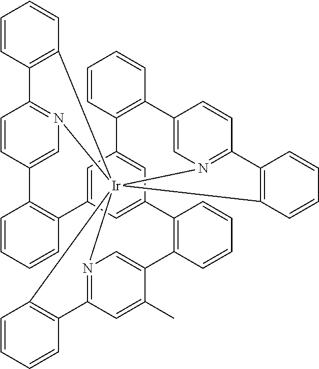

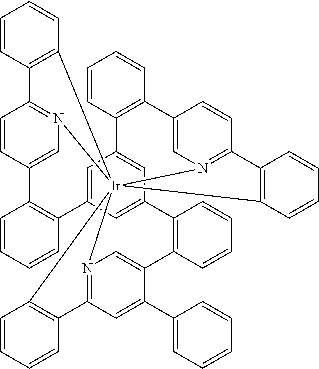

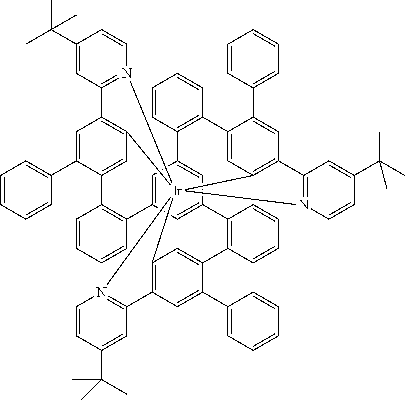

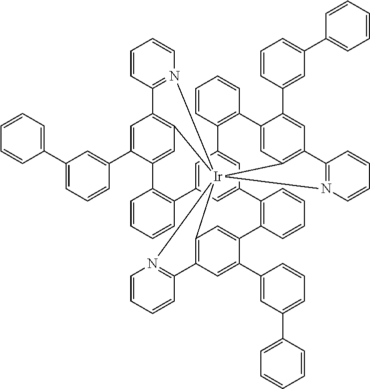

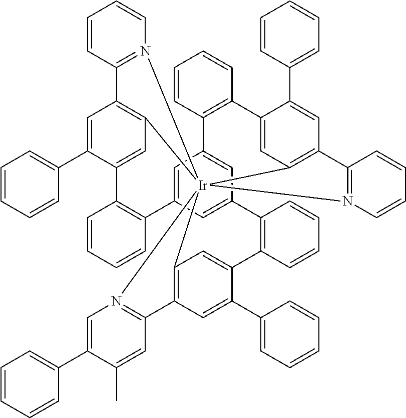

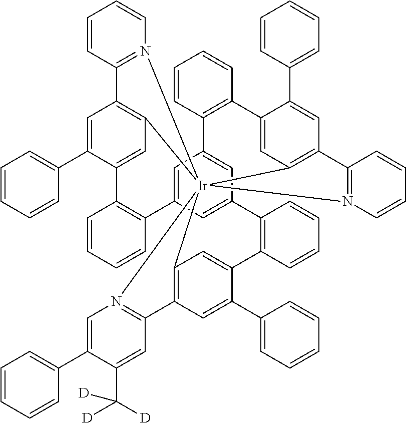

The compounds of the invention are especially also suitable as matrix materials for phosphorescent emitters in organic electroluminescent devices, as described, for example, in WO 98/24271, US 2011/0248247 and US 2012/0223633. In these multicolor display components, an additional blue emission later is applied by vapor deposition over the full area to all pixels, including those having a color other than blue.

In a further embodiment of the invention, the organic electroluminescent device of the invention does not contain any separate hole injection layer and/or hole transport layer and/or hole blocker layer and/or electron transport layer, meaning that the emitting layer directly adjoins the hole injection layer or the anode, and/or the emitting layer directly adjoins the electron transport layer or the electron injection layer or the cathode, as described, for example, in WO 2005/053051. It is additionally possible to use a metal complex identical or similar to the metal complex in the emitting layer as hole transport or hole injection material directly adjoining the emitting layer, as described, for example, in WO 2009/030981.

In the further layers of the organic electroluminescent device of the invention, it is possible to use any materials as typically used according to the prior art. The person skilled in the art will therefore be able, without exercising inventive skill, to use any materials known for organic electroluminescent devices in combination with the inventive compounds of formula (I) or the above-recited preferred embodiments.

Additionally preferred is an organic electroluminescent device, characterized in that one or more layers are coated by a sublimation process. In this case, the materials are applied by vapor deposition in vacuum sublimation systems at an initial pressure of less than 10⁻⁵ mbar, preferably less than 10⁻⁶ mbar. However, it is also possible that the initial pressure is even lower, for example less than 10⁻⁷ mbar.

Preference is likewise given to an organic electroluminescent device, characterized in that one or more layers are coated by the OVPD (organic vapor phase deposition) method or with the aid of a carrier gas sublimation. In this case, the materials are applied at a pressure between 10⁻⁵ mbar and 1 bar. A special case of this method is the OVJP (organic vaporjet printing) method, in which the materials are applied directly by a nozzle and thus structured.

Preference is additionally given to an organic electroluminescent device, characterized in that one or more layers are produced from solution, for example by spin-coating, or by any printing method, for example screen printing, flexographic printing, offset printing, LITI (light-induced thermal imaging, thermal transfer printing), inkjet printing or nozzle printing. For this purpose, soluble compounds are needed, which are obtained, for example, through suitable substitution.

Formulations for application of a compound of formula (I) or the preferred embodiments thereof that are set out above are novel. The present invention therefore further provides a formulation comprising at least one solvent and a compound of formula (I) or the preferred embodiments thereof that are set out above. The present invention further provides a formulation comprising at least one solvent and a compound of formula (I) or the preferred embodiments thereof that are set out above, and a compound of at least one of the formulae (H-1), (H-2), (H-3), (H-4) and (H-5).

In addition, hybrid methods are possible, in which, for example, one or more layers are applied from solution and one or more further layers are applied by vapor deposition.

Those skilled in the art are generally aware of these methods and are able to apply them without exercising inventive skill to organic electroluminescent devices comprising the compounds of the invention.

The compounds of the invention and the organic electroluminescent devices of the invention have the particular feature of an improved lifetime over the prior art. At the same time, the further electronic properties of the electroluminescent devices, such as efficiency or operating voltage, remain at least equally good. In a further variant, the compounds of the invention and the organic electroluminescent devices of the invention especially feature improved efficiency and/or operating voltage and higher lifetime compared to the prior art.

The electronic devices of the invention, especially organic electroluminescent devices, are notable for one or more of the following surprising advantages over the prior art:
1. Electronic devices, especially organic electroluminescent devices, comprising compounds of formula (I) or the preferred embodiments recited above and hereinafter, especially as matrix material or as electron-conducting materials, have a very good lifetime. In this context, these compounds especially bring about low roll-off, i.e. a small drop in power efficiency of the device at high luminances.
2. Electronic devices, especially organic electroluminescent devices, comprising compounds of formula (I) or the preferred embodiments recited above and hereinafter, as electron-conducting materials and/or matrix materials, have excellent efficiency. In this context, compounds of the invention having structures of formula (I) or the preferred embodiments recited above and hereinafter bring about a low operating voltage when used in electronic devices.
3. The inventive compounds of formula (I) or the preferred embodiments recited above and hereinafter exhibit very high stability and lifetime.
4. With compounds of formula (I) or the preferred embodiments recited above and hereinafter, it is possible to avoid the formation of optical loss channels in electronic devices, especially organic electroluminescent devices. As a result, these devices feature a high PL efficiency and hence high EL efficiency of emitters, and excellent energy transmission of the matrices to dopants.
5. Compounds of formula (I) or the preferred embodiments recited above and hereinafter have excellent glass film formation.
6. Compounds of formula (I) or the preferred embodiments recited above and hereinafter form very good films from solutions.
7. Electronic devices, especially organic electroluminescent devices comprising compounds of formula (I) or the preferred embodiments detailed above and hereinafter, in combination with host materials of one or more of the formulae (H-1) to (H-5), especially as matrix material, have an improved lifetime and higher efficiency.
8. The compounds of formula (I) or the preferred embodiments recited above and hereinafter have a low triplet level T₁ which may, for example, be in the range of −2.22 eV to −2.9 eV.

These abovementioned advantages are not accompanied by an inordinately high deterioration in the further electronic properties.

It should be pointed out that variations of the embodiments described in the present invention are covered by the scope of this invention. Any feature disclosed in the present invention may, unless this is explicitly ruled out, be exchanged for alternative features which serve the same purpose or an equivalent or similar purpose. Thus, any feature disclosed in the present invention, unless stated otherwise, should be considered as an example of a generic series or as an equivalent or similar feature.

All features of the present invention may be combined with one another in any manner, unless particular features and/or steps are mutually exclusive. This is especially true of preferred features of the present invention. Equally, features of non-essential combinations may be used separately (and not in combination).

It should also be pointed out that many of the features, and especially those of the preferred embodiments of the present invention, should themselves be regarded as inventive and not merely as some of the embodiments of the present invention. For these features, independent protection may be sought in addition to or as an alternative to any currently claimed invention.

The technical teaching disclosed with the present invention may be abstracted and combined with other examples.

The invention is illustrated in detail by the examples which follow, without any intention of restricting it thereby. The person skilled in the art will be able to use the information given to execute the invention over the entire scope disclosed and to prepare further compounds of the invention without exercising inventive skill and to use them in electronic devices or to employ the process of the invention.

### EXAMPLES

The syntheses which follow, unless stated otherwise, are conducted under a protective gas atmosphere in dried solvents. The solvents and reagents can be purchased, for example, from Sigma-ALDRICH or ABCR. For the compounds known from the literature, the corresponding CAS numbers are also reported in each case.

### Synthesis Examples

### a) 2,6-Diphenylpyrimidine-4,5-diamine

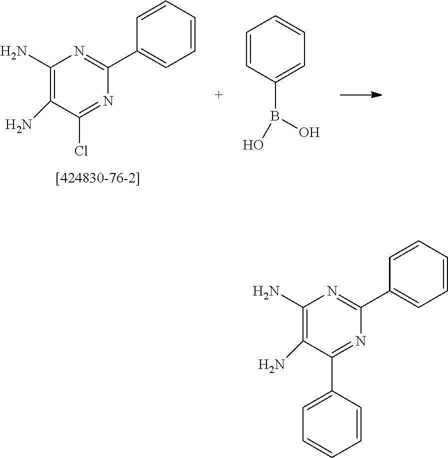 [SMILES: Nc1nc(-c2ccccc2)nc(Cl)c1N ; OB(O)c1ccccc1 ; Nc1nc(-c2ccccc2)nc(-c2ccccc2)c1N]

33 g (150 mmol) of 6-chloro-2-phenylpyrimidine-4,5-diamine, 20.7 g (170 mmol) of phenylboronic acid and 36 g (340 mmol) of sodium carbonate are suspended in 1000 ml of toluene and 280 ml of water. To this suspension is added 1.8 g (1.5 mmol) of tetrakis(triphenylphosphine)palladium(0), and the reaction mixture is heated under reflux for 16 h. After cooling, the organic phase is removed, filtered through silica gel and then concentrated to dryness. The product is purified via column chromatography on silica gel with toluene/heptane (1:2).

The yield is 26 g (102 mmol), corresponding to 68% of theory.

The following compounds are prepared in an analogous manner:

| | Reactant 1 | Reactant 2 | Product | Yield |
|---|---|---|---|---|
| 2a | 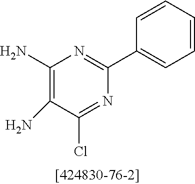 [SMILES: Nc1nc(-c2ccccc2)nc(Cl)c1N] | 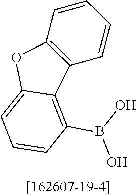 [SMILES: OB(O)c1cccc2oc3ccccc3c12] | 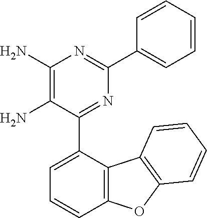 [SMILES: Nc1nc(-c2ccccc2)nc(-c2cccc3oc4ccccc4c23)c1N] | 59% |
| 3a | 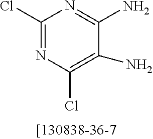 | 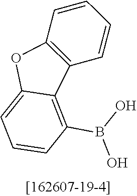 [SMILES: OB(O)c1cccc2oc3ccccc3c12] | 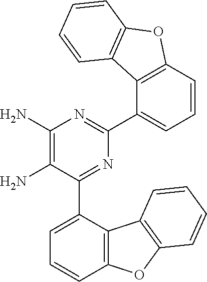 [SMILES: Nc1nc(-c2cccc3oc4ccccc4c23)nc(-c2cccc3oc4ccccc4c23)c1N] | 66% |
| 4a | 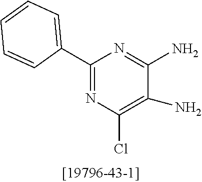 [SMILES: Nc1nc(-c2ccccc2)nc(Cl)c1N] | 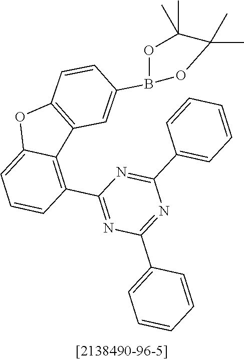 [SMILES: CC1(C)OB(c2ccc3oc4cccc(-c5nc(-c6ccccc6)nc(-c6ccccc6)n5)c4c3c2)OC1(C)C] | 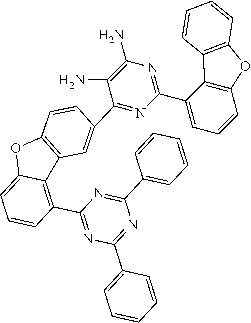 [SMILES: Nc1nc(-c2cccc3oc4ccccc4c23)nc(-c2ccc3oc4cccc(-c5nc(-c6ccccc6)nc(-c6ccccc6)n5)c4c3c2)c1N] | 60% |
| 5a | 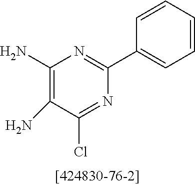 [SMILES: Nc1nc(-c2ccccc2)nc(Cl)c1N] | 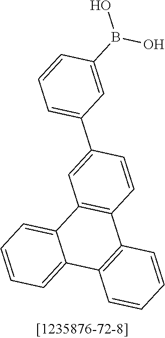 [SMILES: OB(O)c1cccc(-c2ccc3c4ccccc4c4ccccc4c3c2)c1] | 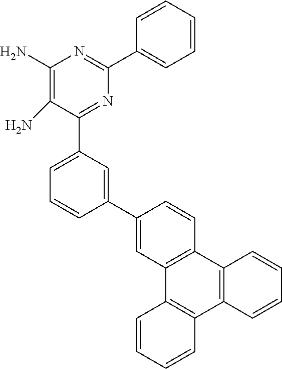 [SMILES: Nc1nc(-c2ccccc2)nc(-c2cccc(-c3ccc4c5ccccc5c5ccccc5c4c3)c2)c1N] | 59% |
| 6a | 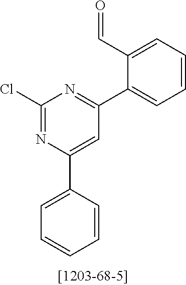 [SMILES: O=Cc1ccccc1-c1cc(-c2ccccc2)nc(Cl)n1] | 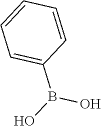 | 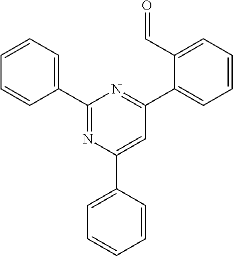 [SMILES: O=Cc1ccccc1-c1cc(-c2ccccc2)nc(-c2ccccc2)n1] | 64% |
| 7a | 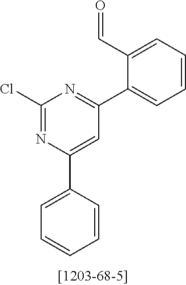 [SMILES: O=Cc1ccccc1-c1cc(-c2ccccc2)nc(Cl)n1] | 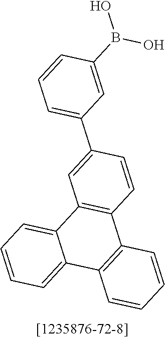 [SMILES: OB(O)c1cccc(-c2ccc3c4ccccc4c4ccccc4c3c2)c1] | 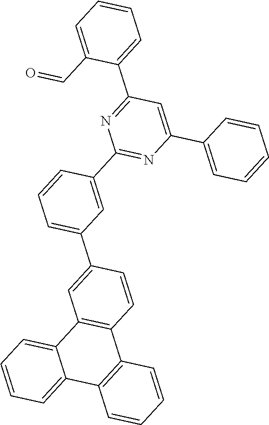 [SMILES: O=Cc1ccccc1-c1cc(-c2ccccc2)nc(-c2cccc(-c3ccc4c5ccccc5c5ccccc5c4c3)c2)n1] | 57% |
| 8a | 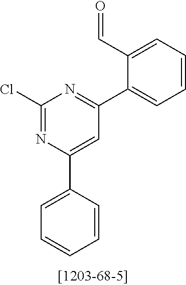 [SMILES: O=Cc1ccccc1-c1cc(-c2ccccc2)nc(Cl)n1] | 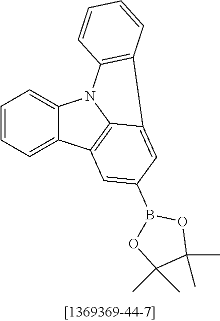 [SMILES: CC1(C)OB(c2cc3c4ccccc4n4c5ccccc5c(c2)c34)OC1(C)C] | 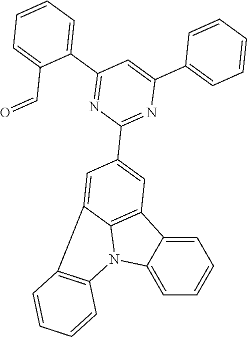 [SMILES: O=Cc1ccccc1-c1cc(-c2ccccc2)nc(-c2cc3c4ccccc4n4c5ccccc5c(c2)c34)n1] | 52% |
| 9a | 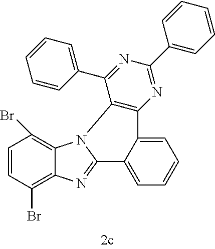 [SMILES: Brc1ccc(Br)c2c1nc1c3ccccc3c3nc(-c4ccccc4)nc(-c4ccccc4)c3n12] | 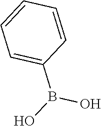 | 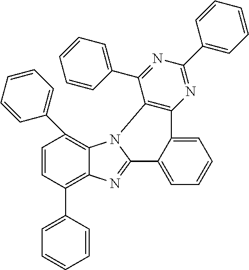 [SMILES: c1ccc(-c2nc(-c3ccccc3)c3c(n2)c2ccccc2c2nc4c(-c5ccccc5)ccc(-c5ccccc5)c4n23)cc1] | 57% |
| 10a | 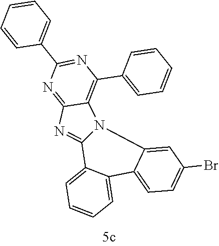 [SMILES: Brc1ccc2c3ccccc3c3nc4nc(-c5ccccc5)nc(-c5ccccc5)c4n3c2c1] | 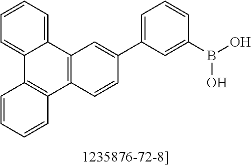 [SMILES: OB(O)c1cccc(-c2ccc3c4ccccc4c4ccccc4c3c2)c1] | 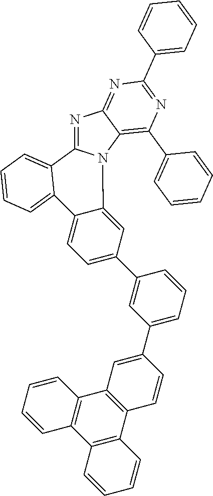 [SMILES: c1ccc(-c2nc(-c3ccccc3)c3c(n2)nc2c4ccccc4c4ccc(-c5cccc(-c6ccc7c8ccccc8c8ccccc8c7c6)c5)cc4n23)cc1] | 59% |
| 11a | 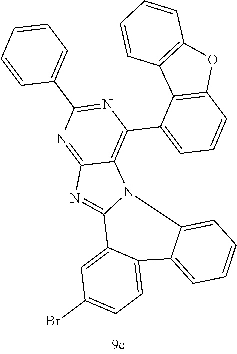 [SMILES: Brc1ccc2c3ccccc3n3c(nc4nc(-c5ccccc5)nc(-c5cccc6oc7ccccc7c56)c43)c2c1] | 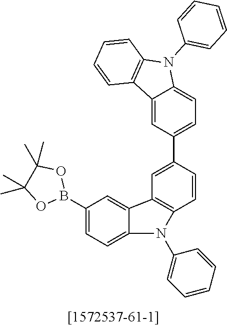 [SMILES: CC1(C)OB(c2ccc3c(c2)c2cc(-c4ccc5c(c4)c4ccccc4n5-c4ccccc4)ccc2n3-c2ccccc2)OC1(C)C] | 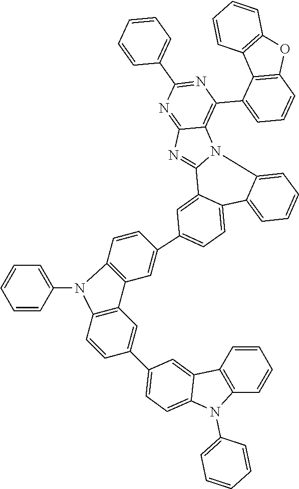 [SMILES: c1ccc(-c2nc(-c3cccc4oc5ccccc5c34)c3c(n2)nc2c4cc(-c5ccc6c(c5)c5cc(-c7ccc8c(c7)c7ccccc7n8-c7ccccc7)ccc5n6-c5ccccc5)ccc4c4ccccc4n23)cc1] | 61% |
| 12a | 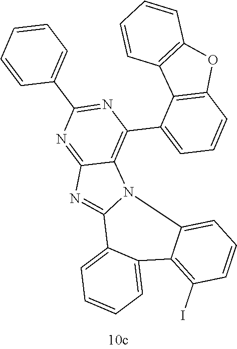 [SMILES: Ic1cccc2c1c1ccccc1c1nc3nc(-c4ccccc4)nc(-c4cccc5oc6ccccc6c45)c3n21] | 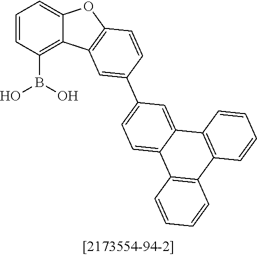 [SMILES: OB(O)c1cccc2oc3ccc(-c4ccc5c6ccccc6c6ccccc6c5c4)cc3c12] | 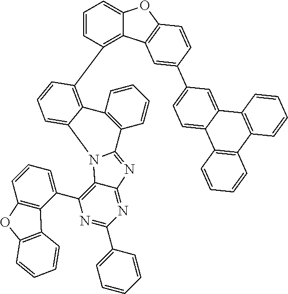 [SMILES: c1ccc(-c2nc(-c3cccc4oc5ccccc5c34)c3c(n2)nc2c4ccccc4c4c(-c5cccc6oc7ccc(-c8ccc9c%10ccccc%10c%10ccccc%10c9c8)cc7c56)cccc4n23)cc1] | 65% |
| 13a | 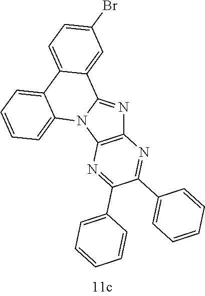 [SMILES: Brc1ccc2c3ccccc3n3c4nc(-c5ccccc5)c(-c5ccccc5)nc4nc3c2c1] | 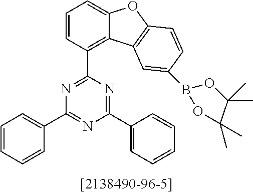 [SMILES: CC1(C)OB(c2ccc3oc4cccc(-c5nc(-c6ccccc6)nc(-c6ccccc6)n5)c4c3c2)OC1(C)C] | 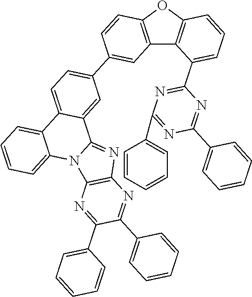 [SMILES: c1ccc(-c2nc(-c3ccccc3)nc(-c3cccc4oc5ccc(-c6ccc7c8ccccc8n8c9nc(-c%10ccccc%10)c(-c%10ccccc%10)nc9nc8c7c6)cc5c34)n2)cc1] | 63% |
| 14a | 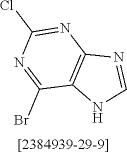 [SMILES: Clc1nc(Br)c2[nH]cnc2n1] | 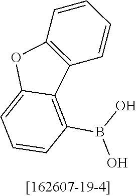 [SMILES: OB(O)c1cccc2oc3ccccc3c12] | 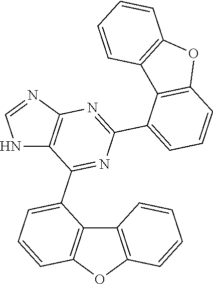 [SMILES: c1ccc2c(c1)oc1cccc(-c3nc(-c4cccc5oc6ccccc6c45)c4[nH]cnc4n3)c12] | 73% |
| 15a | 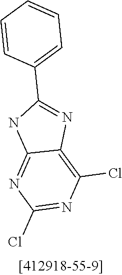 [SMILES: Clc1nc(Cl)c2nc(-c3ccccc3)[nH]c2n1] | 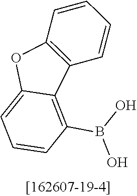 [SMILES: OB(O)c1cccc2oc3ccccc3c12] | 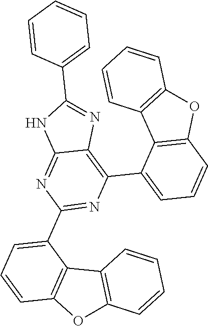 [SMILES: c1ccc(-c2nc3c(-c4cccc5oc6ccccc6c45)nc(-c4cccc5oc6ccccc6c45)nc3[nH]2)cc1] | 78% |
| 16a | 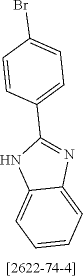 [SMILES: Brc1ccc(-c2nc3ccccc3[nH]2)cc1] | 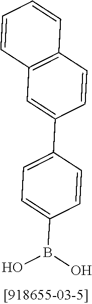 [SMILES: OB(O)c1ccc(-c2ccc3ccccc3c2)cc1] | 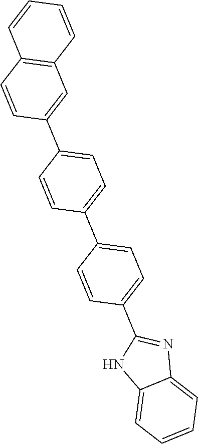 [SMILES: c1ccc2cc(-c3ccc(-c4ccc(-c5nc6ccccc6[nH]5)cc4)cc3)ccc2c1] | 65% |

### b) 2,6-diphenyl-8-(2-phenylphenyl)-9H-purine

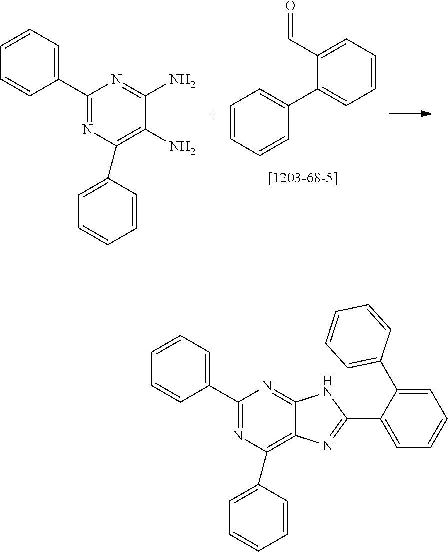 [SMILES: Nc1nc(-c2ccccc2)nc(-c2ccccc2)c1N ; O=Cc1ccccc1-c1ccccc1 ; c1ccc(-c2nc(-c3ccccc3)c3nc(-c4ccccc4-c4ccccc4)[nH]c3n2)cc1]

A solution of 5 g (28 mmol) of o-phenylbenzaldehyde and 7.3 g (28 mmol) of 2,6-diphenylpyrimidine-4,5-diamine in 50 ml of DMF is heated to 80° C. The reaction mixture was left to stir for 8 h, and the resultant solution was brought to room temperature and then extracted with ethyl acetate (EtOAc). The organic layer is washed with salt solution, dried over anhydrous Na₂SO₄, and concentrated under reduced pressure.

The crude product was purified by silica gel column chromatography with n-hexane-EtOAc.

Yield: 8.8 g (20.7 mmol), 75% of theory.

The following compounds can be obtained analogously:

| | Reactant 1 | Reactant 2 |
|---|---|---|
| 1b | 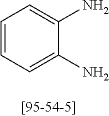 | 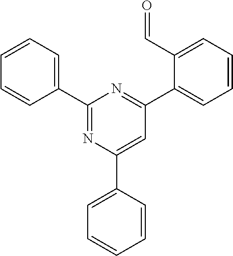 [SMILES: O=Cc1ccccc1-c1cc(-c2ccccc2)nc(-c2ccccc2)n1] |
| 2b | 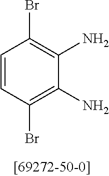 | 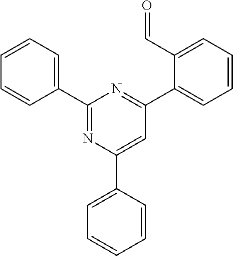 [SMILES: O=Cc1ccccc1-c1cc(-c2ccccc2)nc(-c2ccccc2)n1] |
| 3b | 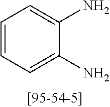 | 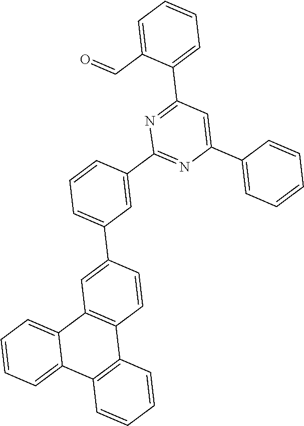 [SMILES: O=Cc1ccccc1-c1cc(-c2ccccc2)nc(-c2cccc(-c3ccc4c5ccccc5c5ccccc5c4c3)c2)n1] |
| 4b | 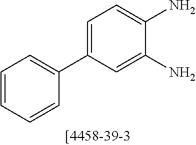 [SMILES: Nc1ccc(-c2ccccc2)cc1N] | 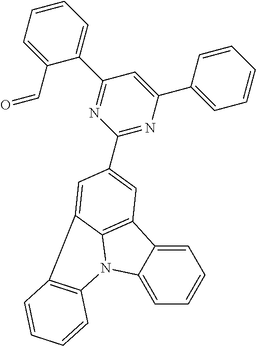 [SMILES: O=Cc1ccccc1-c1cc(-c2ccccc2)nc(-c2cc3c4ccccc4n4c5ccccc5c(c2)c34)n1] |
| 5b | 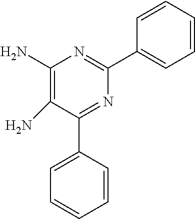 [SMILES: Nc1nc(-c2ccccc2)nc(-c2ccccc2)c1N] | 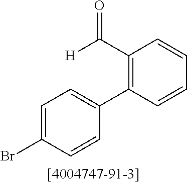 [SMILES: O=Cc1ccccc1-c1ccc(Br)cc1] |
| 6b | 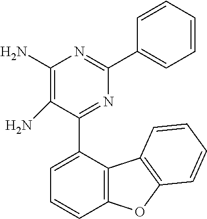 [SMILES: Nc1nc(-c2ccccc2)nc(-c2cccc3oc4ccccc4c23)c1N] | 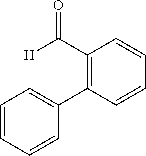 [SMILES: O=Cc1ccccc1-c1ccccc1] |
| 7b | 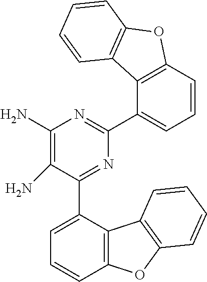 [SMILES: Nc1nc(-c2cccc3oc4ccccc4c23)nc(-c2cccc3oc4ccccc4c23)c1N] | 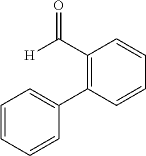 [SMILES: O=Cc1ccccc1-c1ccccc1] |
| 8b | 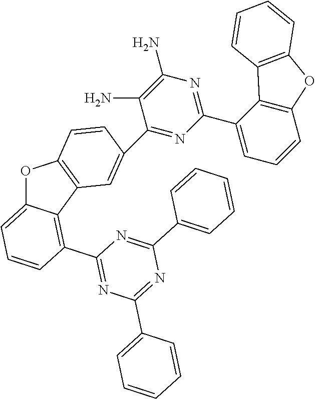 [SMILES: Nc1nc(-c2cccc3oc4ccccc4c23)nc(-c2ccc3oc4cccc(-c5nc(-c6ccccc6)nc(-c6ccccc6)n5)c4c3c2)c1N] | 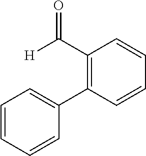 [SMILES: O=Cc1ccccc1-c1ccccc1] |
| 9b | 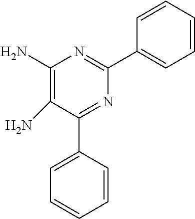 [SMILES: Nc1nc(-c2ccccc2)nc(-c2ccccc2)c1N] | 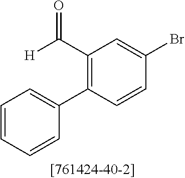 [SMILES: O=Cc1cc(Br)ccc1-c1ccccc1] |
| 10b | 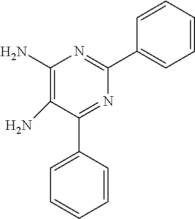 [SMILES: Nc1nc(-c2ccccc2)nc(-c2ccccc2)c1N] | 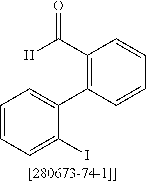 [SMILES: O=Cc1ccccc1-c1ccccc1I] |
| 11b | 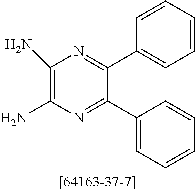 [SMILES: Nc1nc(-c2ccccc2)c(-c2ccccc2)nc1N] | 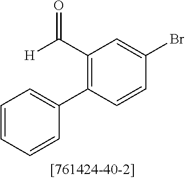 [SMILES: O=Cc1cc(Br)ccc1-c1ccccc1] |
| 12b | 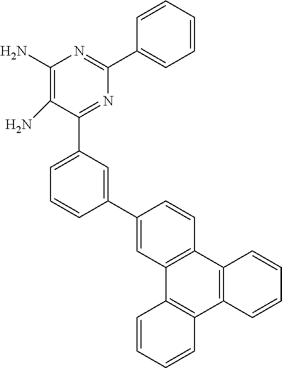 [SMILES: Nc1nc(-c2ccccc2)nc(-c2cccc(-c3ccc4c5ccccc5c5ccccc5c4c3)c2)c1N] | 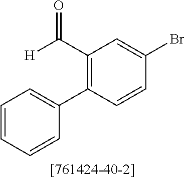 [SMILES: O=Cc1cc(Br)ccc1-c1ccccc1] |

| | Product | Yield |
|---|---|---|
| 1b | 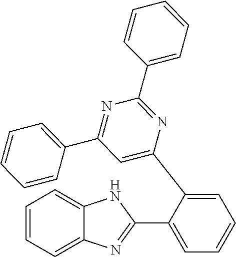 [SMILES: c1ccc(-c2cc(-c3ccccc3-c3nc4ccccc4[nH]3)nc(-c3ccccc3)n2)cc1] | 60% |
| 2b | 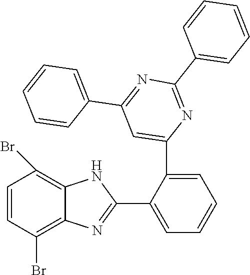 [SMILES: Brc1ccc(Br)c2[nH]c(-c3ccccc3-c3cc(-c4ccccc4)nc(-c4ccccc4)n3)nc12] | 62% |
| 3b | 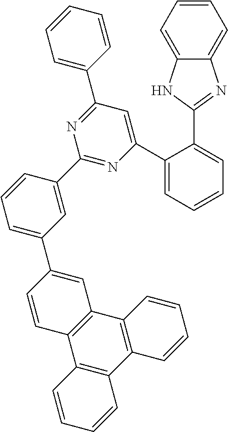 [SMILES: c1ccc(-c2cc(-c3ccccc3-c3nc4ccccc4[nH]3)nc(-c3cccc(-c4ccc5c6ccccc6c6ccccc6c5c4)c3)n2)cc1] | 64% |
| 4b | 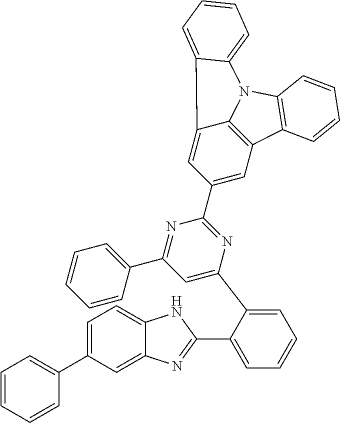 [SMILES: c1ccc(-c2ccc3[nH]c(-c4ccccc4-c4cc(-c5ccccc5)nc(-c5cc6c7ccccc7n7c8ccccc8c(c5)c67)n4)nc3c2)cc1] | 59% |
| 5b | 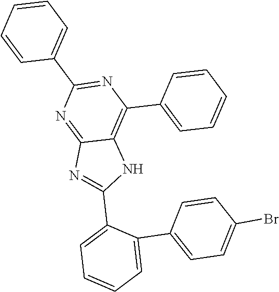 [SMILES: Brc1ccc(-c2ccccc2-c2nc3nc(-c4ccccc4)nc(-c4ccccc4)c3[nH]2)cc1] | 66% |
| 6b | 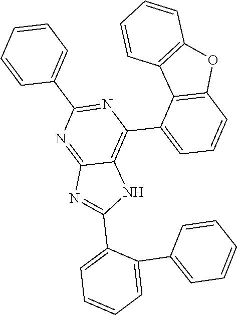 [SMILES: c1ccc(-c2nc(-c3cccc4oc5ccccc5c34)c3[nH]c(-c4ccccc4-c4ccccc4)nc3n2)cc1] | 63% |
| 7b | 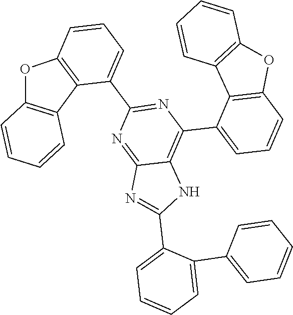 [SMILES: c1ccc(-c2ccccc2-c2nc3nc(-c4cccc5oc6ccccc6c45)nc(-c4cccc5oc6ccccc6c45)c3[nH]2)cc1] | 67% |
| 8b | 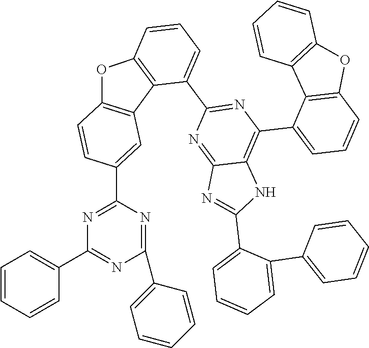 [SMILES: c1ccc(-c2nc(-c3ccccc3)nc(-c3ccc4oc5cccc(-c6nc(-c7cccc8oc9ccccc9c78)c7[nH]c(-c8ccccc8-c8ccccc8)nc7n6)c5c4c3)n2)cc1] | 54% |
| 9b | 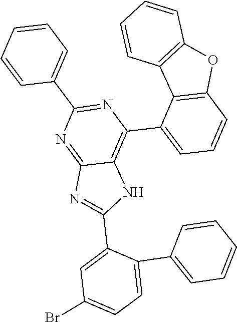 [SMILES: Brc1ccc(-c2ccccc2)c(-c2nc3nc(-c4ccccc4)nc(-c4cccc5oc6ccccc6c45)c3[nH]2)c1] | 61% |
| 10b | 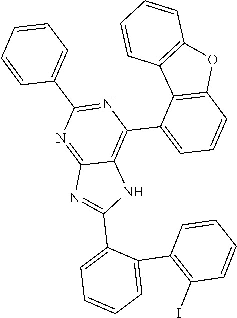 [SMILES: Ic1ccccc1-c1ccccc1-c1nc2nc(-c3ccccc3)nc(-c3cccc4oc5ccccc5c34)c2[nH]1] | 57% |
| 11b | 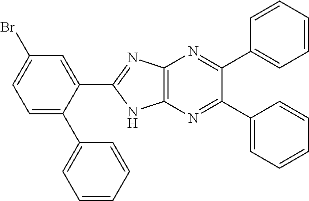 [SMILES: Brc1ccc(-c2ccccc2)c(-c2nc3nc(-c4ccccc4)c(-c4ccccc4)nc3[nH]2)c1] | 63% |
| 12 | 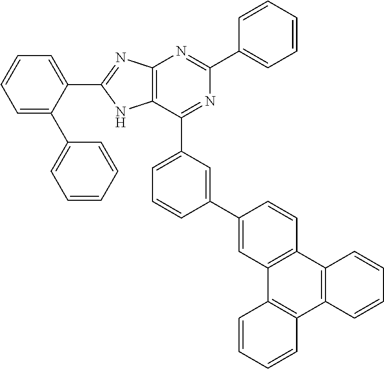 [SMILES: c1ccc(-c2nc(-c3cccc(-c4ccc5c6ccccc6c6ccccc6c5c4)c3)c3[nH]c(-c4ccccc4-c4ccccc4)nc3n2)cc1] | 60% |

### c) Cyclization

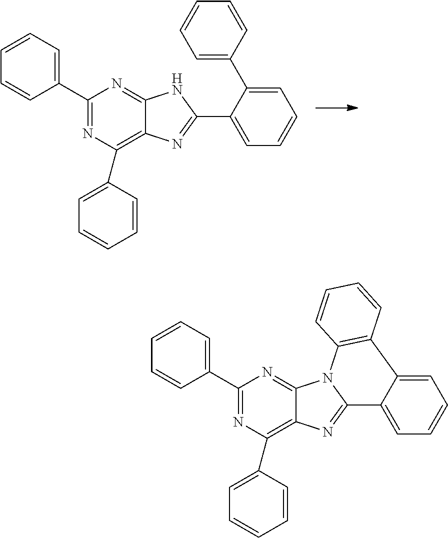 [SMILES: c1ccc(-c2nc(-c3ccccc3)c3nc(-c4ccccc4-c4ccccc4)[nH]c3n2)cc1 ; c1ccc(-c2nc(-c3ccccc3)c3nc4c5ccccc5c5ccccc5n4c3n2)cc1]

To a solution of 7.2 g (17.6 mmol) of 2,6-diphenyl-8-(2-phenylphenyl)-9H-purine in 200 ml of 1,1,1,3,3,3-hexafluoro-2-propanol (HFIP) is gradually added, while stirring at room temperature, 15.2 g (35 mmol) of PhI(OCOCF₃)₂ (PIFA). The reaction mixture is left to stir.

After the reaction has ended (24 h), the solvent is concentrated to dryness. The crude residue was purified by silica gel column chromatography (20% EtOAc in hexane).

Yield: 6.6 g (16 mmol), 90% of theory

The following compounds can be prepared analogously

| | Reactant 1 | Product | Yield |
|---|---|---|---|
| 1c | 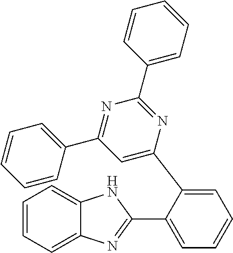 [SMILES: c1ccc(-c2cc(-c3ccccc3-c3nc4ccccc4[nH]3)nc(-c3ccccc3)n2)cc1] | 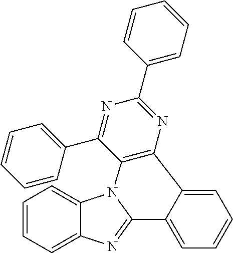 [SMILES: c1ccc(-c2nc(-c3ccccc3)c3c(n2)c2ccccc2c2nc4ccccc4n23)cc1] | 64% |
| 2c | 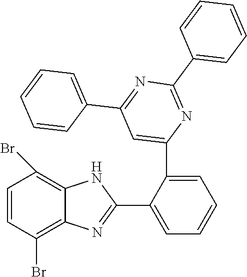 [SMILES: Brc1ccc(Br)c2[nH]c(-c3ccccc3-c3cc(-c4ccccc4)nc(-c4ccccc4)n3)nc12] | 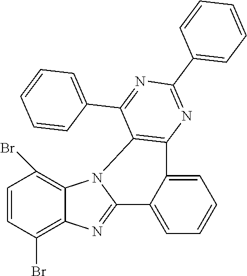 [SMILES: Brc1ccc(Br)c2c1nc1c3ccccc3c3nc(-c4ccccc4)nc(-c4ccccc4)c3n12] | 58% |
| 3c | 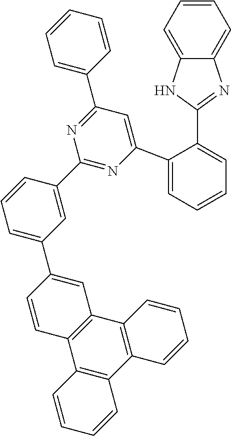 [SMILES: c1ccc(-c2cc(-c3ccccc3-c3nc4ccccc4[nH]3)nc(-c3cccc(-c4ccc5c6ccccc6c6ccccc6c5c4)c3)n2)cc1] | 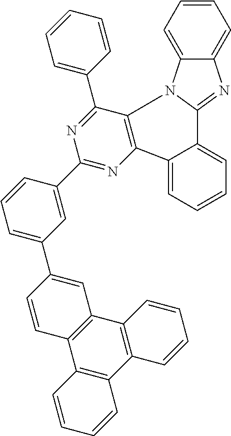 [SMILES: c1ccc(-c2nc(-c3cccc(-c4ccc5c6ccccc6c6ccccc6c5c4)c3)nc3c4ccccc4c4nc5ccccc5n4c23)cc1] | 59% |
| 4c | 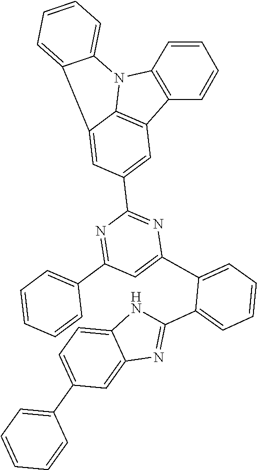 [SMILES: c1ccc(-c2ccc3[nH]c(-c4ccccc4-c4cc(-c5ccccc5)nc(-c5cc6c7ccccc7n7c8ccccc8c(c5)c67)n4)nc3c2)cc1] | 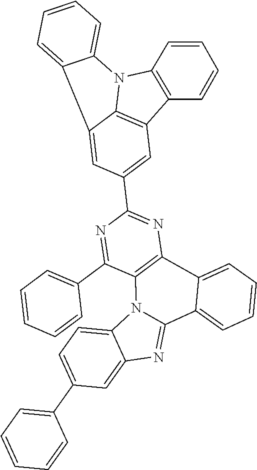 [SMILES: c1ccc(-c2ccc3c(c2)nc2c4ccccc4c4nc(-c5cc6c7ccccc7n7c8ccccc8c(c5)c67)nc(-c5ccccc5)c4n32)cc1] | 63% |
| 5c | 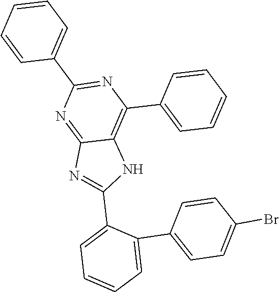 [SMILES: Brc1ccc(-c2ccccc2-c2nc3nc(-c4ccccc4)nc(-c4ccccc4)c3[nH]2)cc1] | 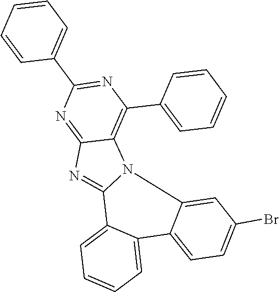 [SMILES: Brc1ccc2c3ccccc3c3nc4nc(-c5ccccc5)nc(-c5ccccc5)c4n3c2c1] | 61% |
| 6c | 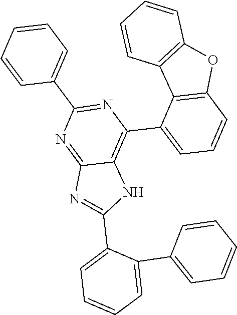 [SMILES: c1ccc(-c2nc(-c3cccc4oc5ccccc5c34)c3[nH]c(-c4ccccc4-c4ccccc4)nc3n2)cc1] | 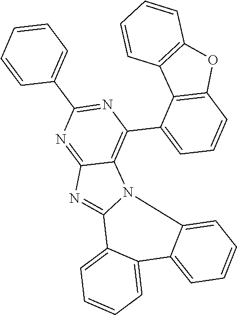 [SMILES: c1ccc(-c2nc(-c3cccc4oc5ccccc5c34)c3c(n2)nc2c4ccccc4c4ccccc4n23)cc1] | 60% |
| 7c | 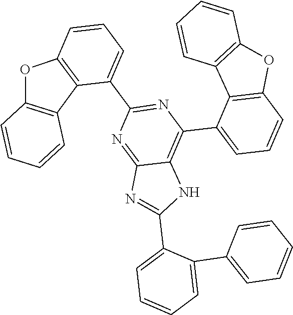 [SMILES: c1ccc(-c2ccccc2-c2nc3nc(-c4cccc5oc6ccccc6c45)nc(-c4cccc5oc6ccccc6c45)c3[nH]2)cc1] | 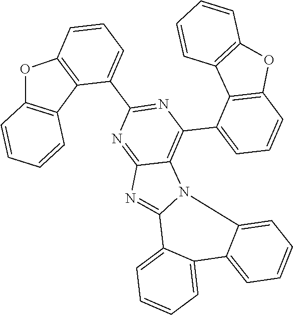 [SMILES: c1ccc2c(c1)oc1cccc(-c3nc(-c4cccc5oc6ccccc6c45)c4c(n3)nc3c5ccccc5c5ccccc5n34)c12] | 57% |
| 8c | 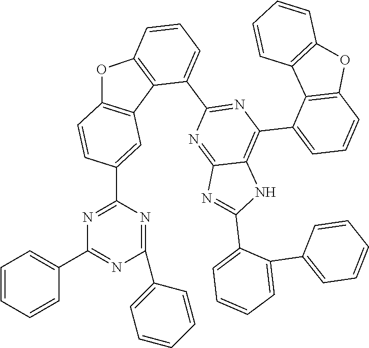 [SMILES: c1ccc(-c2nc(-c3ccccc3)nc(-c3ccc4oc5cccc(-c6nc(-c7cccc8oc9ccccc9c78)c7[nH]c(-c8ccccc8-c8ccccc8)nc7n6)c5c4c3)n2)cc1] | 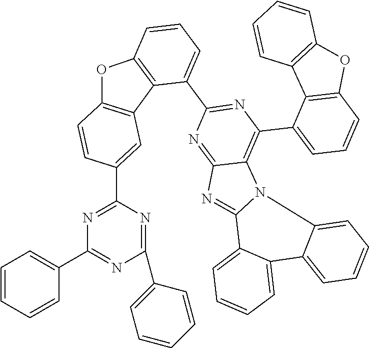 [SMILES: c1ccc(-c2nc(-c3ccccc3)nc(-c3ccc4oc5cccc(-c6nc(-c7cccc8oc9ccccc9c78)c7c(n6)nc6c8ccccc8c8ccccc8n67)c5c4c3)n2)cc1] | 53% |
| 9c | 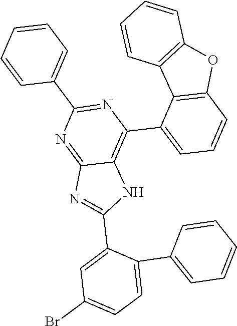 [SMILES: Brc1ccc(-c2ccccc2)c(-c2nc3nc(-c4ccccc4)nc(-c4cccc5oc6ccccc6c45)c3[nH]2)c1] | 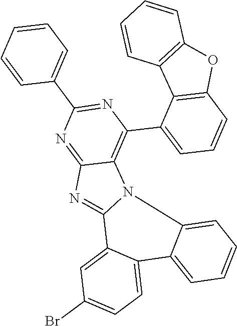 [SMILES: Brc1ccc2c3ccccc3n3c(nc4nc(-c5ccccc5)nc(-c5cccc6oc7ccccc7c56)c43)c2c1] | 64% |
| 10c | 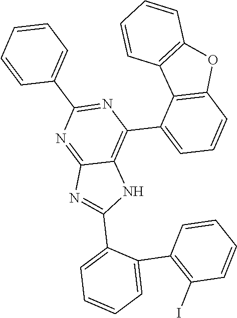 [SMILES: Ic1ccccc1-c1ccccc1-c1nc2nc(-c3ccccc3)nc(-c3cccc4oc5ccccc5c34)c2[nH]1] | 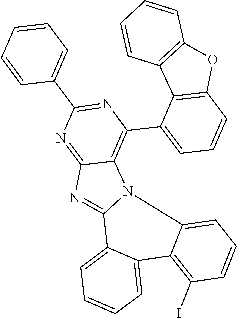 [SMILES: Ic1cccc2c1c1ccccc1c1nc3nc(-c4ccccc4)nc(-c4cccc5oc6ccccc6c45)c3n21] | 58% |
| 11c | 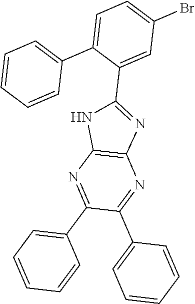 [SMILES: Brc1ccc(-c2ccccc2)c(-c2nc3nc(-c4ccccc4)c(-c4ccccc4)nc3[nH]2)c1] | 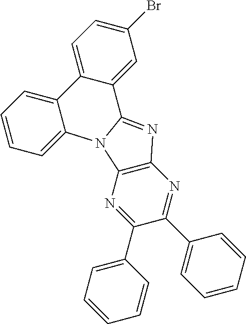 [SMILES: Brc1ccc2c3ccccc3n3c4nc(-c5ccccc5)c(-c5ccccc5)nc4nc3c2c1] | 57% |
| 12c | 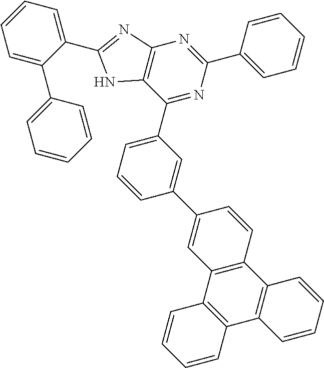 [SMILES: c1ccc(-c2nc(-c3cccc(-c4ccc5c6ccccc6c6ccccc6c5c4)c3)c3[nH]c(-c4ccccc4-c4ccccc4)nc3n2)cc1] | 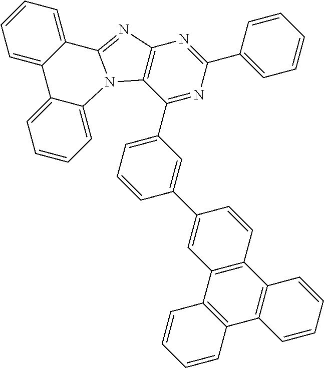 [SMILES: c1ccc(-c2nc(-c3cccc(-c4ccc5c6ccccc6c6ccccc6c5c4)c3)c3c(n2)nc2c4ccccc4c4ccccc4n23)cc1] | 53% |

### d) Cyclization

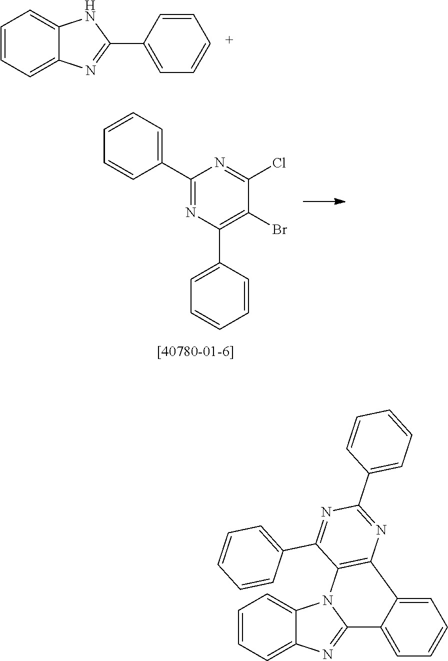 [SMILES: c1ccc(-c2nc3ccccc3[nH]2)cc1 ; Clc1nc(-c2ccccc2)nc(-c2ccccc2)c1Br ; c1ccc(-c2nc(-c3ccccc3)c3c(n2)c2ccccc2c2nc4ccccc4n23)cc1]

Under protective gas, 3.9 g (20 mmol, 1.0 eq.) of 2-phenylbenzimidazole, 10.3 g (30 mmol, 1.5 eq.) of 5-bromo-4-chloro-2,6-diphenylpyrimidine and 8.2 g (60 mmol, 3.0 eq.) of K₂CO₃, 0.5 (2 mmol) of Pd(OAc)₂ and finally 1.9 g (4 mmol) of Xphos were added to 200 ml of DMF. The mixture is heated at 160° C. for 80 h and then comes to room temperature.

This is followed by quenching with 5 ml of water and dilution with 8 ml of ethyl acetate. The organic phase is separated and concentrated under reduced pressure. The crude product is then separated by flash chromatography on silica gel (3-10% ethyl acetate/petroleum ether).

Yield: 6.5 g (15.6 mmol), 77% of theory

The following compounds can be obtained analogously:

| | Reactant 1 | Reactant 2 | Product | Yield |
|---|---|---|---|---|
| 1d | 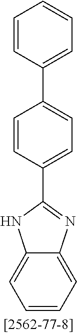 [SMILES: c1ccc(-c2ccc(-c3nc4ccccc4[nH]3)cc2)cc1] | 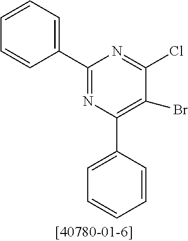 [SMILES: Clc1nc(-c2ccccc2)nc(-c2ccccc2)c1Br] | 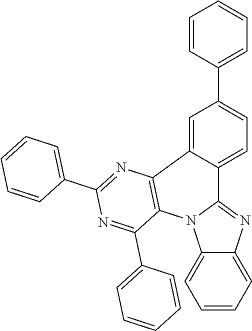 [SMILES: c1ccc(-c2ccc3c(c2)c2nc(-c4ccccc4)nc(-c4ccccc4)c2n2c4ccccc4nc32)cc1] | 70% |
| 2d | 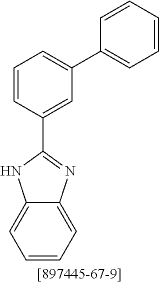 [SMILES: c1ccc(-c2cccc(-c3nc4ccccc4[nH]3)c2)cc1] | 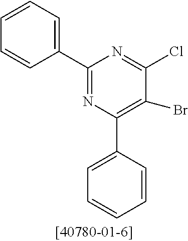 [SMILES: Clc1nc(-c2ccccc2)nc(-c2ccccc2)c1Br] | 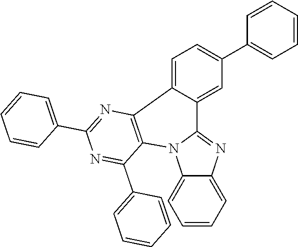 [SMILES: c1ccc(-c2ccc3c(c2)c2nc4ccccc4n2c2c(-c4ccccc4)nc(-c4ccccc4)nc32)cc1] | 73% |
| 3d | 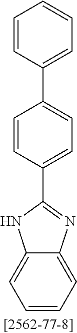 [SMILES: c1ccc(-c2ccc(-c3nc4ccccc4[nH]3)cc2)cc1] | 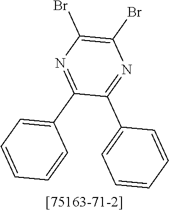 [SMILES: Brc1nc(-c2ccccc2)c(-c2ccccc2)nc1Br] | 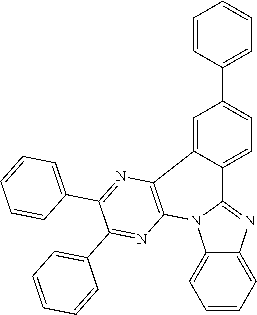 [SMILES: c1ccc(-c2ccc3c(c2)c2nc(-c4ccccc4)c(-c4ccccc4)nc2n2c4ccccc4nc32)cc1] | 71% |
| 4d | 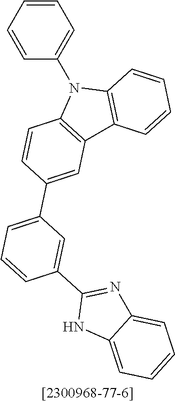 [SMILES: c1ccc(-n2c3ccccc3c3cc(-c4cccc(-c5nc6ccccc6[nH]5)c4)ccc32)cc1] | 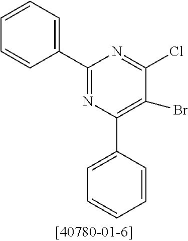 [SMILES: Clc1nc(-c2ccccc2)nc(-c2ccccc2)c1Br] | 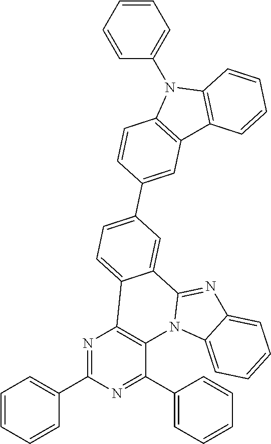 [SMILES: c1ccc(-c2nc(-c3ccccc3)c3c(n2)c2ccc(-c4ccc5c(c4)c4ccccc4n5-c4ccccc4)cc2c2nc4ccccc4n23)cc1] | 53% |
| 5d | 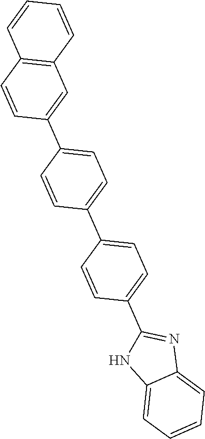 [SMILES: c1ccc2cc(-c3ccc(-c4ccc(-c5nc6ccccc6[nH]5)cc4)cc3)ccc2c1] | 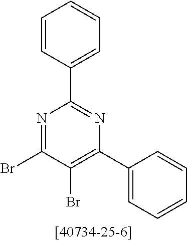 [SMILES: Brc1nc(-c2ccccc2)nc(-c2ccccc2)c1Br] | 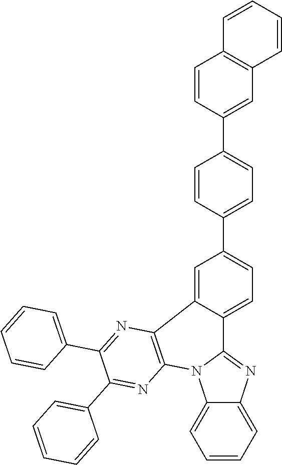 [SMILES: c1ccc(-c2nc3c4cc(-c5ccc(-c6ccc7ccccc7c6)cc5)ccc4c4nc5ccccc5n4c3nc2-c2ccccc2)cc1] | 54% |

### e) Cyclization

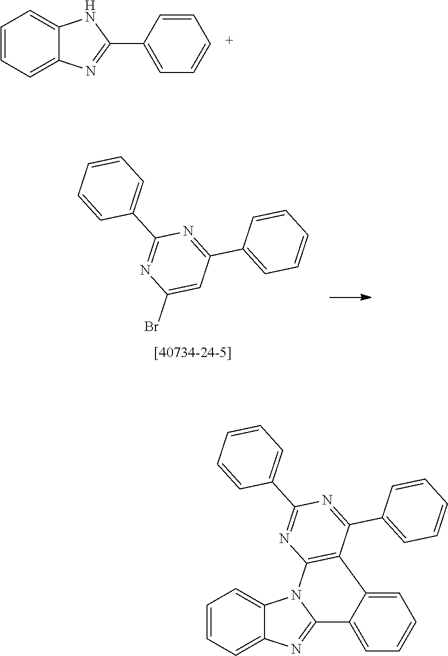 [SMILES: c1ccc(-c2nc3ccccc3[nH]2)cc1 ; Brc1cc(-c2ccccc2)nc(-c2ccccc2)n1 ; c1ccc(-c2nc(-c3ccccc3)c3c4ccccc4c4nc5ccccc5n4c3n2)cc1]

7.7 g (40 mmol, 1.0 eq.) of 2-phenylbenzimidazole, 37.2 g (120 mmol, 3.0 eq.) of 5-bromo-2,4-diphenylpyrimidine, 16.5 mg (120 mmol, 3.0 eq.) of K₂CO₃, and then 700 mg (4 mmol) of PdCl₂ or 920 mg (4 mmol) of Pd(OAc)₂ and 3.8 g (8 mmol) of Xphos is added. Finally, 400 ml of DMF is added to the mixture at room temperature. The mixture is heated under reflux at 160° C. for 72 h. The mixture is cooled down to room temperature, quenched with 1000 ml of water and diluted with 800 ml of ethyl acetate admixed. The organic phase is separated and dried over anhydrous sodium sulfate, filtered and concentrated under reduced pressure. The crude product is then by flash chromatography on silica gel (3-10% ethyl acetate/petroleum ether).

Yield: 8.31 g (19 mmol), 50% of theory

The following compounds can be obtained analogously

| | Reactant 1 | Reactant 2 | Product | Yield |
|---|---|---|---|---|
| 1e: | 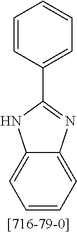 [SMILES: c1ccc(-c2nc3ccccc3[nH]2)cc1] | 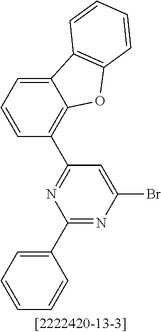 [SMILES: Brc1cc(-c2cccc3c2oc2ccccc23)nc(-c2ccccc2)n1] | 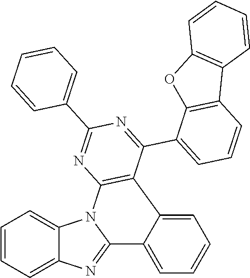 [SMILES: c1ccc(-c2nc(-c3cccc4c3oc3ccccc34)c3c4ccccc4c4nc5ccccc5n4c3n2)cc1] | 41% |
| 2e | 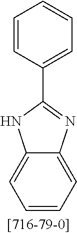 [SMILES: c1ccc(-c2nc3ccccc3[nH]2)cc1] | 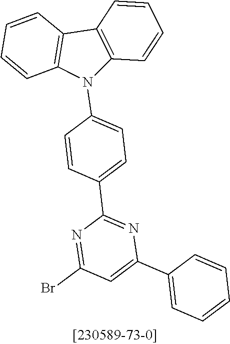 [SMILES: Brc1cc(-c2ccccc2)nc(-c2ccc(-n3c4ccccc4c4ccccc43)cc2)n1] | 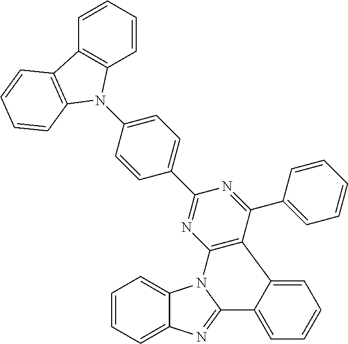 [SMILES: c1ccc(-c2nc(-c3ccc(-n4c5ccccc5c5ccccc54)cc3)nc3c2c2ccccc2c2nc4ccccc4n23)cc1] | 46% |
| 3e | 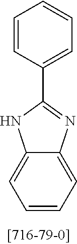 [SMILES: c1ccc(-c2nc3ccccc3[nH]2)cc1] | 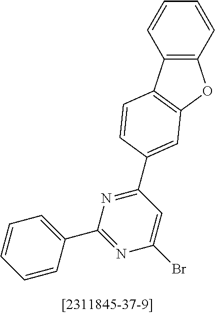 [SMILES: Brc1cc(-c2ccc3c(c2)oc2ccccc23)nc(-c2ccccc2)n1] | 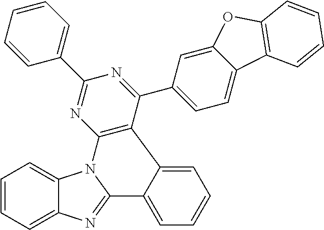 [SMILES: c1ccc(-c2nc(-c3ccc4c(c3)oc3ccccc34)c3c4ccccc4c4nc5ccccc5n4c3n2)cc1] | 45% |
| 4e | 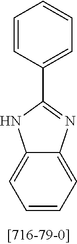 [SMILES: c1ccc(-c2nc3ccccc3[nH]2)cc1] | 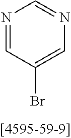 | 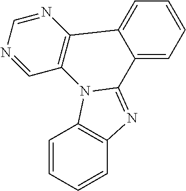 [SMILES: c1ccc2c(c1)nc1c3ccccc3c3ncncc3n21] | 41% |

### f) 2,6,7-Triphenylpurine

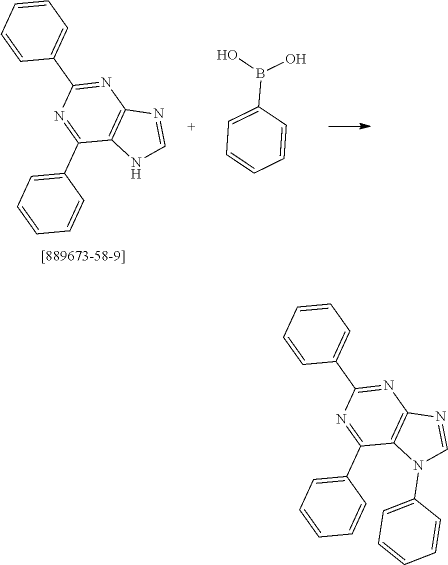 [SMILES: c1ccc(-c2nc(-c3ccccc3)c3[nH]cnc3n2)cc1 ; OB(O)c1ccccc1 ; c1ccc(-c2nc(-c3ccccc3)c3c(ncn3-c3ccccc3)n2)cc1]

9 g (33 mmol) of 2,6-diphenyl-7H-purine (0.33 mmol), 12.2 (100 mmol), 11.8 g (66 mmol) of phenylboronic acid, phenanthroline, 6 g (33 mmol) of anhydrous copper(II) acetate and 25 g of dried 4 A molecular sieves in 500 ml of dry CH₂Cl₂ are heated under reflux condenser under standard atmospheric conditions for 4 days. After cooling, 500 ml of methanol is added to the mixture, which is filtered through Celite, the solvent is concentrated, and purification is effected by flash chromatography on silica gel.

Yield: 9.2 g (26 mmol), 80% of theory.

The following compounds can be obtained analogously:

| | Reactant 1 | Reactant 2 | Product | Yield |
|---|---|---|---|---|
| 1f | 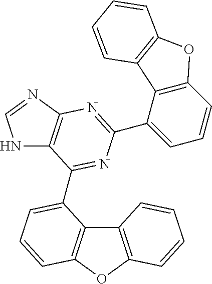 [SMILES: c1ccc2c(c1)oc1cccc(-c3nc(-c4cccc5oc6ccccc6c45)c4[nH]cnc4n3)c12] | 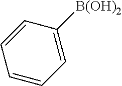 | 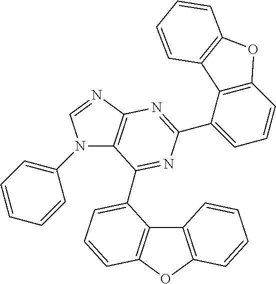 [SMILES: c1ccc(-n2cnc3nc(-c4cccc5oc6ccccc6c45)nc(-c4cccc5oc6ccccc6c45)c32)cc1] | 74% |
| 2f | 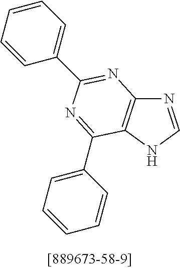 [SMILES: c1ccc(-c2nc(-c3ccccc3)c3[nH]cnc3n2)cc1] | 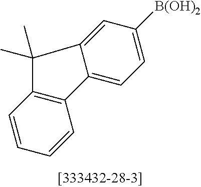 [SMILES: CC1(C)c2ccccc2-c2ccc(B(O)O)cc21] | 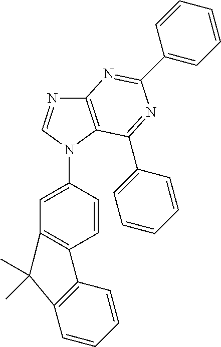 [SMILES: CC1(C)c2ccccc2-c2ccc(-n3cnc4nc(-c5ccccc5)nc(-c5ccccc5)c43)cc21] | 69% |

### g) Cyclization

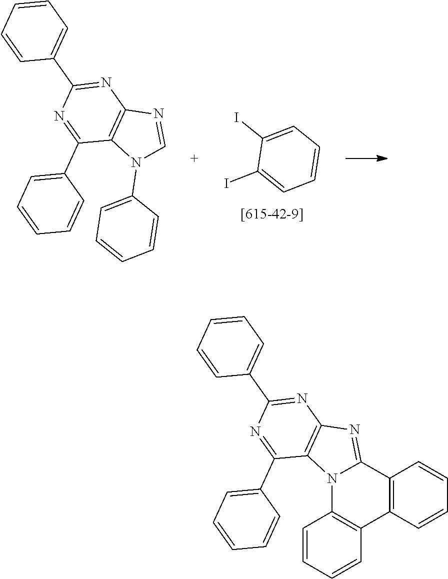 [SMILES: c1ccc(-c2nc(-c3ccccc3)c3c(ncn3-c3ccccc3)n2)cc1 ; Ic1ccccc1I ; c1ccc(-c2nc(-c3ccccc3)c3c(n2)nc2c4ccccc4c4ccccc4n23)cc1]

A mixture of 15.8 g (50 mmol) of Aliquat 100 and 18.4 g (200 mmol) of KOAc is stirred in 2000 ml of dry degassed DMF for 20 min. Under protective gas, 17.4 g (50 mmol) of 2,6,7-triphenylpurine, 1.1 g (5 mmol) of Pd(OAc)₂ and 32 g (100 mmol) of 1,2-iodobenzene are added to that solution. The mixture is heated to 140° C. for 25 h. The solvent is then concentrated under reduced pressure. The product is isolated by flash column chromatography.

Yield 6.3 g (15 mmol); 30% of theory.

The following compounds can be obtained analogously:

| | Reactant 1 | Reactant 2 | Product | Yield |
|---|---|---|---|---|
| 1g | 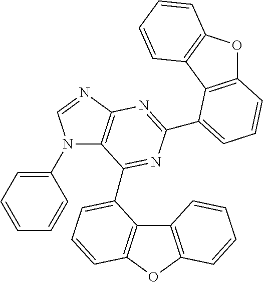 [SMILES: c1ccc(-n2cnc3nc(-c4cccc5oc6ccccc6c45)nc(-c4cccc5oc6ccccc6c45)c32)cc1] | 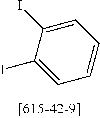 | 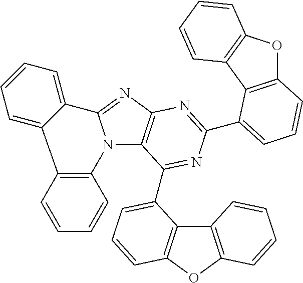 [SMILES: c1ccc2c(c1)oc1cccc(-c3nc(-c4cccc5oc6ccccc6c45)c4c(n3)nc3c5ccccc5c5ccccc5n34)c12] | 74% |
| 2g | 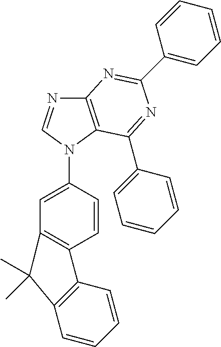 [SMILES: CC1(C)c2ccccc2-c2ccc(-n3cnc4nc(-c5ccccc5)nc(-c5ccccc5)c43)cc21] | 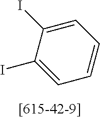 | 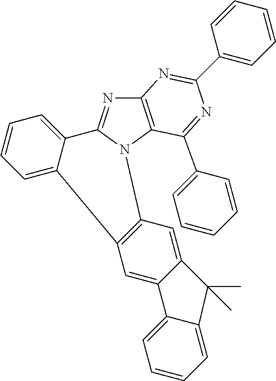 [SMILES: CC1(C)c2ccccc2-c2cc3c4ccccc4c4nc5nc(-c6ccccc6)nc(-c6ccccc6)c5n4c3cc21] | 37% |

### h) Cyclization

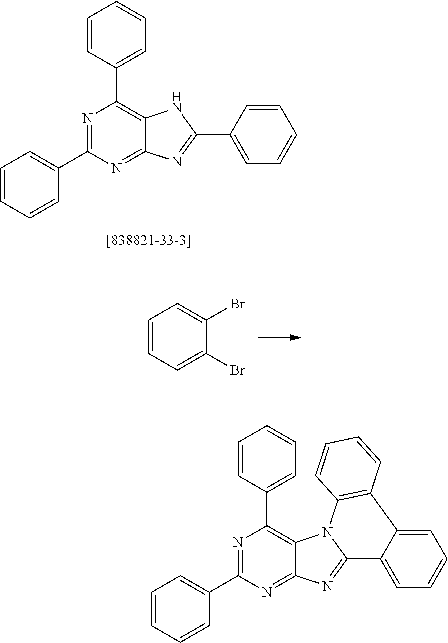 [SMILES: c1ccc(-c2nc(-c3ccccc3)c3[nH]c(-c4ccccc4)nc3n2)cc1 ; Brc1ccccc1Br ; c1ccc(-c2nc(-c3ccccc3)c3c(n2)nc2c4ccccc4c4ccccc4n23)cc1]

In a baked-out flask under protective gas, 2.8 g (2.5, 10% mol) of Pd(PPh₃)₄, 1.46 g (2.5, 10% mol) of Xantphos, 24.3 g (75 mmol) of Cs₂CO₃ and 10.5 g (30 mmol, 1.2 eq.) of 2,6,8-triphenyl-9H-purine and 5.8 g (25 mmol) of 1,2-dibromophenyl in 200 ml of DMF are stirred at room temperature for 10 minutes and then heated to 140° C. for 24 h. Thereafter, the reaction mixture was cooled down to room temperature, 1000 ml of CH₂Cl₂ was added and the mixture was filtered through Celite. The solution is concentrated, and the resulting residue is purified by column chromatography on silica gel.

Yield 6.3 g (15 mmol); 30% of theory.

The following compounds can be obtained analogously:

| | Reactant 1 | Reactant 2 | Product | Yield |
|---|---|---|---|---|
| 1h | 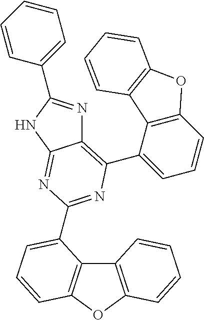 [SMILES: c1ccc(-c2nc3c(-c4cccc5oc6ccccc6c45)nc(-c4cccc5oc6ccccc6c45)nc3[nH]2)cc1] | 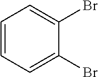 | 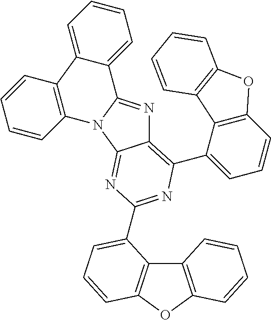 [SMILES: c1ccc2c(c1)oc1cccc(-c3nc(-c4cccc5oc6ccccc6c45)c4nc5c6ccccc6c6ccccc6n5c4n3)c12] | 31% |
| 2h | 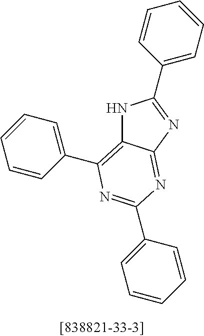 [SMILES: c1ccc(-c2nc(-c3ccccc3)c3[nH]c(-c4ccccc4)nc3n2)cc1] | 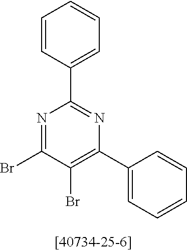 [SMILES: Brc1nc(-c2ccccc2)nc(-c2ccccc2)c1Br] | 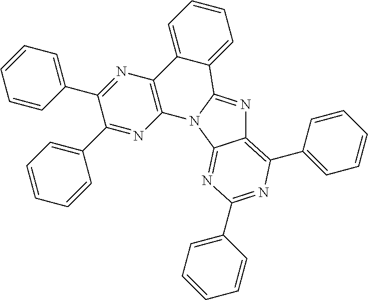 [SMILES: c1ccc(-c2nc(-c3ccccc3)c3nc4c5ccccc5c5nc(-c6ccccc6)c(-c6ccccc6)nc5n4c3n2)cc1] | 27% |

### i) 2-(5-Bromo-2,6-dichloropyrimidin-4-yl)-1-phenylbenzimidazole

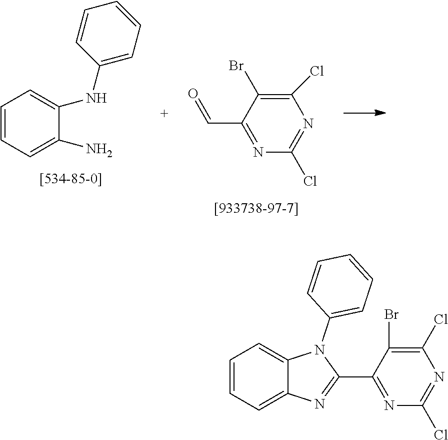 [SMILES: Nc1ccccc1Nc1ccccc1 ; O=Cc1nc(Cl)nc(Cl)c1Br ; Clc1nc(Cl)c(Br)c(-c2nc3ccccc3n2-c2ccccc2)n1]

To a solution of 9.23 g (50 mmol) of 2-aminodiphenylamine in 100 ml of water is added 25.5 g (100 mmol) of 5-bromo-2,6-dichloropyrimidine-4-carboxaldehyde. The mixture is heated under reflux for 22 h. After cooling, the reaction mixture is extracted with 3×50 ml EtOAc (ethyl acetate), and the organic phase is dried with MgSO₄. After filtration and concentration, the residue was purified by column chromatography on silica gel (SiO₂, hexane:EtOAc=4:1).

Yield: 11.8 (28 mmol), 56% of theory.

The following compounds can be obtained analogously:

| | Reactant 1 | Reactant 2 | Product | Yield |
|---|---|---|---|---|
| 1i | 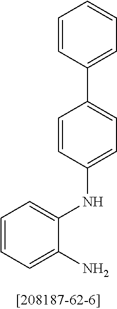 [SMILES: Nc1ccccc1Nc1ccc(-c2ccccc2)cc1] | 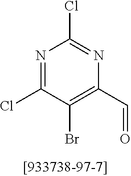 [SMILES: O=Cc1nc(Cl)nc(Cl)c1Br] | 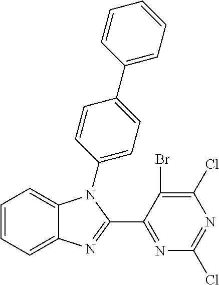 [SMILES: Clc1nc(Cl)c(Br)c(-c2nc3ccccc3n2-c2ccc(-c3ccccc3)cc2)n1] | 53% |

### j) Cyclization

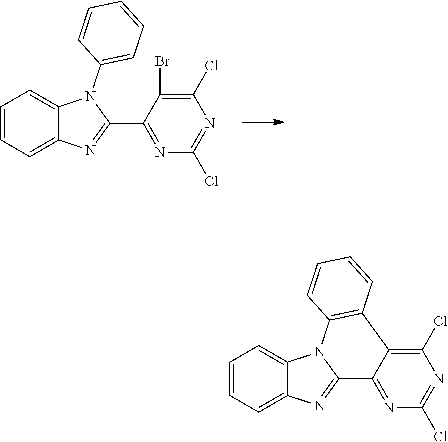 [SMILES: Clc1nc(Cl)c(Br)c(-c2nc3ccccc3n2-c2ccccc2)n1 ; Clc1nc(Cl)c2c3ccccc3n3c4ccccc4nc3c2n1]

In a quartz flask, 20 g (47 mmol) of 2-(5-bromo-2,6-dichloropyrimidin-4-yl)-1-phenylbenzimidazole is dissolved in dichloromethane (800 ml). The mixture was irradiated overnight with two lamps at λ=254 nm (each 4 W). Thereafter, hexane is added to the mixture in order to induce crystallization. The product was filtered, washed with ethanol or EtOAc, and recrystallized in chloroform/hexanes 2:1.

Yield: 12.4 (36 mmol), 80% of theory.

The following compounds can be obtained analogously:

| | Reactant 1 | Product | Yield |
|---|---|---|---|
| 1j | 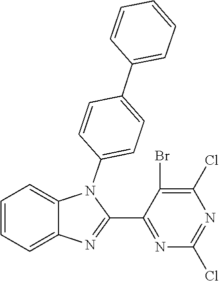 [SMILES: Clc1nc(Cl)c(Br)c(-c2nc3ccccc3n2-c2ccc(-c3ccccc3)cc2)n1] | 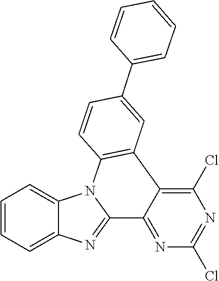 [SMILES: Clc1nc(Cl)c2c3cc(-c4ccccc4)ccc3n3c4ccccc4nc3c2n1] | 77% |

### k) Suzuki Reaction

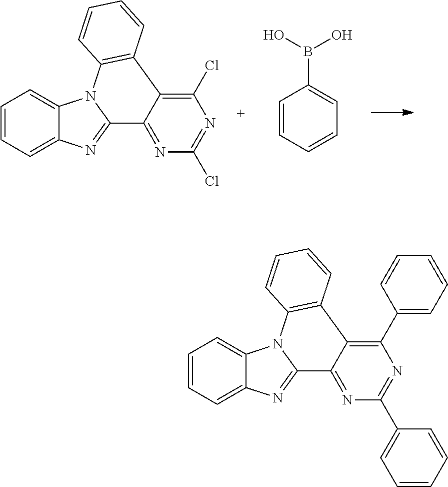 [SMILES: Clc1nc(Cl)c2c3ccccc3n3c4ccccc4nc3c2n1 ; OB(O)c1ccccc1 ; c1ccc(-c2nc(-c3ccccc3)c3c4ccccc4n4c5ccccc5nc4c3n2)cc1]

25 g (75 mmol) of compound j, 10.3 g (85 mmol) of phenylboronic acid and 18 g (170 mmol) of sodium carbonate are suspended in 500 ml of toluene and 160 ml of water. To this suspension is added 0.9 g (0.75 mmol) of tetrakis(triphenylphosphine)palladium(0), and the reaction mixture is heated under reflux for 16 h. After cooling, the organic phase is removed, filtered through silica gel and then concentrated to dryness. The product is purified via column chromatography on silica gel with toluene/heptane (1:1). The yield is 20 g (48 mmol), corresponding to 65% of theory.

The following compounds can be obtained analogously:

| | Reactant 1 | Reactant 2 |
|---|---|---|
| 1k | 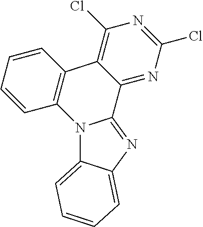 [SMILES: Clc1nc(Cl)c2c3ccccc3n3c4ccccc4nc3c2n1] | 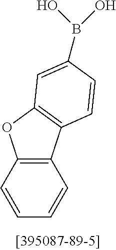 [SMILES: OB(O)c1ccc2c(c1)oc1ccccc12] |
| 2k | 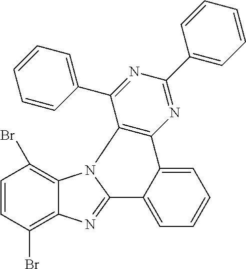 [SMILES: Brc1ccc(Br)c2c1nc1c3ccccc3c3nc(-c4ccccc4)nc(-c4ccccc4)c3n12] | 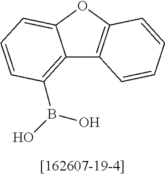 [SMILES: OB(O)c1cccc2oc3ccccc3c12] |
| 3k | 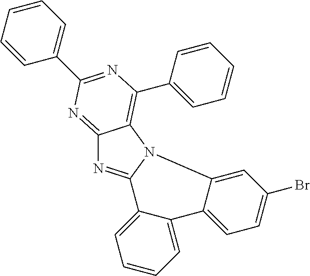 [SMILES: Brc1ccc2c3ccccc3c3nc4nc(-c5ccccc5)nc(-c5ccccc5)c4n3c2c1] | 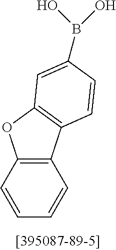 [SMILES: OB(O)c1ccc2c(c1)oc1ccccc12] |
| 4k | 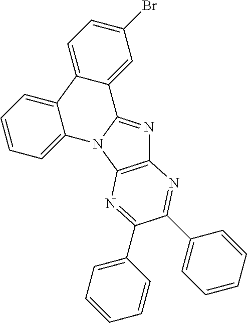 [SMILES: Brc1ccc2c3ccccc3n3c4nc(-c5ccccc5)c(-c5ccccc5)nc4nc3c2c1] | 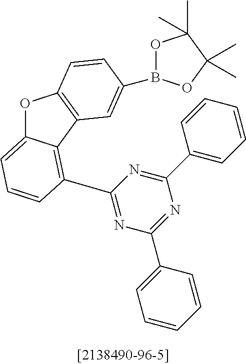 [SMILES: CC1(C)OB(c2ccc3oc4cccc(-c5nc(-c6ccccc6)nc(-c6ccccc6)n5)c4c3c2)OC1(C)C] |
| 5k | 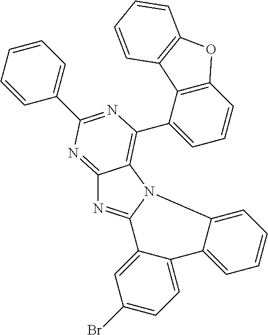 [SMILES: Brc1ccc2c3ccccc3n3c(nc4nc(-c5ccccc5)nc(-c5cccc6oc7ccccc7c56)c43)c2c1] | 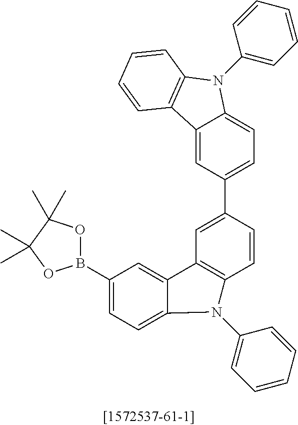 [SMILES: CC1(C)OB(c2ccc3c(c2)c2cc(-c4ccc5c(c4)c4ccccc4n5-c4ccccc4)ccc2n3-c2ccccc2)OC1(C)C] |
| 6k | 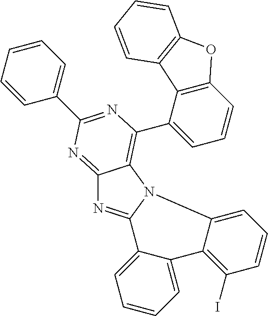 [SMILES: Ic1cccc2c1c1ccccc1c1nc3nc(-c4ccccc4)nc(-c4cccc5oc6ccccc6c45)c3n21] | 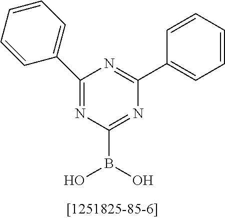 [SMILES: OB(O)c1nc(-c2ccccc2)nc(-c2ccccc2)n1] |

| | Product | Yield |
|---|---|---|
| 1k | 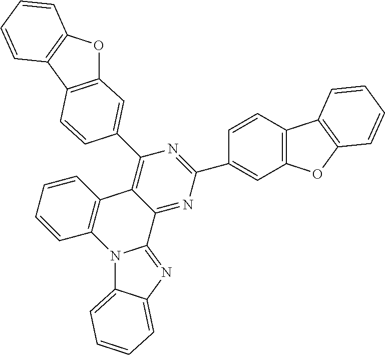 [SMILES: c1ccc2c(c1)nc1c3nc(-c4ccc5c(c4)oc4ccccc45)nc(-c4ccc5c(c4)oc4ccccc45)c3c3ccccc3n21] | 67% |
| 2k | 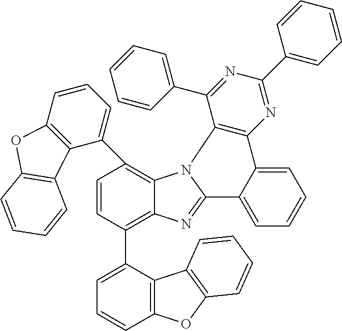 [SMILES: c1ccc(-c2nc(-c3ccccc3)c3c(n2)c2ccccc2c2nc4c(-c5cccc6oc7ccccc7c56)ccc(-c5cccc6oc7ccccc7c56)c4n23)cc1] | 71% |
| 3k | 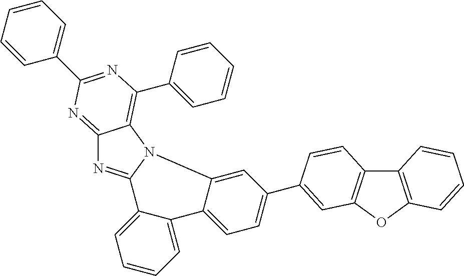 [SMILES: c1ccc(-c2nc(-c3ccccc3)c3c(n2)nc2c4ccccc4c4ccc(-c5ccc6c(c5)oc5ccccc56)cc4n23)cc1] | 74% |
| 4k | 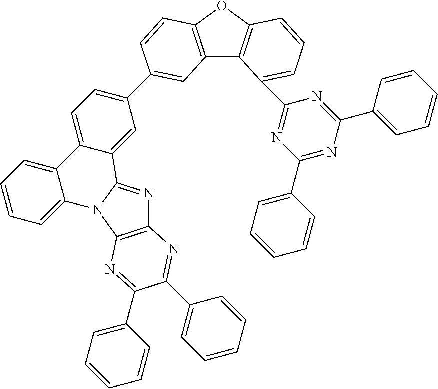 [SMILES: c1ccc(-c2nc(-c3ccccc3)nc(-c3cccc4oc5ccc(-c6ccc7c8ccccc8n8c9nc(-c%10ccccc%10)c(-c%10ccccc%10)nc9nc8c7c6)cc5c34)n2)cc1] | 65% |
| 5k | 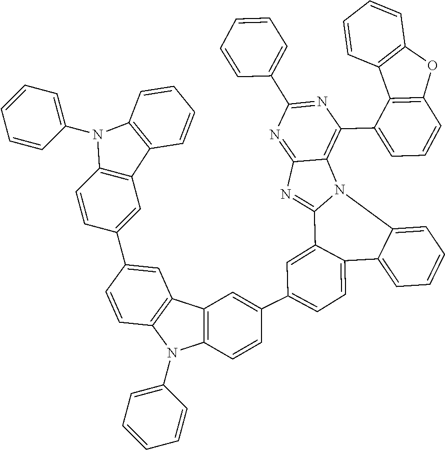 [SMILES: c1ccc(-c2nc(-c3cccc4oc5ccccc5c34)c3c(n2)nc2c4cc(-c5ccc6c(c5)c5cc(-c7ccc8c(c7)c7ccccc7n8-c7ccccc7)ccc5n6-c5ccccc5)ccc4c4ccccc4n23)cc1] | 74% |
| 6k | 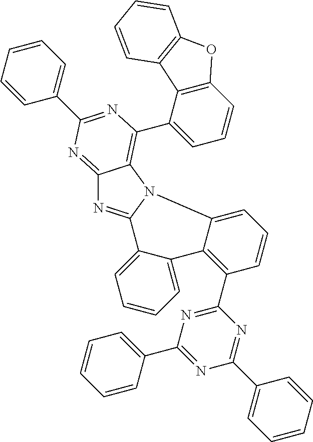 [SMILES: c1ccc(-c2nc(-c3ccccc3)nc(-c3cccc4c3c3ccccc3c3nc5nc(-c6ccccc6)nc(-c6cccc7oc8ccccc8c67)c5n43)n2)cc1] | 78% |

### l) Buchwald

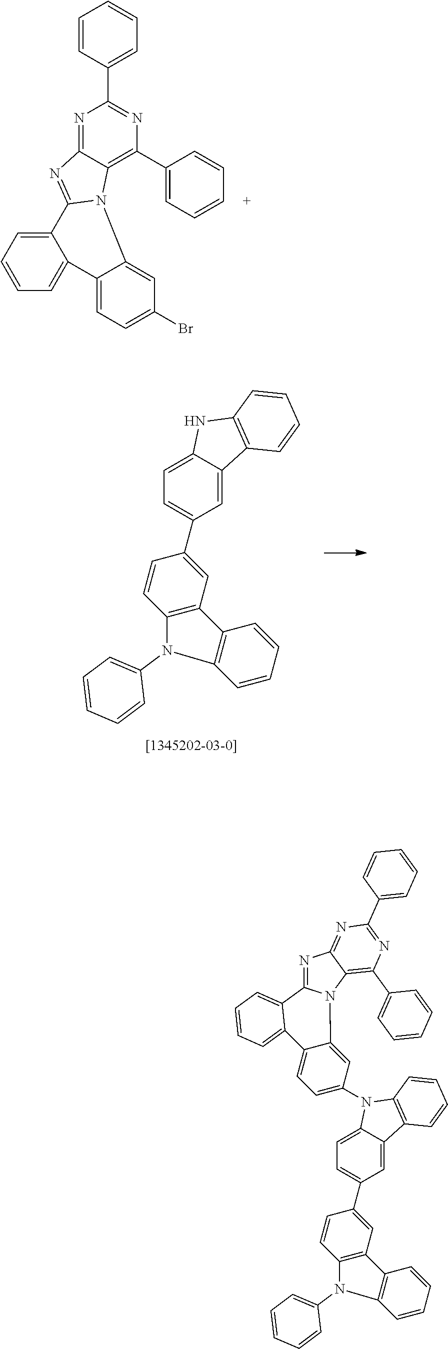 [SMILES: Brc1ccc2c3ccccc3c3nc4nc(-c5ccccc5)nc(-c5ccccc5)c4n3c2c1 ; c1ccc(-n2c3ccccc3c3cc(-c4ccc5[nH]c6ccccc6c5c4)ccc32)cc1 ; c1ccc(-c2nc(-c3ccccc3)c3c(n2)nc2c4ccccc4c4ccc(-n5c6ccccc6c6cc(-c7ccc8c(c7)c7ccccc7n8-c7ccccc7)ccc65)cc4n23)cc1]

40.4 g (50 mmol) of 9-phenyl-3,3′-bi-9H-carbazole and 25 g (50 mmol) of compound 5c are dissolved in 400 ml of toluene under an argon atmosphere. 1.0 g (5 mmol) of tri-tert-butylphosphine is added to the flask and the mixture is stirred under an argon atmosphere. Subsequently, 0.6 g (2 mmol) of Pd(OAc)₂ is added to the flask and the mixture is stirred under an argon atmosphere, and then 9.5 g (99 mmol) of sodium tert-butoxide is added to the flask. The reaction mixture is stirred under reflux for 24 h. After cooling, the organic phase is separated, washed three times with 200 ml of water, dried over MgSO₄ and filtered, and the solvent is removed under reduced pressure. The residue is purified by column chromatography using silica gel (eluent: DCM/heptane (1:3)). The residue is subjected to hot extraction with toluene and recrystallized from toluene/n-heptane and finally sublimed under high vacuum (p=5×10-Smbar) (purity 99.9%).

The yield is 33 g (40 mmol), corresponding to 80% of theory.

### m) 6-Phenyl-2,9,11-tris(trifluoromethyl)purino[8,9-a]isoquinoline

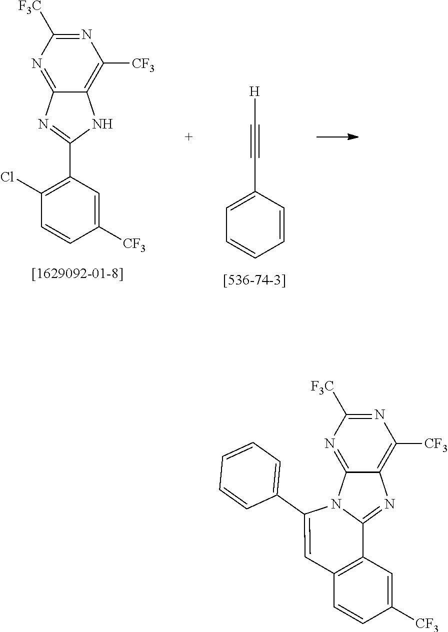 [SMILES: FC(F)(F)c1ccc(Cl)c(-c2nc3nc(C(F)(F)F)nc(C(F)(F)F)c3[nH]2)c1 ; C#Cc1ccccc1 ; FC(F)(F)c1ccc2cc(-c3ccccc3)n3c4nc(C(F)(F)F)nc(C(F)(F)F)c4nc3c2c1]

43 g (100 mmol) of 8-[2-chloro-5-(trifluoromethyl)phenyl]-2,6-bis(trifluoromethyl)-9H-purine is initially charged in 400 ml of triethylamine. While stirring, the following are added successively: 20.4 g (200 mmol) of phenylethyne, 6 mmol of triphenylphosphine, 6 mmol of copper(I) iodide and 3 mmol of palladium(II) acetate. Then the reaction mixture is stirred at 80° C. for 20 h.

After cooling, the reaction mixture is diluted with 400 ml of dichloromethane, the solids are separated off by filtration through a Celite bed, and the filtrate is concentrated to dryness. The residue is taken up in 300 ml of dichloromethane, and the solution is washed three times with 100 ml of conc. ammonia solution and three times with 100 ml each time of water, and dried over magnesium sulfate. After the solvent has been removed under reduced pressure, the crude product is applied to silica gel and packed onto a silica gel column. After concentration, the compound is recrystallized in toluene and finally sublimed under high vacuum (p=5×10⁻⁷ mbar) (purity 99.9%).

The yield is 18 g (36 mmol), corresponding to 37% of theory.

### n), 6,8,10-Tetraphenylpurino[8,7-a]isoquinoline

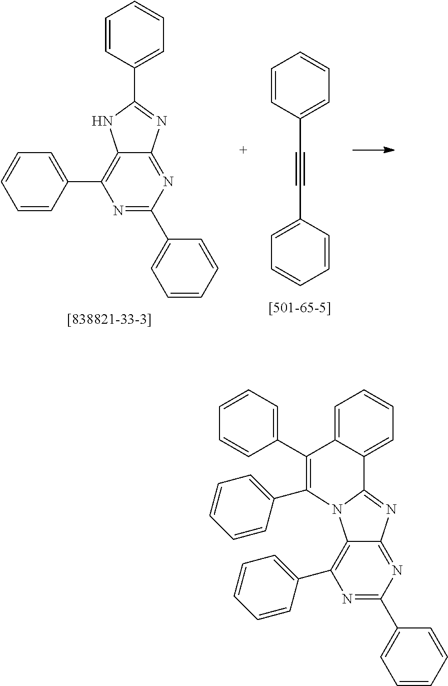

To a well-stirred mixture of 34.8 g (100 mmol) of 2,6,8-triphenyl-9H-purine, 18.1 g (120 mmol) of diphenylethyne and 120 mmol of copper(II) acetate in 1000 ml of DMF are added 2 mmol of pentamethylcyclopentadienylrhodium(II) chloro dimer and 8 mmol of tetraphenylcyclopentadiene, and the mixture was stirred at 80° C. for 20 h.

After cooling, the mixture is filtered through a Celite bed, 1000 ml of dichloromethane is added to the organic phase, and the organic phase is washed five times with 500 ml of water, dried over magnesium sulfate and then concentrated to dryness under reduced pressure. The residue is chromatographed on silica gel (eluent: ethyl acetate—n-heptane). After concentration, the compound is recrystallized in toluene and finally sublimed under high vacuum (p=5×10⁻⁷ mbar) (purity 99.9%).

The yield is 22.5 g (43 mmol), corresponding to 44% of theory.

### o) 8,10-diphenylpurino[8,7-a]isoquinoline

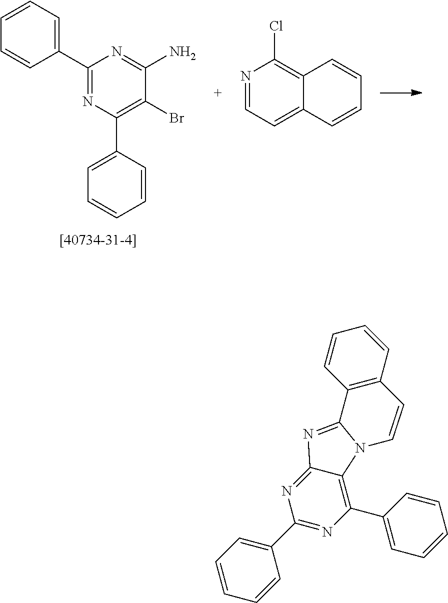 [SMILES: Nc1nc(-c2ccccc2)nc(-c2ccccc2)c1Br ; Clc1nccc2ccccc12 ; c1ccc(-c2nc(-c3ccccc3)c3c(n2)nc2c4ccccc4ccn23)cc1]

A well-stirred mixture of 9.68 g (60 mmol) of 1-chloroisoquinoline, 19.5 g (60 mmol) of 5-bromo-2,6-diphenyl-4-pyrimidineamine, 625 mmol of potassium carbonate, 100 g of glass beads (diameter 3 mm), 5 mmol of triphenylphosphine and 1 mmol of palladium(II) acetate in 800 ml of o-xylene is stirred under reflux for 3-48 h until the 1-chloroisoquinoline derivative has been consumed. After cooling, the mixture is filtered through a silica gel bed and washed through with 1000 ml of THF, and the filtrate is concentrated to dryness. The residue is heated at boiling in 50 ml of ethyl acetate, and 400 ml of n-heptane is added gradually. After cooling, the crystallized solids are silted off with suction, washed twice with 50 ml each time of n-heptane and dried under reduced pressure. After concentration, the compound is recrystallized in toluene and finally sublimed under high vacuum (p=5×10⁻⁷ mbar) (purity 99.9%).

The yield is 17.6 g (47 mmol), corresponding to 79% of theory.

Production of the Electroluminescent Devices

Examples E1 to E30 which follow present the use of the materials of the invention in electroluminescent devices.

Pretreatment for examples E1-E30: Glass plates coated with structured ITO (indium tin oxide) of thickness 50 nm are treated prior to coating with an oxygen plasma, followed by an argon plasma. These plasma-treated glass plates form the substrates to which the OLEDs are applied.

The OLEDs basically have the following layer structure: substrate/optional interlayer (IL)/hole injection layer (HIL)/hole transport layer (HTL)/electron blocker layer (EBL)/emission layer (EML)/optional hole blocker layer (HBL)/electron transport layer (ETL)/optional electron injection layer (EIL) and finally a cathode. The cathode is formed by an aluminum layer of thickness 100 nm. The exact structure of the OLEDs can be found in table 1. The materials required for production of the OLEDs are shown in table 2. The data of the OLEDs are listed in tables 3 and 4.

All materials are applied by thermal vapor deposition in a vacuum chamber. In this case, the emission layer always consists of at least one matrix material (host material) and an emitting dopant (emitter) which is added to the matrix material(s) in a particular proportion by volume by co-evaporation. Details given in such a form as EG1:IC2:TER5 (55%:35%:10%) mean here that the material EG1 is present in the layer in a proportion by volume of 55%, IC2 in a proportion of 35% and TER5 in a proportion of 10%. Analogously, the electron transport layer may also consist of a mixture of two materials.

The OLEDs are characterized in a standard manner. For this purpose, the electroluminescence spectra, the current efficiency (CE, measured in cd/A) and the external quantum efficiency (EQE, measured in %) are determined as a function of luminance, calculated from current-voltage-luminance characteristics assuming Lambertian emission characteristics, as is the lifetime. Electroluminescence spectra are determined at a luminance of 1000 cd/m², and these are used to calculate the CIE 1931 x and y color coordinates. The parameter U1000 in table 3/4 refers to the voltage which is required for a luminance of 1000 cd/m². CE1000 and EQE1000 respectively denote the current efficiency and external quantum efficiency that are attained at 1000 cd/m².

The lifetime LT is defined as the time after which the luminance drops from the starting luminance to a certain proportion L1 in the course of operation with constant current density j₀. A figure of L₁=95% in table 3 means that the lifetime reported in the LD column corresponds to the time after which the luminance falls to 95% of its starting value.

Use and Benefit of the Materials of the Invention in OLEDs

A mixture of two host materials is typically used in the emission layer of OLEDs in order to achieve optimal charge balance and hence very good performance data of the OLED. With regard to simplified production of OLEDs, a reduction in the materials to be used is desirable. The use of just one host material in the emission layer is thus advantageous.

By the use of the inventive compounds EG1 to EG13 in examples E7 to E22 as matrix material in the emission layer of phosphorescent green OLEDs, it is possible to show that use as a single material (E1) and particularly in a mixture with a second host material IC2 and IC3 (E8 and E22) gives improved performance data of the OLEDs compared to the prior art (E1 to E6), particularly with regard to lifetime and efficiency.

Table 4 summarizes the results of some examples. When the inventive compounds (EG2, EG4) are used as electron transport material, significantly lower voltage and better efficiency and lifetime are achieved than with the substance SdT1 and SdT2 according to the prior art (examples E23 to E30).

TABLE 1
Structure of the electroluminescent devices

| | | HIL | HTL | EBL | EML | HBL | ETL | EIL |
|---|---|---|---|---|---|---|---|---|
| Ex. | IL | thickness | thickness | thickness | thickness | thickness | thickness | thickness |
| E1 | | HATCN | SpMA1 | SpMA3 | Sdt1: | ST2 | ST2:LiQ | LiQ 1 nm |
| | | 5 nm | 125 nm | 10 nm | TEG1 | 10 nm | (50%:50%) | |
| | | | | | (97%:3%) | | 30 nm | |
| | | | | | 35 nm | | | |
| E2 | | HATCN | SpMA1 | SpMA3 | SdT4: | ST2 | ST2:LiQ | LiQ 1 nm |
| | | 5 nm | 125 nm | 10 nm | TEG1 | 10 nm | (50%:50%) | |
| | | | | | (97%:3%) | | 30 nm | |
| | | | | | 35 nm | | | |
| E3 | | HATCN | SpMA1 | SpMA3 | SdT1:IC2: | ST2 | ST2:LiQ | LiQ 1 nm |
| | | 5 nm | 125 nm | 10 nm | TEG1 | 10 nm | (50%:50%) | |
| | | | | | (44%:44%: | | 30 nm | |
| | | | | | 12%) | | | |
| | | | | | 30 nm | | | |
| E4 | | HATCN | SpMA1 | SpMA3 | SdT2:IC2: | ST2 | ST2:LiQ | LiQ 1 nm |
| | | 5 nm | 125 nm | 10 nm | TEG1 | 10 nm | (50%:50%) | |
| | | | | | (44%:44%: | | 30 nm | |
| | | | | | 12%) | | | |
| | | | | | 30 nm | | | |
| E5 | | HATCN | SpMA1 | SpMA3 | SdT3:IC2: | ST2 | ST2:LiQ | LiQ 1 nm |
| | | 5 nm | 125 nm | 10 nm | TEG1 | 10 nm | (50%:50%) | |
| | | | | | (44%:44%: | | 30 nm | |
| | | | | | 12%) | | | |
| | | | | | 30 nm | | | |
| E6 | | HATCN | SpMA1 | SpMA3 | SdT4:IC2: | ST2 | ST2:LiQ | LiQ 1 nm |
| | | 5 nm | 125 nm | 10 nm | TEG1 | 10 nm | (50%:50%) | |
| | | | | | (44%:44%: | | 30 nm | |
| | | | | | 12%) | | | |
| | | | | | 30 nm | | | |
| E7 | | HATCN | SpMA1 | SpMA3 | EG10: | ST2 | ST2:LiQ | LiQ 1 nm |
| | | 5 nm | 125 nm | 10 nm | TEG1 | 10 nm | (50%:50%) | |
| | | | | | (97%:3%) | | 30 nm | |
| | | | | | 35 nm | | | |
| E8 | | HATCN | SpMA1 | SpMA3 | EG1:IC2: | ST2 | ST2:LIQ | LiQ 1 nm |
| | | 5 nm | 125 nm | 10 nm | TEG1 | 10 nm | (50%:50%) | |
| | | | | | (44%:44%: | | 30 nm | |
| | | | | | 12%) | | | |
| | | | | | 30 nm | | | |
| E9 | | HATCN | SpMA1 | SpMA3 | EG2:IC2: | ST2 | ST2:LiQ | LiQ 1 nm |
| | | 5 nm | 125 nm | 10 nm | TEG1 | 10 nm | (50%:50 | |
| | | | | | (44%:44 | | %) 30 nm | |
| | | | | | %:12%) | | | |
| | | | | | 30 nm | | | |
| E10 | | HATCN | SpMA1 | SpMA3 | EG3:IC3: | ST2 | ST2:LiQ | LiQ 1 nm |
| | | 5 nm | 125 nm | 10 nm | TEG1 | 10 nm | (50%:50%) | |
| | | | | | (44%:44%: | | 30 nm | |
| | | | | | 12%) | | | |
| | | | | | 30 nm | | | |
| E11 | | HATCN | SpMA1 | SpMA3 | EG4:IC2: | ST2 | ST2:LiQ | LiQ 1 nm |
| | | 5 nm | 125 nm | 10 nm | TEG1 | 10 nm | (50%:50%) | |
| | | | | | (44%:44%: | | 30 nm | |
| | | | | | 12%) | | | |
| | | | | | 30 nm | | | |
| E12 | | HATCN | SpMA1 | SpMA3 | EG5:IC3: | ST2 | ST2:LiQ | LiQ 1 nm |
| | | 5 nm | 125 nm | 10 nm | TEG1 | 10 nm | (50%:50%) | |
| | | | | | (44%:44%: | | 30 nm | |
| | | | | | 12%) | | | |
| | | | | | 30 nm | | | |
| E13 | | HATCN | SpMA1 | SpMA3 | EG6:IC3: | ST2 | ST2:LiQ | LiQ 1 nm |
| | | 5 nm | 125 nm | 10 nm | TEG1 | 10 nm | (50%:50%) | |
| | | | | | (44%:44%: | | 30 nm | |
| | | | | | 12%) | | | |
| | | | | | 30 nm | | | |
| E14 | | HATCN | SpMA1 | SpMA3 | EG6:IC2: | ST2 | ST2:LiQ | LiQ 1 nm |
| | | 5 nm | 125 nm | 10 nm | TEG1 | 10 nm | (50%:50%) | |
| | | | | | (44%:44%: | | 30 nm | |
| | | | | | 12%) | | | |
| | | | | | 30 nm | | | |
| E15 | | HATCN | SpMA1 | SpMA3 | EG7:IC2: | ST2 | ST2:LiQ | LiQ 1 nm |
| | | 5 nm | 125 nm | 10 nm | TEG1 | 10 nm | (50%:50%) | |
| | | | | | (44%:44%: | | 30 nm | |
| | | | | | 12%) | | | |
| | | | | | 30 nm | | | |
| E16 | | HATCN | SpMA1 | SpMA3 | EG8:IC2: | ST2 | ST2:LiQ | LiQ 1 nm |
| | | 5 nm | 125 nm | 10 nm | TEG1 | 10 nm | (50%:50%) | |
| | | | | | (44%:44%: | | 30 nm | |
| | | | | | 12%) | | | |
| | | | | | 30 nm | | | |
| E17 | | HATCN | SpMA1 | SpMA3 | EG9:IC2: | ST2 | ST2:LiQ | LiQ 1 nm |
| | | 5 nm | 125 nm | 10 nm | TEG1 | 10 nm | (50%:50%) | |
| | | | | | (44%:44%: | | 30 nm | |
| | | | | | 12%) | | | |
| | | | | | 30 nm | | | |
| E18 | | HATCN | SpMA1 | SpMA3 | EG10:IC2: | ST2 | ST2:LiQ | LiQ 1 nm |
| | | 5 nm | 125 nm | 10 nm | TEG1 | 10 nm | (50%:50%) | |
| | | | | | (44%:44%: | | 30 nm | |
| | | | | | 12%) | | | |
| | | | | | 30 nm | | | |
| E19 | | HATCN | SpMA1 | SpMA3 | EG10:IC3: | ST2 | ST2:LiQ | LiQ 1 nm |
| | | 5 nm | 125 nm | 10 nm | TEG1 | 10 nm | (50%:50%) | |
| | | | | | (44%:44%: | | 30 nm | |
| | | | | | 12%) | | | |
| | | | | | 30 nm | | | |
| E20 | | HATCN | SpMA1 | SpMA3 | EG11:IC3: | ST2 | ST2:LiQ | LiQ 1 nm |
| | | 5 nm | 125 nm | 10 nm | TEG1 | 10 nm | (50%:50%) | |
| | | | | | (44%:44%: | | 30 nm | |
| | | | | | 12%) | | | |
| | | | | | 30 nm | | | |
| E21 | | HATCN | SpMA1 | SpMA3 | EG12:IC3: | ST2 | ST2:LiQ | LiQ 1 nm |
| | | 5 nm | 125 nm | 10 nm | TEG1 | 10 nm | (50%:50%) | |
| | | | | | (44%:44%: | | 30 nm | |
| | | | | | 12%) | | | |
| | | | | | 30 nm | | | |
| E22 | | HATCN | SpMA1 | SpMA3 | EG13:IC3: | ST2 | ST2:LiQ | LiQ 1 nm |
| | | 5 nm | 125 nm | 10 nm | TEG1 | 10 nm | (50%:50%) | |
| | | | | | (44%:44%: | | 30 nm | |
| | | | | | 12%) | | | |
| | | | | | 30 nm | | | |
| E23 | SpA1 | | HATCN | SpMA1 | M2:SEB | — | SdT1:LiQ | — |
| | 140 nm | | 5 nm | 20 nm | (95%:5%) | | (50%:50%) | |
| | | | | | 20 nm | | 30 nm | |
| E24 | SpA1 | | HATCN | SpMA1 | IC1:TEG1 | IC1 | SdT1:LiQ | |
| | 70 nm | | 5 nm | 90 nm | (90%:10%) | 10 nm | (50%:50%) | |
| | | | | | 30 nm | | 30 nm | |
| E25 | SpA1 | | HATCN | SpMA1 | M2:SEB | — | SdT2:LiQ | — |
| | 140nm | | 5 nm | 20 nm | (95%:5%) | | (50%:50%) | |
| | | | | | 20 nm | | 30 nm | |
| E26 | SpA1 | | HATCN | SpMA1 | IC1:TEG1 | IC1 | SdT2:LiQ | — |
| | 70 nm | | 5 nm | 90 nm | (90%:10%) | 10 nm | (50%:50%) | |
| | | | | | 30 nm | | 30 nm | |
| E27 | SpA1 | | HATCN | SpMA1 | M2:SEB | — | EG2:LiQ | — |
| | 140 nm | | 5 nm | 20 nm | (95%:5%) | | (50%:50%) | |
| | | | | | 20 nm | | 30 nm | |
| E28 | SpA1 | | HATCN | SpMA1 | IC1:TEG1 | IC1 | EG2:LiQ | — |
| | 70 nm | | 5 nm | 90 nm | (90%:10%) | 10 nm | (50%:50%) | |
| | | | | | 30 nm | | 30 nm | |
| E29 | SpA1 | | HATCN | SpMA1 | M2:SEB | — | EG4:LiQ | — |
| | 140 nm | | 5 nm | 20 nm | (95%:5%) | | (50%:50%) | |
| | | | | | 20 nm | | 30 nm | |
| E30 | SpA1 | | HATCN | SpMA1 | IC1:TEG1 | IC1 | EG4:LiQ | — |
| | 70 nm | | 5 nm | 90 nm | (90%:10%) | 10 nm | (50%:50%) | |
| | | | | | 30 nm | | 30 nm | |

TABLE 2
Structural formulae of the materials for the electroluminscent devices

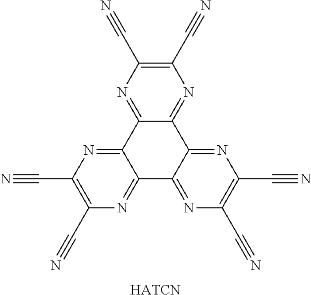

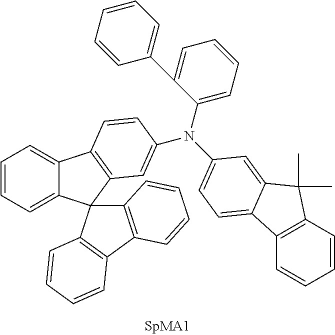 [SMILES: CC1(C)c2ccccc2-c2ccc(N(c3ccc4c(c3)C3(c5ccccc5-c5ccccc53)c3ccccc3-4)c3ccccc3-c3ccccc3)cc21]

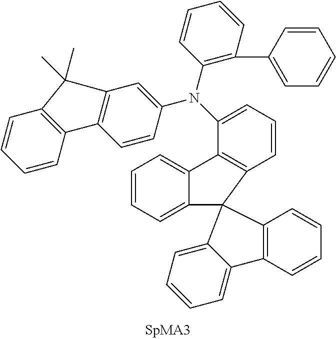 [SMILES: CC1(C)c2ccccc2-c2ccc(N(c3ccccc3-c3ccccc3)c3cccc4c3-c3ccccc3C43c4ccccc4-c4ccccc43)cc21]

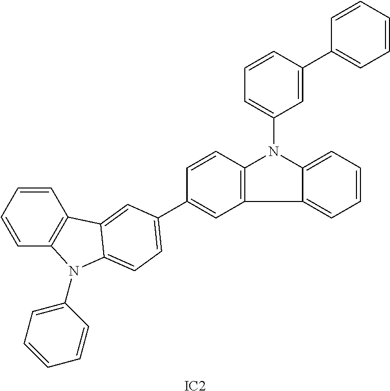 [SMILES: c1ccc(-c2cccc(-n3c4ccccc4c4cc(-c5ccc6c(c5)c5ccccc5n6-c5ccccc5)ccc43)c2)cc1 ; "IC2"]

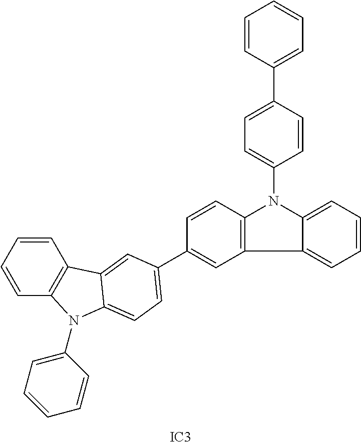 [SMILES: c1ccc(-c2ccc(-n3c4ccccc4c4cc(-c5ccc6c(c5)c5ccccc5n6-c5ccccc5)ccc43)cc2)cc1 ; "IC3"]

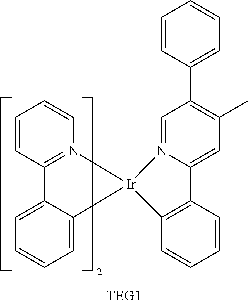

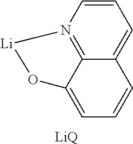

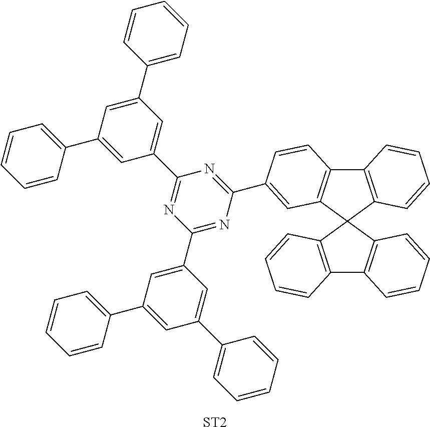 [SMILES: c1ccc(-c2cc(-c3ccccc3)cc(-c3nc(-c4cc(-c5ccccc5)cc(-c5ccccc5)c4)nc(-c4ccc5c(c4)C4(c6ccccc6-c6ccccc64)c4ccccc4-5)n3)c2)cc1 ; "ST2"]

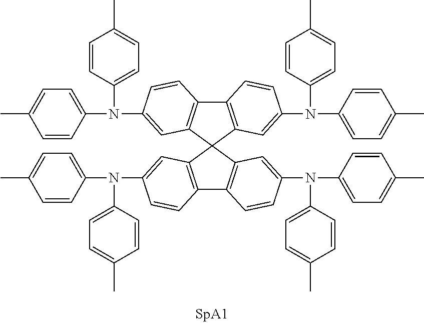

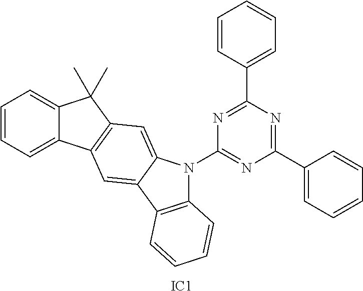

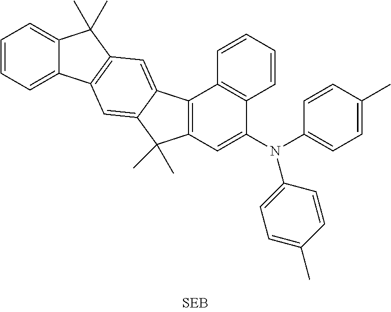 [SMILES: Cc1ccc(N(c2ccc(C)cc2)c2cc3c(c4ccccc24)-c2cc4c(cc2C3(C)C)-c2ccccc2C4(C)C)cc1]

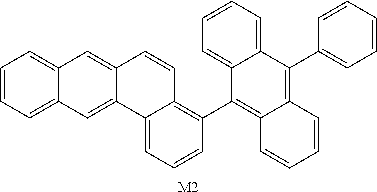

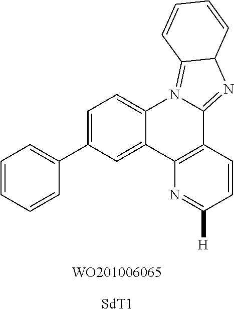

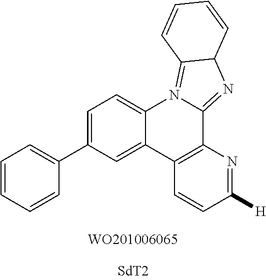

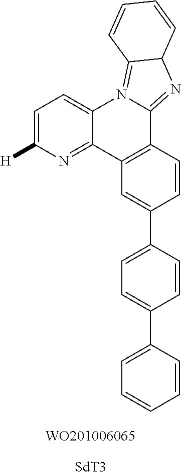

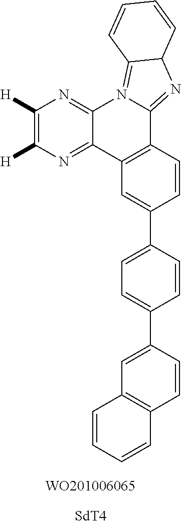

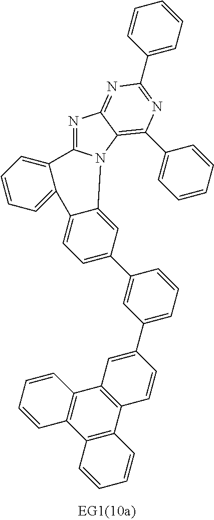 [SMILES: c1ccc(-c2nc(-c3ccccc3)c3c(n2)nc2c4ccccc4c4ccc(-c5cccc(-c6ccc7c8ccccc8c8ccccc8c7c6)c5)cc4n23)cc1]

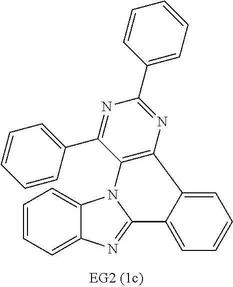 [SMILES: c1ccc(-c2nc(-c3ccccc3)c3c(n2)c2ccccc2c2nc4ccccc4n23)cc1]

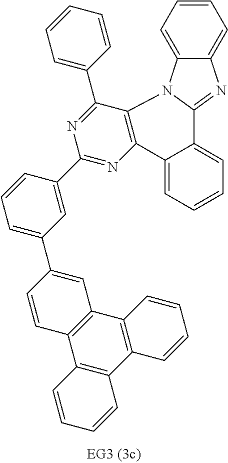 [SMILES: c1ccc(-c2nc(-c3cccc(-c4ccc5c6ccccc6c6ccccc6c5c4)c3)nc3c4ccccc4c4nc5ccccc5n4c23)cc1]

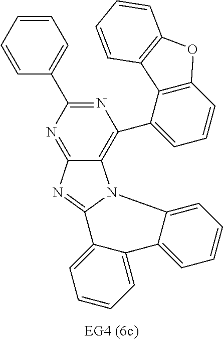 [SMILES: c1ccc(-c2nc(-c3cccc4oc5ccccc5c34)c3c(n2)nc2c4ccccc4c4ccccc4n23)cc1]

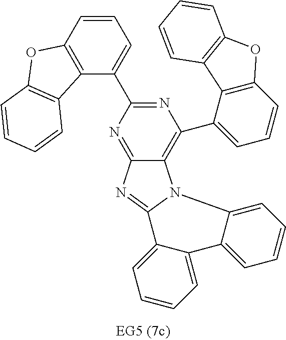 [SMILES: c1ccc2c(c1)oc1cccc(-c3nc(-c4cccc5oc6ccccc6c45)c4c(n3)nc3c5ccccc5c5ccccc5n34)c12]

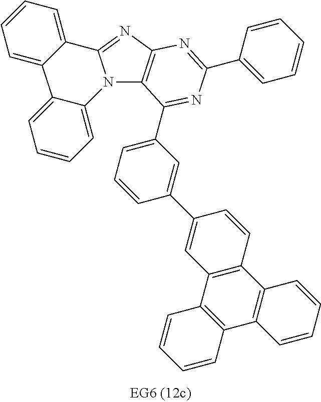 [SMILES: c1ccc(-c2nc(-c3cccc(-c4ccc5c6ccccc6c6ccccc6c5c4)c3)c3c(n2)nc2c4ccccc4c4ccccc4n23)cc1]

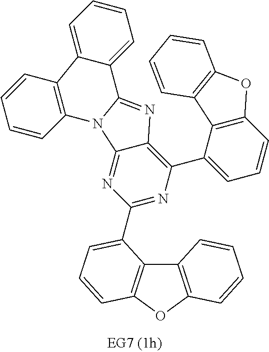 [SMILES: c1ccc2c(c1)oc1cccc(-c3nc(-c4cccc5oc6ccccc6c45)c4nc5c6ccccc6c6ccccc6n5c4n3)c12]

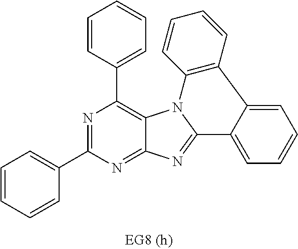 [SMILES: c1ccc(-c2nc(-c3ccccc3)c3c(n2)nc2c4ccccc4c4ccccc4n23)cc1]

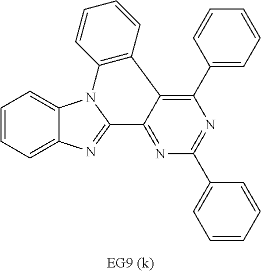 [SMILES: c1ccc(-c2nc(-c3ccccc3)c3c4ccccc4n4c5ccccc5nc4c3n2)cc1]

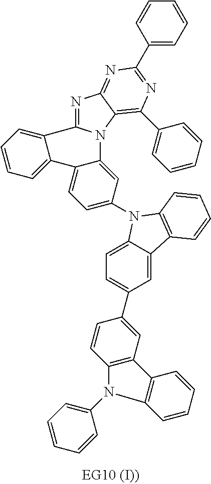 [SMILES: c1ccc(-c2nc(-c3ccccc3)c3c(n2)nc2c4ccccc4c4ccc(-n5c6ccccc6c6cc(-c7ccc8c(c7)c7ccccc7n8-c7ccccc7)ccc65)cc4n23)cc1]

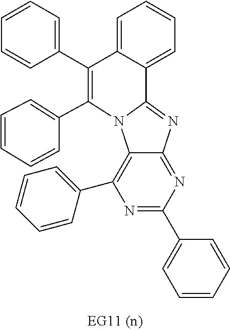 [SMILES: c1ccc(-c2nc(-c3ccccc3)c3c(n2)nc2c4ccccc4c(-c4ccccc4)c(-c4ccccc4)n23)cc1]

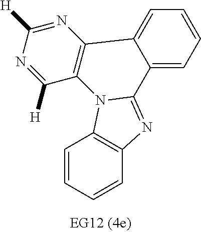

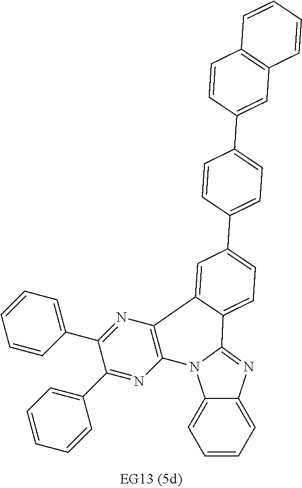 [SMILES: c1ccc(-c2nc3c4cc(-c5ccc(-c6ccc7ccccc7c6)cc5)ccc4c4nc5ccccc5n4c3nc2-c2ccccc2)cc1]

TABLE 3
Performance data of the electroluminescent devices

| | U1000 | CE1000 | EQE 1000 | CIE x/y at | j₀ | L1 | LD |
|---|---|---|---|---|---|---|---|
| Ex. | (V) | (cd/A) | (%) | 1000 cd/m² | (mA/cm²) | (%) | (h) |
| E1 | 5.1 | 75 | 13.4 | 0.34/0.64 | 20 | 95 | 310 |
| E2 | 5.0 | 70 | 12.0 | 0.34/0.63 | 20 | 95 | 270 |
| E3 | 4.8 | 61 | 14.5 | 0.33/0.64 | 20 | 95 | 280 |
| E4 | 4.2 | 76 | 14.8 | 0.32/0.63 | 20 | 95 | 325 |
| E5 | 4.7 | 78 | 13.4 | 0.33/0.63 | 20 | 95 | 350 |
| E6 | 4.5 | 71 | 15.0 | 0.32/0.64 | 20 | 95 | 360 |
| E7 | 4.0 | 65 | 14.8 | 0.34/0.64 | 20 | 95 | 500 |
| E8 | 3.3 | 68 | 19.0 | 0.34/0.63 | 20 | 95 | 1000 |
| E9 | 3.6 | 61 | 17.5 | 0.33/0.64 | 20 | 95 | 970 |
| E10 | 3.2 | 72 | 18.8 | 0.32/0.63 | 20 | 95 | 870 |
| E11 | 3.3 | 77 | 18.4 | 0.33/0.63 | 20 | 95 | 1200 |
| E12 | 3.8 | 71 | 17.8 | 0.32/0.64 | 20 | 95 | 994 |
| E13 | 3.6 | 67 | 18.1 | 0.33/0.64 | 20 | 95 | 990 |
| E14 | 3.7 | 62 | 18.2 | 0.32/0.64 | 20 | 95 | 870 |
| E15 | 3.3 | 70 | 19.1 | 0.32/0.64 | 20 | 80 | 1100 |
| E16 | 3.1 | 67 | 18.7 | 0.33/0.63 | 20 | 80 | 820 |
| E17 | 3.7 | 60 | 18.2 | 0.32/0.64 | 20 | 80 | 770 |
| E18 | 3.9 | 75 | 18.5 | 0.32/0.63 | 20 | 80 | 783 |
| E19 | 3.8 | 63 | 15.6 | 0.33/0.63 | 20 | 80 | 846 |
| E20 | 3.4 | 60 | 18.7 | 0.32/0.64 | 20 | 80 | 750 |
| E21 | 4.1 | 75 | 16 | 0.33/0.64 | 20 | 80 | 300 |
| E22 | 3.6 | 63 | 15.6 | 0.33/0.62 | 20 | 80 | 720 |

TABLE 4
Performance data of the electroluminescent devices

| | U1000 | CE1000 | PE1000 | EQE | CIE x/y at | | L1 | LT |
|---|---|---|---|---|---|---|---|---|
| Ex. | (V) | (cd/A) | (lm/W) | 1000 | 1000 cd/m² | L₀; j₀ | % | (h) |
| E23 | 6 | 7.7 | 5 | 6.2% | 0.13/0.14 | 6000 cd/m² | 80 | 29 |
| E24 | 5.4 | 64 | 51 | 12% | 0.31/0.64 | 20 mA/cm² | 80 | 48 |
| E25 | 6 | 7 | 5 | 6.5% | 0.13/0.14 | 6000 cd/m² | 80 | 28 |
| E26 | 5.0 | 65 | 52 | 13% | 0.31/0.64 | 20 mA/cm² | 80 | 43 |
| E27 | 4.0 | 8 | 5 | 8.8% | 0.13/0.14 | 6000 cd/m² | 80 | 130 |
| E28 | 4.5 | 66 | 64 | 17.7% | 0.33/0.63 | 20 mA/cm² | 80 | 155 |
| E29 | 3.6 | 8 | 6 | 8.9% | 0.14/0.14 | 6000 cd/m² | 80 | 145 |
| E30 | 3.4 | 62 | 63 | 18% | 0.32/0.64 | 20 mA/cm² | 80 | 151 |

Compounds having aryl groups adjacent to nitrogen atoms in a six-membered ring (EG1-EG11 and EG13) have a surprisingly longer lifetime than compounds having the same aryl groups that have a hydrogen atom adjacent to the nitrogen atom (SdT1 to SdT4 and EG12).

Surprisingly, compounds in which the two nitrogen atoms are in meta positions have better performance data than compounds in which the nitrogen atoms are in para positions. Thus, these compounds are especially notable for a lower operating voltage and for an improved quantum efficiency.

1.-18. (canceled)

19. A compound comprising at least one structure of the formula (I): 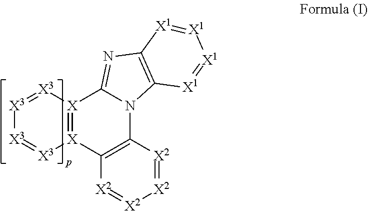
where the symbols and indices used are as follows:
X is N, CR or, if p=1, C;
X¹ is the same or different at each instance and is N, CAr^{a} or CR¹, with the proviso that not more than two of the X¹ groups in one cycle are N;
X² is the same or different at each instance and is N, CAr^{b} or CR², with the proviso that not more than two of the X² groups in one cycle are N;
X³ is the same or different at each instance and is N, CAr^{c} or CR³, with the proviso that not more than two of the X³ groups in one cycle are N;
p is 0 or 1, where the aromatic or heteroaromatic 6-membered ring with the X³ radicals is absent if p=0;
Ar^{a} is the same or different at each instance and is an aromatic or heteroaromatic ring system which has 5 to 40 aromatic ring atoms and may be substituted by one or more R¹ radicals;
Ar^{b} is the same or different at each instance and is an aromatic or heteroaromatic ring system which has 5 to 40 aromatic ring atoms and may be substituted by one or more R² radicals;
Ar^{c} is the same or different at each instance and is an aromatic or heteroaromatic ring system which has 5 to 40 aromatic ring atoms and may be substituted by one or more R³ radicals;
R is the same or different at each instance and is H, D, F, Cl, Br, I, N(R⁴)₂, N(Ar′)₂, CN, NO₂, OR⁴, OAr′, SR⁴, SAr′, COOR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R⁴ radicals, where one or more nonadjacent CH₂ groups may be replaced by Si(R⁴)₂, C═O, NR⁴, O, S or CONR⁴, or an aromatic or heteroaromatic ring system which has 5 to 60 aromatic ring atoms, and may be substituted in each case by one or more R⁴ radicals; at the same time, two R radicals together or one R radical together with one R² radical may also form an aliphatic, heteroaliphatic, aromatic or heteroaromatic ring system;
R¹ is the same or different at each instance and is H, D, F, Cl, Br, I, N(R⁴)₂, N(Ar′)₂, CN, NO₂, OR⁴, OAr′, SR⁴, SAr′, COOR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R⁴ radicals, where one or more nonadjacent CH₂ groups may be replaced by Si(R⁴)₂, C═O, NR⁴, O, S or CONR⁴, or an aromatic or heteroaromatic ring system which has 5 to 60 aromatic ring atoms, and may be substituted in each case by one or more R⁴ radicals; at the same time, two R¹ radicals together or one R¹ radical together with one R² radical may also form an aliphatic, heteroaliphatic, aromatic or heteroaromatic ring system;
R² is the same or different at each instance and is H, D, F, Cl, Br, I, N(R⁴)₂, N(Ar′)₂, CN, NO₂, OR⁴, OAr′, SR⁴, SAr′, COOR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R⁴ radicals, where one or more nonadjacent CH₂ groups may be replaced by Si(R⁴)₂, C═O, NR⁴, O, S or CONR⁴, or an aromatic or heteroaromatic ring system which has 5 to 60 aromatic ring atoms, and may be substituted in each case by one or more R⁴ radicals; at the same time, two R² radicals together or one R² radical together with one R, R¹, R³ radical may also form an aromatic, heteroaromatic, aliphatic or heteroaliphatic ring system;
R³ is the same or different at each instance and is H, D, F, Cl, Br, I, N(R⁴)₂, N(Ar′)₂, CN, NO₂, OR⁴, OAr′, SR⁴, SAr′, COOR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R⁴ radicals, where one or more nonadjacent CH₂ groups may be replaced by Si(R⁴)₂, C═O, NR⁴, O, S or CONR⁴, or an aromatic or heteroaromatic ring system which has 5 to 60 aromatic ring atoms, and may be substituted in each case by one or more R⁴ radicals; at the same time, two R³ radicals together or one R³ radical together with one R² radical may also form an aromatic, heteroaromatic, aliphatic or heteroaliphatic ring system;
Ar′ is the same or different at each instance and is an aromatic or heteroaromatic ring system which has 5 to 40 aromatic ring atoms and may be substituted by one or more R⁴ radicals;
R⁴ is the same or different at each instance and is H, D, F, Cl, Br, I, N(R⁵)₂, CN, NO₂, OR⁵, SR⁵, Si(R⁵)₃, B(OR⁵)₂, C(═O)R⁵, P(═O)(R⁵)₂, S(═O)R⁵, S(═O)₂R⁵, OSO₂R⁵, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R⁵ radicals and where one or more nonadjacent CH₂ groups may be replaced by Si(R⁵)₂, C═O, NR⁵, O, S or CONR⁵, or an aromatic or heteroaromatic ring system which has 5 to 40 aromatic ring atoms and may be substituted in each case by one or more R² radicals; at the same time, two or more R⁴ radicals together may form an aromatic, heteroaromatic, aliphatic or heteroaliphatic ring system;
R⁵ is the same or different at each instance and is H, D, F or an aliphatic, aromatic or heteroaromatic organic radical, in which one or more hydrogen atoms may also be replaced by F;
characterized in that
in at least one of the rings having the X¹, X² or X³ groups, two nonadjacent X¹, X² or X³ groups in one ring are N.

20. The compound as claimed in claim 19, characterized in that, in at least one of the rings having the X¹, X² or X³ groups, two nonadjacent X¹, X² or X³ groups in one ring are N, and the X¹, X² or X³ groups adjacent to the respective N in a ring having at least two nonadjacent nitrogen atoms are CAr^{a}, CAr^{b}, CAr^{c} or CR¹, CR², CR³, where R¹, R², R³ is an aromatic or heteroaromatic ring system which has 5 to 60 aromatic ring atoms, and may be substituted in each case by one or more R⁴ radicals.

21. The compound as claimed in claim 19, characterized in that, in at least one of the rings having the X¹, X² or X³ groups, two nonadjacent X¹, X² or X³ groups in one ring are N, where the two nonadjacent X¹, X² or X³ groups in one ring that are N are in meta positions to one another.

22. The compound as claimed in claim 19, comprising at least one structure of the formulae (II-1) to (II-25): 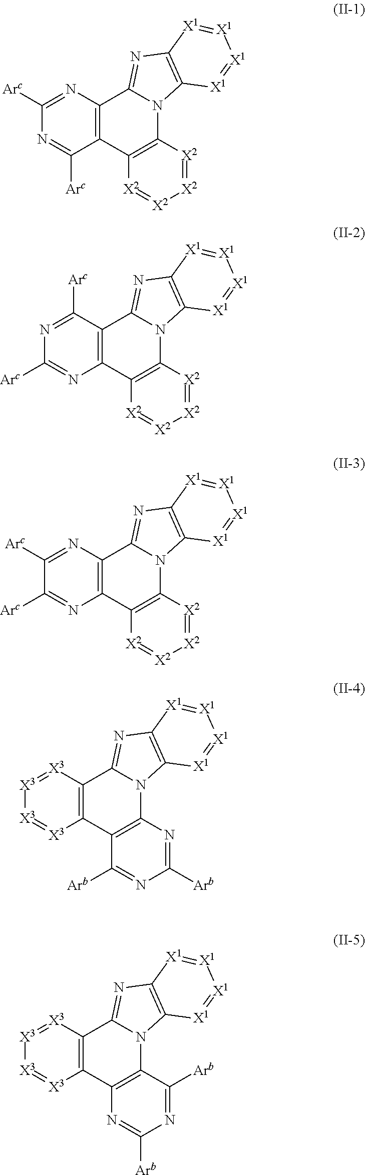 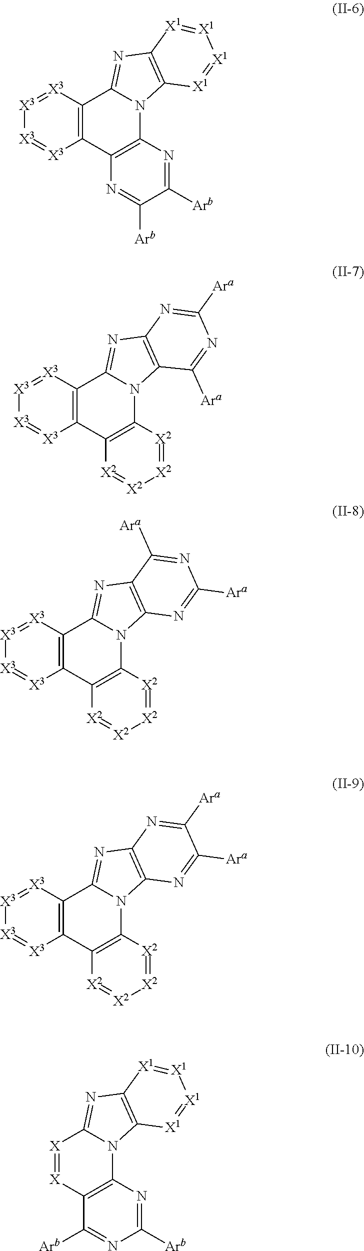 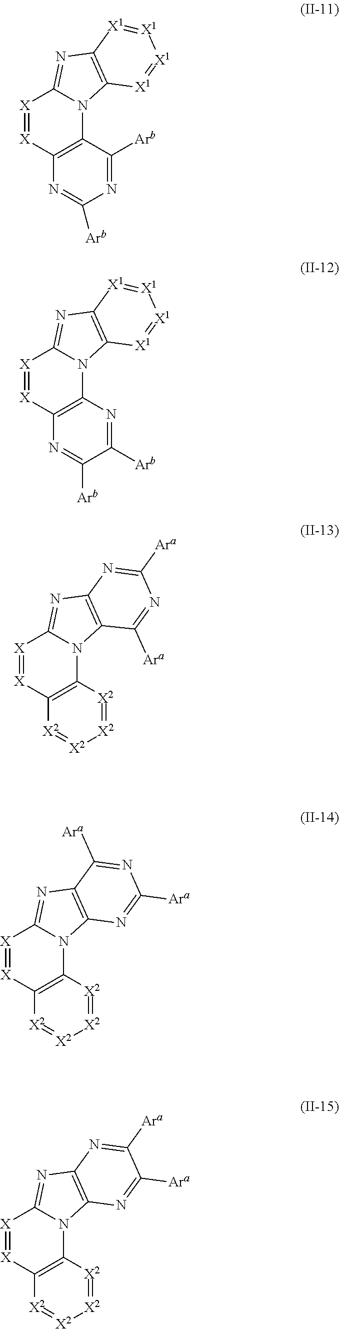 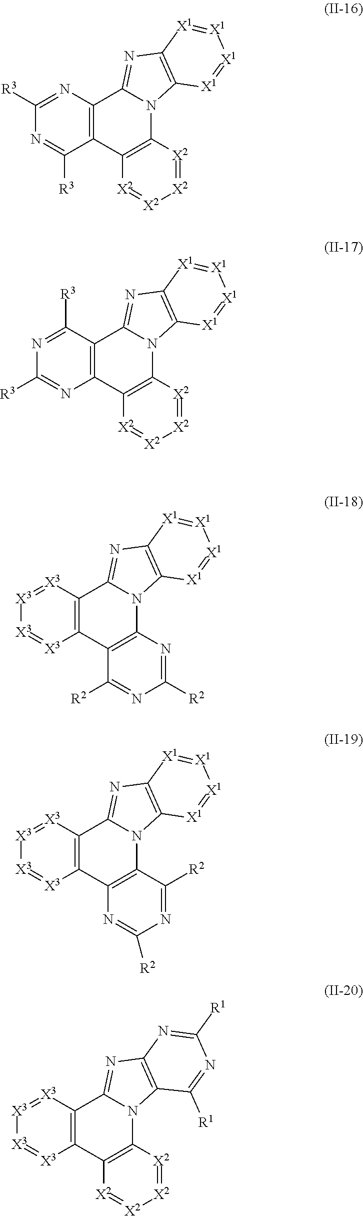 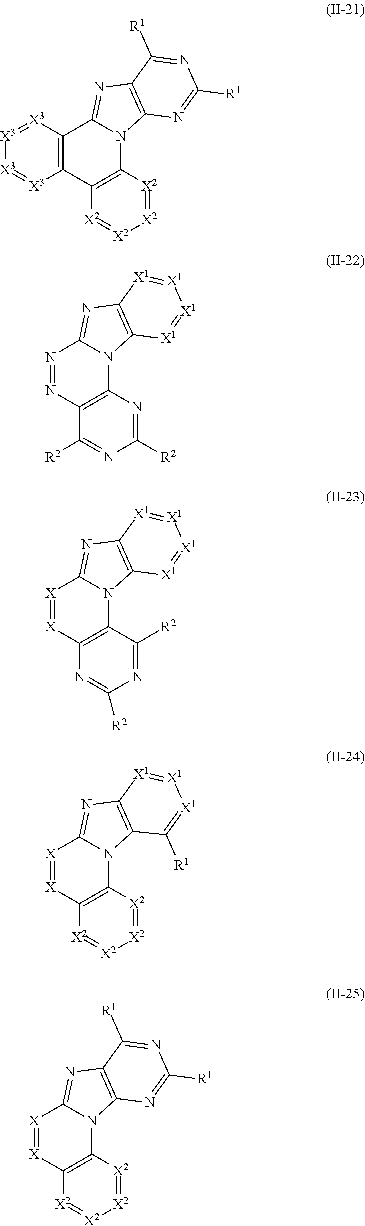
where X, X¹, X², X³, R¹, R², R³, Ar^{a}, Ar^{b} and Ar^{c} have the definitions given in claim 19.

23. The compound as claimed in claim 19, comprising at least one structure of the formulae (III-1) to (III-50): 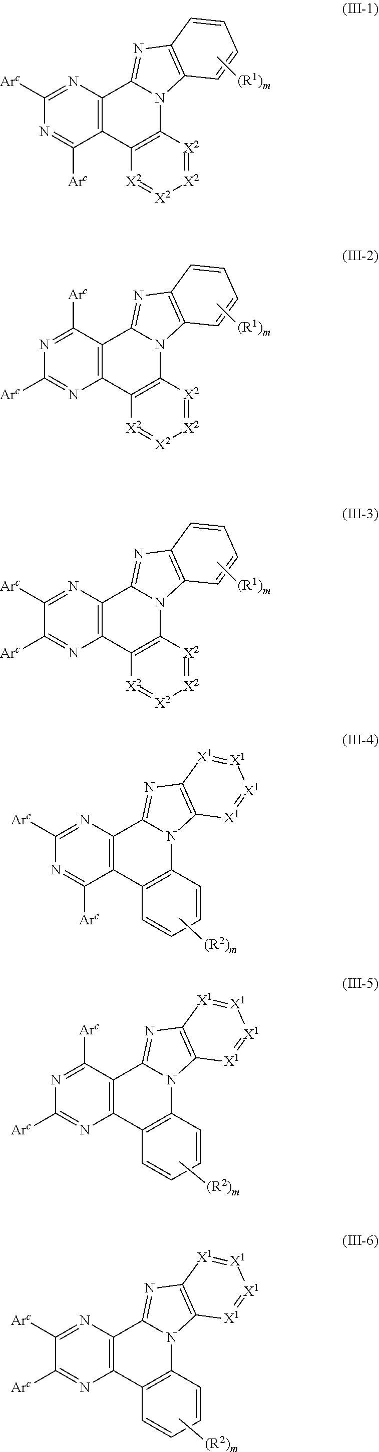 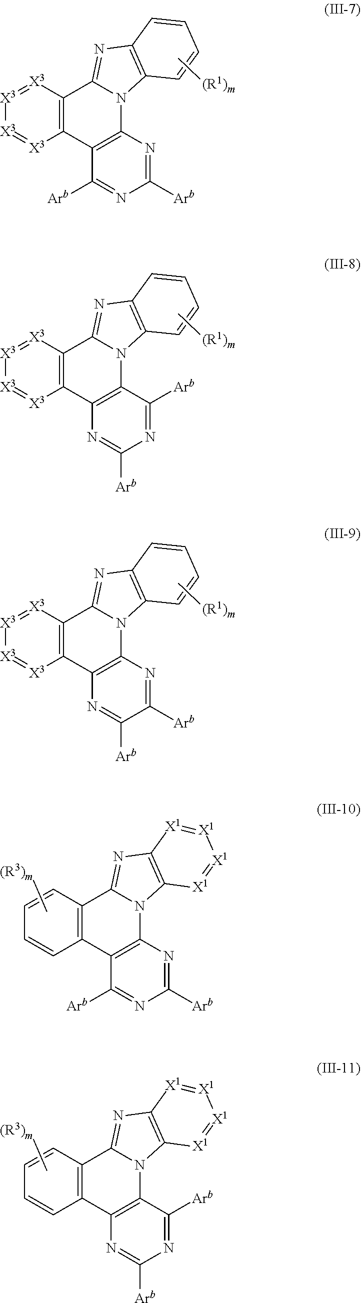 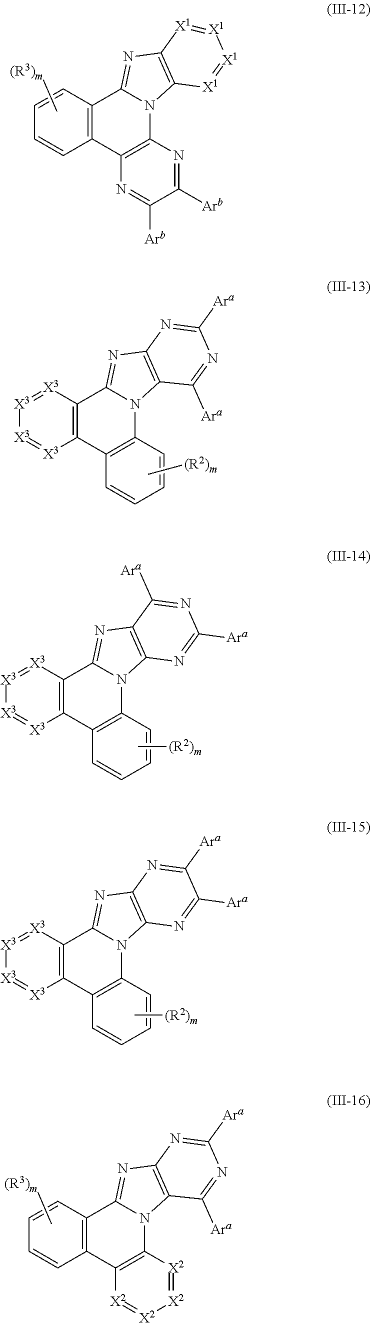 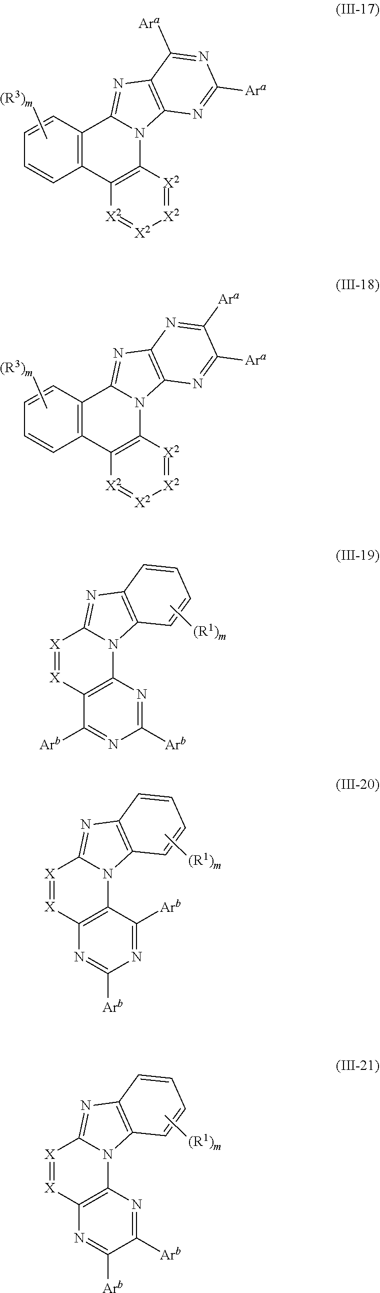 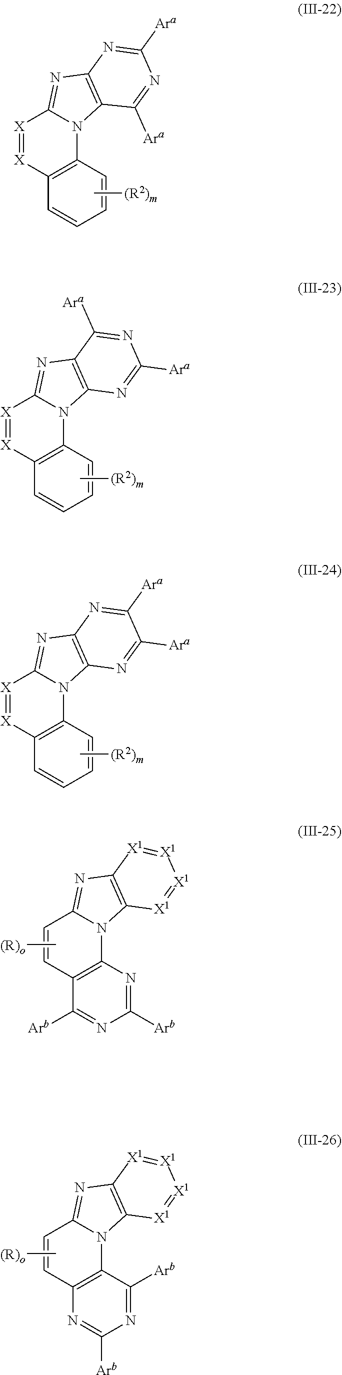 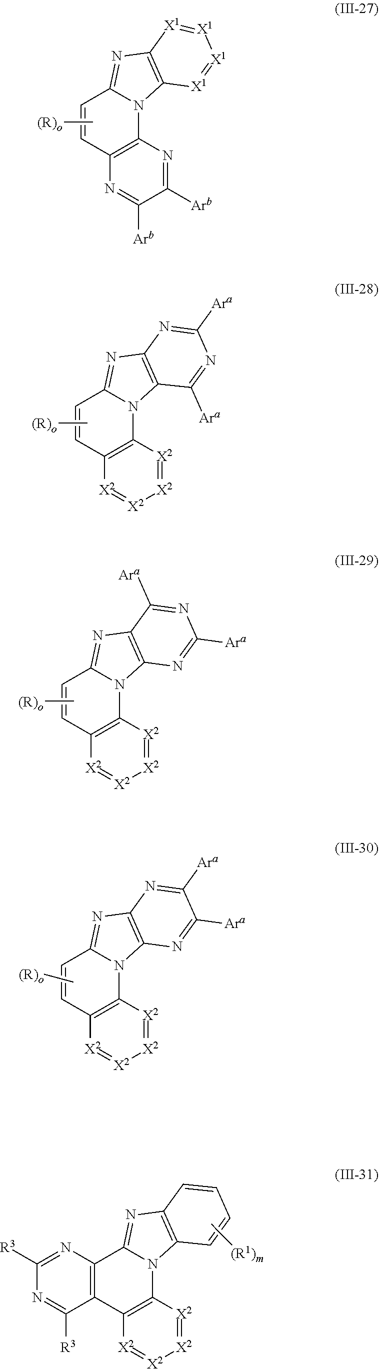 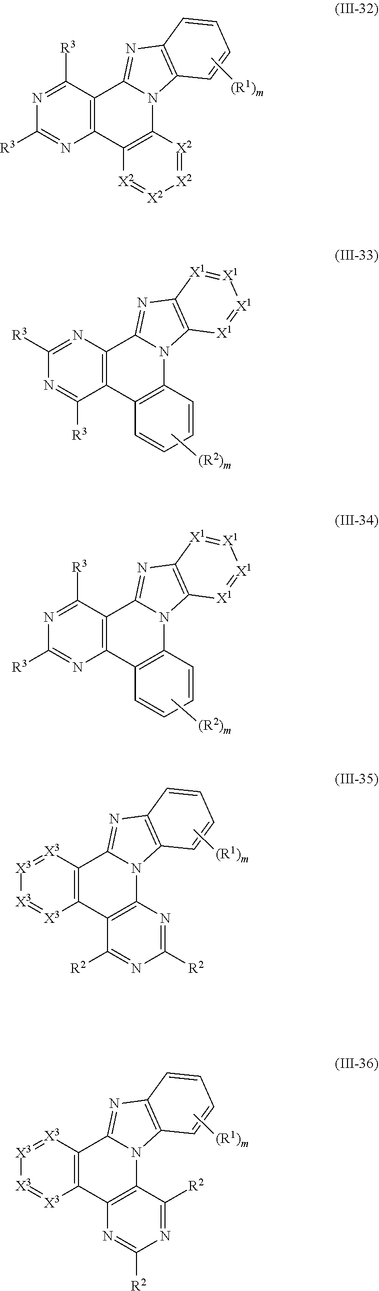 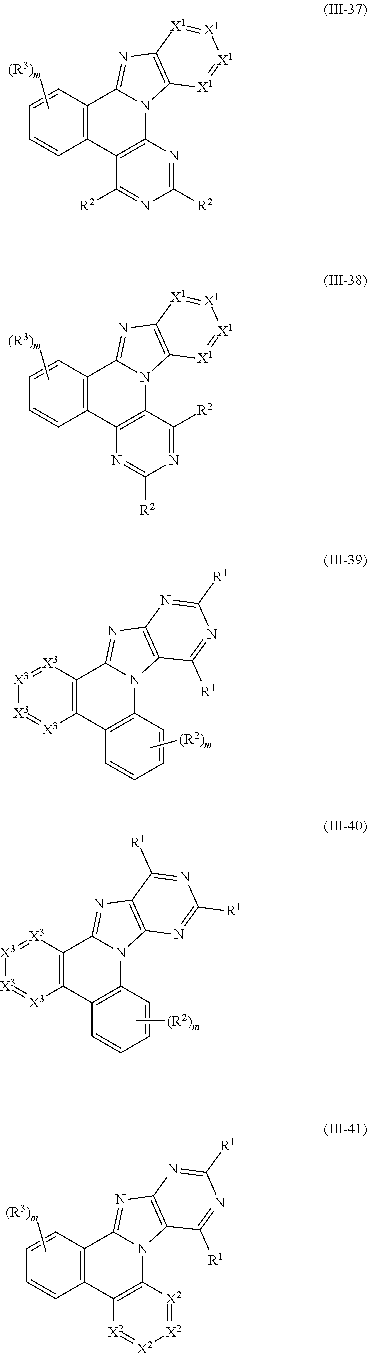 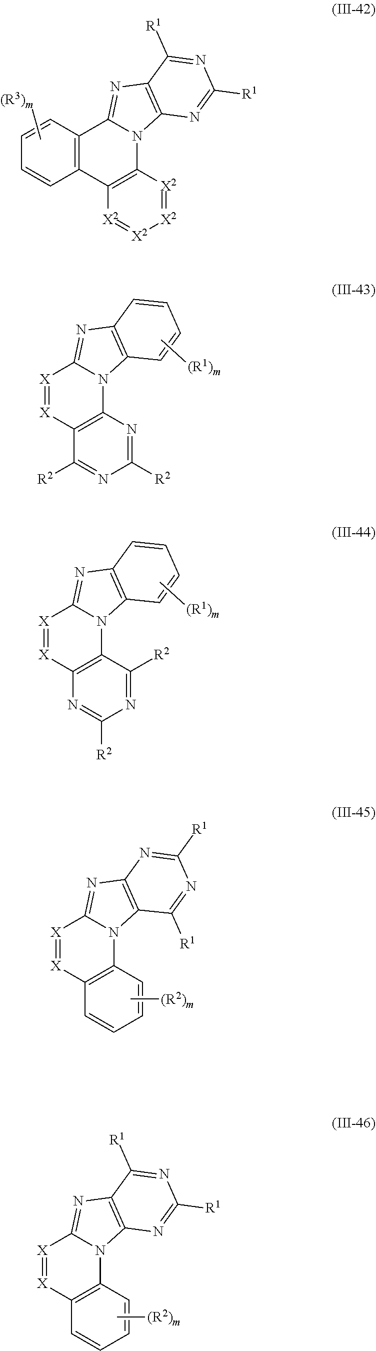 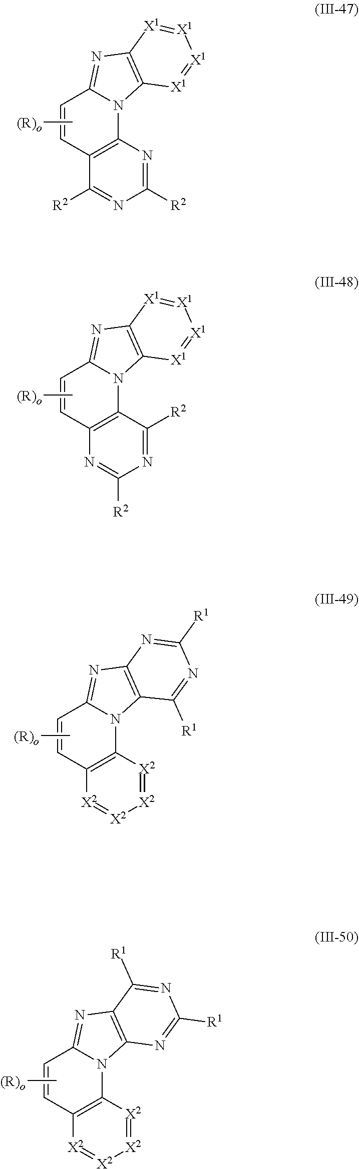
where R, R¹, R², R³, X, X¹, X², X³, Ar^{a}, Ar^{b} and Ar^{c} have the definitions given in claim 19, the index o is 0, 1 or 2, the index m is 0, 1, 2, 3 or 4

24. The compound as claimed in claim 19, comprising at least one structure of the formulae (IV-1) to (IV-25): 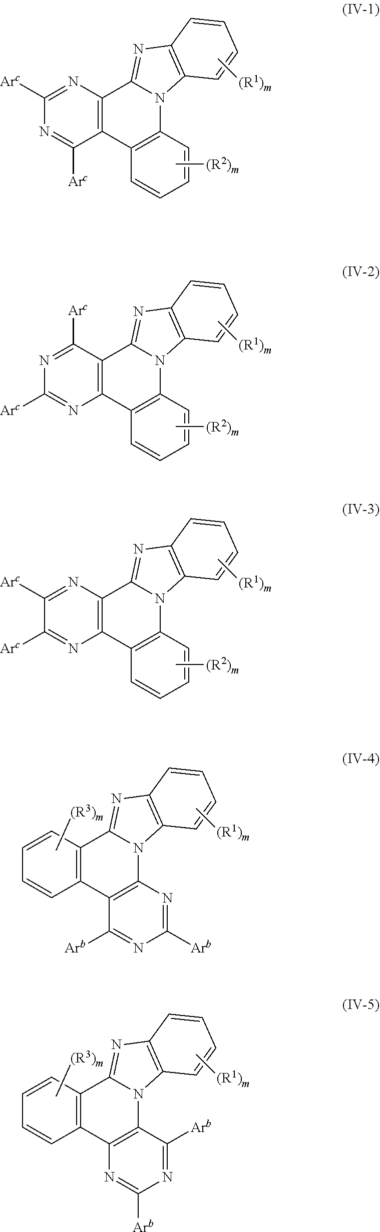 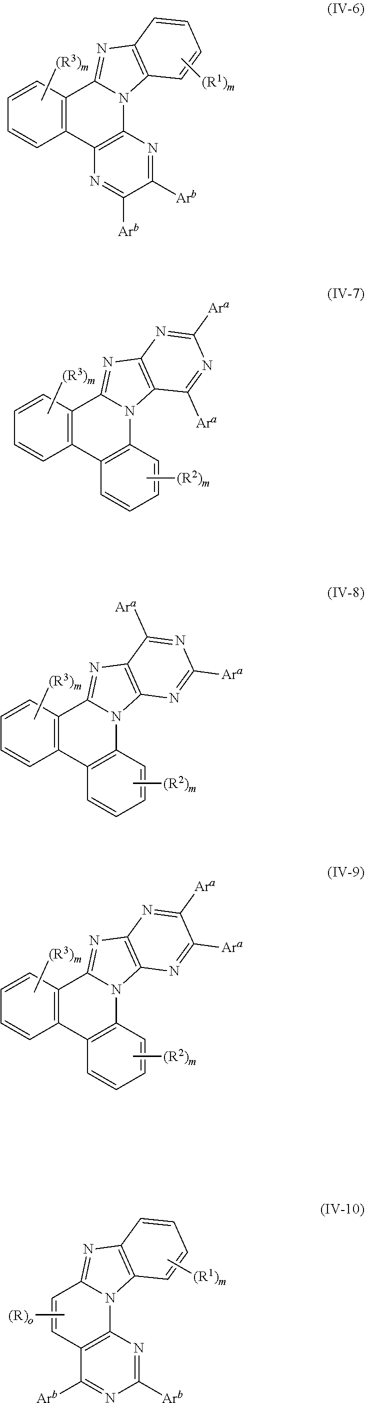 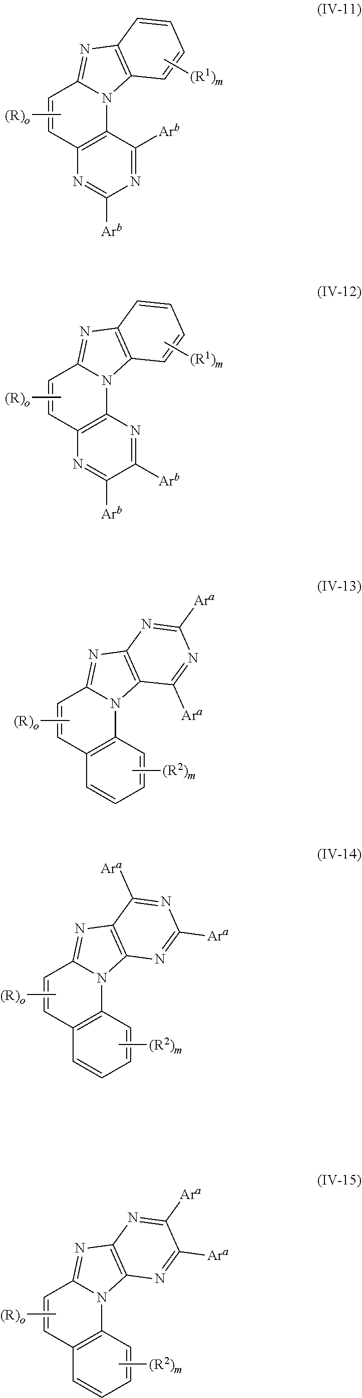 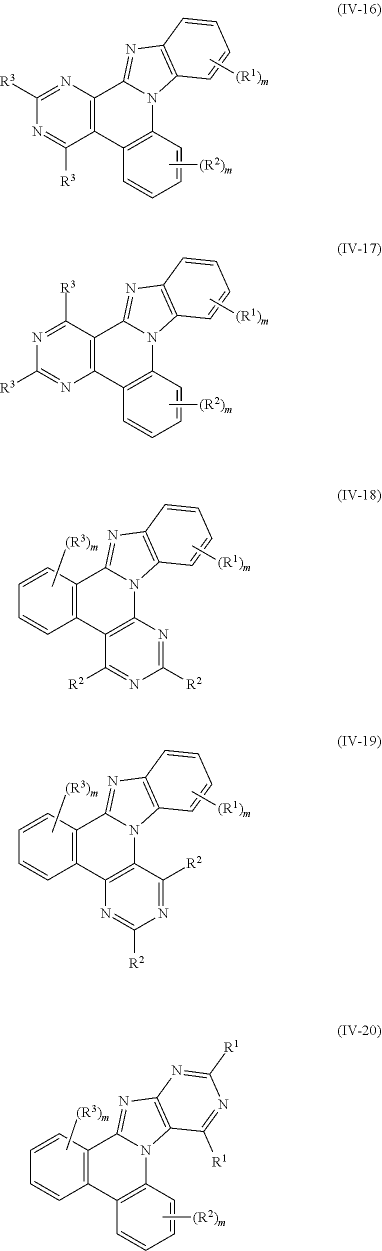 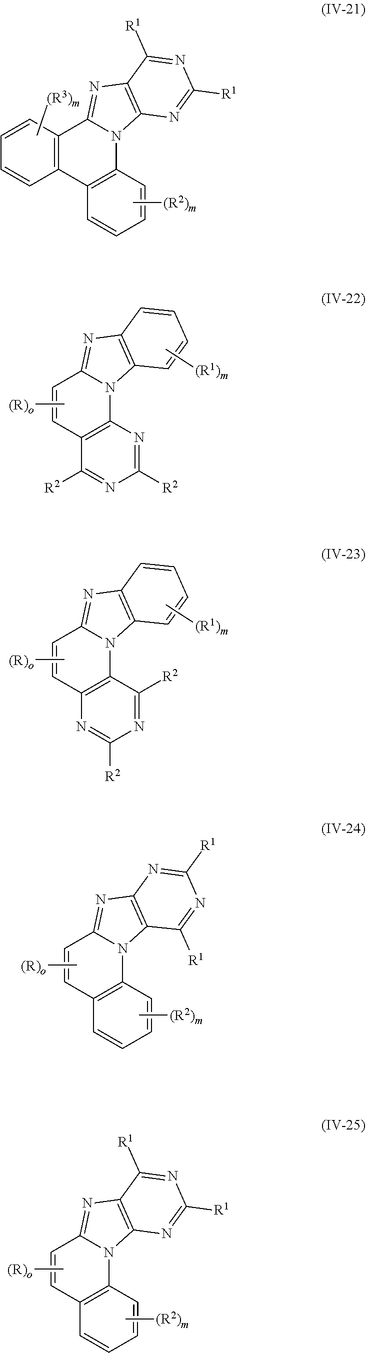
where R, R¹, R², R³, Ar^{a}, Ar^{b} and Ar^{c} have the definitions given in claim 19, the index o is 0, 1 or 2 and the index m is 0, 1, 2, 3 or 4.

25. The compound as claimed in claim 19, comprising at least one structure of the formulae (V-1) to (V-15): 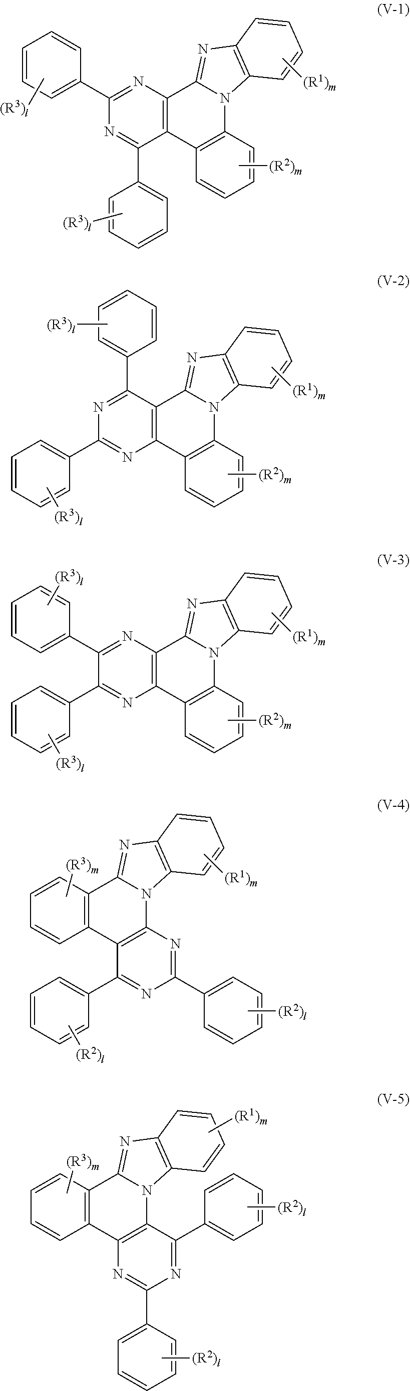 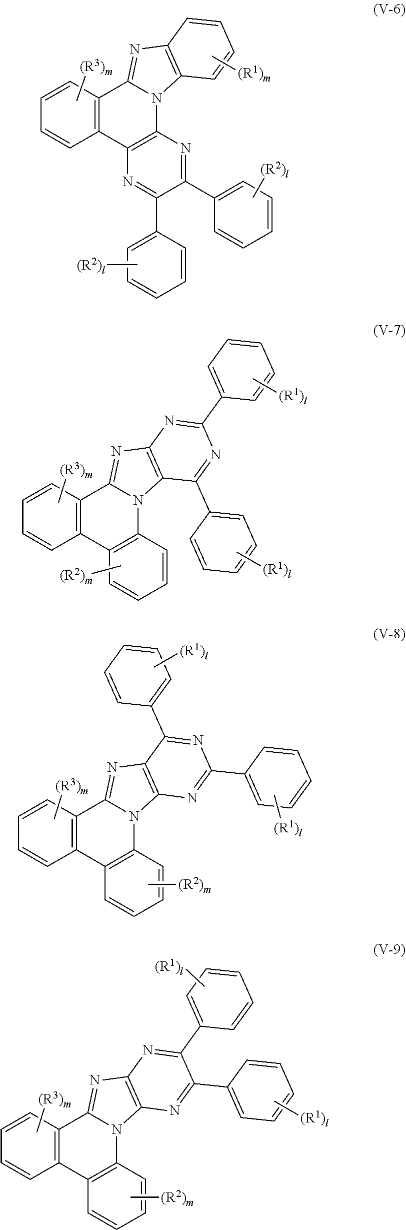 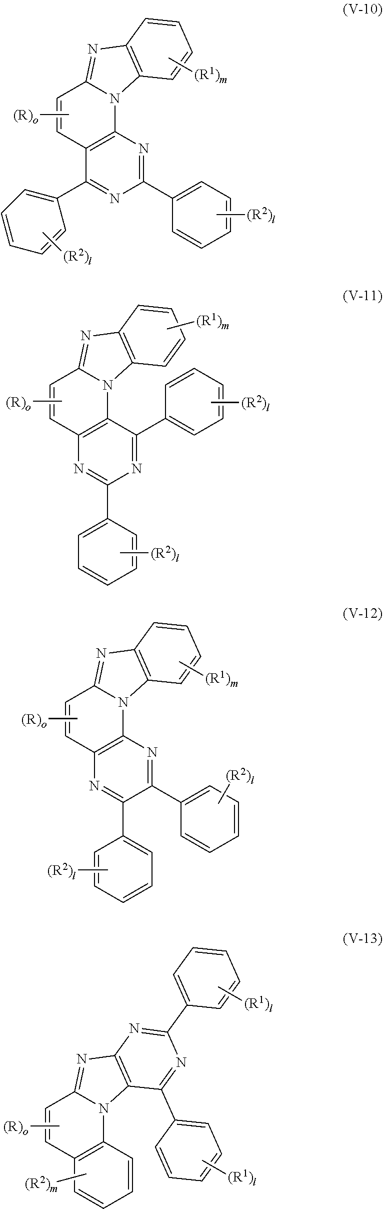 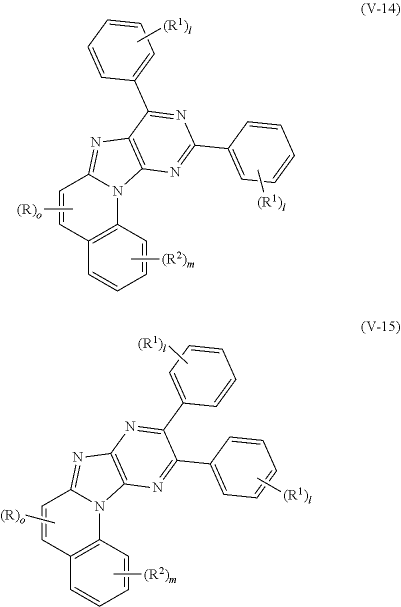
where R, R¹, R² and R³ have the definitions given in claim 19, the index o is 0, 1 or 2, the index m is 0, 1, 2, 3 or 4, and the index 1 is 0, 1, 2, 3, 4 or 5.

26. The compound as claimed in claim 19, characterized in that Ar^{a}, Ar^{b}, Ar^{c} is the same or different at each instance and is selected from phenyl, biphenyl, terphenyl, quaterphenyl, fluorene, spirobifluorene, naphthalene, indole, benzofuran, benzothiophene, carbazole, dibenzofuran, dibenzothiophene, indenocarbazole, indolocarbazole, pyridine, pyrimidine, pyrazine, pyridazine, triazine, quinoline, isoquinoline, quinazoline, quinoxaline, phenanthrene and triphenylene, each of which may be substituted by one or more R¹, R², R³ radicals.

27. The compound as claimed in claim 19, characterized in that R, R¹, R² and/or R³ is the same or different at each instance and is selected from the group consisting of H, D or an aromatic or heteroaromatic ring system selected from the groups of the following formulae Ar-1 to Ar-75, and/or the Ar^{a}, Ar^{b}, Ar^{c} and/or Ar′ group is the same or different at each instance and is selected from the groups of the following formulae Ar-1 to Ar-75: 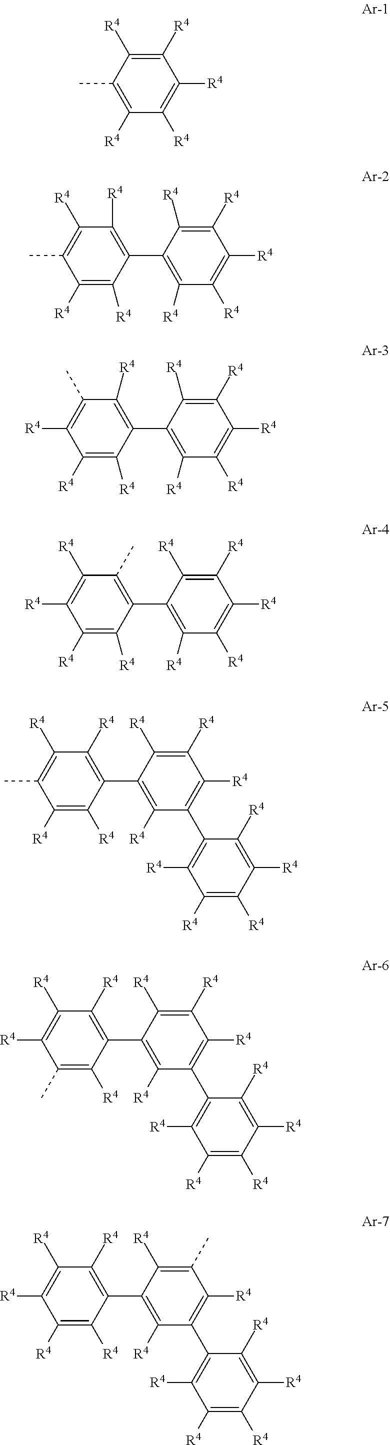 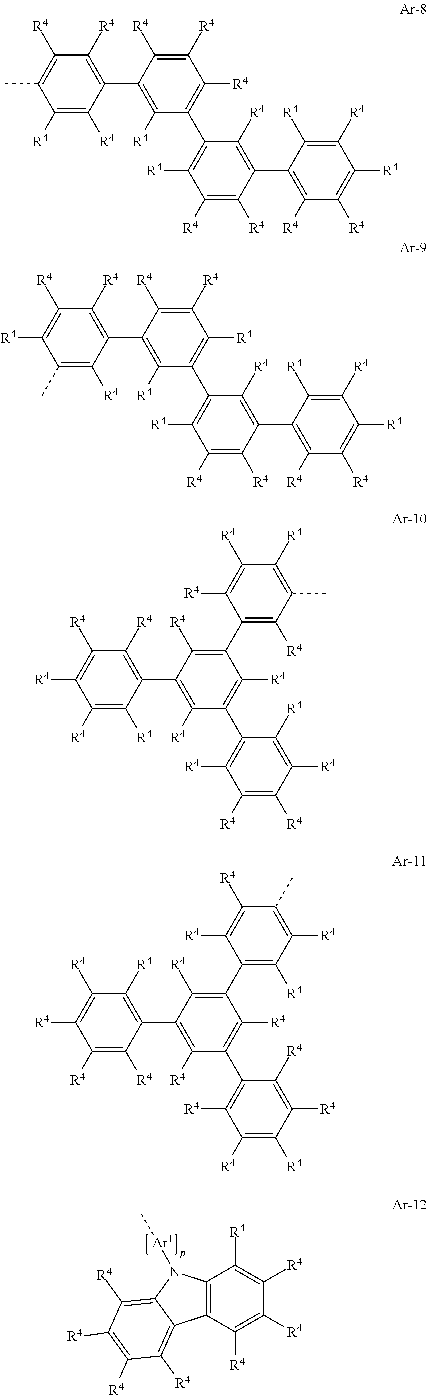 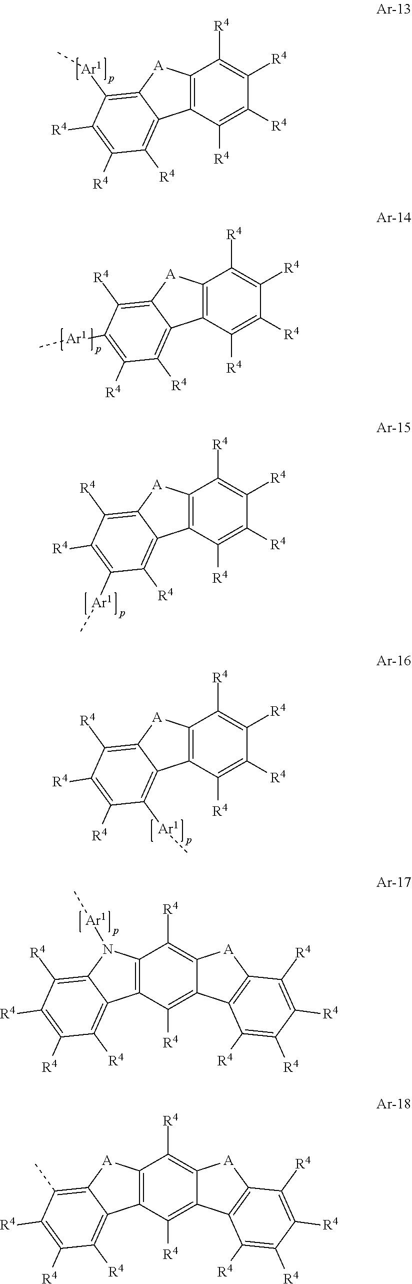 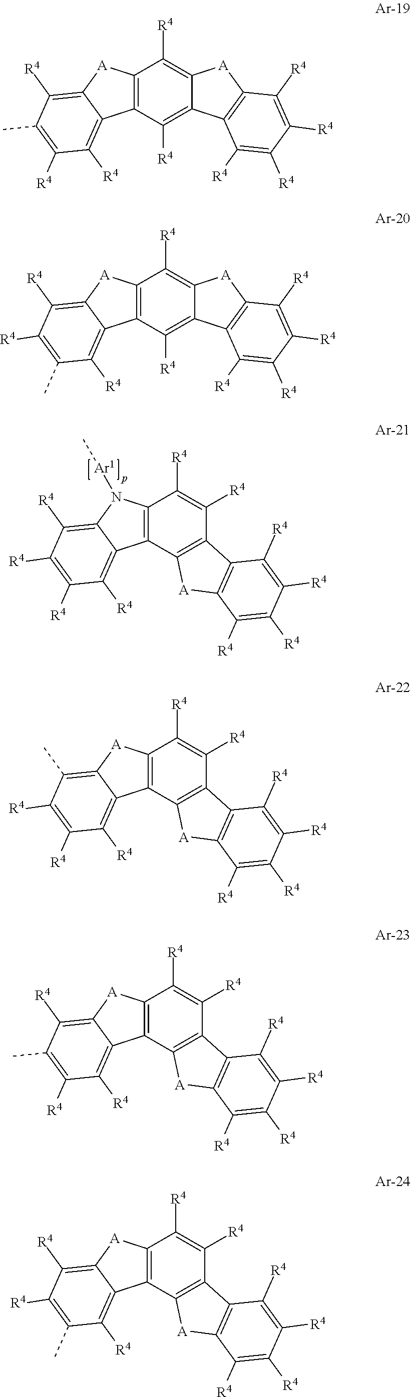 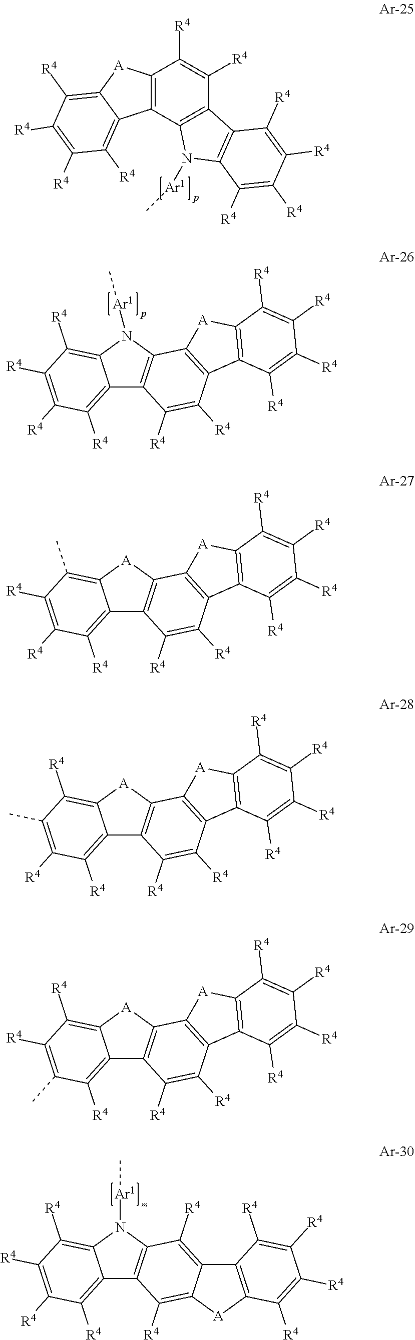 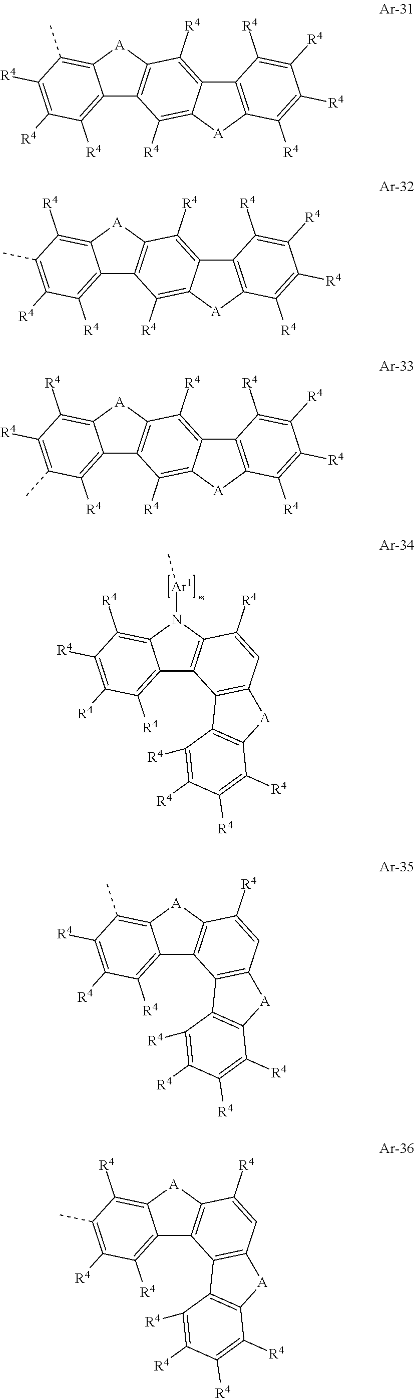 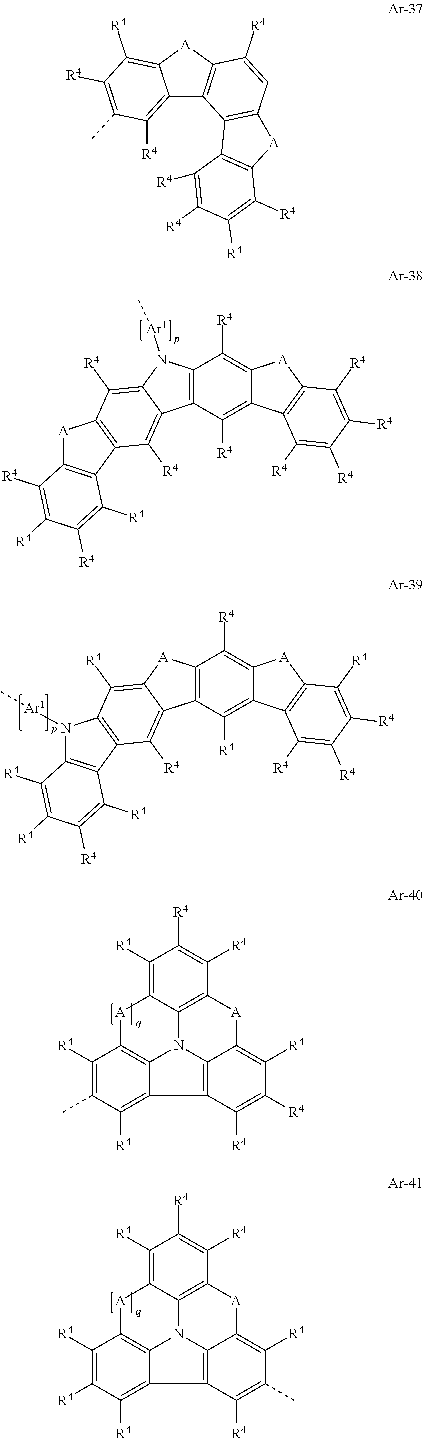 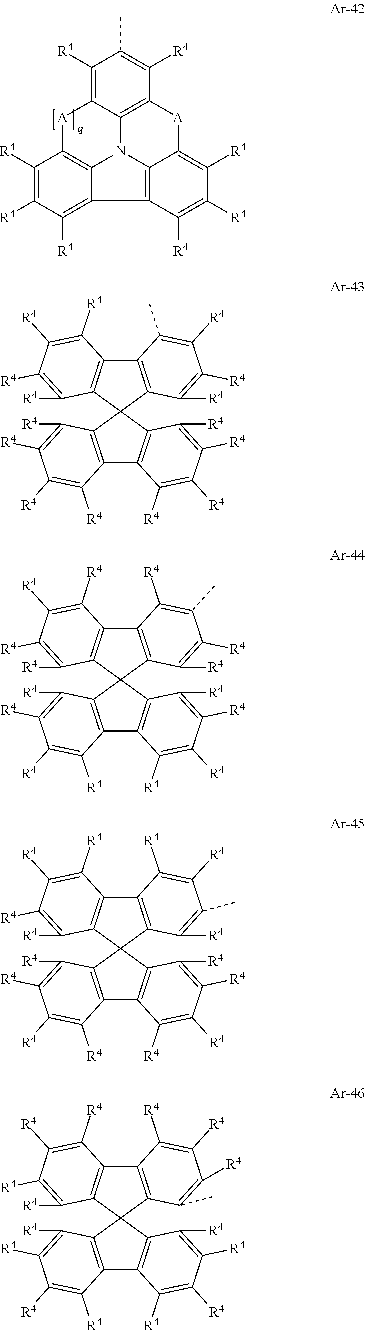 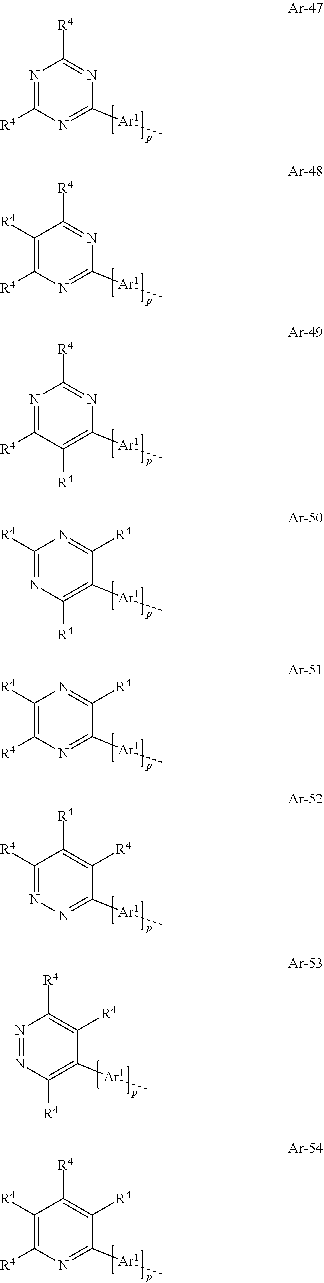 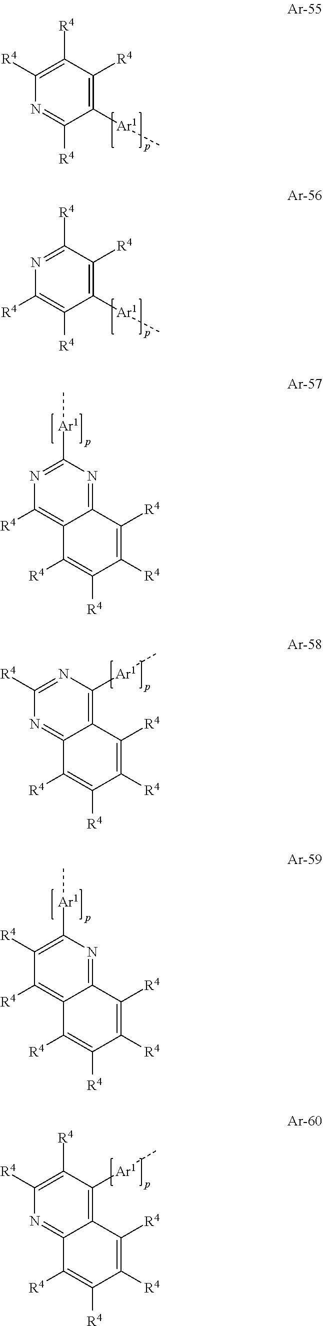 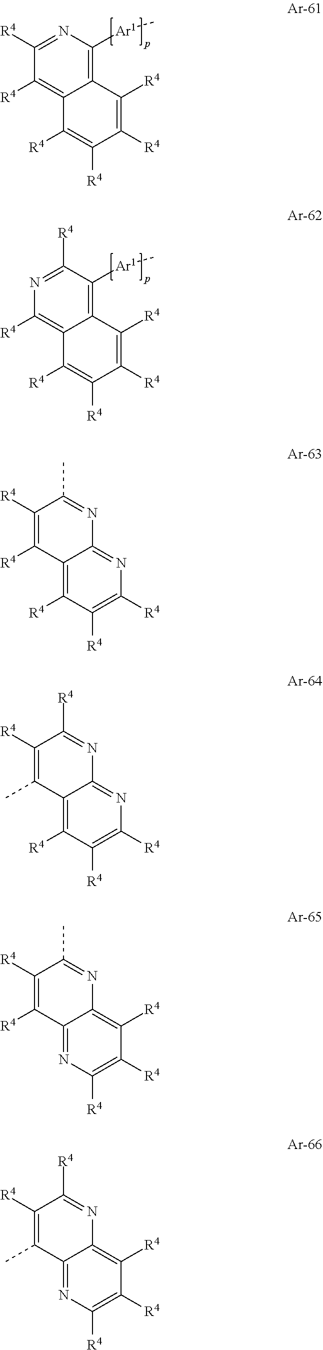 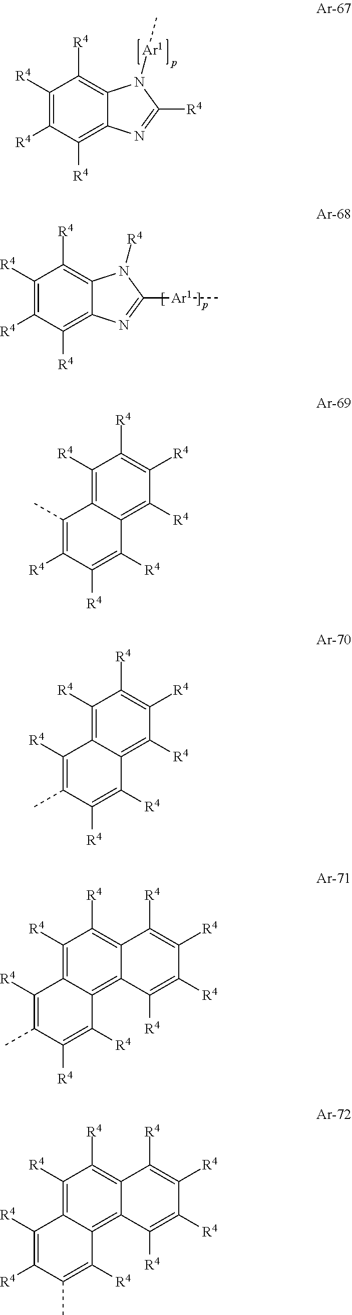 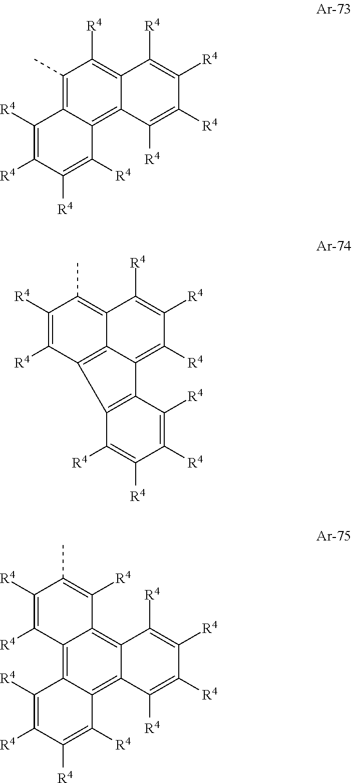
where R⁴ has the definitions given above, the dotted bond represents the bond of the corresponding group and in addition:
Ar¹ is the same or different at each instance and is a bivalent aromatic or heteroaromatic ring system which has 6 to 18 aromatic ring atoms and may be substituted in each case by one or more R⁴ radicals;
A is the same or different at each instance and is C(R⁴)₂, NR⁴, O or S;
p is 0 or 1, where p=0 means that the Ar¹ group is absent and that the corresponding aromatic or heteroaromatic group is bonded directly to the corresponding radical;
q is 0 or 1, where q=0 means that no A group is bonded at this position and R⁴ radicals are bonded to the corresponding carbon atoms instead.

28. The compound as claimed in claim 27, characterized in that R, R¹, R² and/or R³ is the same or different at each instance and is selected from the group consisting of H, D or an aromatic or heteroaromatic ring system selected from the groups of the following formulae (Ar-1), (Ar-2), (Ar-3), (Ar-12), (Ar-13), (Ar-14), (Ar-15), (Ar-16), (Ar-69), (Ar-70), and (Ar-75).

29. A process for preparing the compound as claimed in claim 19, which comprises reacting an aromatic or heteroaromatic compound with an aromatic or heteroaromatic diamino compound by a coupling reaction.

30. A composition comprising at least one matrix compound as claimed in claim 19 and at least one further matrix material, wherein the further matrix material is selected from compounds of one of the formulae (H-1), (H-2), (H-3), (H-4) and (H-5): 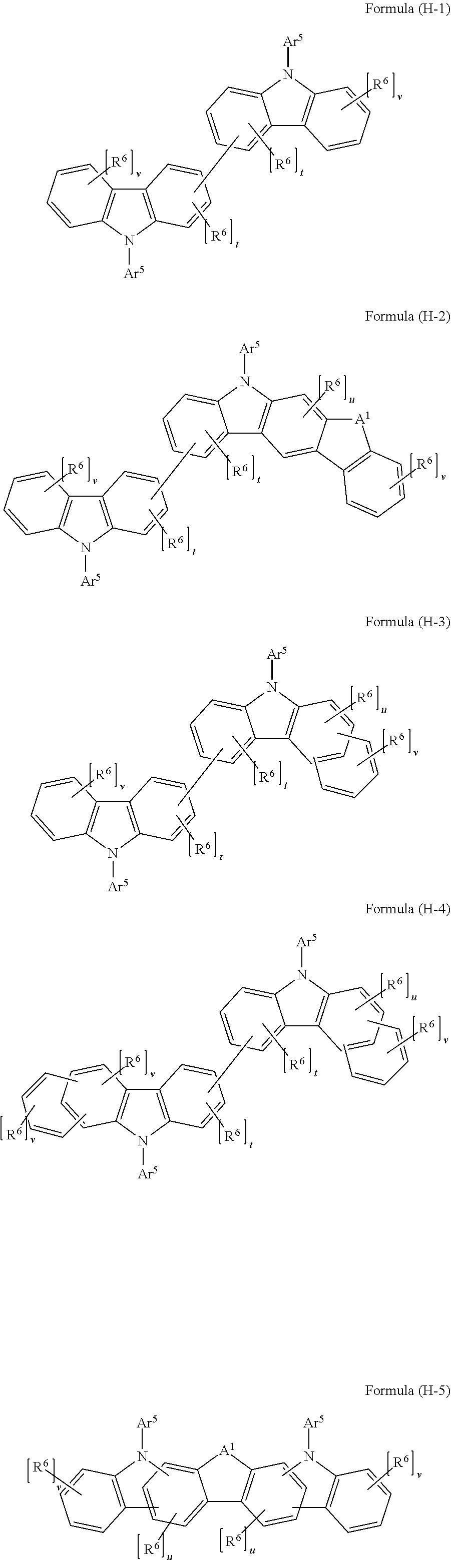
where the symbols and indices used are as follows:
R⁶ is the same or different at each instance and is H, D, F, Cl, Br, I, N(R⁷)₂, N(Ar′)₂, CN, NO₂, OR⁷, SR⁷, COOR⁷, C(═O)N(R⁷)₂, Si(R⁷)₃, B(OR⁷)₂, C(═O)R⁷, P(═O)(R⁷)₂, S(═O)R⁷, S(═O)₂R⁷, OSO₂R⁷, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R⁷ radicals, where one or more nonadjacent CH₂ groups may be replaced by Si(R⁷)₂, C═O, NR⁷, O, S or CONR⁷, or an aromatic or heteroaromatic ring system which has 5 to 60 aromatic ring atoms, and may be substituted in each case by one or more R⁷ radicals; at the same time, two R⁶ radicals together may also form an aromatic, heteroaromatic, aliphatic or heteroaliphatic ring system;
Ar″ is the same or different at each instance and is an aromatic or heteroaromatic ring system which has 5 to 40 aromatic ring atoms and may be substituted by one or more R⁷ radicals;
A¹ is C(R⁷)₂, NR⁷, O or S;
Ar⁵ is the same or different at each instance and is an aromatic or heteroaromatic ring system which has 5 to 40 aromatic ring atoms and may be substituted by one or more R⁷ radicals;
R⁷ is the same or different at each instance and is H, D, F, Cl, Br, I, N(R⁸)₂, CN, NO₂, OR⁸, SR⁸, Si(R⁸)₃, B(OR⁸)₂, C(═O)R⁸, P(═O)(R⁸)₂, S(═O)R⁸, S(═O)₂R⁸, OSO₂R′, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R⁸ radicals and where one or more nonadjacent CH₂ groups may be replaced by Si(R⁸)₂, C═O, NR⁸, O, S or CONR⁸, or an aromatic or heteroaromatic ring system which has 5 to 40 aromatic ring atoms and may be substituted in each case by one or more R⁸ radicals; at the same time, two or more R⁷ radicals together may form an aromatic, heteroaromatic, aliphatic or heteroaliphatic ring system;
R⁸ is the same or different at each instance and is H, D, F or an aliphatic, aromatic or heteroaromatic organic radical, especially a hydrocarbyl radical, having 1 to 20 carbon atoms, in which one or more hydrogen atoms may also be replaced by F;
v is the same or different at each instance and is 0, 1, 2, 3 or 4;
t is the same or different at each instance and is 0, 1, 2 or 3;
u is the same or different at each instance and is 0, 1 or 2.

31. The composition as claimed in claim 30, characterized in that the compound has a proportion by mass in the composition in the range from 10% by weight to 95% by weight, based on the total mass of the composition.

32. The composition as claimed in claim 30, characterized in that the compounds of one of the formulae (H-1), (H-2), (H-3), (H-4) and (H-5) have a proportion by mass in the composition in the range from 5% by weight to 90% by weight, based on the overall composition.

33. A formulation comprising at least one compound as claimed in claim 19 and at least one further compound.

34. A formulation comprising at least one composition as claimed in claim 29 and at least one further compound.

35. An electroluminescent device comprising at least one compound as claimed in claim 19

36. An electroluminescent device comprising the composition as claimed in claim 29.

37. An organic electroluminescent device, characterized in that the compound as claimed in claim 19 is used as matrix material in an emitting layer and/or electron transport layer and/or in a hole blocker layer and/or in an electron blocker layer.

38. An electronic device comprising the compound as claimed in claim 19 is used as matrix material for phosphorescent emitters in combination with a further matrix material, where the further matrix material is selected from compounds of one of the formulae (H-1), (H-2), (H-3), (H-4) and (H-5): 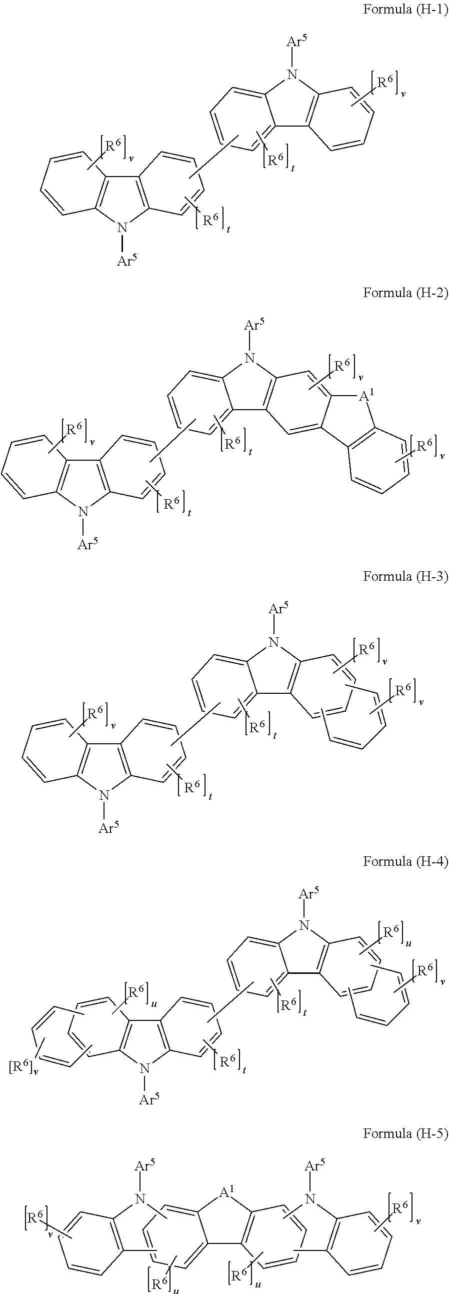
where the symbols and indices used are as follows:
R⁶ is the same or different at each instance and is H, D, F, Cl, Br, I, N(R⁷)₂, N(Ar′)₂, CN, NO₂, OR⁷, SR⁷, COOR⁷, C(═O)N(R⁷)₂, Si(R⁷)₃, B(OR⁷)₂, C(═O)R⁷, P(═O)(R⁷)₂, S(═O)R⁷, S(═O)₂R⁷, OSO₂R⁷, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R⁷ radicals, where one or more nonadjacent CH₂ groups may be replaced by Si(R⁷)₂, C═O, NR⁷, O, S or CONR⁷, or an aromatic or heteroaromatic ring system which has 5 to 60 aromatic ring atoms, and may be substituted in each case by one or more R⁷ radicals; at the same time, two R⁶ radicals together may also form an aromatic, heteroaromatic, aliphatic or heteroaliphatic ring system;
Ar″ is the same or different at each instance and is an aromatic or heteroaromatic ring system which has 5 to 40 aromatic ring atoms and may be substituted by one or more R⁷ radicals;
A¹ is C(R⁷)₂, NR⁷, O or S;
Ar⁵ is the same or different at each instance and is an aromatic or heteroaromatic ring system which has 5 to 40 aromatic ring atoms and may be substituted by one or more R⁷ radicals;
R⁷ is the same or different at each instance and is H, D, F, Cl, Br, I, N(R⁸)₂, CN, NO₂, OR⁸, SR⁸, Si(R⁸)₃, B(OR⁸)₂, C(═O)R⁸, P(═O)(R⁸)₂, S(═O)R⁸, S(═O)₂R⁸, OSO₂R′, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R⁸ radicals and where one or more nonadjacent CH₂ groups may be replaced by Si(R⁸)₂, C═O, NR⁸, O, S or CONR⁸, or an aromatic or heteroaromatic ring system which has 5 to 40 aromatic ring atoms and may be substituted in each case by one or more R⁸ radicals; at the same time, two or more R⁷ radicals together may form an aromatic, heteroaromatic, aliphatic or heteroaliphatic ring system;
R⁸ is the same or different at each instance and is H, D, F or an aliphatic, aromatic or heteroaromatic organic radical, especially a hydrocarbyl radical, having 1 to 20 carbon atoms, in which one or more hydrogen atoms may also be replaced by F;
v is the same or different at each instance and is 0, 1, 2, 3 or 4;
t is the same or different at each instance and is 0, 1, 2 or 3;
u is the same or different at each instance and is 0, 1 or 2.
